(19) United States
(12) Patent Application Publication
(10) Pub. No.: US 2009/0275583 A1
(43) Pub. Date: Nov. 5, 2009

(54) ANTIVIRAL COMPOUNDS AND USE THEREOF

(75) Inventors: Kraig Yager, Salt Lake City, UT (US); In Chul Kim, Salt Lake City, UT (US); Michael Saunders, Salt Lake City, UT (US); David Gerrish, Salt Lake City, UT (US); Dange Vijay Kumar, Salt Lake City, UT (US); Mark B. Anderson, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 12/422,767

(22) Filed: Apr. 13, 2009

Related U.S. Application Data
(63) Continuation of application No. PCT/US2007/081438, filed on Oct. 15, 2007.
(60) Provisional application No. 60/851,645, filed on Oct. 13, 2006.
(60) Provisional application No. 60/866,016, filed on Nov. 15, 2006.

(51) Int. Cl. A61K 31/5375; C07D 265/30; A61P 31/18

(52) U.S. Cl. 514/237.8; 544/162

(57) ABSTRACT

The invention relates to compounds, pharmaceutical compositions and methods useful for treating viral infection.

### CROSS REFERENCE TO RELATED APPLICATIONS

This application is a continuation application of PCT/US/2007/081438 filed on Oct. 15, 2007, which claims the benefit of U.S. Provisional Application Ser. No. 60/851,645, filed on Oct. 13, 2006 and U.S. Provisional Application Ser. No. 60/866,016, filed on Nov. 15, 2006, each of which is incorporated herein by reference in its entirety.

### FIELD OF THE INVENTION

The present invention relates generally to methods, compounds, and pharmaceutical compositions for treating (and delaying the onset of) diseases, and particularly viral infection such as HIV infection and AIDS.

### BACKGROUND OF THE INVENTION

Viral infection of humans is a major health problem, and viral infection of domesticated animals is a major economic concern. Combating viral infection has proven to be highly effective in some cases like smallpox where the disease was essentially eradicated with the advent of smallpox vaccination. Although smallpox was essentially eradicated by about 1980, there is considerable justified fear of the emergence of a new epidemic of smallpox since there are existing stockpiles of the virus and bioterrorism has moved beyond the realm of possibility to reality. Other viral infections have been much more difficult to fight. Hepatitis B and C, human immunodeficiency virus (HIV), herpes simplex viruses, and influenza are just a few prominent members of a list of viruses that pose significant health threats worldwide. Additionally, emerging viral infections continue to threaten the world with human epidemics, as is illustrated by the recent outbreak of severe acute respiratory syndrome (SARS) which has now been associated with coronavirus infection. Treatments currently available for many viral infections are often associated with adverse side effects. In addition, antiviral therapeutics directed towards specific viral gene products frequently have the effect of driving the selection of viruses resistant to such therapeutics, and viral strains resistant to current methods of treatment are an increasing problem. Accordingly, there is a clear and ever-present need for new antiviral treatments.

A number of articles and patent publications disclose derivative compounds of betulinic acid that are useful for treating HIV infection, including U.S. Patent Publication No. 2006135495, Huang et al., Antimicrobial Agents and Chemotherapy, 48:633-665 (2004), and Sun et al., J. Med. Chem., 45:4271-4275 (2002).

### BRIEF SUMMARY OF THE INVENTION

The present invention provides compounds of Formulae I-V:

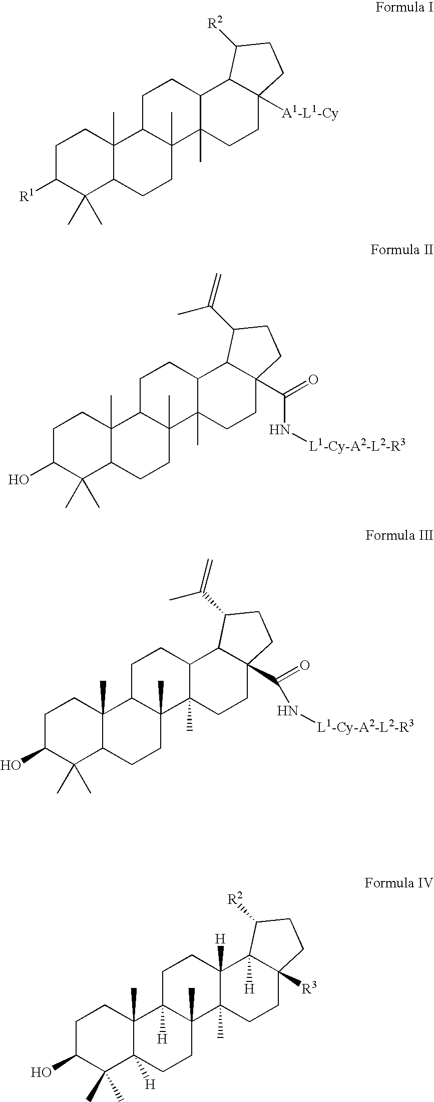

and pharmaceutically acceptable salts and stereoisomers thereof, wherein R¹, R², R³, A¹, A², L¹, L², and Cy are as defined herein.

The compounds of the present invention are effective viral inhibitors, particularly HIV fusion inhibitors, and are useful in inhibiting viral infection, particularly HIV infection and transmission. Thus, in a related aspect, the present invention provides methods for inhibiting viral fusion, particularly HIV fusion to host cells, and consequently HIV infectivity, by contacting HIV susceptible cells, in vitro or in a patient's body, an amount of a compound of the present invention sufficient to inhibit the infectivity of HIV virus into the cells. Therefore, the present invention also provides a method for treating viral infection, particularly HIV infection and AIDS, by administering to a patient in need of such treatment a therapeutically effective amount of a compound of the present invention.

Also provided in the present invention is a pharmaceutical composition having one or more compounds of the present invention and one or more pharmaceutically acceptable excipients. A method for treating viral infection, particularly HIV infection and AIDS, by administering to a patient in need of the treatment the pharmaceutical composition is also encompassed.

In addition, the present invention further provides methods for inhibiting, or reducing the likelihood of, viral transmission, particularly HIV transmission, or delaying the onset of the symptoms associated with viral infection, particularly HIV infection, or delaying the onset, or the onset of symptoms of, AIDS, comprising administering an effective amount of a compound of the present invention, preferably in a pharmaceutical composition or medicament to an individual having viral infection, particularly an HIV infection, or at risk of HIV infection, or at risk of developing symptoms of HIV infection or AIDS.

The compounds of the present invention for use in the instant invention can be provided as a pharmaceutical composition with one or more salts, carriers, or excipients. Some of the compounds for use in the invention have chiral centers, and the invention therefore includes the use of all stereoisomers, enantiomers, diastereomers, and mixtures thereof.

The compounds of the present invention can be used in combination therapies. Thus, combination therapy methods are also provided for treating HIV infection, inhibiting or reducing the likelihood of, HIV transmission, or delaying the onset of the symptoms associated with HIV infection, or delaying the onset of AIDS. Such methods comprise administering to a patient in need thereof a compound of the present invention, and together or separately, at least one other anti-HIV compound. For the convenience of combination therapy, the compound of the present invention is administered together in the same formulation with such other anti-HIV compound. Thus, the present invention also provides a pharmaceutical composition or medicament for the combination therapy, comprising an effective amount of a first compound according to the present invention and an effective amount of at least one other anti-HIV compound, which is different from the first compound. Examples of antiviral compounds include, but are not limited to, protease inhibitors, nucleoside reverse transcriptase inhibitors, non-nucleoside reverse transcriptase inhibitors, integrase inhibitors, fusion inhibitors, immunomodulators, and vaccines.

The foregoing and other advantages and features of the invention, and the manner in which they are accomplished, will become more readily apparent upon consideration of the following detailed description of the invention taken in conjunction with the accompanying examples, which illustrate preferred and exemplary embodiments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The invention provides compounds of Formulae I

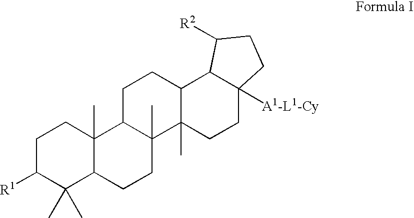

and pharmaceutically acceptable salts and stereoisomers thereof, wherein

### R¹ is ═O or —OH;

R² is a C₁₋₆ alkyl or C₁₋₆ alkenyl, optionally substituted with one or two substitutents independently chosen from —OH and heterocycle;
A¹ is C-amido, amino, alkylamido, or alkylamino;

L¹ is a bond or an alkyl of 1-10 carbons, of which any carbon can be replaced with a C₃-C₆cycloalkyl, optionally substituted with one or more substituents chosen from hydroxy, halo, alkyl, alkoxy, haloalkyl, haloalkoxy, —N(C₁₋₃ alkyl)₂, —NH(C₁₋₃ alkyl), —COOH, —COO(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Cy is an aryl, heteroaryl, heterocycle, or cycloalkyl optionally substituted with one or more substituents chosen from hydro, hydroxy, halo, alkyl, alkoxy, alkylthio, arylthio, thiocarbonyl, O-carboxy, C-carboxy, a —COOH bioisostere; O-carbamyl, O-thiocarbamyl, N-carbamyl, N-thiocarbamyl, ester, haloalkyl, haloalkoxy, cycloalkyl, aryl, heteroaryl, heterocycle, —CH(CH₃)COOH; —CH₂COOH, —C(CH₃)₂COOH, —C(CH₃)(CH₂CH₃)COOH, —CH(CH₂CH₃)COOH, —CH═C(CH₃)COOH, —C(CH₂CH₃)₂COOH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, or —NO₂; with the provisos that (1) when L¹ is —CH₂—, Cy cannot be an un-substituted phenyl or a phenyl substituted with hydroxy or methoxy, and (2) when L¹ is substituted by —COOH or —COOCH₃, Cy cannot be an un-substituted phenyl, a hydroxyphenyl, or indoline;
or Cy can be substituted with A²-L²-R³ wherein:
A² is an alkyl wherein one or more carbons of A² can be replaced with —NH—, —C(═O)—, or —N(CH₃)—, or wherein A² is a cycloalkyl, heterocycle, aryl, heteroaryl, or —O—;
L² is bond or an alkyl of 1, 2, 3, 4, 5, or 6 carbons, of which any carbon can be replaced with a C₃-C₆ cycloalkyl, optionally substituted with one or more alkyl, cycloalkyl, heterocycle, aryl, heteroaryl, or halo; and
R³ is an alkoxy, carboxy, —COOH bioisostere, carboxy alkyl, carbonyl, C-amido, amide ester, ester, cycloalkyl, heterocycle, aryl, heteroaryl, or alkyl of 1-10 carbons, each optionally substituted with one or more substituents chosen from hydro, hydroxy, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —CH(CH₃)COOH; —CH₂COOH, —C(CH₃)₂COOH, —C(CH₃)(CH₂CH₃)COOH, —CH(CH₂CH₃)COOH, —CH═C(CH₃)COOH, —C(CH₂CH₃)₂COOH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —SO₂NH₂, N-(2-Dimethylamino-ethyl)-N-methyl-formamide, N-(2-Dimethylamino-ethyl)-formamide, N-(2-Morpholin-4-yl-ethyl)-formamide, N-Methyl-N-(2-pyridin-4-yl-ethyl)-formamide, 3-Imidazol-1-yl-propan-1-ol, 2-[(2-Dimethylamino-ethyl)-methyl-amino]-ethanol, 2-Morpholin-4-yl-propan-1-ol, or Propane-1,3-diol.

In some embodiments of Formula I, R¹ is —OH.

In some embodiments of Formula I, R² is an alkyl of 1, 2, 3, 4, 5, or 6 carbons, optionally substituted with one or more ═O, —OH, alkyl, heterocycle, heteroaryl, aryl, or cycloalkyl. In some embodiments of Formula I, R² is —C(═CH₂)CH₃. In other embodiments of Formula I, R² is an isopropyl group —C(CH₃)₂.

In some embodiments of Formula I, A¹ is C-amido.

In some embodiments of Formula I, L¹ is C₁₋₂ alkyl, optionally substituted with one or more C₁₋₃ alkyl, carboxy, carbonyl, C₁₋₃ alkoxy, or a —COOH bioisostere. In certain embodiments, L¹ is an unsubstituted alkyl of 1 or 2 carbons.

In some embodiments of Formula I, Cy is an aryl having substituents as defined for Cy above. In some embodiments, Cy is an aryl substituted with A²-L²-R³ as defined above. In some embodiments, Cy is a phenyl having substituents as defined for Cy above. In some embodiments, Cy is a phenyl substituted with A²-L²-R³ as defined above. In specific embodiments, Cy is a phenyl substituted with fluorine, optionally substituted with one or more substituents as defined for Cy above. In certain embodiments, Cy is furan, optionally substituted as defined for Cy above. In other embodiments, Cy is pyridine, optionally substituted as defined for Cy above. In specific embodiments Cy is biphenyl, optionally substituted as defined for Cy above. In some embodiments of Formula I, A² is —C(═O)NH— or —NHC(═O)—. In other embodiments, A² is oxygen. In some embodiments of Formula I, L² is C₁₋₂ alkyl optionally substituted with one or more methyl. In specific embodiments, L² is a bond.

In some embodiments of Formula I, R³ is chosen from —COOH, a —COOH bioisostere; —COOCH₃, —C(═O)NHCH₃, —C(═O)NH₂, —NHC(═O)CH₃, —NHCOOCH₂(C₆H₅), —NHCOOCH₂CH₃, or —COOCH₂CH₃. In some embodiments, R³ is a phenyl optionally substituted with one or more alkoxy, carboxy, a —COOH bioisostere, hydroxy, methyl, methoxy, halide, amino, —COOCH₃, —O(CH₂)COOH, —SO₂NH₂, 2H-tetrazole, N-(2-Dimethylamino-ethyl)-N-methyl-formamide, N-(2-Dimethylamino-ethyl)-formamide, N-(2-Morpholin-4-yl-ethyl)-formamide, N-Methyl-N-(2-pyridin-4-yl-ethyl)-formamide, 3-Imidazol-1-yl-propan-1-ol, 2-[(2-Dimethylamino-ethyl)-methyl-amino]-ethanol, 2-Morpholin-4-yl-propan-1-ol, or Propane-1,3-diol. In some embodiments, R³ is furan or thiophene optionally substituted with one or more —COOH, —COOCH₃, or a —COOH bioisostere. In specific embodiments, R³ is —COOH or a —COOH bioisostere. In one embodiment, R³ is —COOH.

In some embodiments of Formula I, R¹ is OH or ═O;

R² is chosen from —C(CH₃)═CH₂, —C(CH₂OH)CH₃(OH), —C(CH₂N-cyclo C₄H₈)CH₃OH, or —CH₂(CH₃)₂;
A¹ is chosen from an —C(═O)NH—, —CH₂NH—, or —CH₂C(═O)NH—;
L¹ is a bond, or —CH₂— or —(CH₂)₂—, of which any carbon can be replaced with a three-membered cycloalkyl, optionally substituted with one or more —CH₃ or —COOH;
Cy is chosen from phenyl, biphenyl, pyridine, or furan, optionally substituted with one or more —CH₃, —OH, —COOH, —OCH₃, —F, or —COOCH₃, with the provisos that (1) when L¹ is —CH₂—, Cy cannot be an un-substituted phenyl or phenyl substituted with hydroxyl or methoxy, and (2) when L¹ is substituted by —COOH or —COOCH₃, Cy cannot be an un-substituted phenyl, a hydroxyphenyl, or indoline;
or Cy can be substituted with A²-L²-R³ wherein,
A2 is chosen from —C(═O)NH—, —CH₂NHC(═O)—, —CH₂NH—, —O—, —C(═O)NHCH₃—, piperazine, or 1,2,4-oxadiazole;
L² is a bond or an alkyl of 1, 2, 3, 4, 5 or 6 carbons, of which any carbon can be replaced with a three-membered cycloalkyl, optionally substituted with one or more —CH₃ or —F; and
R³ is chosen from propane, carboxy, biphenyl, phenyl, furan, thiophene, pyridine, benzimidazole, oxazolidin-2-one, —COOH, —COOCH₃, —C(═O)NHCH₃, —C(═O)NH₂, —NHC(═O)CH₃, —NHCOOCH₂(C₆H₅), —NHCOOCH₂CH₃, or —COOCH₂CH₃, each optionally substituted with one or more —OH, —CH₃, —OCH₃, —COOH, —COOCH₃, —C(═O)NH₂, —C(OH)CH₂COOH, —F, —Br, —NH₂, —N(CH₃)₂, —O(CH₂)ₙCOOH, —SO₂NH₂, 2H-tetrazole, N-(2-Dimethylamino-ethyl)-N-methyl-formamide, N-(2-Dimethylamino-ethyl)-formamide, N-(2-Morpholin-4-yl-ethyl)-formamide, N-Methyl-N-(2-pyridin-4-yl-ethyl)-formamide, 3-Imidazol-1-yl-propan-1-ol, 2-[(2-Dimethylamino-ethyl)-methyl-amino]-ethanol, 2-Morpholin-4-yl-propan-1-ol, or Propane-1,3-diol.

In specific embodiments, the invention provides compounds according to Formula II:

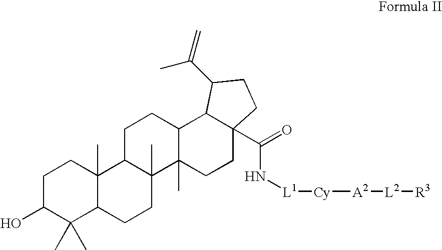

and pharmaceutically acceptable salts and stereoisomers thereof,
wherein
L¹ is a bond or an alkyl of 1 or 2 carbons, of which any carbon of L¹ can be replaced with a cyclopropyl, optionally substituted with one or more methyl groups;
Cy is chosen from phenyl, biphenyl, pyridine, or furan, optionally substituted with one or more —CH₃, —OH, —OCH₃, —COOH, —COOCH₃, or —F;
A² is chosen from piperazine, —C(═O)NH—, —CH₂NHC(═O)—, —CH₂NH—, —O—, —C(═O)NHCH₃—, or 1,2,4-oxadiazole;

L² is a bond or an alkyl of 1, 2, 3, 4, 5 or 6 carbons, of which any carbon can be replaced with a three-membered cycloalkyl, optionally substituted with one or more —CH₃ or —F; and
R³ is chosen from, propane, carboxy, biphenyl, phenyl, furan, thiophene, pyridine, benzimidazole, oxazolidin-2-one, —C(═O)NH₂, —NHC(═O)CH₃, —C(═O)NHCH₃, —NHCOOCH₂(C₆H₅), —COOCH₃, —COOCH₂CH₃, or —NHCOOCH₂CH₃, each optionally substituted with one or more —OH, —CH₃, —OCH₃, —COOH, —COOCH₃, —C(═O)NH₂, —C(OH)CH₂COOH, —F, —Br, —NH₂, —N(CH₃)₂, —O(CH₂)COOH, —SO₂NH₂, 2H-tetrazole, N-(2-Dimethylamino-ethyl)-N-methyl-formamide, N-(2-Dimethylamino-ethyl)-formamide, N-(2-Morpholin-4-yl-ethyl)-formamide, N-Methyl-N-(2-pyridin-4-yl-ethyl)-formamide, 3-Imidazol-1-yl-propan-1-ol, 2-[(2-Dimethylamino-ethyl)-methyl-amino]-ethanol, 2-Morpholin-4-yl-propan-1-ol, or Propane-1,3-diol; with the provisos that (1) when L¹ is —CH₂—, Cy cannot be an un-substituted phenyl or phenyl substituted with —OH or —OCH₃, and (2) when L¹ is substituted by —COOH or —COOCH₃, Cy cannot be an un-substituted phenyl, hydroxyphenyl, or indoline.

In some embodiments of Formula II, L¹ is an unsubstituted alkyl of 1 or 2 carbons.

In some embodiments of Formula II, Cy is a phenyl having substituents as defined for Cy above. In specific embodiments, Cy is a phenyl substituted with fluorine, optionally substituted with one or more substituents as defined for Cy above. In certain embodiments, Cy is furan, optionally substituted as defined for Cy above. In other embodiments, Cy is pyridine, optionally substituted as defined for Cy above. In specific embodiments Cy is biphenyl, optionally substituted as defined for Cy above.

In some embodiments of Formula II, A² is —C(═O)NH— or —NHC(═O)—. In other embodiments, A2 is —O—. In some embodiments of Formula II, L² is an alkyl of 1 or 2 carbons optionally substituted with one or more methyl groups. In specific embodiments, A² directly links to R³ with no L².

In some embodiments of Formula II, R³ is chosen from —COOH, —COOCH₃, —C(═O)NHCH₃, —C(═O)NH₂, —NHC(═O)CH₃, —NHCOOCH₂(C₆H₅), —NHCOOCH₂CH₃, or —COOCH₂CH₃. In some embodiments, R³ is a phenyl optionally substituted with one or more carboxy, hydroxy, methyl, methoxy, halide, amine, —COOCH₃, —O(CH₂)COOH, —SO₂NH₂, 2H-tetrazole, N-(2-Dimethylamino-ethyl)-N-methyl-formamide, N-(2-Dimethylamino-ethyl)-formamide, N-(2-Morpholin-4-yl-ethyl)-formamide, N-Methyl-N-(2-pyridin-4-yl-ethyl)-formamide, 3-Imidazol-1-yl-propan-1-ol, 2-[(2-Dimethylamino-ethyl)-methyl-amino]-ethanol, 2-Morpholin-4-yl-propan-1-ol, or Propane-1,3-diol. In some embodiments, R³ is furan or thiophene optionally substituted with one or more —COOH or —COOCH₃. In specific embodiments, R³ is —COOH.

In one embodiment, the stereochemistry of the core betulin moiety is preserved. For example, a compound of the invention may have a conformation according to Formula III:

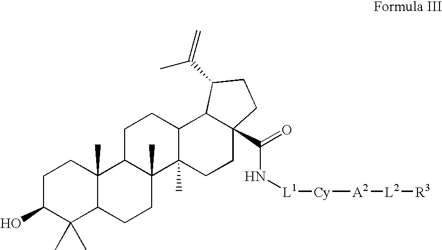

wherein L¹, Cy, A², L², and R³ are as defined for Formula II above.

The invention also provides a compound of formula IV

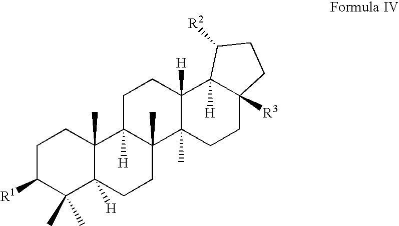

and pharmaceutically acceptably salts and stereoisomers thereof, wherein:

### R¹ is —OH or ═O;

R² is optionally substituted carbonyl, isopropenyl or isopropyl, wherein each can be optionally substituted with one or two substituents independently selected from hydroxyl, halo, cyano, C₁₋₆ alkoxy (optionally substituted with hydroxyl, C-carboxyl or O-carboxyl), C₁₋₆ alkylthio (optionally substituted with hydroxyl, C-carboxyl or O-carboxyl) and —N(R²¹R²²) wherein R²¹ and R²² are independently H, C₁₋₆ alkyl, C₁₋₆ hydroxyalkyl, or R²¹ and R²² together with the nitrogen they are attached to form a 3 to 6-membered heterocycle; preferably R² is isopropenyl, isopropyl, 3′-C₁₋₃ alkoxy-isopropenyl, 3′-C₁₋₃ hydroxyalkylthio-isopropenyl, 1′-cyano-isopropenyl, 3′-(1-pyrrolidin)-isopropenyl;
R³ is represented by

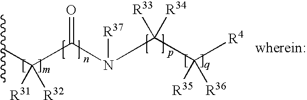

R³¹ and R³² are independently (meaning that R³¹ and R³² are not necessarily identical at each unit —C(R³¹)(R³²)—) H or methyl or ethyl, or R³¹ and R³² can be taken together with the carbon they are attached to form a C₃₋₅ cycloalkyl (e.g., cyclopropyl, cyclobutyl or cyclopentyl);

R³³ is H, halo (e.g., F), —COOR^{33a} (R^{33a} is H or C₁₋₆ alkyl such as methyl, ethyl, propyl and isopropyl), methyl or ethyl, and R³⁴ is H, halo (e.g., F), methyl or ethyl, or R³³ and R³⁴ can be taken together with the carbon they are attached to form a C₃₋₅ cycloalkyl (e.g., cyclopropyl, cyclobutyl or cyclopentyl);

R³⁵ and R³⁶ are independently H, halo (e.g., F), methyl or ethyl, or R³⁵ and R³⁶ can be taken together with the carbon they are attached to form a C₃₋₅ cycloalkyl (e.g., cyclopropyl, cyclobutyl or cyclopentyl);

R³⁷ is H or methyl or ethyl, and preferably H;

m is an integer of 0 or 1, preferably 0;

n is an integer of 0 or 1;

p is an integer of 0 or 1 or 2; and

q is an integer of 0 or 1 or 2; and preferably m+n+p+q is from 1 to 4, more preferably is 2 or 3; and

R⁴ is an aryl or heteroaryl (e.g., phenyl, pyridinyl, furanyl, and thiophenyl) substituted with a first substitutent R¹¹, and optionally one or more (e.g., 1, 2, or 3 or 4) other substituents, said one or more other substituents being independently chosen from the group consisting of halo (e.g., F, Cl, Br), hydroxyl, optionally substituted C₁₋₁₀ alkyl (preferably C₁₋₄ alkyl, e.g. methyl, ethyl, optionally substituted with 1-3 halo, e.g., F), optionally substituted C₁₋₁₀ alkoxy (preferably C₁₋₄ alkoxy, e.g. methoxy, ethoxy, optionally substituted with 1-3 halo, e.g., F), O-carboxy and C-carboxy; wherein said first substituent R¹¹ is chosen from

(a) C-carboxy (Preferably when said first substitutent is C-carboxy, either R⁴ is heteroaryl, or R⁴ is additionally substituted with said one or more other substituents.)

(b) O-carboxy,

(c) sulfonamido optionally substituted with a substituted or unsubstituted arylalkyl or heteroarylalkyl;

(d) one of the following groups (i)-(vii):

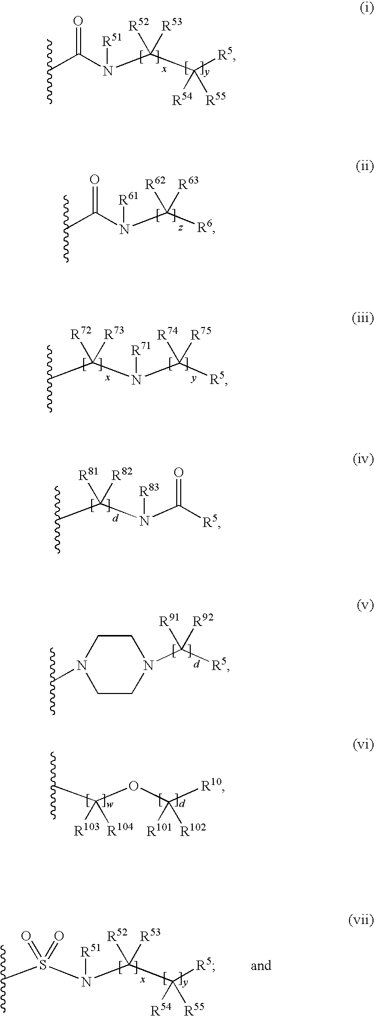

(e) R⁵, which is an aryl, arylalkyl, heteroaryl or heteroarylalkyl (e.g., phenyl, biphenyl, pyridinyl, furanyl, and thiophenyl, etc.) (preferably aryl or heteroaryl), each being optionally substituted with one or more (e.g., 1, 2 or 3) substituents. For example, the one or more optional substituents can each be independently chosen from:

(1) halo (e.g., F, Cl, Br, I);

(2) hydroxyl; cyano;

(3) C-carboxy, O-carboxy, or carboxyalkyl;

(4) optionally substituted alkyl (preferably C₁₋₆, more preferably C₁₋₃ alkyl) or cycloalkyl (preferably C₃₋₆ cycloalkyl), for example, substituted with one or more (e.g., 1, 2, 3 or 4) substituents independently chosen from the group consisting of:
(A) hydroxyl; (B) halo; (C) amino;
(D) C₁₋₆ alkoxy; and
(E) arylalkoxy or heteroarylalkoxy optionally substituted with hydroxyl, halo, C-carboxy, amino, or C₁₋₆ alkoxy.

(5) —N(R^{ca})C(═O)R^{cb}, —N(R^{ca})C(═O)N(R^{cc})(R^{cd}), or —OC(═O)N(R^{cc})(R^{cd}), wherein R^{ca} is H or methyl or ethyl (preferably H), and R^{cb}, R^{cc} and R^{cd} are each independently H, OH(R^{cc} and R^{cd} are not both OH) or a chemical moiety chosen from the group of C₁₋₁₀ alkyl, C₂₋₁₀ alkenyl, C₂₋₁₀ alkynyl, C₁₋₁₀ hydroxyalkyl, C₁₋₁₀ alkoxy, C₁₋₁₀ alkylthiol, C₂₋₁₀ alkenyloxy, C₂₋₁₀ alkynyloxy, C₁₋₁₀ alkoxyalkyl, C₁₋₁₀ alkylthioalkyl, carboxyalkyl, carbocycle, heterocycle, aryl and heteroaryl, or R^{cc} and R^{cd} together with the nitrogen atom to which they are both linked form a 3, 4, 5 or 6-membered optionally substituted heterocycle (e.g., piperidinyl, pyrrolidinyl, and morpholinyl), wherein the chemical moiety is optionally substituted with one or more substituents (e.g., 1, 2, 3 or 4 substituents independently chosen from the group of halo, hydroxyl, optionally substituted C₁₋₆ alkoxy, C₁₋₆ alkylthiol, optionally substituted carbocycle or heterocycle, optionally substituted aryl or heteroaryl, C-carboxy, O-carboxy, carboxyalkyl, and amino);

(6) —N(R^{ab})(R^{ac}) or —SO₂N(R^{ab})(R^{ac}), wherein R^{ab} and R^{ac} are independently H, OH (R^{ab} and R^{ac} are not both OH) or optionally substituted C₁₋₆ alkyl (preferably C₁₋₃ alkyl), or R^{ab} and R^{ac} taken together with the nitrogen they are attached to form a 3, 4, 5 or 6-membered optionally substituted heterocycle; for example, the optionally substituted C₁₋₆ alkyl may include one or more (1, 2, or 3) substituents each independently being hydroxyl, halo, C-carboxy, O-carboxy, amino, optionally substituted heterocycle (e.g., 4-morpholinyl or 3-piperidinyl, with one or more substituents such as C-carboxy) or optionally substituted heteroaryl.

(7) optionally substituted C₁₋₆ alkoxy; for example, the C₁₋₆ alkoxy can be optionally substituted with 1, 2 or 3 substituents each being independently chosen from the group consisting of:
(A) hydroxyl;
(B) halo (e.g., F, Cl, Br, I);
(C) —CO₂R^{aa}, —O(C═O)R^{aa}, —C₁₋₆ alkyl-CO₂R^{aa}, or wherein R^{aa} is H or C₁₋₃ alkyl, preferably methyl or ethyl;
(D) —N(R^{ca})C(═O)R^{cb}, —N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle (e.g., piperidinyl, pyrrolidinyl, and morpholinyl);
(E) optionally substituted heterocycle (e.g.,

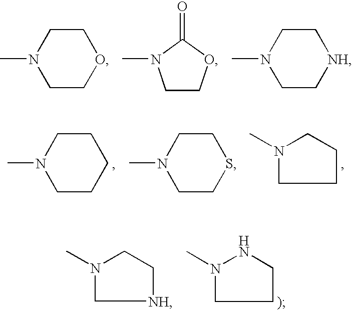

for example with one or more substituents (e.g., 1, 2, or 3) each being independently halo (e.g., F, Cl, Br, I), hydroxyl, C₁₋₆ alkyl, C₁₋₃ haloalkyl, C-carboxyl, and sulfonyl;
(F) heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, C-carboxy, C₁₋₃ hydroxyalkyl, C₁₋₃ haloalkyl, or —N(R^{ae})(R^{af}) or —SO₂N(R^{ae})(R^{af}), wherein R^{ae} and R^{af} are independently H, OH (R^{ae} and R^{af} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered optionally substituted heterocycle; and
(G) —N(R^{ag})(R^{ah}) where R^{ag} and R^{ah} are independently H, hydroxyl, C₁₋₆ alkyl, optionally substituted C₁₋₆ hydroxyalkyl or aminoalkyl, or R^{ag} and R^{ah} can be taken together with the nitrogen they are attached to form a 3, 4, 5 or 6-membered optionally substituted heterocycle; and

(9) —CON(R^{ak})(R^{al}) wherein R^{ak} and R^{al} are independently H, OH (R^{ak} and R^{al} are not both OH) or a chemical moiety chosen from the group consisting of C₁₋₁₀ alkyl, C₂₋₁₀ alkenyl, C₂₋₁₀ alkynyl, C₁₋₁₀ hydroxyalkyl, C₁₋₁₀ alkoxy, C₁₋₁₀ alkylthiol, C₂₋₁₀ alkenyloxy, C₂₋₁₀ alkynyloxy, C₁₀ alkoxyalkyl, C₁₀ alkylthioalkyl, carboxyalkyl, aminoalkyl, carbocycle, heterocycle, aryl, arylakly, heteroaryl, heteroarylalkyl, or R^{ak} and R^{al} together with the nitrogen atom to which they are both linked form a 3, 4, 5 or 6-membered heterocycle (e.g., piperidinyl, pyrrolidinyl, and morpholinyl), wherein the chemical moiety is optionally substituted with one or more substituents (e.g., 1, 2, 3 or 4 substituents. For example, the chemical moiety can be optionally substituted with 1, 2, or 3 or 4 substituents each being independently
(A) hydroxyl;
(B) halo;
(C)C₁₋₆ hydroxyalkyl;
(D) C₁₋₆ alkoxy;
(E) C₁₋₆ alkylthiol;
(F) C-carboxy, O-carboxy, or carboxyalkyl;
(G) —N(R^{am})(R^{an}) where R^{am} and R^{an} are independently H, hydroxyl, optionally substituted C₁₋₆ alkyl, optionally substituted C₁₋₆ hydroxyalkyl, or R^{am} and R^{an} together with the nitrogen atom they are attached to form a 3, 4, 5 or 6-membered optionally substituted heterocycle;
(H) heterocycle (e.g.,

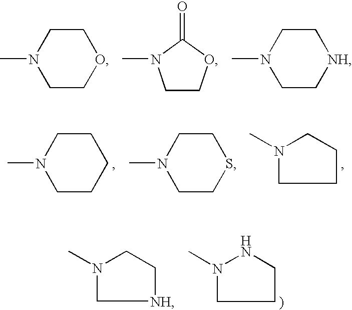

optionally substituted with one or more substituents (e.g., 1, 2, or 3 substituents each being independently halo, (e.g., F, Cl, Br, I), hydroxyl, C₁₋₆ alkyl, C₁₋₃ haloalkyl, C-carboxy, O-carboxy or carboxyalkyl); and
(I) aryl or heteroaryl (preferably having at least one nitrogen atom, e.g., imidazolyl, pyridinyl, etc.), optionally substituted with 1, 2, or 3 substituents each being independent halo (e.g., F, Cl, Br, I), hydroxyl, optionally substituted C₁₋₆ alkyl (e.g., methyl, C₁₋₆ haloalkyl), C-carboxy, O-carboxy carboxyalkyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and R^{ap} taken together with the nitrogen they are attached to form a 3, 4, 5 or 6-membered heterocycle; and

(10) R⁵ as defined herein.

In group (i), R⁵ is as defined above, R⁵¹ is H or methyl or ethyl; R⁵² and R⁵³ at each occurrence are independently chosen from the group consisting of H, F, hydroxyl, C₁₋₆ alkyl, C-carboxy, C-amido; and R⁵² and R⁵³ can be taken together with the carbon they are attached to form a cyclopryl; R⁵⁴ and R⁵⁵ at each occurrence are independently H, methyl, ethyl, F, or hydroxyl, or R⁵⁴ and R⁵⁵ can be taken together with the carbon they are attached to form a cyclopryl, or R⁵¹ and R⁵⁴ can be taken together with the nitrogen atom R⁵¹ is attached to, and the carbon atom(s) in the aliphatic chain between R⁵⁴ and the nitrogen atom, to form a 3, 4, 5 or 6-membered heterocycle; x is 0 or 1, and y is 0 or 1 or 2; and preferably, R⁵² and R⁵³ are independently H, methyl, or together with the carbon they are attached to form a cyclopryl, while R⁵⁴ and R⁵⁵ are independently H or F; and also preferably, R⁵² and R⁵³ are independently H or F, while R⁵⁴ and R⁵⁵ are independently H, F, methyl, or together with the carbon they are attached to form a cyclopryl.

In group (ii), R⁶ is chosen from the group consisting of:

(1) hydroxyl;

(2) C-carboxy;

(3) O-carboxy;

(4) optionally substituted carboxyalkyl (preferably having 4-10 carbon atoms);

(5) —N(R^{ca})C(═O)R^{cb}, —N(R^{ca})C(═O)N(R^{cc})(R^{cd}), —C(═O)N(R^{cc})(R^{cd}) or —OC(═O)N(R^{cc})(R^{cd}), wherein R^{ca} is H or methyl or ethyl (preferably H), and R^{cb}, R^{cc} and R^{cd} are each independently H, OH(R^{cc} and R^{cd} are not both OH) or a chemical moiety chosen from the group of C₁₋₁₀ alkyl, C₂₋₁₀ alkenyl, C₂₋₁₀ alkynyl, C₁₋₁₀ hydroxyalkyl, C₁₋₁₀ alkoxy, C₁₋₁₀ alkylthiol, C₂₋₁₀ alkenyloxy, C₂₋₁₀ alkynyloxy, C₁₋₁₀ alkoxyalkyl, C₁₀ alkylthioalkyl, arylalkoxy, heteroarylalkoxy, carboxyalkyl, C₁₋₆ alkoxy-C₁₋₆ alkyl-, carbocycle, heterocycle, aryl and heteroaryl, or R^{cc} and R^{cd} together with the nitrogen atom to which they are both linked form a 3, 4, 5 or 6-membered optionally substituted heterocycle (e.g., piperidinyl, pyrrolidinyl, and morpholinyl), wherein the chemical moiety is optionally substituted with one or more substituents (e.g., 1, 2, 3 or 4 substituents independently chosen from the group of halo, hydroxyl, optionally substituted C₁₋₆ alkoxy, C₁₋₆ alkylthiol, optionally substituted carbocycle or heterocycle, optionally substituted aryl or heteroaryl, C-carboxy, O-carboxy, carboxyalkyl, and amino);

(6) —N(R^{ab})(R^{ac}) or —SO₂N(R^{ab})(R^{ac}), wherein R^{ab} and R^{ac} are independently H, OH (R^{ab} and R^{ac} are not both OH) or optionally substituted C₁₋₆ alkyl (preferably C₁₋₃ alkyl), or R^{ab} and R^{ac} taken together with the nitrogen they are attached to form a 3, 4, 5 or 6-membered optionally substituted heterocycle; for example, the optionally substituted C₁₋₆ alkyl may include one or more (1, 2, or 3) substituents each independently being hydroxyl, halo, C-carboxy, O-carboxy, optionally substituted heterocycle (e.g., 4-morpholinyl or 3-piperidinyl, with one or more substituents such as C-carboxy) or optionally substituted heteroaryl;

(7) —CON(R^{ak})(R^{al}) wherein R^{ak} and R^{al} are independently H, or C₁₋₆ alkyl that is optionally substituted with 1, 2, or 3 substituents each being independently
(A) hydroxyl;
(B) halo;
(C)—N(R^{am})(R^{an}) where R^{am} and R^{an} are independently H, C₁₋₃ alkyl, hydroxyl, or C₁₋₃ hydroxylalkyl;
(D) heterocycle (e.g.,

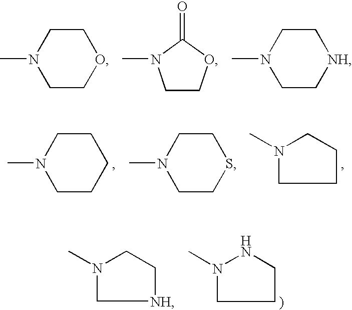

optionally substituted with 1, 2, or 3 substituents each being independently halo (e.g., F, Cl, Br, I), C₁₋₆ alkyl, or C₁₋₃ haloalkyl; and
(E) aryl or heteroaryl (preferably having at least one nitrogen atom, e.g., imidazolyl, pyridinyl, etc.), optionally substituted with 1, 2, or 3 substituents each being independent halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and Rap taken together with the nitrogen they are attached to form a 3, 4, 5 or 6-membered heterocycle; and

(8) optionally substituted carbocycle (preferably cycloalkyl, e.g., cyclohexyl) or heterocycle; for example, suitable optional substituents include, halo, C₁₋₆ alkyl, C₁₋₆ haloalkyl, hydroxyl, C₁₋₆ hydroxyalkyl, C-carboxy, and carboxyalkyl.

In group (ii), R⁶¹ is H or methyl or ethyl; R⁶² and R⁶³ at each occurrence are independently H, C₁₋₆ alkyl, F, hydroxyl, C-carboxy (e.g., methoxycarbonyl), carboxyalkyl, or C-amido, or R⁶² and R⁶³ can be taken together with the carbon they are attached to form a 3, 4, 5 or 6-membered cycloalkyl; and z is 0 to 10, preferably 1, 2, 3, 4, 5, or 6.

In group (iii), R⁵ is as defined above, R⁷¹ is H or methyl or ethyl; R⁷², R⁷³, R⁷⁴, and R⁷⁵ at each occurrence are independently H, methyl, ethyl, or F; x and y are independently an integer of 0, 1 or 2, preferably both x and y are 1. In group (iv), R⁵ is as defined above, R⁸³ is H or methyl or ethyl; R⁸¹ and R⁸² at each occurrence are independently H, methyl, ethyl, or F; d is an integer of 0, 1 or 2 or 3 or 4, and preferably 1.

In group (v), R⁵ is as defined above, R⁹¹ and R⁹² at each occurrence are independently H, methyl, ethyl, or F; d is an integer of 0, 1 or 2, 3 or 4, preferably 1.

In group (vi), R¹⁰ is:

(1) heterocycle (e.g.,

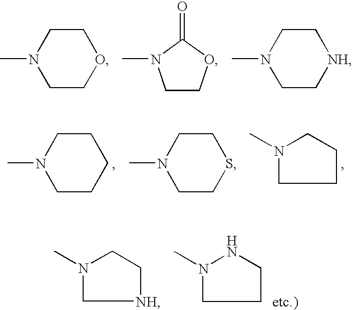

etc.) optionally substituted with 1, 2, or 3 substituents each being independently halo (e.g., F, Cl, Br, I), C₁₋₆ alkyl, or C₁₋₃ haloalkyl;

(2) —CO₂R^{ad} where R^{ad} is H or C₁₋₃ alkyl (preferably methyl); or

(3) optionally substituted aryl or heteroaryl.

R¹⁰¹, R¹⁰², R¹⁰³, and R¹⁰⁴ at each occurrence are independently H, methyl, ethyl, hydroxyl or F;
d is an integer of 0, 1 or 2, 3 or 4, preferably 1-4;
w is an integer of 0, 1, 2, 3 or 4.

In group (vii), R⁵, R⁵¹, R⁵², R⁵³, R⁵⁴, R⁵⁵, x and y are as defined above.

In some embodiments, R² is selected from the group consisting of:

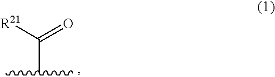

wherein R²¹ is methyl optionally substituted with halo, hydroxyl, cycloalkyl, aryl or heteroaryl;

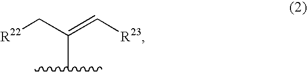

wherein R²² is (A) H, (B) C₁₋₆ alkoxy optionally substituted with 1, 2 or 3 substituents each being independently hydroxyl or C-carboxy; (C) C₁₋₆ alkylthiol optionally substituted with 1, 2 or 3 substituents each being independently hydroxyl or C-carboxy; or (D) —N(R²²¹)(R²²²) wherein R²²¹ and R²²² are independently H, C₁₋₆ alkyl, C₁₋₆ alkoxy, or R²²¹ and R²²² together with the nitrogen atom attached to them form a 3, 4, 5 or 6-membered heterocycle (e.g., 1-pyroolidinyl); and wherein R²³ is H or cyano; and

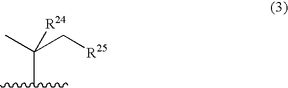

wherein R²⁴ is H or hydroxyl, and R²⁵ is H, hydroxyl, or —N(R²²¹)(R²²²) wherein R²²¹ and R²²² are independently H, C₁₋₆ alkyl, C₁₋₆ alkoxy, or R²²¹ and R²²² together with the nitrogen atom attached to them form a 3, 4, 5 or 6-membered heterocycle (e.g., 1-pyroolidinyl).

In some embodiments, R² is:

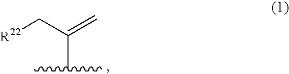

wherein R²² is (A) H, (B) C₁₋₆ alkoxy optionally substituted with 1, 2 or 3 substituents each being independently hydroxyl or C-carboxy; or (C) C₁₋₆ alkylthiol optionally substituted with 1, 2 or 3 substituents each being independently hydroxyl or C-carboxy; or

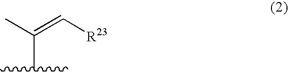

wherein R²³ is H or cyano.

In some embodiments, R² is:

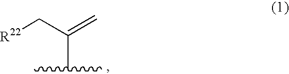

wherein R²² is H, methoxy, ethyoxy, hydroxymethoxy, hydroxyethoxy, methylthiol, ethylthiol, hydroxymethylthiol, or hydroxyethylthiol; or

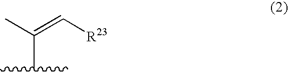

wherein R²³ is H or cyano.

In some embodiments of the compounds of Formula IV, m is 0 and n is 0.

In some embodiments of the compounds of Formula IV, m is 0 and n is 1.

In some embodiments of the compounds of Formula IV, p+q equals at least 1, preferably 2.

In some embodiments of the compounds of Formula IV, m is 0, n is 1, and p+q is 1 or 2.

In some embodiments, R³³ and R³⁴ are independently H, halo, methyl, or R³³ and R³⁴ can be taken together with the carbon atom attached to them to form a cyclopropyl.

In some embodiments, R³⁵ and R³⁶ are independently H, halo, methyl, or R³⁵ and R³⁵ can be taken together with the carbon atom attached to them to form a cyclopropyl.

In some embodiments of the compound of Formula IV, R³ is represented by

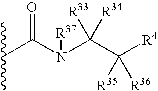

wherein R⁴, R³³, R³⁴, R³⁵, R³⁶, R³⁷ are as defined above. In specific embodiments, R³³ and R³⁴ are both methyl or together with the carbon they are attached to form a cyclopropyl. In other specific embodiments, R³⁵ and R³⁶ are both methyl or together with the carbon they are attached to form a cyclopropyl. In still other specific embodiments, both of R³⁵ and R³⁶ are F, or one F and the other H. In yet other specific embodiments, one of R³³ and R³⁴ is H and the other is methyl, and one of R³⁵ and R³⁶ is H and the other is methyl. In some specific embodiments, one or both of R³³ and R³⁴ are methyl or R³³ and R³⁴ together with the carbon they are attached to form a cyclopropyl, and R³⁵ and R³⁶ are both H. In some specific embodiments, R³³ and R³⁴ are both H, and one or both of R³⁵ and R³⁶ are methyl, or R³⁵ and R³⁶ together with the carbon they are attached to form a cyclopropyl. In other specific embodiments, all of R³³, R³⁴, R³⁵ and R³⁶ are H.

In some embodiments, R³ is represented by

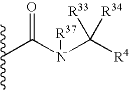

wherein R⁴, R³³, R³⁴, and R³⁷ are as defined above. In some specific embodiments, R³³ and R³⁴ are both H. In some other specific embodiments, one of R³³ and R³⁴ is methyl.

In one embodiment of the compound of Formula IV, R³ is represented by Formula IV′

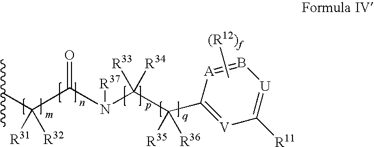

and pharmaceutically acceptably salts and stereoisomers thereof, wherein:

R³¹, R³², R³³, R³⁴, R³⁵, R³⁶, R³⁷, m, n, p, and q are as defined above for Formula IV or various embodiments thereof;

A, B, U and V are each independently C or N; for example, 0, 1, 2, 3 or all of A, B, U and V are N;

f is an integer of 0, 1, 2, 3 or 4; preferably 0 or 1;

R¹² can be attached any of A, B, U and V, and at each occurrence independently is H, halo (e.g., F, Cl, Br), hydroxyl, optionally substituted C₁₋₁₀ alkyl (preferably C₁₋₄ alkyl, e.g. methyl, ethyl, optionally substituted with 1-3 halo, e.g., F), optionally substituted C₁₋₁₀ alkoxy (preferably C₁₋₄ alkoxy, e.g. methoxy, ethoxy, optionally substituted with 1-3 halo, e.g., F), O-carboxy or C-carboxy; and

R¹¹ is as defined above for Formula IV or various embodiments thereof.

In some specific embodiments, R¹² is attached to A.

In some specific embodiments of the compounds of Formula IV′, R¹¹ is represented by

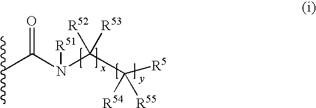

wherein R⁵¹, R⁵², R⁵³, R⁵⁴, R⁵⁵, x, y and R⁵ are as defined above for Formula IV or various embodiments thereof.

In some embodiments, R³ in Formula IV is represented by Formula IV″

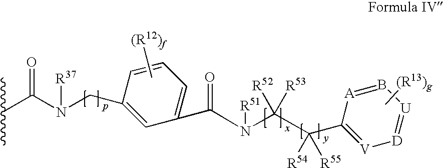

wherein R³⁷, R⁵¹, R⁵², R⁵³, R⁵⁴, R⁵⁵, x, y, f, p, and R¹² are as defined above for Formula IV or various embodiments thereof;
A, B, D, U and V are independently C or N; preferably 0, 1 or 3 of A, B, D, U and V are N;
g is an integer of 0, 1, 2, 3, 4 or 5;
R¹³ can be attached any of A, B, D, U and V, and at each occurance independently is H or

(1) halo (e.g., F, Cl, Br, I);

(2) hydroxyl; cyano;

(3) C-carboxy, O-carboxy, or carboxyalkyl;

(4) optionally substituted alkyl (preferably C₁₋₆, more preferably C₁₋₃ alkyl) or cycloalkyl (preferably C₃₋₆ cycloalkyl), for example, substituted with one or more (e.g., 1, 2, 3 or 4) substituents independently chosen from the group consisting of:
(A) hydroxyl; (B) halo; (C) amino;
(D) C₁₋₆ alkoxy; and
(E) arylalkoxy or heteroarylalkoxy optionally substituted with hydroxyl, halo, C-carboxy, amino, and C₁₋₆ alkoxy.

(5) —N(R^{ca})C(═O)R^{cb}, —N(R^{ca})C(═O)N(R^{cc})(R^{cd}), or —OC(═O)N(R^{cc})(R^{cd}), wherein R^{ca} is H or methyl or ethyl (preferably H), and R^{cb}, R^{cc} and R^{cd} are each independently H, OH(R^{cc} and R^{cd} are not both OH) or a chemical moiety chosen from the group of C₁₋₁₀ alkyl, C₂₋₁₀ alkenyl, C₂₋₁₀ alkynyl, C₁₋₁₀ hydroxyalkyl, C₁₋₁₀ alkoxy, C₁₋₁₀ alkylthiol, C₂₋₁₀ alkenyloxy, C₂₋₁₀ alkynyloxy, C₁₋₁₀ alkoxyalkyl, C₁₋₁₀ alkylthioalkyl, carboxyalkyl, carbocycle, heterocycle, aryl and heteroaryl, or R^{cc} and R^{cd} together with the nitrogen atom to which they are both linked form a 3, 4, 5 or 6-membered optionally substituted heterocycle (e.g., piperidinyl, pyrrolidinyl, and morpholinyl), wherein the chemical moiety is optionally substituted with one or more substituents (e.g., 1, 2, 3 or 4 substituents independently chosen from the group of halo, hydroxyl, optionally substituted C₁₋₆ alkoxy, C₁₋₆ alkylthiol, optionally substituted carbocycle or heterocycle, optionally substituted aryl or heteroaryl, C-carboxy, O-carboxy, carboxyalkyl, and amino);

(6) —N(R^{ab})(R^{ac}) or —SO₂N(R^{ab})(R^{ac}), wherein R^{ab} and R^{ac} are independently H, OH (R^{ab} and R^{ac} are not both OH) or optionally substituted C₁₋₆ alkyl (preferably C₁₋₃ alkyl), or R^{ab} and R^{ac} taken together with the nitrogen they are attached to form a 3, 4, 5 or 6-membered optionally substituted heterocycle; for example, the optionally substituted C₁₋₆ alkyl may include one or more (1, 2, or 3) substituents each independently being hydroxyl, halo, C-carboxy, O-carboxy, amino, optionally substituted heterocycle (e.g., 4-morpholinyl or 3-piperidinyl, with one or more substituents such as C-carboxy) or optionally substituted heteroaryl.

(7) optionally substituted C₁₋₆ alkoxy; for example, the C₁₋₆ alkoxy can be optionally substituted with 1, 2 or 3 substituents each being independently chosen from the group consisting of:
(A) hydroxyl;
(B) halo (e.g., F, Cl, Br, I);
(C) —CO₂R^{aa}, —O(C═O)R^{aa}, —C₁₋₆ alkyl-CO₂R^{aa}, or wherein R^{aa} is H or C₁₋₃ alkyl, preferably methyl or ethyl;
(D) —N(R^{ca})C(═O)R^{cb}, —N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle (e.g., piperidinyl, pyrrolidinyl, and morpholinyl);
(E) optionally substituted heterocycle (e.g.,

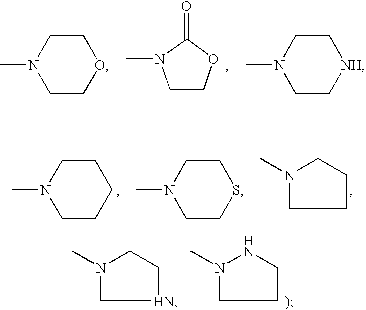

for example with one or more substituents (e.g., 1, 2, or 3) each being independently halo (e.g., F, Cl, Br, I), hydroxyl, C₁₋₆ alkyl, C₁₋₃ haloalkyl, C-carboxyl, and sulfonyl;
(F) heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, C-carboxy, C₁₋₃ hydroxyalkyl, C₁₋₃ haloalkyl, or —N(R^{ae})(R^{af}) or —SO₂N(R^{ae})(R^{af}), wherein R^{ae} and R^{af} are independently H, OH (R^{ae} and R^{af} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered optionally substituted heterocycle; and
(G) —N(R^{ag})(R^{ah}) where R^{ag} and R^{ah} are independently H, hydroxyl, C₁₋₆ alkyl, optionally substituted C₁₋₆ hydroxyalkyl or aminoalkyl, or R^{ag} and R^{ah} can be taken together with the nitrogen they are attached to form a 3, 4, 5 or 6-membered optionally substituted heterocycle; and

(9) —CON(R^{ak})(R^{al}) wherein R^{ak} and R^{al} are independently H, OH (R^{ak} and R^{al} are not both OH) or a chemical moiety chosen from the group consisting of C₁₋₁₀ alkyl, C₂₋₁₀ alkenyl, C₂₋₁₀ alkynyl, C₁₋₁₀ hydroxyalkyl, C₁₋₁₀ alkoxy, C₁₋₁₀ alkylthiol, C₂₋₁₀ alkenyloxy, C₂₋₁₀ alkynyloxy, C₁₋₁₀ alkoxyalkyl, C₁₋₁₀ alkylthioalkyl, carboxyalkyl, aminoalkyl, carbocycle, heterocycle, aryl, arylakly, heteroaryl, heteroarylalkyl, or R^{ak} and R^{al} together with the nitrogen atom to which they are both linked form a 3, 4, 5 or 6-membered heterocycle (e.g., piperidinyl, pyrrolidinyl, and morpholinyl), wherein the chemical moiety is optionally substituted with one or more substituents (e.g., 1, 2, 3 or 4 substituents. For example, the chemical moiety can be optionally substituted with 1, 2, or 3 or 4 substituents each being independently
(A) hydroxyl;
(B) halo;
(C) C₁₋₆ hydroxyalkyl;
(D) C₁₋₆ alkoxy;
(E) C₁₋₆ alkylthiol;
(F) C-carboxy, O-carboxy, or carboxyalkyl;
(G) —N(R^{am})(R^{an}) where R^{am} and R^{an} are independently H, hydroxyl, optionally substituted C₁₋₆ alkyl, optionally substituted C₁₋₆ hydroxyalkyl, or R^{am} and R^{an} together with the nitrogen atom they are attached to form a 3, 4, 5 or 6-membered optionally substituted heterocycle;
(H) heterocycle (e.g.,

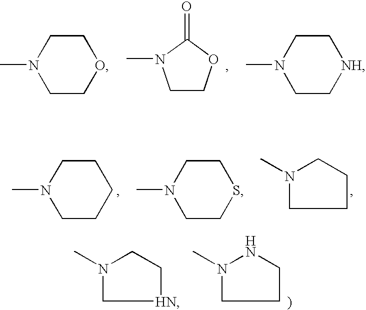

optionally substituted with one or more substituents (e.g., 1, 2, or 3 substituents each being independently halo, (e.g., F, Cl, Br, I), hydroxyl, C₁₋₆ alkyl, C₁₋₃ haloalkyl, C-carboxy, O-carboxy or carboxyalkyl); and
(I) aryl or heteroaryl (preferably having at least one nitrogen atom, e.g., imidazolyl, pyridinyl, etc.), optionally substituted with 1, 2, or 3 substituents each being independent halo (e.g., F, Cl, Br, I), hydroxyl, optionally substituted C₁₋₆ alkyl (e.g., methyl, C₁₋₆ haloalkyl), C-carboxy, O-carboxy carboxyalkyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and Rap taken together with the nitrogen they are attached to form a 3, 4, 5 or 6-membered heterocycle; or

(10) carbocycle, heterocycle, aryl, heteroaryl, arylalkyl or heteroarylalkyl optionally substituted by R¹³.

In another embodiment, the invention provides a compound of formula V

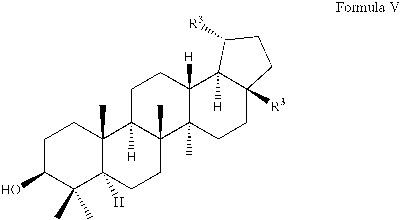

and pharmaceutically acceptably salts and stereoisomers thereof, wherein:
R² is isopropenyl or isopropyl, optionally substituted with one or two substituents independently selected from hydroxyl, halo, amino, and pyrrolidinyl, piperidinyl, and preferably R² is isopropenyl, isopropyl, 1′-hydroxyisopropyl, 2′-hydroxyisopryl, 1′,2′-dihydroxyisopropyl, and 1′-pyrrolidinyl-2′-hydroxyisopropyl;
R³ is represented by

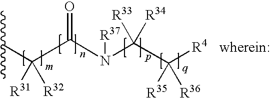

R³¹ and R³² are independently (meaning that R³¹ and R³² are not necessarily identical at each unit —C(R³¹)(R³²)—) H or methyl or ethyl, or R³¹ and R³² can be taken together with the carbon they are attached to form a C₃₋₅ cycloalkyl (e.g., cyclopropyl, cyclobutyl or cyclopentyl);
R³³ is H, halo (e.g., F), —COOR^{33a} (R^{33a} is H or C₁₋₆ alkyl such as methyl, ethyl, propyl and isopropyl), methyl or ethyl, and R³⁴ is H, halo (e.g., F), methyl or ethyl, or R³³ and R³⁴ can be taken together with the carbon they are attached to form a C₃₋₅ cycloalkyl (e.g., cyclopropyl, cyclobutyl or cyclopentyl);
R³⁵ and R³⁶ are independently (meaning that R³⁵ and R³⁶ are not necessarily identical at each unit —C(R³⁵)(R³⁶)—) H, halo (e.g., F), methyl or ethyl, or R³³ and R³⁴ can be taken together with the carbon they are attached to form a C₃₋₅ cycloalkyl (e.g., cyclopropyl, cyclobutyl or cyclopentyl);
R³⁷ is H or methyl or ethyl, and preferably H;
m is an integer of 0 or 1;
n is an integer of 0 or 1;
p is an integer of 0 or 1 or 2;
q is an integer of 0 or 1 or 2; and preferably m+n+p+q is from 1 to 4, more preferably is 2 or 3; and
R⁴ is an aryl or heteroaryl (e.g., phenyl, pyridinyl, furanyl, and thiophenyl) substituted with a first substitutent and optionally one or more (e.g., 1, 2, or 3 or 4) other substituents, said one or more other substituents being independently chosen from halo (e.g., F, Cl, Br), hydroxyl, C₁₋₁₀ alkyl (preferably C₁₋₄ alkyl, e.g. methyl, ethyl, optionally substituted 1-3 halo, e.g., F), C₁₋₁₀ alkoxy (preferably C₁₋₄ alkoxy, e.g. methoxy, ethoxy, optionally substituted 1-3 halo, e.g., F), carboxyl, and C₁₋₆ alkoxycarbonyl; wherein said first substituent is chosen from carboxyl, C₁₋₆ alkoxycarbonyl,

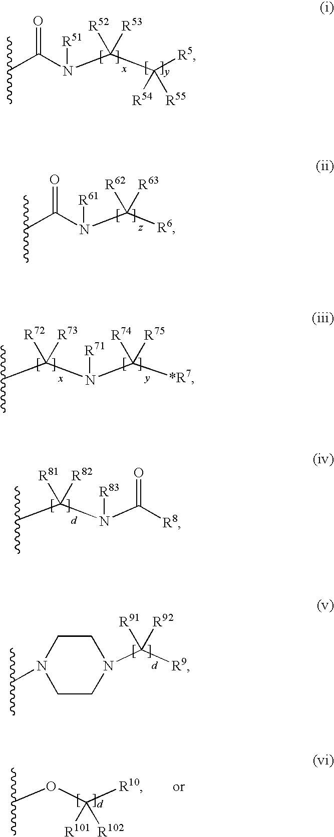

(vii) an aryl or heteroaryl optionally substituted with one or more substituent groups each being independently chosen from:

(a) halo (e.g., F, Cl, Br, I); (b) hydroxyl;

(c) C₁₋₆ alkyl (preferably C₁₋₃ alkyl) or C₃₋₆ cycloalkyl, optionally substituted with OH or halo (preferably F, e.g., monofluoro, difluoro, or trifluoro);

(d) —CO₂R^{aa}, —O(C═O)R^{aa}, —C₁₋₆ alkylene-CO₂R^{aa}, or wherein R^{aa} is H or C₁₋₃ alkyl, preferably methyl or ethyl;

(e) —N(R^{ab})(R^{ac}) or —SO₂N(R^{ab})(R^{ac}), wherein R^{ab} and R^{ac} are each independently H, OH (R^{ab} and R^{ac} are not both OH), or optionally substituted C₁₋₃ or C₁₋₆ alkyl, or R^{ab} and R^{ac} taken together with the nitrogen they are attached to form a 3, 4, 5 or 6-membered heterocycle optionally substituted with one or more substitutents;

(f) —N(R^{ca})C(═O)R^{cb}, N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are each independently H, OH(R^{cc} and R^{cd} are not both OH), or a chemical moiety chosen from the group of C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, carbocycle, heterocycle, aryl and heteroaryl, or R^{cc} and R^{cd} together with the nitrogen atom to which they are both linked form a 3, 4, 5 or 6-membered optionally substituted heterocycle (e.g., piperidinyl, pyrrolidinyl, and morpholinyl), wherein the chemical moiety is optionally substituted with one or more substituents (e.g., halo, hydroxyl, C₁₋₆ alkoxy, carbocycle, heterocycle, aryl, heteroaryl, carboxyl, or alkoxy carbonyl);

(g) optionally substituted C₁₋₆ alkoxy, for example, having 1, 2 or 3 substituents each being independently chosen from the group consisting of:
(A) hydroxyl; halo (e.g., F, Cl, Br, I);
(B) —CO₂R^{ad} where R^{ad} is H or C₁₋₃ alkyl (preferably methyl); heterocycle (e.g.,

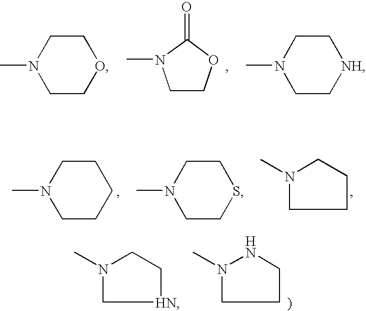

optionally substituted with 1, 2, or 3 substituents (e.g., each substituent being independently halo (e.g., F, Cl, Br, I), C₁₋₆ alkyl, or C₁₋₃ haloalkyl);
(C) heteroaryl (e.g., imidazolyl) optionally substituted with 1, 2, or 3 substituents (for example, the substituents can be independently, halo (e.g., F, Cl, Br, I), hydroxyl, C₁₋₆ alkyl (preferably methyl), C₁₋₆ alkoxy, carboxyl, C₁₋₃ alkoxycarbonyl, C₁₋₃ hydroxyalkyl, C₁₋₃ haloalkyl, or —N(R^{ae})(R^{af}) or —SO₂N(R^{ae})(R^{af}) wherein R^{ae} and R^{af} are each independently H, OH (R^{ae} and R^{af} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered optionally substituted heterocycle); and
(D) —N(R^{ag})(R^{ah}) where R^{ag} and R^{ah} are each independently H, hydroxyl, optionally substituted C₁₋₆ alkyl, or —N(R^{ai})(R^{aj}) where R^{ai} and R^{aj} are independently H or optionally substituted C₁₋₃ alkyl or R^{ag} and R^{ah} can be taken together with the nitrogen they are attached to form a 3, 4, 5 or 6-membered optionally substituted heterocycle, and/or R^{ai} and R^{aj} can be taken together with the nitrogen they are attached to form a 3, 4, 5 or 6-membered optionally heterocycle; and

(h) —CON(R^{ak})(R^{al}) wherein R^{ak} and R^{al} are each independently H or C₁₋₆ alkyl that is optionally substituted with 1, 2, or 3 substituents, and examples of such substituents include:
(A) hydroxyl; (B) halo;
(C)—N(R^{am})(R^{an}) where R^{am} and R^{an} are independently H, hydroxyl, or optionally substituted C₁₋₃ alkyl (e.g., C₁₋₃ hydroxylalkyl);
(D) heterocycle (e.g.,

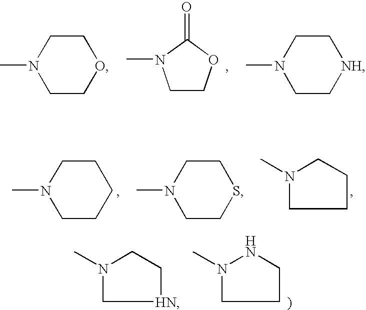

optionally substituted with one or more substituents (e.g., substituted with 1, 2, or 3 substituents each being independently halo (e.g., F, Cl, Br, I), C₁₋₆ alkyl, or C₁₋₃ haloalkyl); and
(E) aryl or heteroaryl (preferably having at least one nitrogen atom, e.g., imidazolyl, pyridinyl, etc.) optionally substituted with 1, 2, or 3 substituents (for example, each substituent may be independently halo (e.g., F, Cl, Br, I), hydroxyl, carboxyl, optionally substituted C₁₋₆ alkyl (preferably methyl), C₁₋₃ haloalkyl, C₁₋₃ alkyoxycarbonyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and Rap taken together with the nitrogen they are attached to form a 3, 4, 5 or 6-membered heterocycle.

In group (i), R⁵ is an aryl, arylalkyl, heteroaryl or heteroarylalkyl (e.g., phenyl, biphenyl, pyridinyl, furanyl, and thiophenyl, etc.) (preferably aryl or heteroaryl), each being optionally substituted with one or more (e.g., 1, 2 or 3) substituents. For example, the one or more optional substituents can each be independently chosen from:

(a) halo (e.g., F, Cl, Br, I);
(b) hydroxyl;
(c) —CO₂R³, —O(C═O)R^{aa}, —C₁₋₆ alkylene-CO₂R^{aa}, wherein R^{aa} is H or optionally substituted C₁₋₆ alkyl, preferably methyl or ethyl;
(d) C₁₋₆ alkyl (preferably C₁₋₃ alkyl) or C₃₋₆ cycloalkyl, optionally substituted with one or more (e.g., 1, 2, 3 or 4) substituents (e.g., hydroxyl, optionally substituted C₁₋₆ alkoxy, halo, amino, heterocycle, etc.);
(e) —N(R^{ca})C(═O)R^{cb}, N(R^{ca})C(═O)N(R^{cc})(R^{cd}), or —OC(═O)N(R^{cc})(R^{cd}), where R^{ca} is H or methyl or ethyl (preferably H); and R^{cb}, R^{cc} and R^{cd} are each independently H, OH (R^{cc} and R^{cd} are not both OH) or a chemical moiety chosen from the group of C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, carbocycle, heterocycle, aryl and heteroaryl, or R^{cc} and R^{cd} together with the nitrogen atom to which they are both linked form a 3, 4, 5 or 6-membered optionally substituted heterocycle (e.g., piperidinyl, pyrrolidinyl, and morpholinyl), wherein the chemical moiety is optionally substituted with one or more substituents (e.g., halo, hydroxyl, C₁₋₆ alkoxy, carbocycle, heterocycle, aryl, heteroaryl, carboxyl, or alkoxy carbonyl);
(f) —C(═O)N(R^{cc})(R^{cd}) wherein R^{cc} and R^{cd} are each independently H, OH(R^{cc} and R^{cd} are not both OH) or a chemical moiety chosen from the group of C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, carbocycle, heterocycle, aryl and heteroaryl, or R^{cc} and R^{cd} together with the nitrogen atom to which they are both linked form a 3, 4, 5 or 6-membered optionally substituted heterocycle (e.g., piperidinyl, pyrrolidinyl, and morpholinyl), wherein the chemical moiety is optionally substituted with one or more substituents (e.g., each substituent being independently halo, hydroxyl, optionally substituted C₁₋₆ alkoxy, optionally substituted carbocycle or heterocycle, optionally substituted aryl or heteroaryl, carboxyl, or alkoxycarbonyl);
(g) —N(R^{ab})(R^{ac}) or —SO₂N(R^{ab})(R^{ac}), wherein R^{ab} and R^{ac} are independently H, OH (R^{ab} and R^{ac} are not both OH) or optionally substituted C₁₋₆ alkyl (preferably C₁₋₃ alkyl), or R^{ab} and R^{ac} taken together with the nitrogen they are attached to form a 3, 4, 5 or 6-membered optionally substituted heterocycle; For example, the optionally substituted C₁₋₆ alkyl may include one or more (1, 2, or 3) substituents each independently being hydroxyl, halo, carboxyl, alkoxycarbonyl, optionally substituted heterocycle or optionally substituted heteroaryl.
(h) optionally substituted C₁₋₆ alkoxy; For example, the C₁₋₆ alkoxy can be optionally substituted with 1, 2 or 3 substituents each being independently chosen from the group consisting of:

(A) hydroxyl; halo (e.g., F, Cl, Br, I);

(B) —CO₂R^{aa}, —O(C═O)R^{aa}, —C₁₋₆ alkyl-CO₂R^{aa}, or wherein R^{aa} is H or C₁₋₃ alkyl, preferably methyl or ethyl;

(C) —N(R^{ca})C(═O)R^{cb}, —N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle (e.g., piperidinyl, pyrrolidinyl, and morpholinyl);

(D) heterocycle (e.g.,

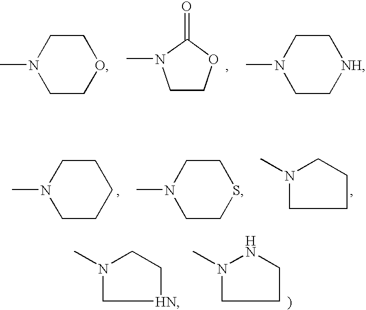

optionally substituted with one or more substituents (e.g., 1, 2, or 3 substituents each being independently halo, (e.g., F, Cl, Br, I), hydroxyl, C₁₋₆ alkyl, C₁₋₃ haloalkyl, carboxyl or alkoxycarbonyl);

(E) heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, carboxyl, C₁₋₃ alkoxycarbonyl, C₁₋₃ hydroxyalkyl, C₁₋₃ haloalkyl, or —N(R^{ae})(R^{af}) or —SO₂N(R^{ae})(R^{af}), wherein R^{ae} and R^{af} are independently H, OH (R^{ae} and R^{af} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered optionally substituted heterocycle; and

(F) —N(R^{ag})(R^{ah}) where R^{ag} and R^{ah} are independently H, hydroxyl, C₁₋₆ alkyl, C₁₋₆ hydroxyalkyl, or —N(R^{ai})(R^{aj}) where R^{ai} and R^{aj} are independently H or C₁₋₃ alkyl, or R^{ag} and R^{ah} can be taken together with the nitrogen they are attached to form a 3, 4, 5 or 6-membered optionally substituted heterocycle, and/or R^{ai} and R^{aj} can be taken together with the nitrogen they are attached to form a 3, 4, 5 or 6-membered optionally substituted heterocycle; and

(i) —CON(R^{ak})(R^{al}) wherein R^{ak} and R^{al} are independently H, or C₁₋₆ alkyl that is optionally substituted with one or more substituents. For example, the C₁₋₆ alkyl can be optionally substituted with 1, 2, or 3 substituents each being independently

(A) hydroxyl;

(B) halo;

(C) —N(R^{am})(R^{an}) where R^{am} and R^{an} are independently H, C₁₋₃ alkyl, hydroxyl, or C₁₋₃ hydroxylalkyl;

(D) heterocycle (e.g.,

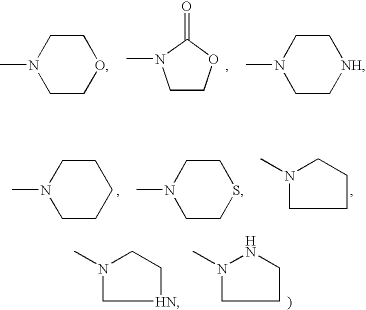

optionally substituted with one or more substituents (e.g., 1, 2, or 3 substituents each being independently halo, (e.g., F, Cl, Br, I), hydroxyl, C₁₋₆ alkyl, C₁₋₃ haloalkyl, carboxyl or alkoxycarbonyl);

(E) aryl or heteroaryl (preferably having at least one nitrogen atom, e.g., imidazolyl, pyridinyl, etc.), optionally substituted with 1, 2, or 3 substituents each being independent halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and Rap taken together with the nitrogen they are attached to form a 3, 4, 5 or 6-membered heterocycle.

In group (i), R⁵¹ is H or methyl or ethyl; R⁵² and R⁵³ at each occurrence are independently H, methyl, ethyl, F, hydroxyl, —COOH, —CO₂-methyl, —CO₂-ethyl, or —CO₂NH₂, or R⁵² and R⁵³ can be taken together with the carbon they are attached to form a cyclopryl; R⁵⁴ and R⁵⁵ at each occurrence are independently H, methyl, ethyl, F, or hydroxyl, or R⁵⁴ and R⁵⁵ can be taken together with the carbon they are attached to form a cyclopryl; x is 0 or 1, and y is 0 or 1; and preferably, R⁵² and R⁵³ are independently H, methyl, or together with the carbon they are attached to form a cyclopryl, while R⁵⁴ and R⁵⁵ are independently H or F; and also preferably, R⁵² and R⁵³ are independently H or F, while R⁵⁴ and R⁵⁵ are independently H, F, methyl, or together with the carbon they are attached to form a cyclopryl.

In group (ii), R⁶ is chosen from the group consisting of:

(a) —CO₂R^{aa}, —O(C═O)R^{aa}, —C₁₋₆ alkyl-CO₂R^{aa}, or wherein R^{aa} is H or C₁₋₃ alkyl, preferably methyl or ethyl;
(b) —N(R^{ca})C(═O)R^{cb}, —N(R^{ca})C(═O)N(R^{cc})(R^{cd}), C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle (e.g., piperidinyl, pyrrolidinyl, and morpholinyl);
(c) —N(R^{ab})(R^{ac}) or —SO₂N(R^{ab})(R^{ac}), where R^{ab} and R^{ac} are independently H, OH (R^{ab} and R^{ac} are not both OH), C₁₋₃ alkyl, C₁₋₆ hydroxyalkyl, or C₁₋₆ alkyl (preferably C₁₋₃ alkyl), or R^{ab} and R^{ac} taken together with the nitrogen they are attached to form a 3, 4, 5 or 6-membered heterocycle, or —COR^{ba} where R^{ba} is C₁₋₆ alkyl optionally substituted with 1, 2, or 3 hydroxyl or halo, or R^{ba} is C₁₋₆ alkoxy optionally substituted with 1 or 2 or 3 substituents independently chosen from hydroxyl, amino, aryl or heteroaryl that is optionally substituted with 1, 2, or 3 substituents each being independent halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{ao})(R^{ap}) or —SO₂N(R^{ao})(R^{ap}) wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and Rap taken together with the nitrogen they are attached to form a 3, 4, 5 or 6-membered heterocycle; and
(d) —CON(R^{ak})(R^{al}) wherein R^{ak} and R^{al} are independently H, or C₁₋₆ alkyl that is optionally substituted with 1, 2, or 3 substituents each being independently

(A) hydroxyl;

(B) halo;

(C) —N(R^{am})(R^{an}) where R^{am} and R^{an} are independently H, C₁₋₃ alkyl, hydroxyl, or C₁₋₃ hydroxylalkyl;

(D) heterocycle (e.g.,

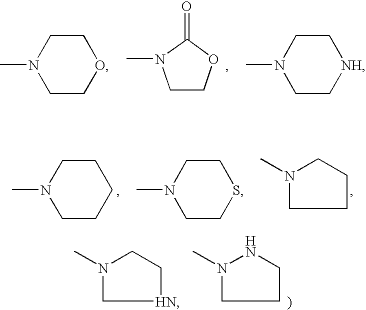

optionally substituted with 1, 2, or 3 substituents each being independently halo (e.g., F, Cl, Br, I), C₁₋₆ alkyl, or C₁₋₃ haloalkyl; and

(E) aryl or heteroaryl (preferably having at least one nitrogen atom, e.g., imidazolyl, pyridinyl, etc.), optionally substituted with 1, 2, or 3 substituents each being independent halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and Rap taken together with the nitrogen they are attached to form a 3, 4, 5 or 6-membered heterocycle. In group (ii), R⁶¹ is H or methyl or ethyl; R⁶² and R⁶³ at each occurrence are independently H, methyl, ethyl, F, hydroxyl, —C₁₋₆ alkyl-COOH, —C₁₋₆ alkyl-CO₂-methyl, —C₁₋₆ alkyl-CO₂-ethyl, or —C₁₋₆ alkyl-CO₂NH₂, or R⁶² and R⁶³ can be taken together with the carbon they are attached to form a C₃₋₆ cycloalkyl; and z is 0 to 10, preferably 1, 2, 3, 4, 5, or 6.

In group (iii), R⁷ is an aryl or heteroaryl optionally substituted with one or more (e.g., 1, 2, or 3) substituents each being independently chosen from:

(a) halo (e.g., F, Cl, Br, I);

(b) hydroxyl;

(c) C₁₋₆ alkyl (preferably C₁₋₃ alkyl) or C₃₋₆ cycloalkyl, optionally substituted with OH or halo (preferably F, e.g., monofluoro, difluoro, or trifluoro);

(d) —CO₂R^{aa}, —O(C═O)R^{aa}, —C₁₋₆ alkyl-CO₂R^{aa}, or wherein R^{aa} is H or C₁₋₃ alkyl, preferably methyl or ethyl;

(e) —N(R^{ca})C(═O)R^{cb}, —N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle (e.g., piperidinyl, pyrrolidinyl, and morpholinyl);

(f) —N(R^{ab})(R^{ac}) or —SO₂N(R^{ab})(R^{ac}), wherein R^{ab} and R^{ac} are independently H, OH (R^{ab} and R^{ac} are not both OH), C₁₋₃ alkyl, C₁₋₆ hydroxyalkyl, or C₁₋₆ alkyl (preferably C₁₋₃ alkyl), or R^{ab} and R^{ac} taken together with the nitrogen they are attached to form a 3, 4, 5 or 6-membered heterocycle;

(g) C₁₋₆ alkoxy optionally substituted with 1, 2 or 3 substituents each being independently chosen from the group consisting of:
(A) hydroxyl;
(B) halo (e.g., F, Cl, Br, I);
(C)—CO₂R^{ad} where R^{ad} is H or C₁₋₃ alkyl (preferably methyl);
(D) heterocycle (e.g.,

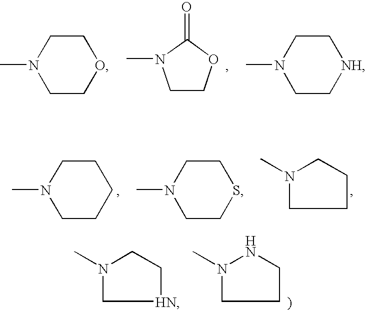

optionally substituted with 1, 2, or 3 substituents each being independently halo (e.g., F, Cl, Br, I), C₁₋₆ alkyl, or C₁₋₃ haloalkyl;

(E) heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, carboxyl, C₁₋₃ alkoxycarbonyl, C₁₋₃ hydroxyalkyl, C₁₋₃ haloalkyl, or —N(R^{ae})(R^{af}) or —SO₂N(R^{ae})(R^{af}), wherein R^{ae} and R^{af} are independently H, OH (R^{ae} and R^{af} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered heterocycle; and

(F) —N(R^{ag})(R^{ah}) where R^{ag} and R^{ah} are independently H, hydroxyl, C₁₋₆ alkyl, C₁₋₆ hydroxyalkyl, or —N(R^{ai})(R^{aj}) where R^{al} and R^{al} are independently H or C₁₋₃ alkyl, or R^{ag} and R^{ah} can be taken together with the nitrogen they are attached to form a 3, 4, 5 or 6-membered heterocycle, and/or R^{al} and R^{al} can be taken together with the nitrogen they are attached to form a 3, 4, 5 or 6-membered heterocycle; and

(h) —CON(R^{ak})(R^{al}) wherein R^{ak} and R^{al} are independently H, or C₁₋₆ alkyl that is optionally substituted with 1, 2, or 3 substituents each being independently
(A) hydroxyl;
(B) halo;
(C)—N(R^{am})(R^{an}) where R^{am} and R^{an} are independently H, C₁₋₃ alkyl, hydroxyl, or C₁₋₃ hydroxylalkyl;
(D) heterocycle (e.g.,

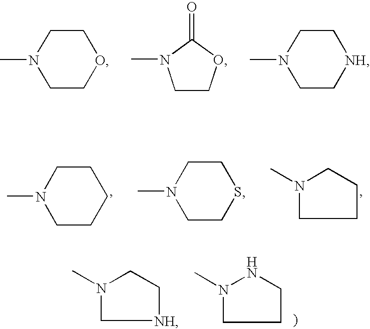

optionally substituted with 1, 2, or 3 substituents each being independently halo (e.g., F, Cl, Br, I), C₁₋₆ alkyl, or C₁₋₃ haloalkyl; and

(E) aryl or heteroaryl (preferably having at least one nitrogen atom, e.g., imidazolyl, pyridinyl, etc.), optionally substituted with 1, 2, or 3 substituents each being independent halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and Rap taken together with the nitrogen they are attached to form a 3, 4, 5 or 6-membered heterocycle.

In group (iii), R⁷¹ is H or methyl or ethyl; R⁷², R⁷³, R⁷⁴, and R⁷⁵ at each occurrence are independently H, methyl, ethyl, or F; x and y are independently an integer of 0, 1 or 2, preferably both x and y are 1.

In group (iv), R⁸ is an aryl or heteroaryl optionally substituted with one or more (e.g., 1, 2, or 3) substituents each being independently chosen from:

(a) halo (e.g., F, Cl, Br, I);
(b) hydroxyl;
(c) C₁₋₆ alkyl (preferably C₁₋₃ alkyl) or C₃₋₆ cycloalkyl, optionally substituted with OH or halo (preferably F, e.g., monofluoro, difluoro, or trifluoro);
(d) —CO₂R^{aa}, —O(C═O)R^{aa}, —C₁₋₆ alkyl-CO₂R^{aa}, or wherein R^{aa} is H or C₁₋₃ alkyl, preferably methyl or ethyl;
(e) —N(R^{ca})C(═O)R^{cb}, —N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle (e.g., piperidinyl, pyrrolidinyl, and morpholinyl);
(f) —N(R^{ab})(R^{ac}) or —SO₂N(R^{ab})(R^{ac}), wherein R^{ab} and R^{ac} are independently H, OH (R^{ab} and R^{ac} are not both OH), C₁₋₃ alkyl, C₁₋₆ hydroxyalkyl, or C₁₋₆ alkyl (preferably C₁₋₃ alkyl), or R^{ab} and R^{ac} taken together with the nitrogen they are attached to form a 3, 4, 5 or 6-membered heterocycle;
(g) C₁₋₆ alkoxy optionally substituted with 1, 2 or 3 substituents each being independently chosen from the group consisting of:

(A) hydroxyl;

(B) halo (e.g., F, Cl, Br, I);

(C)—CO₂R^{aa}, —O(C═O)R^{aa}, —C₁₋₆ alkyl-CO₂R^{aa}, or wherein R^{aa} is H or C₁₋₃ alkyl, preferably methyl or ethyl;

(D) —N(R^{ca})C(═O)R^{cb}, N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle (e.g., piperidinyl, pyrrolidinyl, and morpholinyl);

(E) heterocycle (e.g.,

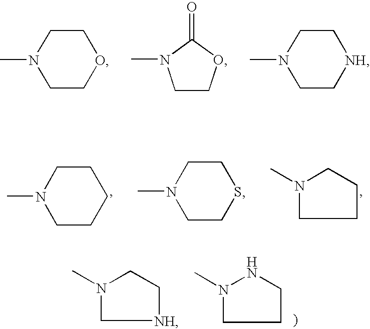

optionally substituted with 1, 2, or 3 substituents each being independently halo (e.g., F, Cl, Br, I), C₁₋₆ alkyl, or C₁₋₃ haloalkyl;

(F) heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, carboxyl, C₁₋₃ alkoxycarbonyl, C₁₋₃ hydroxyalkyl, C₁₋₃ haloalkyl, or —N(R^{ae})(R^{af}) or —SO₂N(R^{ae})(R^{af}), wherein R^{ae} and R^{af} are independently H, OH (R^{ae} and R^{af} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered heterocycle; and

(G) —N(R^{ag})(R^{ah}) where R^{ag} and R^{ah} are independently H, hydroxyl, C₁₋₆ alkyl, C₁₋₆ hydroxyalkyl, or —N(R^{ai})(R^{aj}) where R^{ai} and R^{aj} are independently H or C₁₋₃ alkyl, or R^{ag} and R^{ah} can be taken together with the nitrogen they are attached to form a 3, 4, 5 or 6-membered heterocycle, and/or R^{al} and R^{al} can be taken together with the nitrogen they are attached to form a 3, 4, 5 or 6-membered heterocycle; and

(h) —CON(R^{ak})(R^{al}) wherein R^{ak} and R^{al} are independently H, or C₁₋₆ alkyl that is optionally substituted with 1, 2, or 3 substituents each being independently

(A) hydroxyl;

(B) halo;

(C) —N(R^{am})(R^{an}) where R^{am} and R^{an} are independently H, C₁₋₃ alkyl, hydroxyl, or C₁₋₃ hydroxylalkyl;

(D) heterocycle (e.g.,

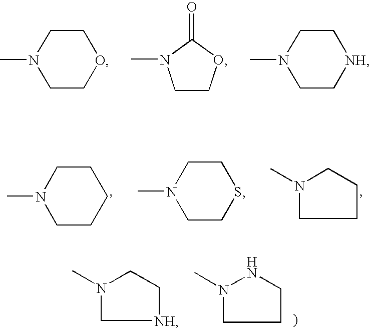

optionally substituted with 1, 2, or 3 substituents each being independently halo (e.g., F, Cl, Br, I), C₁₋₆ alkyl, or C₁₋₃ haloalkyl; and

(E) aryl or heteroaryl (preferably having at least one nitrogen atom, e.g., imidazolyl, pyridinyl, etc.), optionally substituted with 1, 2, or 3 substituents each being independent halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and R^{ap} taken together with the nitrogen they are attached to form a 3, 4, 5 or 6-membered heterocycle.

In group (iv), R³ is H or methyl or ethyl; R⁸¹ and R⁸² at each occurrence are independently H, methyl, ethyl, or F; d is an integer of 0, 1 or 2 or 3 or 4, preferably 1.

In group (v), R⁹ is an aryl or heteroaryl optionally substituted with one or more (e.g., 1, 2, or 3) substituents each being independently chosen from:

(a) halo (e.g., F, Cl, Br, I);
(b) hydroxyl;
(c) C₁₋₆ alkyl (preferably C₁₋₃ alkyl) or C₃₋₆ cycloalkyl, optionally substituted with OH or halo (preferably F, e.g., monofluoro, difluoro, or trifluoro);
(d) —CO₂R^{aa}, —O(C═O)R^{aa}, —C₁₋₆ alkyl-CO₂R^{aa}, or wherein R^{aa} is H or C₁₋₃ alkyl, preferably methyl or ethyl;
(e) —N(R^{ca})C(═O)R^{cb}, —N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle (e.g., piperidinyl, pyrrolidinyl, and morpholinyl);
(f) —N(R^{ab})(R^{ac}) or —SO₂N(R^{ab})(R^{ac}), wherein R^{ab} and R^{ac} are independently H, OH (R^{ab} and R^{ac} are not both OH), C₁₋₃ alkyl, C₁₋₆ hydroxyalkyl, or C₁₋₆ alkyl (preferably C₁₋₃ alkyl), or R^{ab} and R^{ac} taken together with the nitrogen they are attached to form a 3, 4, 5 or 6-membered heterocycle;
(g)C₁₋₆ alkoxy optionally substituted with 1, 2 or 3 substituents each being independently chosen from the group consisting of:

(A) hydroxyl;

(B) halo (e.g., F, Cl, Br, I);

(C) —CO₂R^{aa}, —O(C═O)R^{aa}, —C₁₋₆ alkyl-CO₂R^{aa}, or wherein R^{aa} is H or C₁₋₃ alkyl, preferably methyl or ethyl;

(D) —N(R^{ca})C(═O)R^{cb}, —N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle (e.g., piperidinyl, pyrrolidinyl, and morpholinyl);

(E) heterocycle (e.g.,

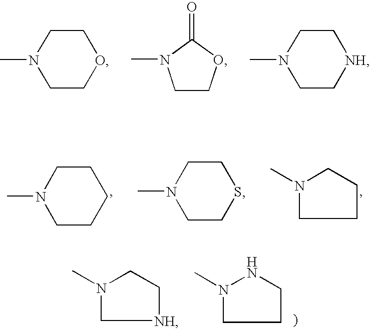

optionally substituted with 1, 2, or 3 substituents each being independently halo (e.g., F, Cl, Br, I), C₁₋₆ alkyl, or C₁₋₃ haloalkyl;

(F) heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, carboxyl, C₁₋₃ alkoxycarbonyl, C₁₋₃ hydroxyalkyl, C₁₋₃ haloalkyl, or —N(R^{ae})(R^{af}) or —SO₂N(R^{ae})(R^{af}), wherein R^{ae} and R^{af} are independently H, OH (R^{ae} and R^{af} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered heterocycle; and

(G) —N(R^{ag})(R^{ah}) where R^{ag} and R^{ah} are independently H, hydroxyl, C₁₋₆ alkyl, C₁₋₆ hydroxyalkyl, or —N(R^{ai})(R^{aj}) where R^{ai} and R^{aj} are independently H or C₁₋₃ alkyl, or R^{ag} and R^{ah} can be taken together with the nitrogen they are attached to form a 3, 4, 5 or 6-membered heterocycle, and/or R^{al} and R^{al} can be taken together with the nitrogen they are attached to form a 3, 4, 5 or 6-membered heterocycle; and

(h) —CON(R^{ak})(R^{al}) wherein R^{ak} and R^{al} are independently H, or C₁₋₆ alkyl that is optionally substituted with 1, 2, or 3 substituents each being independently

(A) hydroxyl;

(B) halo;

(C) —N(R^{am})(R^{an}) where R^{am} and R^{an} are independently H, C₁₋₃ alkyl, hydroxyl, or C₁₋₃ hydroxylalkyl;

(D) heterocycle (e.g.,

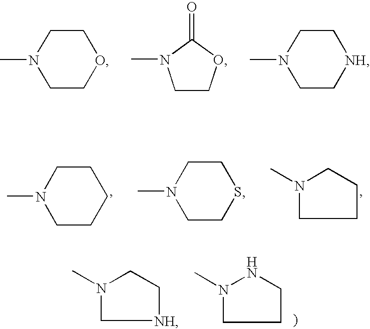

optionally substituted with 1, 2, or 3 substituents each being independently halo (e.g., F, Cl, Br, I), C₁₋₆ alkyl, or C₁₋₃ haloalkyl; and

(E) aryl or heteroaryl (preferably having at least one nitrogen atom, e.g., imidazolyl, pyridinyl, etc.), optionally substituted with 1, 2, or 3 substituents each being independent halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and Rap taken together with the nitrogen they are attached to form a 3, 4, 5 or 6-membered heterocycle.

In group (v), R⁹¹ and R⁹² at each occurrence are independently H, methyl, ethyl, or F; d is an integer of 0, 1 or 2, 3 or 4, preferably 1.

In group (vi), R¹⁰ is:

(a) heterocycle (e.g.,

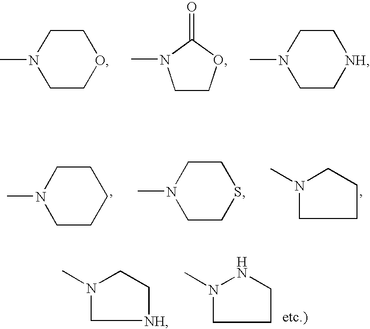

optionally substituted with 1, 2, or 3 substituents each being independently halo (e.g., F, Cl, Br, I), C₁₋₆ alkyl, or C₁₋₃ haloalkyl; or
(b) —CO₂R^{ad} where R^{ad} is H or C₁₋₃ alkyl (preferably methyl).

In group (vi), R¹⁰¹ and R¹⁰² at each occurrence are independently H, methyl, ethyl, or F; d is an integer of 0, 1 or 2, 3 or 4, preferably 1-4.

In some embodiments of the compound of Formula V, R³ is represented by

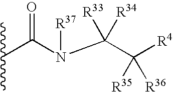

wherein R⁴, R³³, R³⁴, R³⁵, R³⁶, R³⁷ are as defined above. In specific embodiments, R³³ and R³⁴ are both methyl or together with the carbon they are attached to form a cyclopropyl. In other specific embodiments, R³⁵ and R³⁶ are both methyl or together with the carbon they are attached to form a cyclopropyl. In still other specific embodiments, both of R³⁵ and R³⁶ are F, or one F and the other H. In yet other specific embodiments, one of R³³ and R³⁴ is H and the other is methyl, and one of R³⁵ and R³⁶ is H and the other is methyl.

In some embodiments, R³ is represented by

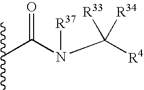

wherein R⁴, R³³, R³⁴, and R³⁷ are as defined above.

In some embodiments, R³ is represented by

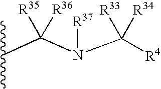

wherein R⁴, R³³, R³⁴R³⁵R³⁶, and R³⁷ are as defined above.

In preferred embodiments of the compound of the present invention according to Formula V, R³ is represented by

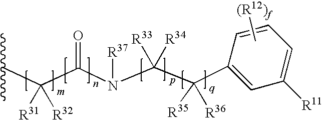

wherein m, n, p, R³¹, R³²R³³, R³⁴, R³⁵, R³⁶, and R³⁷ are as defined above;

R¹¹ is

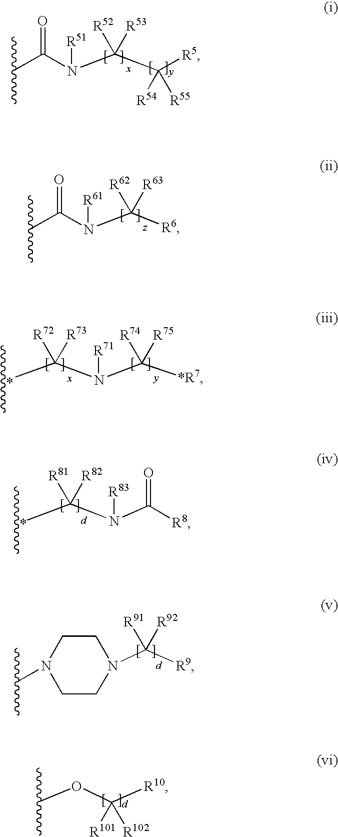

as defined herein above, or
(vii) an aryl or heteroaryl where the aryl or heteroaryl is substituted with one or more substituent groups each being independently chosen from:
(a) halo (e.g., F, Cl, Br, I);
(b) hydroxyl;
(c) C₁₋₆ alkyl (preferably C₁₋₃ alkyl) or C₃₋₆ cycloalkyl, optionally substituted with OH or halo (preferably F, e.g., monofluoro, difluoro, or trifluoro);
(d) —CO₂R^{aa}, wherein R^{aa} is H or C₁₋₃ alkyl, preferably methyl or ethyl;
(e) —N(R^{ab})(R^{ac}) or —SO₂N(R^{ab})(R^{ac}), wherein R^{ab} and R^{ac} are independently H, OH (R^{ab} and R^{ac} are not both OH), C₁₋₃ alkyl, C₁₋₆ hydroxyalkyl, or C₁₋₆ alkyl (preferably C₁₋₃ alkyl), or R^{ab} and R^{ac} taken together with the nitrogen they are attached to form a 3, 4, 5 or 6-membered heterocycle;
(f) C₁₋₆ alkoxy optionally substituted with 1, 2 or 3 substituents each being independently chosen from the group consisting of:

(A) hydroxyl;

(B) halo (e.g., F, Cl, Br, I);

(C)—CO₂R^{aa}, —O(C═O)R^{aa}, —C₁₋₆ alkyl-CO₂R^{aa}, or wherein R^{aa} is H or C₁₋₃ alkyl, preferably methyl or ethyl;

(D) —N(R^{ca})C(═O)R^{cb}, N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH (R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle (e.g., piperidinyl, pyrrolidinyl, and morpholinyl);

(E) heterocycle (e.g.,

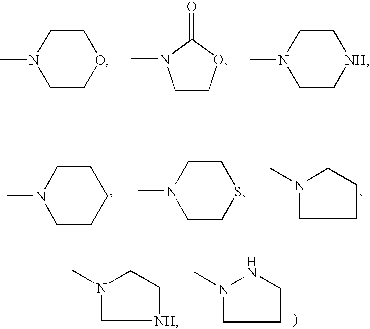

optionally substituted with 1, 2, or 3 substituents each being independently halo (e.g., F, Cl, Br, I), C₁₋₆ alkyl, or C₁₋₃ haloalkyl;

(F) heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, carboxyl, C₁₋₃ alkoxycarbonyl, C₁₋₃ hydroxyalkyl, C₁₋₃ haloalkyl, or —N(R^{ae})(R^{af}) or —SO₂N(R^{ae})(R^{af}), wherein R^{ae} and R^{af} are independently H, OH (R^{ae} and R^{af} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered heterocycle; and

(G) —N(R^{ag})(R^{ah}) where R^{ag} and R^{ah} are independently H, hydroxyl, C₁₋₆ alkyl, C₁₋₆ hydroxyalkyl, or —N(R^{al})(R^{al}) where R^{al} and R^{al} are independently H or C₁₋₃ alkyl, or R^{ag} and R^{ah} can be taken together with the nitrogen they are attached to form a 3, 4, 5 or 6-membered heterocycle, and/or R^{al} and R^{al} can be taken together with the nitrogen they are attached to form a 3, 4, 5 or 6-membered heterocycle; and

(g) —CON(R^{ak})(R^{al}) wherein R^{ak} and R^{al} are independently H, or C₁₋₆ alkyl that is optionally substituted with 1, 2, or 3 substituents each being independently

(A) hydroxyl;

(B) halo;

(C)—N(R^{am})(R^{an}) where R^{am} and R^{an} are independently H, C₁₋₃ alkyl, hydroxyl, or C₁₋₃ hydroxylalkyl;

(D) heterocycle (e.g.,

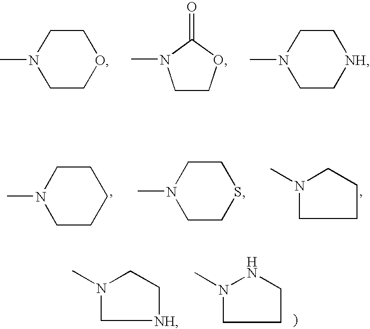

optionally substituted with 1, 2, or 3 substituents each being independently halo (e.g., F, Cl, Br, I), C₁₋₆ alkyl, or C₁₋₃ haloalkyl; and

(E) aryl or heteroaryl (preferably having at least one nitrogen atom, e.g., imidazolyl, pyridinyl, etc.), optionally substituted with 1, 2, or 3 substituents each being independent halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and R^{ap} taken together with the nitrogen they are attached to form a 3, 4, 5 or 6-membered heterocycle; and R¹² at each occurrence is independently halo (e.g., F, Cl, Br), hydroxyl, C₁₋₆ alkyl, C₁₋₆ alkoxy, carboxyl, and alkoxycarbonyl; and f is an integer of 0, 1, 2, or 3 or 4, preferably 1 or 2. In preferred embodiments, R¹² is para to R¹¹, and/or ortho to R¹¹ counterclockwise.

In some embodiments, in the compounds of the present invention according to Formula V, R³ is represented by:

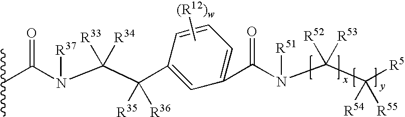

wherein R⁵, R¹², R³³, R³⁴, R³⁵, R³⁶, R³⁷, R⁵¹, R⁵², R⁵³, R⁵⁴, R⁵⁵, and x, y, and w are as defined above, preferably R¹² is para to R¹¹, and/or ortho to R¹¹ counterclockwise.

A pharmaceutically acceptable salt of the compound of the present invention is exemplified by a salt with an inorganic acid and/or a salt with an organic acid that are known in the art. In addition, pharmaceutically acceptable salts include acid salts of inorganic bases, such as salts containing alkaline cations, alkaline earth cations, as well as acid salts of organic bases. Their hydrates, solvates, and the like are also encompassed in the compound of the present invention. In addition, N-oxide compounds are also encompassed in the compound of the present invention.

Additionally, the compounds of the present invention can contain asymmetric carbon atoms and can therefore exist in racemic and optically active forms. Thus, optical isomers or enantiomers, racemates, and diastereomers are also encompassed. The methods of present invention include the use of all such isomers and mixtures thereof. The present invention encompasses any isolated racemic or optically active form of compounds described above, or any mixture thereof, which possesses anti-viral activity.

In preferred embodiments of the invention, the stereochemistry of the compounds is equivalent to that of the natural product from which the compound was derived (e.g., betulinic acid).

In preferred embodiments, compounds are provided according to any of the above formulae and having an IC₅₀ of less than 2,500 nM, 500 nM, 300 nM, 200 nM, preferably less than 100 nM, and most preferably less than 50 nM, as determined in the P4-MAGI assay in Example 2. Such activities of the exemplary compounds are provided in Table 1 below.

Unless specifically stated otherwise or indicated by a bond symbol (dash or double dash), the connecting point to a recited group will be on the right-most stated group. Thus, for example, a hydroxyalkyl group is connected to the main structure through the alkyl and the hydroxyl is a substituent on the alkyl.

The term “bioisostere”, as used herein, generally refers to compounds or moieties that have chemical and physical properties producing broadly similar biological properties. For example, —COOH bioisosteres include, but are not limited to, a carboxylic acid ester, amide, tetrazole, oxadiazole, isoxazole, hydroxythiadiazole, thiazolidinedione, oxazolidinedione, sulfonamide, sulfonylcarboxamide, phosphonic acid, phosphonamide, phosphinic acid, sulfonic acid, acyl sulfonamide, mercaptoazole, and cyanamide.

As used herein, the term “alkyl” as employed herein by itself or as part of another group refers to a saturated aliphatic hydrocarbon straight chain or branched chain group having, unless otherwise specified, 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc. up to and including 20 carbon atoms). An alkyl group may be in unsubstituted form or substituted form with one or more substituents (generally one to three substitutents except in the case of halogen substituents, e.g., perchloro). For example, a C₁₋₆ alkyl group (“lower alkyl”) refers to a straight or branched aliphatic group containing 1 to 6 carbon atoms (e.g., methyl, ethyl, propyl, isopropyl, sec-butyl, tert-butyl, isobutyl, n-butyl, 3-pentyl, and hexyl), which may be optionally substituted.

The term “alkenyl” as employed herein by itself or as part of another group means a straight or branched chain radical of 2-10 carbon atoms, unless the chain length is limited thereto, including at least one double bond between two of the carbon atoms in the chain. An alkenyl group may be in unsubstituted form or substituted form with one or more substituents (generally one to three substitutents except in the case of halogen substituents, e.g., perchloro or perfluoroalkyls). For example, a C₁₋₆ alkenyl group refers to a straight or branched chain radical containing 1 to 6 carbon atoms and having at least one double bond between two of the carbon atoms in the chain (e.g., ethenyl, 1-propenyl, 2-propenyl, 2-methyl-1-propenyl, 1-butenyl and 2-butenyl, which may be optionally substituted).

The term “alkynyl” as used herein by itself or as part of another group means a straight or branched chain radical of 2-10 carbon atoms, unless the chain length is limited thereto, wherein there is at least one triple bond between two of the carbon atoms in the chain. An alkynyl group may be in unsubstituted form or substituted form with one or more substituents (generally one to three substitutents except in the case of halogen substituents, e.g., perchloro or perfluoroalkyls). For example, a C₁₋₆ alkynyl group refers to a straight or branched chain radical containing 1 to 6 carbon atoms and having at least one triple bond between two of the carbon atoms in the chain (e.g., ethynyl, 1-propynyl, 1-methyl-2-propynyl, 2-propynyl, 1-butynyl and 2-butynyl, which may be optionally substituted).

The term “carbocycle” as used herein by itself or as part of another group means cycloalkyl and non-aromatic partially saturated carbocyclic groups such as cycloalkenyl and cycloalkynyl. A carbocycle may be in unsubstituted form or substituted form with one or more substituents so long as the resulting compound is sufficiently stable and suitable for the treatment method of the present invention.

The term “cycloalkyl” as used herein by itself or as part of another group refers to a fully saturated 3- to 8-membered cyclic hydrocarbon ring (i.e., a cyclic form of an unsubstituted alkyl) alone (“monocyclic cycloalkyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkyl”). Thus, a cycloalkyl may exist as a monocyclic ring, bicyclic ring, polycyclic or a spiral ring. When a cycloalkyl is recited as a substituent on a chemical entity, it is intended that the cycloalkyl moiety is attached to the entity through a carbon atom within the fully saturated cyclic hydrocarbon ring of the cycloalkyl. In contrast, a substituent on a cycloalkyl can be attached to any carbon atom of the cycloalkyl. A cycloalkyl may be in unsubstituted form or substituted form with one or more substituents so long as the resulting compound is sufficiently stable and suitable for the treatment method of the present invention. Examples of cycloalkyl groups include cyclopropyl, cyclobutyl, cyclopentyl, cyclohexyl and cycloheptyl.

The term “cycloalkenyl” as used herein by itself or as part of another group refers to a non-aromatic partially saturated 3- to 8-membered cyclic hydrocarbon ring (i.e., a cyclic form of an unsubstituted alkenyl) alone (“monocyclic cycloalkenyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkenyl”). Thus, a cycloalkenyl may exist as a monocyclic ring, bicyclic ring, polycyclic or a spiral ring. When a cycloalkenyl is recited as a substituent on a chemical entity, it is intended that the cycloalkenyl moiety is attached to the entity through a carbon atom within the fully saturated cyclic hydrocarbon ring of the cycloalkenyl. In contrast, a substituent on a cycloalkenyl can be attached to any carbon atom of the cycloalkyl. A cycloalkenyl group may be unsubstituted or substituted with one or more substitutents. Examples of cycloalkenyl groups include cyclopentenyl, cycloheptenyl and cyclooctenyl.

The term “heterocycle” (or “heterocyclyl” or “heterocyclic”) as used herein by itself or as part of another group means a saturated or partially saturated 3-7 membered non-aromatic cyclic ring formed with carbon atoms and from one to four heteroatoms independently selected from the group consisting of O, N, and S, wherein the nitrogen and sulfur heteroatoms can be optionally oxidized, and the nitrogen can be optionally quaternized (“monocyclic heterocycle”). The term “heterocycle” also encompasses a group having the non-aromatic heteroatom-containing cyclic ring above fused to another monocyclic cycloalkyl, cycloalkynyl, cycloalkenyl, heterocycle, aryl or heteroaryl ring (i.e., sharing an adjacent pair of carbon atoms with such other rings) (“polycyclic heterocylce”). Thus, a heterocycle may exist as a monocyclic ring, bicyclic ring, polycyclic or a spiral ring. When a heterocycle is recited as a substituent on a chemical entity, it is intended that the heterocycle moiety is attached to the entity through an atom within the saturated or partially saturated ring of the heterocycle. In contrast, a substituent on a heterocycle can be attached to any suitable atom of the heterocycle. In a “saturated heterocycle” the non-aromatic heteroatom-containing cyclic ring described above is fully saturated, whereas a “partially saturated heterocyle” contains one or more double or triple bonds within the non-aromatic heteroatom-containing cyclic ring regardless of the other ring it is fused to. A heterocycle may be in unsubstituted form or substituted form with one or more substituents so long as the resulting compound is sufficiently stable and suitable for the treatment method of the present invention.

Some examples of saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “aryl” by itself or as part of another group means an all-carbon aromatic ring with up to 7 carbon atoms in the ring (“monocyclic aryl”). In addition to monocyclic aromatic rings, the term “aryl” also encompasses a group having the all-carbon aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic aryl”). When an aryl is recited as a substituent on a chemical entity, it is intended that the aryl moiety is attached to the entity through an atom within the all-carbon aromatic ring of the aryl. In contrast, a substituent on an aryl can be attached to any suitable atom of the aryl. Examples, without limitation, of aryl groups are phenyl, naphthalenyl and anthracenyl. An aryl may be in unsubstituted form or substituted form with one or more substituents so long as the resulting compound is sufficiently stable and suitable for the treatment method of the present invention.

The term “heteroaryl” as employed herein refers to a stable aromatic ring having up to 7 atoms with 1, 2, 3 or 4 heteroactoms which are oxygen, nitrogen or sulfur or a combination thereof (“monocyclic heteroaryl”). In addition to monocyclic hetero aromatic rings, the term “heteroaryl” also encompasses a group having the monocyclic hetero aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic heteroaryl”). When a heteroaryl is recited as a substituent on a chemical entity, it is intended that the heteroaryl moiety is attached to the entity through an atom within the hetero aromatic ring of the heteroaryl. In contrast, a substituent on a heteroaryl can be attached to any suitable atom of the heteroaryl. A heteroaryl may be in unsubstituted form or substituted form with one or more substituents so long as the resulting compound is sufficiently stable and suitable for the treatment method of the present invention.

Useful heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

As used herein, the term “halo” refers to chloro, fluoro, bromo, and iodo.

As used herein, the term “hydro” refers to a hydrogen atom (—H group).

As used herein, the term “hydroxy” refers to an —OH group.

As used herein, unless otherwise specified, the term “alkoxy” refers to a —O—C₁₋₁₂ alkyl.

As used herein, the term “cycloalkyloxy” refers to an —O-cycloalkyl group.

As used herein, the term “aryloxy” refers to an —O-aryl group.

As used herein, the term “heteroaryloxy” refers to both an —O-heteroaryl group.

Useful acyloxy groups are any C₁₋₆ acyl (alkanoyl) attached to an oxy (—O—) group, e.g., formyloxy, acetoxy, propionoyloxy, butanoyloxy, pentanoyloxy and hexanoyloxy. An acyloxy group may be unsubstituted or substituted form with one or more substituents so long as the resulting compound is sufficiently stable and suitable for the treatment method of the present invention.

As used herein, the term “mercapto” group refers to an —SH group.

As used herein, the term “alkylthio” group refers to an —S-alkyl group.

As used herein, the term “arylthio” group refers to both an —S-aryl group.

The term “arylalkyl” is used herein to mean an above-defined alkyl group substituted by an aryl group defined above. Examples of arylalkyl groups include benzyl, phenethyl and naphthylmethyl, etc. An arylalkyl group may be unsubstituted or substituted with one or more substituents so long as the resulting compound is sufficiently stable and suitable for the treatment method of the present invention.

The term “heteroarylalkyl” is used herein to mean an alkyl group defined above substituted by any heteroaryl groups. A heteroarylalkyl may be unsubstituted or substituted with one or more substituents so long as the resulting compound is sufficiently stable and suitable for the treatment method of the present invention.

The term “arylalkenyl” is used herein to mean an alkenyl group defined above substituted by any aryl groups defined above.

The term “heteroarylalkenyl” is used herein to mean any of the above-defined alkenyl groups substituted by any of the above-defined heteroaryl groups.

The term “arylalkynyl” is used herein to mean any of the above-defined alkynyl groups substituted by any of the above-defined aryl groups.

The term “heteroarylalkynyl” is used herein to mean any of the above-defined alkynyl groups substituted by any of the above-defined heteroaryl groups.

The term “aryloxy” is used herein to mean aryl-O— wherein aryl is as defined above. Useful aryloxy groups include phenoxy and 4-methylphenoxy.

The term “heteroaryloxy” is used herein to mean heteroaryl-O— wherein heteroaryl is as defined above.

The term “arylalkoxy” is used herein to mean an alkoxy group substituted by an aryl group as defined above. Useful arylalkoxy groups include benzyloxy and phenethyloxy.

“Heteroarylalkoxy” is used herein to mean any of the above-defined alkoxy groups substituted by any of the above-defined heteroaryl groups.

Haloalkyl means an alkyl group substituted by one or more (1, 2, 3, 4, 5 or 6) fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Useful acylamino (acylamido) groups are any C₁₋₆ acyl (alkanoyl) attached to an amino nitrogen which is in turn attached to the main structure, e.g., acetamido, chloroacetamido, propionamido, butanoylamido, pentanoylamido and hexanoylamido, as well as aryl-substituted C₁₋₆ acylamino groups, e.g., benzoylamido, and pentafluorobenzoylamido.

As used herein, the term “carbonyl” group refers to a —C(═O)R″ group, where R″ is selected from the group consisting of hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has a “═O” bonded to it; i.e. one of the ring carbon atoms is a —C(═O)-group.

As used herein, the term “thiocarbonyl” group refers to a —C(═S)R″ group, with R″ as defined herein.

As used herein, the term “O-carboxy” group refers to a R″C(═O)O-group, with R″ as defined herein.

As used herein, the term “C-carboxy” group refers to a —C(═O)OR″ groups with R″ as defined herein.

As used herein, the term “ester” is a C-carboxy group, as defined herein, wherein R″ defined above except that it is not hydro (e.g., methyl, ethyl, lower alkyl).

As used herein, the term “C-carboxy salt” refers to a —C(═O)O⁻M⁺ group wherein M⁺ is selected from the group consisting of lithium, sodium, magnesium, calcium, potassium, barium, iron, zinc and quaternary ammonium.

As used herein, the term “acetyl” group refers to a —C(═O)CH₃ group.

As used herein, the term “carboxyalkyl” refers to —(CH₂)ᵣC(═O)OR″ wherein r is 1-6 and R″ is as defined above.

As used herein, the term “carboxyalkyl salt” refers to a —(CH₂)ᵣC(═O)O⁻M⁺ wherein M⁺ is selected from the group consisting of lithium, sodium, potassium, calcium, magnesium, barium, iron, zinc and quaternary ammonium.

As used herein, the term “carboxylic acid” refers to a C-carboxy group in which R″ is hydro.

As used herein, the term “trihalomethanesulfonyl” refers to a X₃ CS(═O)₂— group with X is a halo as defined above.

As used herein, the term “cyano” refers to a —C≡N group.

As used herein, the term “cyanato” refers to a —CNO group.

As used herein, the term “isocyanato” refers to a —NCO group.

As used herein, the term “thiocyanato” refers to a —CNS group.

As used herein, the term “isothiocyanato” refers to a —NCS group.

As used herein, the term “sulfinyl” refers to a —S(═O)R″ group, with R″ as defined herein.

As used herein, the term “sulfonyl” refers to a —S(═O)₂R″ group, with R″ as defined herein.

As used herein, the term “sulfonamido” refers to a —S(═O)₂N(R¹⁷)(R¹⁸), with R¹⁷ and R¹⁸ as defined herein.

As used herein, the term “trihalomethanesulfonamido” refers to a X₃CS(═O)₂ NR¹⁷-group with X is halo as defined above and R¹⁷ as defined herein.

As used herein, the term “O-carbamyl” refers to a —OC(═O)N(R¹⁷)(R¹⁸) group with R¹⁷ and R¹⁸ as defined herein.

As used herein, the term “N-carbamyl” refers to a R¹⁸OC(═O)NR¹⁷-group, with R¹⁷ and R¹⁸ as defined herein.

As used herein, the term “O-thiocarbamyl” refers to a —OC(═S)N(R¹⁷)(R¹⁸) group with R¹⁷ and R¹⁸ as defined herein.

As used herein, the term “N-thiocarbamyl” refers to a R¹⁷OC(═S)NR¹⁸-group, with R¹⁷ and R¹⁸ as defined herein.

As used herein, the term “amino” refers to an —N(R¹⁷)(R¹⁸) group, with R¹⁷ and R¹⁸ as defined herein.

As used herein, the term “aminoalkyl” refers to a moiety wherein an amino group as defined herein attached through the nitrogen atom to an alkyl group as defined above.

As used herein, the term “C-amido” refers to a —C(═O)N(R¹⁷)(R¹⁸) group with R¹⁷ and R¹⁸ as defined herein. An “N-amido” refers to a R¹⁷C(═O)NR¹⁸-group with R¹⁷ and R¹⁸ as defined herein.

As used herein, the term “C-amidoalkyl” refers to a —C₁₋₆ alkyl-CO₂N(R¹⁷)(R¹⁸) group with R¹⁷ and R¹⁸ as defined herein.

As used herein, the term “nitro” refers to a —NO₂ group.

As used herein, the term “quaternary ammonium” refers to a —⁺N(R¹⁷)(R¹⁸)(R¹⁹) group wherein R¹⁷, R¹⁸, and R¹⁹ are as defined herein.

R¹⁷, R¹⁸, and R¹⁹ are independently selected from the group consisting of hydro and unsubstituted lower alkyl.

As used herein, the term “methylenedioxy” refers to a —OCH₂O— group wherein the oxygen atoms are bonded to adjacent ring carbon atoms.

As used herein, the term “ethylenedioxy” refers to a —OCH₂CH₂O— group wherein the oxygen atoms are bonded to adjacent ring carbon atoms.

The present invention provides methods for treating viral infection, particularly HIV infection, delaying the onset of HIV infection, treating AIDS, delay the onset of AIDS, by treating a patient (either a human or another animal) in need of the treatment, with a compound of the present invention. In preferred embodiments of the methods, compounds having an IC₅₀ of less than 2,500 nM, 500 nM, 300 nM, 200 nM, preferably less than 100 nM, and most preferably less than 50 nM, as determined in the P4-MAGI assay in Example 2, are used. Such activities of the exemplary compounds are provided in Table 1 below.

As used herein, the phrase “treating . . . with . . . a compound” means either administering the compound to cells or an animal, or administering to cells or an animal the compound or another agent to cause the presence or formation of the compound inside the cells or the animal. Preferably, the methods of the present invention comprise administering to cells in vitro or to a warm-blood animal, particularly mammal, more particularly a human a pharmaceutical composition comprising an effective amount of a compound according to the present invention.

As used herein, the term “HIV infection” generally encompasses infection of a host animal, particularly a human host, by any member(s) of the human immunodeficiency virus (HIV) family of retroviruses including, but not limited to, HIV-1, HIV-2, HIV I (also known as HTLV-III), HIV II (also known as LAV-1), HIV III (also known as LAV-2), and the like. “HIV” can be used herein to refer to any strains, forms, subtypes, clades and variations in the HIV family. Thus, treating HIV infection will encompass the treatment of a person who is a carrier of any of the HIV family of retroviruses or a person who is diagnosed of active AIDS, as well as the treatment or delay the onset of AIDS or AIDS-related conditions in such persons. A carrier of HIV may be identified by any methods known in the art. For example, a person can be identified as HIV carrier on the basis that the person is anti-HIV antibody positive, or is HIV-positive, or has symptoms of AIDS. That is, “treating HIV infection” should be understood as treating a patient who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD4 T-cells), and AIDS (which is defined by more serious AIDS-defining illnesses and/or a decline in the circulating CD4 T-cell count to below a level that is compatible with effective immune function).

As used herein, the term “delaying the onset of HIV infection” means treating an individual who (1) is at risk of infection by HIV, or (2) is suspected of infection by HIV or of exposure to HIV, or (3) has suspected past exposure to HIV, to delay the onset of acute primary infection syndrome by at least three months. As is known in the art, clinical findings typically associated with acute primary infection syndrome may include an influenza-like illness with fevers, malaise, nausea/vomiting/diarrhea, pharyngitis, lymphadenopathy, myalgias, and neurologic symptoms such as headache, encephalitis, etc. The individuals at risk may be people who perform any of following acts: contact with HIV-contaminated blood, blood transfusion, exchange of body fluids, “unsafe” sex with an infected person, accidental needle stick, injection of drug with contaminated needles or syringes, receiving a tattoo or acupuncture with contaminated instruments, or transmission of the virus from a mother to a baby during pregnancy, delivery or shortly thereafter. The term “delaying the onset of HIV infection” also encompasses treating a person who has not been diagnosed as having HIV infection but is believed to be at risk of infection by HIV, or has been exposed to HIV through contaminated blood, etc.

In addition, the term “delay the onset of AIDS” means delaying the onset of AIDS (which is characterized by more serious AIDS-defining illnesses and/or a decline in the circulating CD4 cell count to below a level that is compatible with effective immune function, i.e. below about 200/μl) and/or AIDS-related conditions, by treating an individual (1) at risk of infection by HIV, or suspected of being infected with HIV, or (2) having HIV infection but not AIDS, to delay the onset of AIDS by at least six months. Individuals at risk of HIV infection may be those who are suspected of past exposure, or considered to be at risk of present or future exposure, to HIV by, e.g., contact with HIV-contaminated blood, blood transfusion, transplantation, exchange of body fluids, “unsafe” sex with an infected person, accidental needle stick, receiving a tattoo or acupuncture with contaminated instruments, or transmission of the virus from a mother to a baby during pregnancy, delivery or shortly thereafter.

The term “treating AIDS” means treating a patient who exhibits more serious AIDS-defining illnesses and/or a decline in the circulating CD4 cell count to below a level that is compatible with effective immune function (typically below about 200/μl). The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

For example, a carrier of HIV can be identified by conventional diagnostic techniques known in the art, and the identified carrier can be treated with a compound of the present invention, preferably in a pharmaceutical composition having a pharmaceutically acceptable carrier.

In one aspect, the present invention provides methods for combination therapy for treating viral infection, particularly HIV infection, delaying the onset of HIV infection, treating AIDS, delay the onset of AIDS, by treating a patient (either a human or another animal) in need of the treatment, with a compound of the present invention together with one or more other anti-HIV agents. Such other anti-HIV agents include those agents targeting a viral protein such as viral protease, reverse transcriptase, integrase, envelope protein (e.g., gp120 and gp41 for anti-fusion or homolog thereof). Thus, examples of such other antiviral compounds include, but are not limited to, protease inhibitors, nucleoside reverse transcriptase inhibitors, non-nucleoside reverse transcriptase inhibitors, integrase inhibitors, fusion inhibitors, and a combination thereof. In the combination therapy, the compound of the present invention can be administered separately from, or together with the one or more other anti-HIV agents. Thus, the present invention also provides a composition comprising a therapeutically effective amount of a compound according to the present invention and one or more the above-described other anti-HIV agents, and optionally a pharmaceutically acceptable carrier.

In another aspect, the present invention also provides a method of treating cancer which comprises treating a patient in need of such treatment with a compound of the present invention. That is, a compound of the present invention can be used in the manufacture of a medicament useful for the treatment of cancer.

In another aspect, the present invention further provides a medicament or a pharmaceutical composition having a therapeutically or prophylactically effective amount of a compound according to the present invention.

Typically, compounds according to the present invention can be effective at an amount of from about 0.01 μg/kg to about 100 mg/kg per day based on total body weight. The active ingredient may be administered at once, or may be divided into a number of smaller doses to be administered at predetermined intervals of time. The suitable dosage unit for each administration can be, e.g., from about 1 μg to about 2000 mg, preferably from about 5 μg to about 1000 mg. In the case of combination therapy, a therapeutically effective amount of one or more other antiviral compounds can be administered in a separate pharmaceutical composition, or alternatively included in the pharmaceutical composition according to the present invention which contains a compound according to the present invention. The pharmacology and toxicology of many of such other antiviral compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In the pharmaceutical compositions, the active agents can be in any pharmaceutically acceptable salt form. As used herein, the term “pharmaceutically acceptable salts” refers to the relatively non-toxic, organic or inorganic salts of the active compounds, including inorganic or organic acid addition salts of the compound.

For oral delivery, the active compounds can be incorporated into a formulation that includes pharmaceutically acceptable carriers such as binders, lubricants, disintegrating agents, and sweetening or flavoring agents, all known in the art. The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels are biodegradable or biosorbable. See, e.g., Phillips et al., J. Pharmaceut. Sci., 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated, so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

### EXAMPLES

Synthesis of compounds of the present invention can be accomplished according to the following general synthetic routes. See Table 1 for representative structures and relevant characterization data.

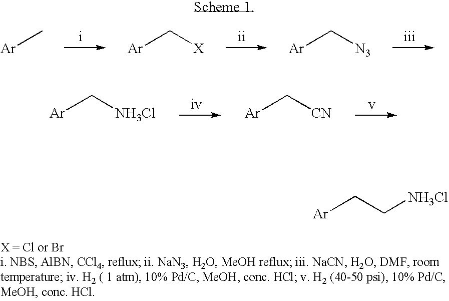

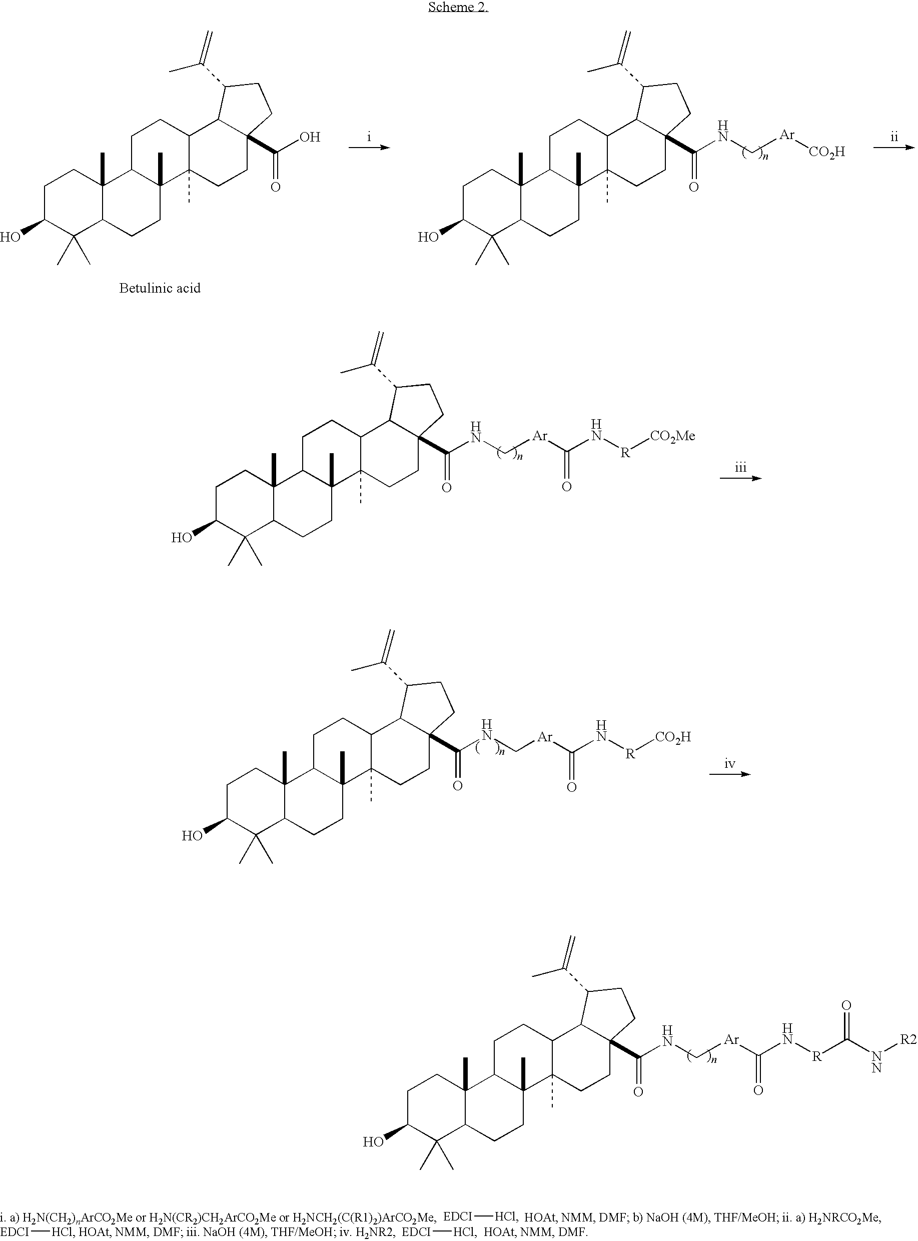

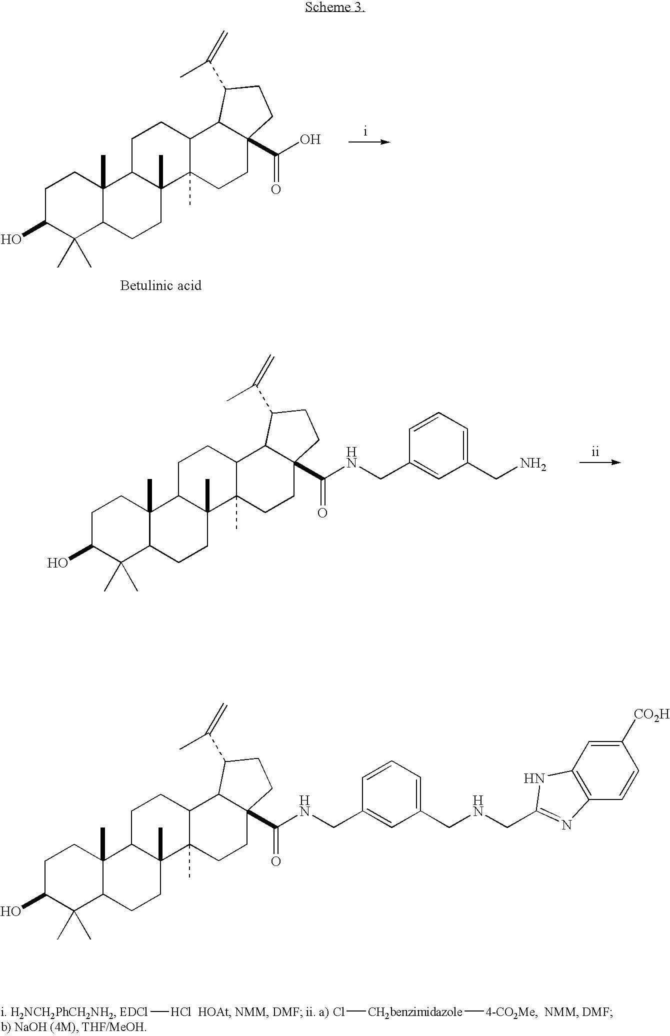

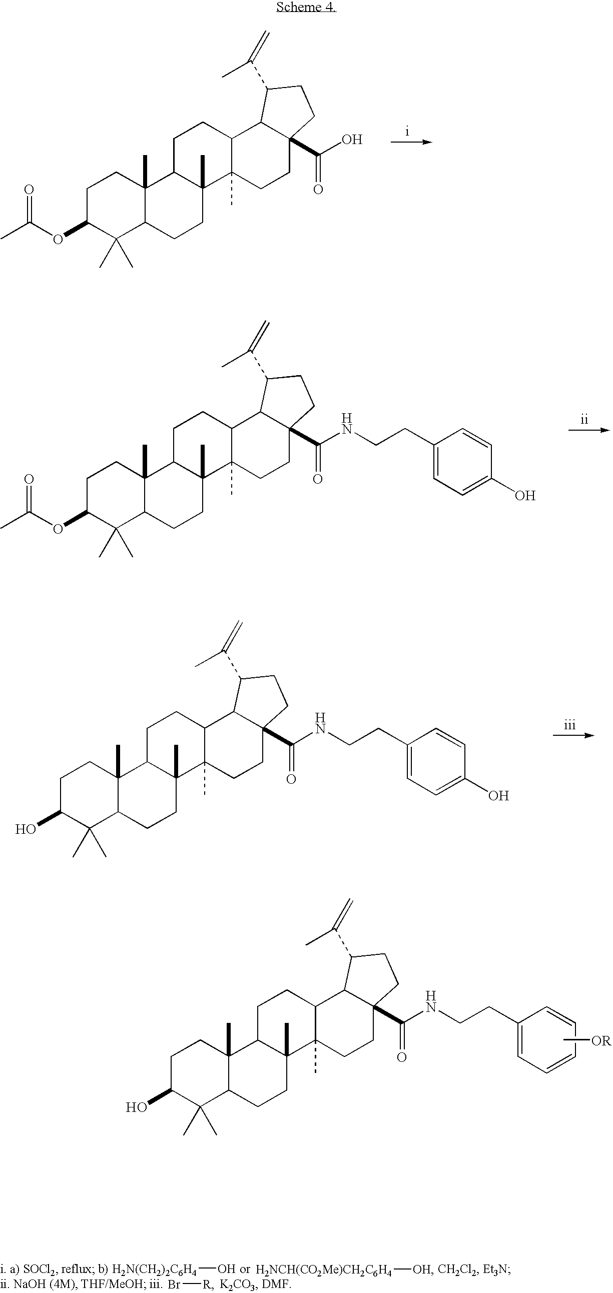

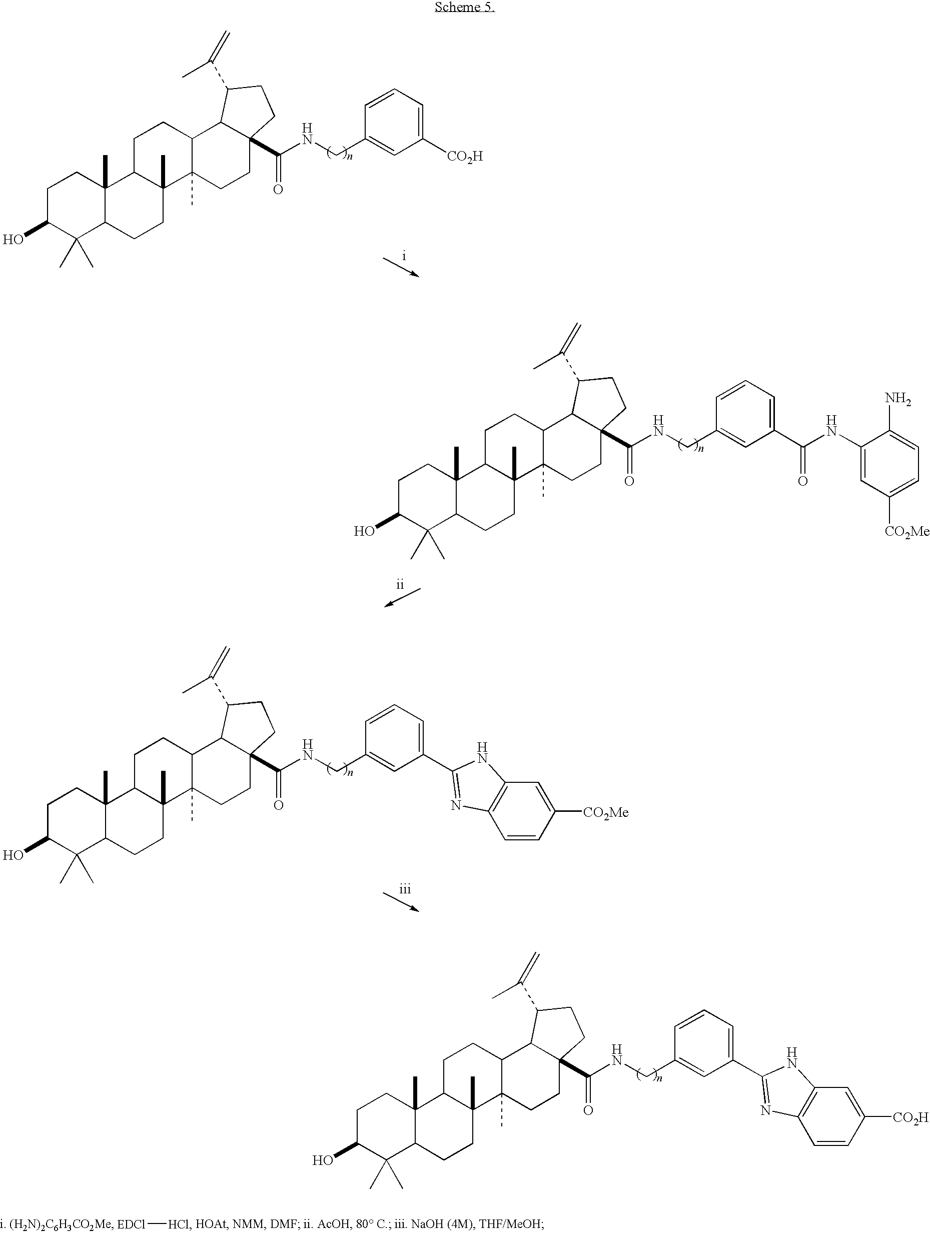

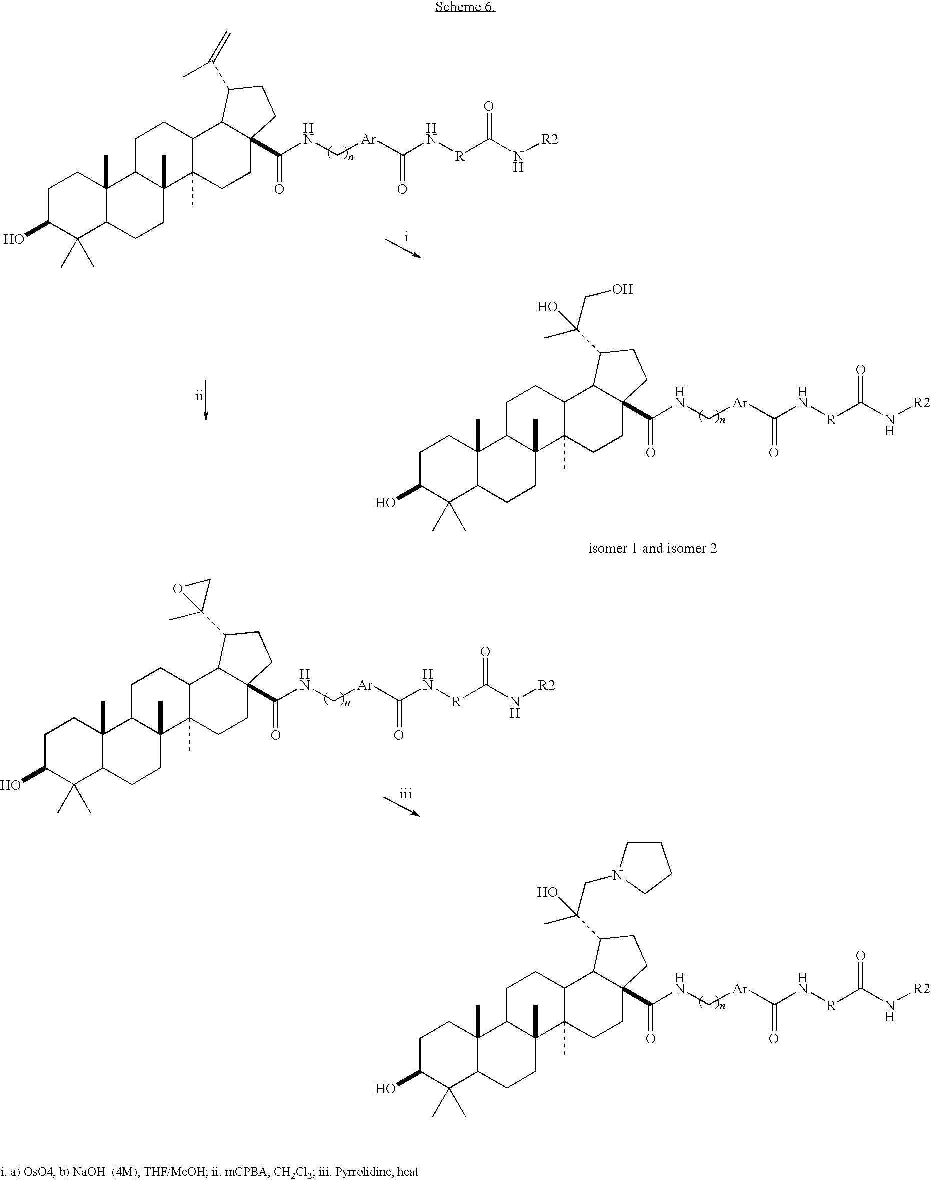

General procedure for HPLC purification: Samples were dissolved in DMSO (˜50 mg/mL), and purified on a Phenomenex Synergi Hydro-RP (00G-4376-P0) HPLC column (250×21.2 mm, 10μ sphere size, 80 Å pore size), the solvent system was 50-90% acetonitrile in water (0.01% trifluoroacetic acid), run isocratic for up to 25 minutes. Fraction collection was based on absorption at 203λ.

### Representative Experimental Procedures

### Synthesis of Aryl Ethyl and Aryl Methyl Amines,

### Represenative Bromination Procedure, Scheme 1, Step i;

3-Bromomethyl-4-fluoro-benzoic acid methyl ester: To a solution of methyl 4-fluoro-3-methyl-benzoate (3 g, 17.84 mmol) in CCl₄ (40 mL) was added N-bromo succinimide (3.8 g, 21.41 mmol) followed by AIBN (0.29 g, 1.78 mmol) at room temperature. The mixture was then heated at reflux temperature for 18 h, cooled and filtered. The filtrate was concentrated, recovered in Et₂O (75 mL) and washed with H₂O (3×20 mL), saturated NaCl solution (2×20 mL), and then dried over MgSO₄. Filtration and removal of solvent under reduced pressure gave 3.55 g of colorless oil that was used without further purification.

The following benzyl and aryl methyl halides were prepared according to this procedure:
methyl 2-bromomethyl-benzoate
methyl 4-bromomethyl-3-fluoro-benzoate
methyl 5-bromomethyl-2-fluoro-benzote
methyl 4-bromomethyl-2,3-difluoro-benzoate
methyl 4-bromomethyl-2-fluoro-benzoate
methyl 3-bromomethyl-2-fluoro-benzoate
methyl 6-bromomethyl-pyridine-2-carboxylate
methyl 2-bromomethyl-isonicotinate
methyl 4-bromomethyl-pyridine-2-carboxylate
methyl 6-bromomethyl-nicotinate
   Represenative Azide Displacement and Catalytic Hydrogenation Procedures, Scheme 1, Steps ii and iii;

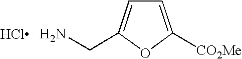

5-Aminomethyl-furan-2-carboxylic acid methyl ester hydrochloride: To a solution of methyl 5-(chloromethyl)-2-furoate (0.63 g, 3.58 mmol) in MeOH (9 mL) was added a solution of NaN₃ (0.35 g, 5.37 mmol.) in H₂O (0.7 mL) at room temperature. The mixture was then heated at reflux temperature for 2 h. The reaction mixture was concentrated then recovered in Et₂O (30 mL) and H₂O (20 mL). The organic layer was separated, washed with saturated NaCl solution (20 mL), and then dried over MgSO₄. Filtration and removal of solvent under reduced pressure gave colorless oil. The crude material was dissolved in a mixture of MeOH (10 mL) and concentrated HCl (0.36 mL) and 10% Pd/C (0.15 g) was added at room temperature. The mixture was placed under hydrogen atmosphere (1 atm) for 2.5 h. Catalyst was removed by filtration through Celite; the pad was washed with MeOH, and the pale yellow solution was concentrated under reduced pressure to give a yellow solid. Trituration with diethyl ether and drying under vacuum gave the desired product that was used without further purification.

The hydrochloride salts listed below were prepared according to this procedure;
methyl 3-aminomethyl-benzoate
methyl 2-aminomethyl-benzoate
methyl 4-aminomethyl-3-fluoro-benzoate
methyl 5-aminomethyl-2-fluoro-benzoate
methyl 4-aminomethyl-2,3-difluoro-benzoate
methyl 4-aminomethyl-2-fluoro-benzoate
methyl 3-aminomethyl-2-fluoro-benzoate
methyl 6-aminomethyl-pyridine-2-carboxylate
methyl 2-aminomethyl-isonicotinate
methyl 4-aminomethyl-pyridine-2-carboxylate
methyl-6-aminomethyl-nicotinate

### Represenative Cyanide Displacement and Catalytic Hydrogenation Procedures, Scheme 1, Steps iv and v;

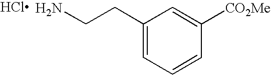

3-(2-Aminoethyl)benzoic acid methyl ester hydrochloride: To a solution of methyl 3-(bromomethyl)benzoate (1.0 g, 4.37 mmol) in DMF (9 mL) was added a solution of NaCN (0.26 g, 1.2 equiv.) in H₂O (1 mL) at room temperature. The mixture was stirred for 2 h at room temperature. The reaction mixture was diluted with EtOAc (30 mL) and H₂O (20 mL). The organic layer was washed with H₂O (3×20 mL) and saturated LiCl solution (20 mL), and then dried over MgSO₄ and concentrated under reduced pressure to give sticky oil. The crude material was dissolved in CH₂Cl₂ (20 mL) and MeOH (20 mL) and 10% Pd/C (0.5 g) and conc. HCl (1.5 mL) were added at room temperature. The mixture was placed under hydrogen gas (50 psi) on a Parr hydrogenation apparatus for 10 h, filtered through a pad of Celite and the pad was washed with 20% MeOH in CH₂Cl₂. The pale yellow solution was concentrated under reduced pressure to give a crude product that was suspended in MeOH (5 mL) and diluted with diethyl ether (100 mL) to give a white precipitate. The precipitate was collected by filtration on sintered glass, washed with 30 mL of Et₂O and dried under suction for 1 h to give the title compound (0.74 g, 78%).
Methyl 2-(2-aminoethyl)benzoate hydrochloride: 2-Cyanomethylbenzoic acid methyl ester (1.0 g, 5.71 mmol) was dissolved in CH₂Cl₂ (20 mL) and MeOH (20 mL) and then 10% Pd/C (0.5 g) and conc. HCl (1.5 mL) were added at room temperature. The mixture was placed under 50 psi of H₂ gas on the Parr hydrogenator for 10 h and then filtered through a pad of Celite and the pad was washed with 20% MeOH in CH₂Cl₂. The pale yellow solution was concentrated under reduced pressure to give crude material which was suspended in MeOH (5 mL) and then diluted with Et₂O (100 mL) to give a white precipitate. The precipitate was filtered on sintered glass filter and washed with Et₂O (30 mL) and dried with suction for 1 h to give the title compound (1.07 g, 87%).

The hydrochloride salts listed below were prepared according to this procedure;
methyl 4-(2-aminoethyl)-benzoate
methyl 3-(2-aminoethyl)-2-fluoro-benzoate
methyl 5-(2-aminoethyl)-2-fluoro-benzoate
methyl 4-(2-aminoethyl)-2,3-difluoro-benzoate
methyl 4-(2-aminoethyl)-2-fluoro-benzoate
methyl 4-(2-aminoethyl)-3-fluoro-benzoate
methyl 3-(2-amino-1-methylethyl)benzoate.

### Synthesis of Miscellaneous Aryl Methyl and Aryl Ethyl Amines;

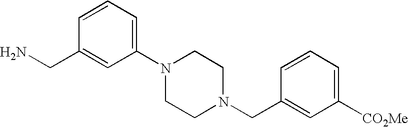

3-[4-(3-Aminomethyl-phenyl)-piperazin-1-ylmethyl]-benzoic acid methyl ester: A mixture of 3-fluoro benzonitrile and piperazine (2.5 equiv) was heated at 145° C. creating a melt. The mixture was stirred for 1.5 h after which time TLC indicated complete conversion. The reaction mixture was cooled to room temperature and the resultant solid was triturated with acetone, collected via filtration, acidified and then diluted with heptanes to again provide a precipitate. The solid was collected via filtration and dried. The derived piperazine adduct was dissolved in DMF (0.5 M) and 3-bromomethyl-benzoic acid methyl ester (1.5 equiv) and Cs₂CO₃ (1.5 equiv) were added followed by heating at 65° C. overnight. The mixture was diluted with ethyl acetate, washed with H₂O and saturated NaCl solution, dried (Na₂SO₄) and concentrated. The crude product was dissolved in a mixture of MeOH and concentrated HCl (3 equiv) and 10% Pd/C (0.15 g) were added. The mixture was placed under H₂ gas (1 atm) for 20 h. Catalyst was removed by filtration through Celite; the pad was washed with MeOH, and the pale yellow solution was concentrated under reduced pressure to give a tan solid that was used without additional purification.

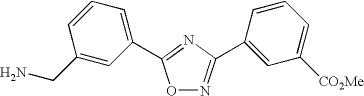

3-[5-(3-aminomethyl-phenyl)-[1,2,4]oxadiazol-3-yl]-benzoic acid methyl ester: To a solution of 3-cyano benzoic acid (1 g, 6.7 mmol) in MeOH (5 mL) and THF (15 mL) was added TMSCHN₂ (6.7 mL, 13.4 mmol, 2M solution) and the mixture was stirred at ambient temperature for 2 h. The solution was concentrated, dissolved in EtOH (15 mL) and hydroxylamine hydrochloride (0.7 g, 10.2 mmol) was added and the mixture stirred at room temperature for 5 hr. Removal of solvent provided 3-(N-hydroxycarbamimidoyl)-benzoic acid methyl ester which was used as is in the subsequent step.

To a stirred solution of 3-cyano-benzoyl chloride (94 mg, 0.51 mmol) in THF was slowly added 3-(N-hydroxycarbamimidoyl)-benzoic acid methyl ester (100 mg, 0.51 mmol) followed by drop-wise addition of ⁱPr₂NEt (0.5 mL). The mixture was then heated at 150° C. for 15 minutes under microwave irradiation. The product, 3-[5-(3-cyano-phenyl)-[1,2,4]oxadiazol-3-yl]-benzoic acid methyl ester, which had crashed out of the reaction mixture was collected by filtration. The structure was confirmed by ¹H-NMR and Mass Spectrometry. This material (50 mg, 0.16 mmol) was dissolved in a mixture of MeOH and concentrated HCl (1 drop) and 10% Pd/C (20 mg) was added. The reaction mixture was maintained under H₂ gas (1 atm) for 6-8 hrs. Removal of catalyst (filtration) and solvent provided the hydrochloride salt of 3-[5-(3-aminomethyl-phenyl)-[1,2,4]oxadiazol-3-yl]-benzoic acid methyl ester (55 mg).

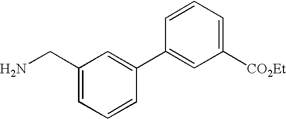

3′-Aminomethyl-biphenyl-3-carboxylic acid ethyl ester: To a stirred solution of 3-bromo-ethyl benzoate (500 mg, 2.18 mmol) in DME (20 mL) was added 3-cyano-phenyl-boronic acid (320 mg, 2.18 mmol), 2 N K₂CO₃ (1.9 mL) followed by tetrakistriphenylphosphine palladium (125 mg) and heated at reflux for 6 hrs. After cooling to room temperature, the reaction mixture was diluted with EtOAc (25 mL) and washed with H₂O (10 mL×2). The organic layer was dried over Na₂SO₄, concentrated, and the residue purified by silica gel column chromatography with EtOAc and hexane as eluents to give 3′-cyano-biphenyl-3-carboxylic acid ethyl ester (383 mg, 70% yield).

To a stirred solution of 3′-cyano-biphenyl-3-carboxylic acid ethyl ester (300 mg, 1.19 mmol) in MeOH (20 mL) was added conc. HCl (1 mL) and 10% Pd/C (100 mg). The reaction mixture was hydrogenated under H₂ gas (50 psi) for 8 h at ambient temperatures. The catalyst was filtered over Celite and was washed with MeOH. The solvent was evaporated to give the hydrochloride salt (182 mg) which was dried overnight under vacuum and used as such in the next step.

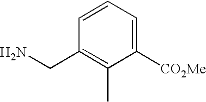

3-Aminomethyl-2-methyl-benzoic acid methyl ester: 2,3-Dimethylbenzoic acid (1.0 g, 6.66 mmol) was dissolved in DMF (15 mL) and then Cs₂CO₃ (1.5 equiv.) and MeI (1.2 equiv.) were added. The mixture was stirred for 2 h at room temperature then diluted with EtOAc and H₂O. The organic layer was washed with H₂O and a saturated LiCl solution, and then dried over MgSO₄ and concentrated under reduced pressure to give sticky oil. The oil was dissolved in CCl₄ (20 mL) and NBS (1.1 equiv.) and AIBN (0.1 equiv.) were added. The mixture was heated at reflux for 4 h then cooled to room temperature to give a white precipitate which was filtered off. The yellow solution was concentrated, the oil was dissolved in DMF (15 mL) and then NaN₃ (1.2 equiv.) was added. The mixture was stirred for 2 h at room temperature then diluted with ethyl acetate and H₂O. The organic layer was washed with H₂O and a saturated lithium chloride solution, dried over MgSO₄, and concentrated under reduced pressure to give sticky oil. The crude oil was dissolved in of CHCl₃ (20 mL) and MeOH (20 mL), and then 10% Pd/C (0.20 g) and conc. HCl (1.5 mL) were added. The mixture was shaken under H₂ gas (50 psi) for 16 h. The Pd/C was filtered through a pad of Celite and the pad was washed with a solution of 20% MeOH in CH₂Cl₂. The pale yellow solution was concentrated under reduced pressure, and the residual was dissolved in MeOH (10 mL) and diluted with Et₂O (100 mL) to give a white precipitate. The precipitate was collected by filteration and dried to give the title compound (0.79 g, 55%).

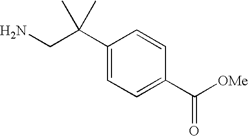

4-(2-Amino-1,1-dimethylethyl)benzoic acid methyl ester: To a solution of 4-cyanomethylbenzoic acid methyl ester (1.0 g, 5.71 mmol) in THF (20 mL) were added potassium tert-butoxide (2.5 equiv.) and MeI (3.0 equiv.). The mixture was stirred for 2 h at 0° C. then diluted with EtOAc and H₂O. The organic layer was washed with H₂O, dried over MgSO₄, and concentrated under reduced pressure to give sticky oil. The general procedure for hydrogenation (using PtO₂ in this instance) was used to prepare the title compound (1.01 g, 73%).

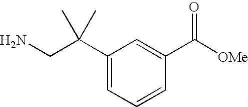

3-(2-Amino-1,1-dimethylethyl)benzoic acid methyl ester: The above procedure was used to prepare the title compound starting from 3-(1-cyanoethyl)benzoic acid

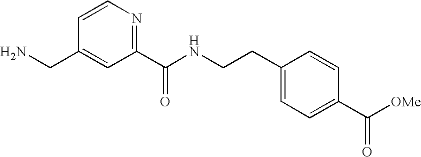

4-{2-[(4-Aminomethylpyridine-2-carbonyl)amino]ethyl}benzoic acid methyl ester: The general procedure (see below) for EDCI (N-(3-dimethylaminopropyl)-N′-ethylcarbodiimide hydrochloride) coupling between 4-(tert-butoxycarbonylaminomethyl)pyridine-2-carboxylic acid and 4-(2-aminoethyl)benzoic acid methyl ester was used to prepare the title compound.

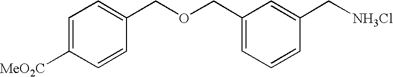

4-(3-Aminomethyl-benzyloxymethyl)-benzoic acid methyl ester: A solution of 4-hydroxymethyl-benzoic acid methyl ester (6.0 mmol, 1 eq) and 3-bromomethyl-benzonitrile (6.0 mmol, 1 eq) in CH₃OCH₂CH₂OCH₃ (20 mL) was cooled to 0° C., and then treated with NaH (18 mmol, 3 eq). After stirring at 0° C. for 20 minutes, the mixture was allowed to warm to room temperature and after 1.5 h the reaction was concentrated and partitioned between CH₂Cl₂ and H₂O. The crude mixture was purified by column chromatography (SiO₂, hexanes-EtOAc) to provide 4-(3-cyano-benzyloxymethyl)-benzoic acid methyl ester as a clear oil (46% yield). To a solution of 4-(3-cyano-benzyloxymethyl)-benzoic acid methyl ester (0.15 mmol, 1 eq) in THF (3 mL), was added 2 M BH₃ dimethyl sulfide complex solution (0.8 mL, 10 eq) and the resultant mixture was stirred at room temperature for 45 minutes and then concentrated. The concentrate was dissolved in MeOH and treated with 4 M HCl in Et₂O to form the HCl salt. The solvent was evaporated to provide the title compound as a tan solid (85% yield).

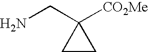

1-Aminomethyl-cyclopropanecarboxylic acid methyl ester: To a solution of 1-formyl-cyclopropanecarboxylic acid methyl ester (2.11 mmol, 1 eq) in MeOH (7 mL) was added NH₄OAc and the mixture was heated at 60° C. for 2.5 h. The solution was cooled (0° C.) and treated with a solution of NaBH₃CN (1.5 eq) in MeOH (3 mL). The mixture was allowed to stir at room temperature for 18 h, quenched with 30% aqueous NH₄OH solution (20 mL) then extracted with CH₂Cl₂ to provided the desired compound (97% yield) which was used without further purification.

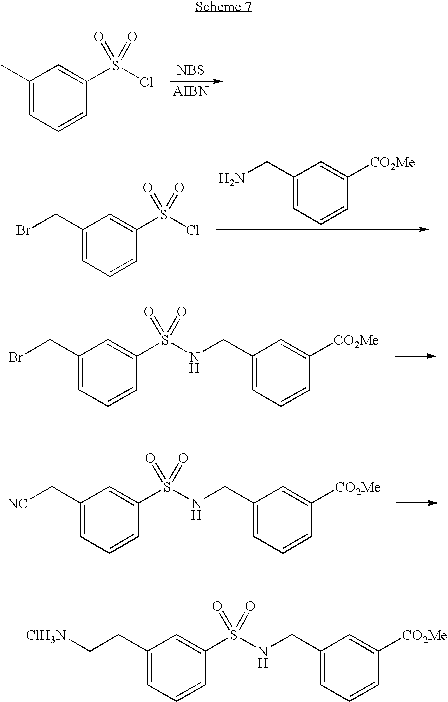

Synthesis of 3-{[3-(2-Amino-ethyl)-benzenesulfonylamino]-methyl}-benzoic acid methyl ester: A solution of 3-Bromomethyl-benzenesulfonyl chloride (prepared using the standard NBS bromination procedure on 3-methyl-benzenesulfonyl chloride) (0.785 mmol, 1.0 equiv.) in THF (5 mL) was treated with methyl 3-aminomethyl benzoate (0.746 mmol, 0.95 equiv.) and Et₃N (1.96 mmol, 2.5 equiv.) The mixture was allowed to stir for 3 h and then concentrated. According to the standard procedures above, the crude product was treated with NaCN and then reduced in the presence of HCl to give the titled hydrochloride salt.

The hydrochloride salts listed below were prepared according to this procedure; 4-{[3-(2-Amino-ethyl)-benzenesulfonylamino]-methyl}-benzoic acid methyl ester

### Synthesis of Betulinic Amides, Scheme 2, Step i;

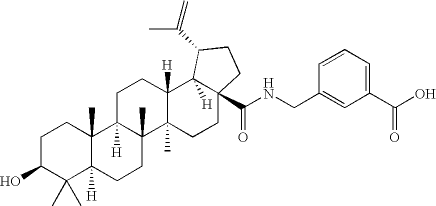

3-{[((1R,3aS,5aR,5bR,7aR,9S,11aR,11bR,13aR,13bR)-9-Hydroxy-1-isopropenyl-5a,5b,8,8,11a-pentamethylicosahydrocyclopenta[a]chrysene-3a-carbonyl)-amino]methyl}benzoic acid: A mixture of betulinic acid (200 mg, 0.438 mmol), methyl 3-aminomethyl-benzoate (107 mg, 1.2 equiv.), EDCI (101 mg, 1.2 equiv.), and 1-hydroxy-7-azabenzotriazole (30 mg, 0.5 equiv.) was dissolved in 2.2 mL of DMF (0.2 M) and then 4-methylmorpholine (0.23 mL, 4.5 equiv.) was added at room temperature. The mixture was stirred for 20 h at room temperature, and then diluted with THF (2.2 mL) and H₂O (2.2 mL), and then LiOH (105 mg, 10 equiv.) was added to the solution. The resulting solution was stirred for 16 h at room temperature. The mixture was cooled to 0° C. and then 2 N aqueous HCl (30 mL) was slowly added. The resulting white precipitate was collected on a sintered glass filter and dried with suction for 1 h to give the title compound (230 mg, 89%).
This material was used in the preparation of compounds 1-48, 117, 119-126 and 138-141, 197.

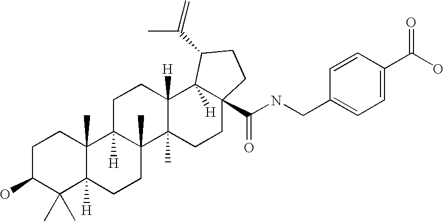

4-{[((1R,3aS,5aR,5bR,7aR,9S,11aR,11bR,13aR,13bR)-9-Hydroxy-1-isopropenyl-5a,5b,8,8,11a-pentamethyl-icosahydro-cyclopenta[a]chrysene-3a-carbonyl)-amino]-methyl}-benzoic acid: The above procedure for the EDCI coupling and hydrolysis was used to prepare the title compound (82% yield).

This material was used in the preparation of compounds 49-53.

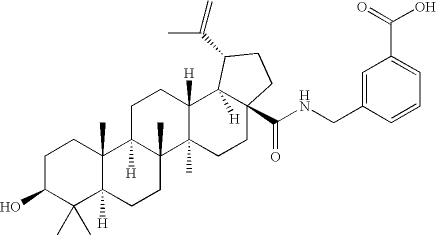

2-{2-[((1R,3aS,5aR,5bR,7aR,9S,11aR,11bR,13aR,13bR)-9-Hydroxy-1-isopropenyl-5a,5b,8,8,11a-pentamethylicosahydrocyclopenta[a]chrysene-3a-carbonyl)amino]ethyl}benzoic acid: The above procedure for the EDCI coupling and hydrolysis was used to prepare the title compound (80% yield).
This material was used in the preparation of compounds 54-55.

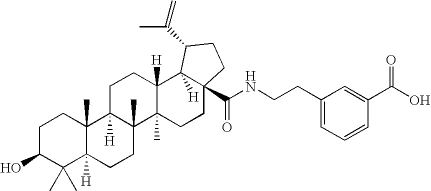

3-{2-[((1R,3aS,5aR,5bR,7aR,9S,11aR,11bR,13aR,13bR)-9-Hydroxy-1-isopropenyl-5a,5b,8,8,11a-pentamethylicosahydrocyclopenta[a]chrysene-3a-carbonyl)amino]ethyl}benzoic acid: The above procedure for the EDCI coupling and hydrolysis was used to prepare the title compound (85% yield).
This material was used to prepare compounds 56-77, 130, and 133-137, 144-179, 181, 183-185, 187-196, 198-205, 207-215, 218-219, 211-247, 252, 255, 256, 264, 272 and 275.

### General Procedures for Preparation of Bis Amides, Scheme 2, Steps ii and iii.

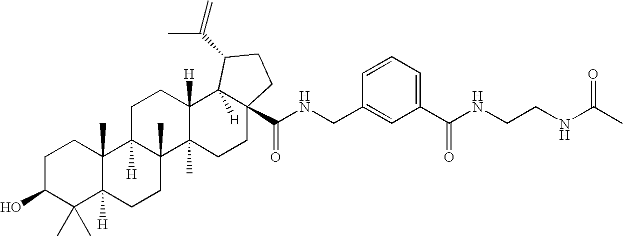

(1R,3aS,5aR,5bR,7aR,9S,11aR,11bR,13aR,13bR)-9-Hydroxy-1-isopropenyl-5a,5b,8,8,11a-pentamethylicosa-hydrocyclopenta[a]chrysene-3a-carboxylic acid 3-(2-acetylaminoethylcarbamoyl)benzylamide (4): A mixture of 3-{[((1R,3aS,5aR,5bR, 7aR,9S,11aR,11bR,13aR,13bR)-9-hydroxy-1-isopropenyl-5a,5b,8,8,11a-pentamethyl-icosa-hydrocyclopenta[a]chrysene-3a-carbonyl)amino]methyl}benzoic acid (100 mg, 0.17 mmol), N-(2-aminoethyl)acetamide (23 mg, 1.2 equiv.), EDCI (47 mg, 1.2 equiv.), and 1-hydroxy-7-azabenzotriazole (12 mg, 0.5 equiv.) was dissolved in DMF (1.2 mL) and then 4-methylmorpholine (0.1 mL, 5 equiv.) was added at room temperature. The mixture was stirred for 20 h at room temperature and then diluted with 1 N HCl solution (10 mL). The resulting white precipitate was collected on a sintered glass filter and then purified by silica gel column chromatography to give 4 (97 mg, 85%).

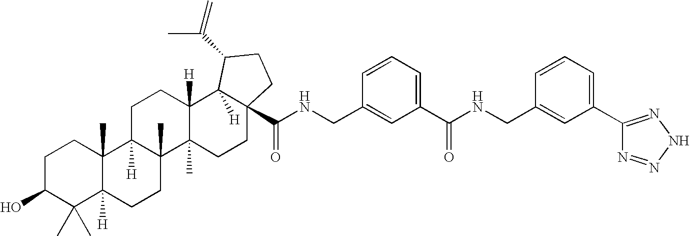

(1R,3aS,5aR,5bR,7aR,9S,11aR,11bR,13aR,13bR)-9-Hydroxy-1-isopropenyl-5a,5b,8,8,11a-pentamethylicosahydrocyclopenta[a]chrysene-3a-carboxylic acid 3-[3-(2H-tetrazol-5-yl)benzylcarbamoyl]benzylamide (21): To a solution of (1R,3aS,5aR,5bR,7aR,9S,11aR,11bR,13aR,13bR)-9-hydroxy-1-isopropenyl-5a,5b,8,8,11a-pentamethyl-icosahydro-cyclopenta[a]chrysene-3a-carboxylic acid 3-(3-cyanobenzylcarbamoyl)benzylamide (48 mg, 0.068 mmol) in DMF (2 mL) were added NaN₃ (1.1 equiv.) and NH₄Cl (1.1 equiv.). The mixture was heated at 120° C. for 6 h, 1 N HCl was added, and the precipitate was collected and purified by silica gel column chromatography to give 21 (30 mg, 61%).

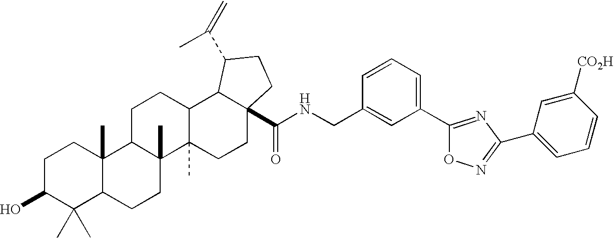

3-[5-(3-{[((1R,3aS,5aR,5bR,9S,11aR)-9-Hydroxy-1-isopropenyl-5a,5b,8,8,11a-pentamethyl-icosahydro-cyclopenta[a]chrysene-3a-carbonyl)-amino]-m ethyl}-phenyl)-[1,2,4]oxadiazol-3-yl]-benzoic acid (127): To a solution of 3-acetoxy betulinic acid (100 mg, 0.2 mmol) in CH₂Cl₂ was added SOCl₂ (0.3 mL) and DMF (1 drop). The mixture was heated at reflux for 2 h, concentrated and the residue recovered from CHCl₃ to remove residual SOCl₂. The derived acid chloride was dissolved in anhydrous CH₂Cl₂ (10 mL) and 3-[5-(3-aminomethyl-phenyl)-[1,2,4]oxadiazol-3-yl]-benzoic acid methyl ester hydrochloride (103 mg, 0.3 mmol) was added followed by drop-wise addition of Et₃N (0.5 mL). The resultant mixture was stirred at ambient temperatures overnight, concentrated, and the residue was dissolved in a mixture of THF, MeOH and 4 M NaOH (1:1:1). After stirring overnight at room temperature, the mixture was concentrated and the residue was purified by reversed phase HPLC providing the title compound (20 mg).

Synthesis of 3′-{[((1R,3aS,5aR,5bR,9S,11aR)-9-Hydroxy-1-isopropenyl-5a,5b,8,8,11a-pentamethyl-icosahydro-cyclopenta[a]chrysene-3a-carbonyl)-amino]-methyl}-biphenyl-3-carboxylic acid (128). To an ice-cold solution of 3-acetoxy-betulinic acid chloride (240 mg, 0.45 mmol), prepared as described for example 127, in dry CH₂Cl₂ (20 mL) was added 3′-aminomethyl-biphenyl-3-carboxylic acid ethyl ester (261 mg, 0.9 mmol) followed by Et₃N (200 μL) during ten minutes. The reaction mixture was allowed to stir at room temperature overnight, concentrated, and then dissolved in MeOH (15 mL) and THF (10 mL) and 4 M NaOH (15 mL) was added. After stirring overnight at room temperature, the mixture was concentrated, treated with 6 M HCl (25 mL) and stirred at room temperature for 1 hr until the product completely crashed out of solution. The precipitated product was collected by filteration and purified by reversed phase HPLC to afford 128 as a white powder (60 mg).

### Representative Synthesis of Betulinic Amide Aryl Ethers, Scheme 4;

Synthesis of 5-(4-{2-[((1R,3aS,5aR,5bR,9S,11aR)-9-hydroxy-1-isopropenyl-5a,5b,8,8,11a-pentamethyl-icosahydro-cyclopenta[a]chrysene-3a-carbonyl)-amino]-ethyl}-phenoxy)-pentanoic acid (112): A solution of phenol derivative 111 (1 equiv) in DMF (3 mL) was treated with Cs₂CO₃ (2 equiv), and 5-bromo-pentanoic acid methyl ester (3 equiv) in a microwave tube. The mixture was heated at 145° C. for 2300 seconds, filtered, concentrated and partitioned between CH₂Cl₂ and H₂O, to give product methyl ester (71% yield). The derived ether (0.35 mmol) was dissolved in a mixture of 4M NaOH (3 mL), MeOH (7 mL) and THF (1 mL) and allowed to stir at room temperature for 20 h. The mixture was concentrated, diluted with H₂O and acidified with 6N HCl (to pH 3). The resulting precipitate was collected by filtration and air-dried to provide the title compound.
Compounds 113-117 and 119-120 were prepared in an analogous fashion.

Synthesis of 5-[(3-{[((1R,3aS,5aR,5bR,9S,11aR)-9-hydroxy-1-isopropenyl-5a,5b,8,8,11a-pentamethyl-icosahydro-cyclopenta[a]chrysene-3a-carbonyl)-amino]-methyl}-benzoylamino)-methyl]-2-(3-imidazol-1-yl-propoxy)-benzoic acid methyl ester (121): A solution of 13 methyl ester (0.46 mmol, 1 equiv.) in DMF (1.75 mL) was treated with dibromopropane (1.15 mmol, 2.5 equiv.) and K₂CO₃ (0.69 mmol, 1.5 equiv.) and heated to 60° C. overnight. The reaction was cooled to room temperature and the product collected by precipitation with 1 N HCl and filtration. The air dried bromide derivative (off-white powder) was carried directly to the next reaction. Bromide derivative (0.074 mmol, 1.0 equiv.) was dissolved in DMF (1 mL) and treated with an excess of imidazole (2.8 mmol, 40 equiv.). The mixture was heated at 60° C. for 1 h, treated with H₂O and the resultant precipitate was collected by filtration and purified by preperative RP-HPLC to give compound 121 as a white powder.

Compounds 122-125 were prepared in an analogous fashion.

### Representative Synthesis of Carboxybenzimidazole Betulinic Amide, Scheme 5;

Synthesis of 2-(3-{2-[((1R,3aS,5aR,5bR,9S,11aR)-9-hydroxy-1-isopropenyl-5a,5b,8,8,11a-pentamethyl-icosahydro-cyclopenta[a]chrysene-3a-carbonyl)-amino]-ethyl}-phenyl)-3H-benzoimidazole-5-carboxylic acid (130): A solution of 61 methyl ester (prepared according to the general amide coupling procedure, see Scheme 2) in AcOH (0.01 M) was heated at 115° C. for 40 h. The reaction mixture was cooled to room temperature and H₂O was added causing the product to precipitate. The white solid was isolated by filtration then purified by preperative TLC. The product obtained was treated with a solution of 4 M NaOH: MeOH:THF (1:1:1) to effect the hydrolysis of the ester. Upon complete hydrolysis, addition of 1 NHCL was used to form a precipitate that was isolated by filtration providing compound 130 as a white solid.

### Synthesis of Dihydroxy and Hydroxy Amino Betulinic Amide Derivatives, Scheme 6.

Synthesis of 5-[3-({[(1R,3aS,5aR,5bR,9S,11aR)-9-hydroxy-1-(1-hydroxy-1-methyl-2-pyrrolidin-1-yl-ethyl)-5a,5b,8,8,11a-pentamethyl-icosahydro-cyclopenta[a]chrysene-3a-carbonyl]-amino}-methyl)-benzoylamino]-pentanoic acid (138): Compound 1 (0.218 mmol, 1.0 equiv.) was dissolved in a mixture of CH₂Cl₂ (5 mL) and THF (3 mL) and cooled to 0° C. in an ice bath. In a separate vial, mCPBA (0.262 mmol, 1.2 equiv.) was dissolved in CH₂Cl₂ (3 mL) and cooled to 0° C. The solution of mCPBA was then added to the solution containing compound 1 and the reaction was stirred at 0° C. for 3 h. The solvent was then removed in vacuo giving crude epoxide which was dissolved in neat pyrrolidine and heated at 80° C. overnight. The residual pyrrolidine was removed in vacuo and the residual solid purified by preperative RP-HPLC to give compound 138 as a white powder.

5-[3-({[(1R,3aS,5aR,5bR,9S,11aR)-1-(1,2-Dihydroxy-1-methyl-ethyl)-9-hydroxy-5a,5 b,8,8,11a-pentamethylicosahydrocyclopenta[a]chrysene-3a-carbonyl]amino}methyl)benzoylamino]pentanoic acid (139 and 140): To a solution of 5-(3-{[((1R,3aS,5aR,5bR,7aR,9S,11aR,11bR,13aR,13bR)-9-hydroxy-1-isopropenyl-5a,5b,8,8,11a-pentamethylicosahydrocyclopenta[a]chrysene-3a-carbonyl)amino]methyl}benzoylamino)pentanoic acid (1) (50 mg, 0.073 mmol) in tert-BuOH-acetone-H₂O (2:2:1, 5 mL) was added OsO₄ (1.2 equiv.). The mixture was stirred for 20 h at room temperature and then extracted with EtOAc. The organic layer was dried (Na₂SO₄), concentrated, and the resulting material purified by silica gel column chromatography to give compound 139, isomer 1 (24 mg, 45%) and compound 140, isomer 2 (16 mg, 30%).

Synthesis of 5-(3-{[((1R,3aS,5aR,5bR,7aR,9S,11aR,11bR,13aR,13bR)-9-hydroxy-1-isopropenyl-5a,5b,8,8,111a-pentamethylicosahydrocyclopenta-[a]chrysene-3a-carbonyl)amino]methyl}benzoylamino)pentanoic acid (141): Compound 1 (200 mg, 0.29 mmol) was dissolved in acetone (5 mL) and then Jones reagent (3 equiv.) was slowly added at 0° C. The mixture was stirred for 1 h at room temperature then quenched with a few drops of 2-propanol and H₂O, and then extracted with EtOAc. The organic layer was washed with H₂O, dried (MgSO₄), and concentrated. The resulting material was purified by silica gel column chromatography to give the title compound (170 mg, 85%).

Synthesis of 3-[(3-{[2-((1R,3aR,5aR,5bR,9S,11aR)-9-Hydroxy-1-isopropenyl-5a,5b,8,8,11a-pentamethylicosahydrocyclopenta[a]chrysen-3a-yl)acetylamino]methyl}-benzoylamino)methyl]benzoic acid (142): A suspension of methoxymethyltriphenylphosphonium chloride (0.35 g, 1.04 mmol) in THF (5 mL) was treated with NaN(SiMe₃)₃ (1 equiv.) at 0° C. for 30 min. A solution of 3-acetoxybetulinic aldehyde (100 mg, 0.21 mmol) in THF (2 mL) was added and the mixture was stirred for 4 h at 0° C., quenched with H₂O, and extracted with Et₂O. The organic layer was washed with H₂O, dried (MgSO₄), and concentrated. The residual oil was dissolved in THF (2 mL) and acetone (5 mL), and then Jones reagent (3 equiv.) was slowly added at 0° C. The mixture was stirred for 4 h at room temperature and then quenched with a few drops of 2-propanol and H₂O, and then extracted with Et₂O. The organic layer was washed with H₂O, dried (MgSO₄), and concentrated to give crude homologated acid (80 mg, 80%). The general procedure for sequential EDCI couplings and hydrolyses was used to prepare the title compound.

Synthesis of 3-[(3-{[((1R,3aS,5aR,5bR,9S,11aR)-9-Hydroxy-1-isopropenyl-5a,5b,8,8,11a-pentamethyl-icosahydro-cyclopenta[a]chrysen-3a-ylmethyl)-amino]-methyl}-benzoylamino)-methyl]-benzoic acid (143). To a stirred solution of 3-acetoxy betulinaldehyde (300 mg, 0.62 mmol) in MeOH (12 ml) and THF (8 mL) was added 3-aminomethyl-benzoic acid methyl ester (200 mg, 1 mmol) and stirred at room temperature for 4 hrs. The reaction mixture was cooled to 0° C. and NaBH₄ (80 mg) was added over a period of 10 min followed by stirring at room temperature for 3 h. The reaction mixture was quenched with EtOAc (5 mL) and the solvent evaporated. The residue was suspended in H₂O (10 mL) and extracted with EtOAc (2×20 mL). The combined EtOAc layers were concentrated. The residue was dissolved in mixture of THF (10 mL) and MeOH (15 mL) and 4 M NaOH (10 mL) was added and the mixture stirred at room temperature overnight. The solvent was removed in vacuo and the mixture was acidified with 6 M HCl which resulted in the formation precipitate. The precipitate collected by filteration, rinsed with H₂O and dried.

To a solution of the above benzoic acid derivative (92 mg, 0.16 mmol) in DMF (5 mL) was added EDCI (45 mg, 0.24 mmol), HOBT (21.6 mg, 0.16 mmol) and N-methyl morpholine (168 mg, 1.6 mmol) and the mixture was stirred at room temp for 30 min. After this time, methyl 3-aminomethyl-benzoate (53 mg, 0.32 mmol) was added and the mixture was stirred at room temperature overnight. Upon complete reaction, 1 N HCl was added to precipitate the product. The precipitate was collected by filteration, dissolved in a mixture of MeOH (3 mL) and THF (3 mL) and 4 M NaOH (3 mL) was added. After stirring at room temperature for 8 h, the mixture was concentrated, acidified with 6 M HCl. The precipitate was collected by filteration and then purified by RP-HPLC to afford compound 143 (20 mg).

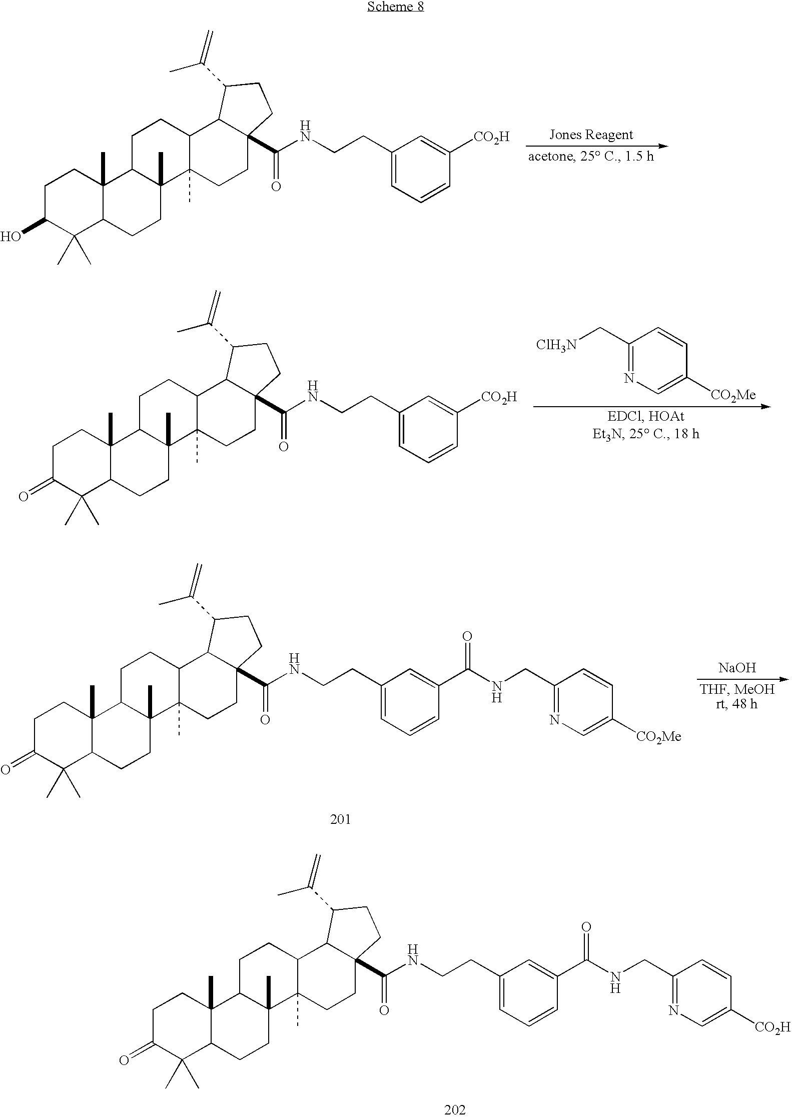

Synthesis of 6-[(3-{2-[((1R,3aS,5aR,5bR,1aR)-1-isopropenyl-5a,5b,8,8,11a-pentamethyl-9-oxo-icosahydro-cyclopenta[a]chrysene-3a-carbonyl)-amino]-ethyl}-benzoylamino)-methyl]-nicotinic acid methyl ester (201): To a solution of 3-{2-[((1R,3aS,5aR,5bR,7aR,9S,11aR,11bR,13aR,13bR)-9-hydroxy-1-isopropenyl-5a,5b,8,8,11a-pentamethylicosahydrocyclopenta[a]chrysene-3a-carbonyl)-amino]ethyl}-benzoic acid (0.50 g, 0.83 mmol) in acetone (20 mL) was added 2.67 M Jones Reagent (04 mL, 1.035 mmol) portionwise at room temperature. After stirring for 1.5 h, the mixture was diluted with acetone (40 mL) and treated with Celite. The mixture was filtered over a bed of Celite and concentrated. Purification (SiO₂, CH₂Cl₂-MeOH; 0-15%) provided the desired keto amide (363 mg, 73%) as a white solid. This material was dissolved in DMF (5 mL) and treated sequentially with EDCI_HCl (173 mg, 0.78 mmol), HOAt (41 mg, 0.30 mmol), 6-aminomethyl-nicotinic acid methyl ester (see above for synthesis, 0.19 g, 0.78 mmol) and Et₃N (0.55 mL, 3.92 mmol). After stirring for 18 h the mixture was concentrated under reduced pressure and purified by chromatography (SiO₂, CH₂Cl₂-MeOH; 0-20%) providing the titled compound as a white solid.

Synthesis of 6-[(3-{2-[((1R,3aS,5aR,5bR,11aR)-1-isopropenyl-5a,5b,8,8,11a-pentamethyl-9-oxo-icosahydro-cyclopenta[a]chrysene-3a-carbonyl)-amino]-ethyl}-benzoylamino)-methyl]-nicotinic acid (202): Base saponification according to the general procedure outlined above.

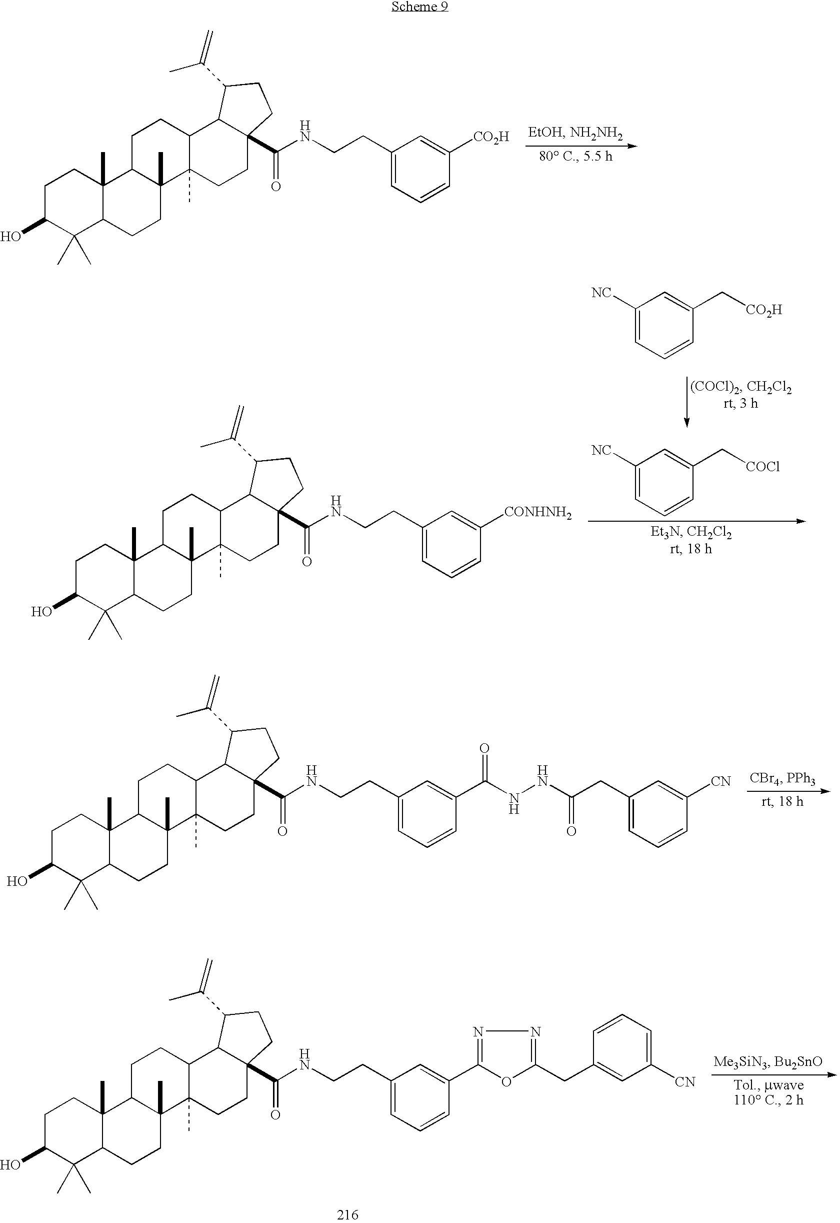

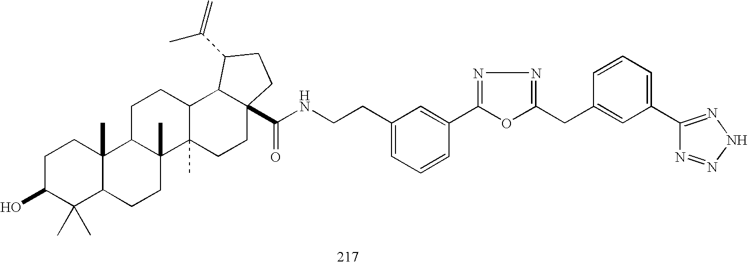

Synthesis of (1R,3aS,5aR,5bR,9S,11aR)-9-hydroxy-1-isopropenyl-5a,5b,8,8,11a-pentamethyl-icosahydro-cyclopenta[a]chrysene-3a-carboxylic acid (2-{3-[5-(4-cyano-benzyl)-[1,3,4]oxadiazol-2-yl]-phenyl}-ethyl)-amide (216): To a solution of 3-{2-[((1R,3aS,5aR,5bR,7aR,9S,11aR,11bR,13aR,13bR)-9-hydroxy-1-isopropenyl-5a,5b,8,8,11a-pentamethylicosahydrocyclopenta[a]chrysene-3a-carbonyl)-amino]ethyl}benzoic acid methyl ester (see above for synthesis, 0.54 mmol, 1 eq) in EtOH was added NH₂NH₂ (5.0 mmol, 10 eq) and the mixture was heated at reflux. After 5.5 h, H₂O was added and the resultant precipitate was collected by filteration to provide crude hydrazide that was used without further purification (98% yield). A solution of (3-cyano-phenyl)-acetic acid (2 mmol, 1 eq) in CH₂Cl₂ (10 mL) was cooled to 0° C. and treated with (COCl)₂ (20 mmol, 10 eq). The mixture was allowed to warm to room temperature and stir for 3 h. The mixture was concentrated, and then recovered from CH₂Cl₂ (3×), providing (3-cyano-phenyl)-acetyl chloride. This material (0.20 mmol, 1 eq) was dissolved in CH₂Cl₂ and treated with the hydrazide (1.3 mmol, 0.65 eq) from step 1 followed by Et₃N (7.0 mmol, 3.5 eq). The mixture was allowed to stir at room temperature for 18 h providing the desired bis-acyl hydrazide as the minor product by LC/MS. To the reaction mixture was then added CBr₄ (0.5 mmol, 0.5 eq) and PPh₃ resin (0.5 mmol, 0.5 eq) and the mixture was stirred at room temperature for 18 h. The mixture was filtered and concentrated and the desired product was purified by HPLC.

Synthesis of (1R,3aS,5aR,5bR,9S,11aR)-9-Hydroxy-1-isopropenyl-5a,5b,8,8,11a-pentamethyl-icosahydro-cyclopenta[a]chrysene-3a-carboxylic acid [2-(3-{5-[3-(2H-tetrazol-5-yl)-benzyl]-[1,3,4]oxadiazol-2-yl}-phenyl)-ethyl]-amide (217): To a solution of (1R,3aS,5aR,5bR,9S,1aR)-9-hydroxy-1-isopropenyl-5a,5b,8,8,11a-pentamethyl-icosahydro-cyclopenta[a]chrysene-3a-carboxylic acid (2-{3-[5-(4-cyano-benzyl)-[1,3,4]oxadiazol-2-yl]-phenyl}-ethyl)-amide (see above, 0.013 mmol, 1 eq), in toluene was added Me₃SiN₃ (0.027 mmol, 2 eq) and Bu₂SnO (0.013 mmol, 1 eq). The reaction vessel was placed in a microwave reactor and heated at 110° C., for 2 h providing the title compound (20% yield) after HPLC purification.

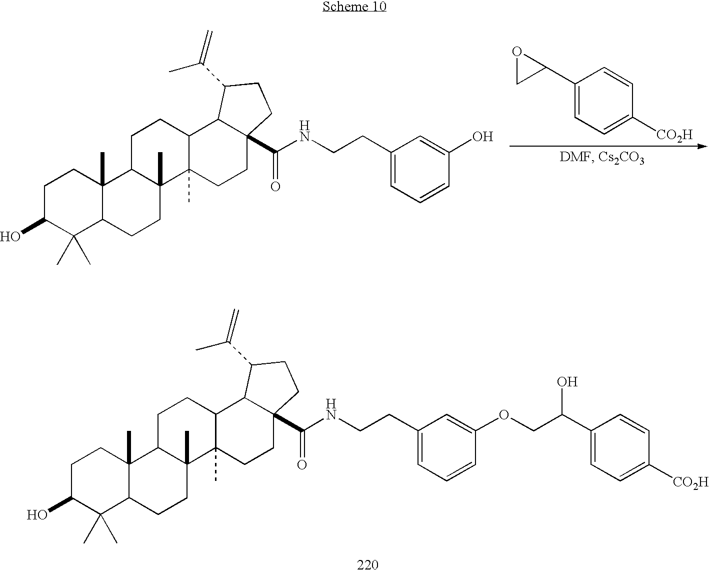

Synthesis of 4-[1-Hydroxy-2-(3-{2-[((1R,3aS,5aR,5bR,9S,11aR)-9-hydroxy-1-isopropenyl-5a,5b,8,8,11a-pentamethyl-icosahydro-cyclopenta[a]chrysene-3a-carbonyl)-amino]-ethyl}-phenoxy)-ethyl]-benzoic acid (220): A solution of (1R,3aS,5aR,5bR,9S,11aR)-9-hydroxy-1-isopropenyl-5a,5b,8,8,11a-pentamethyl-icosahydro-cyclopenta[a]chrysene-3a-carboxylic acid [2-(3-hydroxy-phenyl)-ethyl]-amide (0.174 mmol, 1.0 equiv.) in DMF was treated with 4-oxiranyl-benzoic acid (0.174 mmol, 1.0 equiv.) and Cs₂CO₃ (0.522 mmol, 3.0 equiv.) and heated at 60° C. overnight. The mixture was then cooled to room temperature, treated with H₂O and the precipitated product collected by filtration. Purification by Reversed Phase-HPLC gave 220 as white powder.

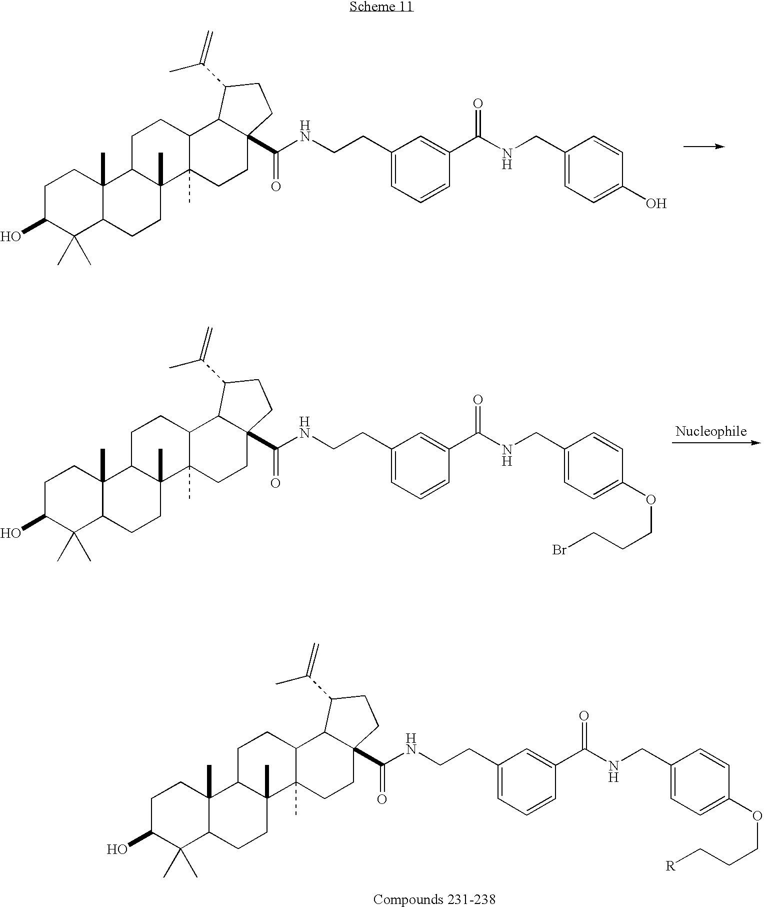

Synthesis of compounds 231-238: A solution of (1R,3aS,5aR,5bR,9S,11aR)-9-hydroxy-1-isopropenyl-5a,5b,8,8,11a-pentamethyl-icosahydro-cyclopenta[a]chrysene-3a-carboxylic acid {2-[3-(4-hydroxy-benzylcarbamoyl)-phenyl]-ethyl}-amide (0.46 mmol, 1 equiv.) in DMF was treated with Br(CH₂)₃Br (1.15 mmol, 2.5 equiv.) and K₂CO₃ (0.69 mmol, 1.5 equiv.) and heated to 60° C. overnight. The reaction was then cooled to room temperature and the product precipitated out by the addition of 1 N HCl. The product was isolated by filtration then dried giving (1R,3aS,5aR,5bR,9S,11aR)-9-hydroxy-1-isopropenyl-5a,5b,8,8,11a-pentamethyl-icosahydro-cyclopenta[a]chrysene-3a-carboxylic acid (2-{3-[4-(3-bromo-propoxy)-benzylcarbamoyl]-phenyl}-ethyl)-amide as an off-white powder that was carried directly to the next reactions. This material (0.074 mmol, 1.0 equiv.) was then dissolved in DMF (1 mL) and treated with imidazole (2.8 mmol, 40 equiv.). The mixture was heated to 60° C. for 1 h then H2O was added causing a precipitate to form. The precipitate was isolated by filtration and purified by preparatory HPLC to give compound 231 as white powder. According to the above procedure compounds 232-238 were obtained.

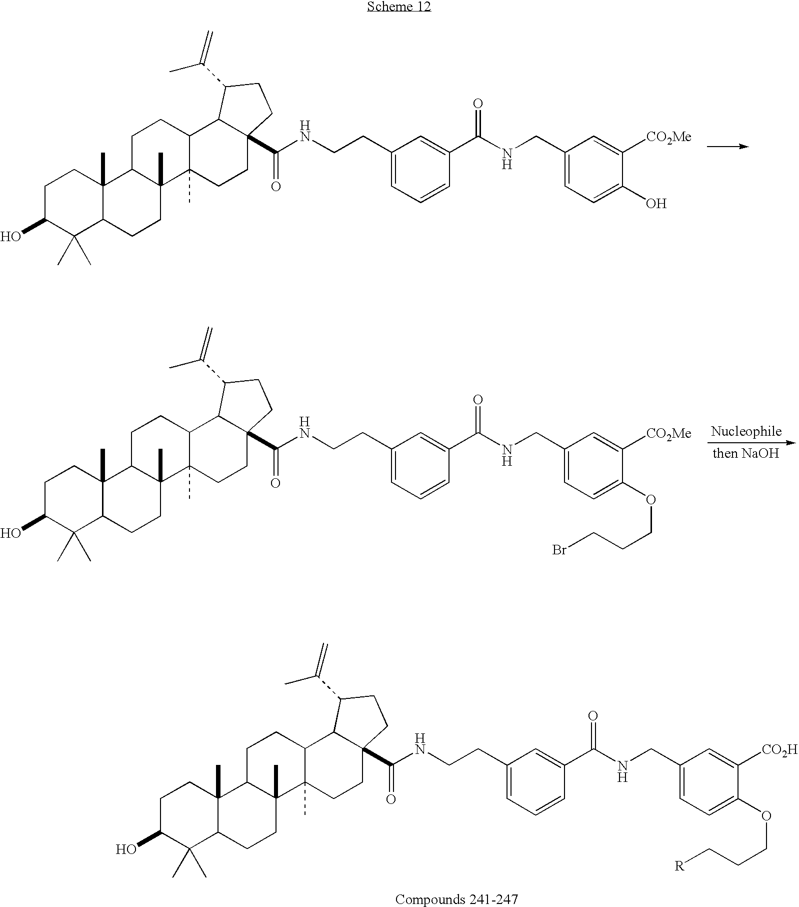

Synthesis of compounds 241-247: A solution of 2-hydroxy-5-[(3-{2-[((1R,3aS,5aR,5bR,9S,11aR)-9-hydroxy-1-isopropenyl-5a,5b,8,8,11a-pentamethyl-icosahydro-cyclopenta[a]chrysene-3a-carbonyl)-amino]-ethyl}-benzoylamino)-methyl]-benzoic acid methyl ester (0.46 mmol, 1 equiv.) in DMF was treated with Br(CH₂)₃Br (1.15 mmol, 2.5 equiv.) and potassium carbonate (0.69 mmol, 1.5 equiv.) and heated to 60° C. overnight. The reaction is then cooled to room temperature and the product precipitated out by the addition of 1 N HCl. The product was isolated by filtration then dried giving 2-(3-bromo-propoxy)-5-[(3-{2-[((1R,3aS,5aR,5bR,9S,11aR)-9-hydroxy-1-isopropenyl-5a,5b,8,8,11a-pentamethyl-icosahydro-cyclopenta[a]chrysene-3a-carbonyl)-amino]-ethyl}-benzoylamino)-methyl]-benzoic acid methyl ester as an off-white powder that was carried directly to the next reactions. This material (0.074 mmol, 1.0 equiv.) was then dissolved in DMF (1 mL) and treated with imidazole (2.8 mmol, 40 equiv.). The mixture was heated to 60° C. for 1 h then water was added causing a precipitate to form. The precipitate was isolated by filtration and the resulting powder dissolved in 1:1 MeOH, THF (2 mL). The solution was then subjected to 4 N NaOH (1 mL) and aged for 2 h. The resulting product was then precipitated from solution by the addition of excess 4 N HCl. The precipitate was isolated by filtration and purified by preparatory HPLC to give compound 241 as a white powder. According to the above procedure compounds 242-247 were obtained.

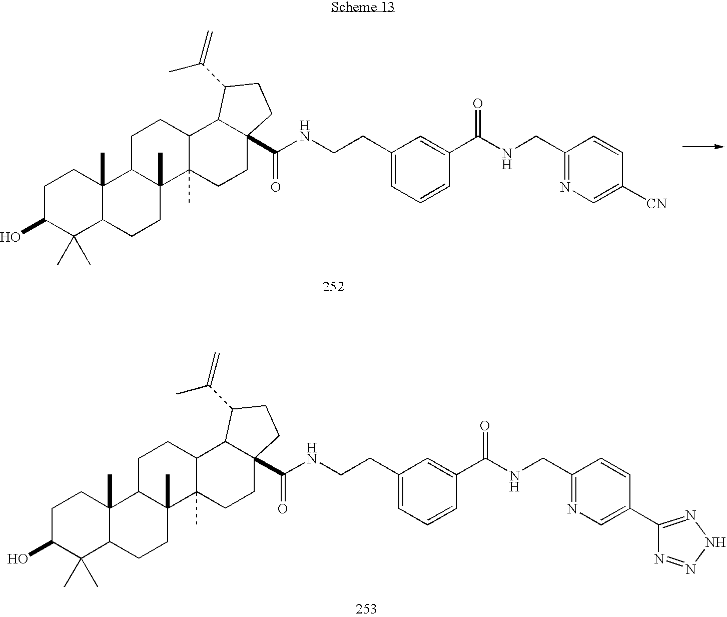

Synthesis of (1R,3aS,5aR,5bR,9S,11aR)-9-hydroxy-1-isopropenyl-5a,5b,8,8,11a-pentamethyl-icosahydro-cyclopenta[a]chrysene-3a-carboxylic acid [2-(3-{[5-(2H-tetrazol-5-yl)-pyridin-2-ylmethyl]-carbamoyl}-phenyl)-ethyl]-amide (253): A solution of compound 252 (0.10 mmol, 1.0 equiv.) in DMF (1.5 mL) was treated with NaN₃ (0.11 mmol, 1.1 equiv.) and ammonium chloride (0.11 mmol, 1.1 equiv.) and heated to 120° C. for 6 h. The reaction was then cooled to room temperature and the product precipitated out by the addition of 1 N HCl. The product was isolated by filtration then purified by preparatory HPLC to give compound 253 as a white powder.

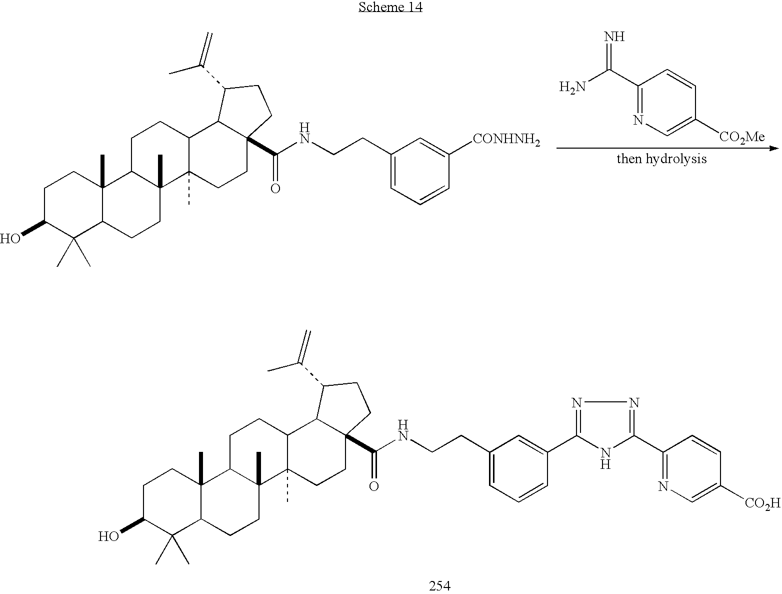

### Synthesis of 6-[5-(3-{2-[((1R,3aS,5aR,5bR,9S,11aR)-9-hydroxy-1-isopropenyl-5a,5b,8,8,11a-pentamethyl-icosahydro-cyclopenta[a]chrysene-3a-carbonyl)-amino]-ethyl}-phenyl)-4H-[1,2,4]triazol-3-yl]-nicotinic acid (254)

A solution of (1R,3aS,5aR,5bR,9S,11aR)-9-hydroxy-1-isopropenyl-5a,5b,8,8,11a-pentamethyl-icosahydro-cyclopenta[a]chrysene-3a-carboxylic acid [2-(3-hydrazinocarbonyl-phenyl)-ethyl]-amide (0.243 mmol, 1.0 equiv.) in NMP was treated with commercially available benzamidine (0.365 mmol, 1.5 equiv.) and heated to 180° C. in a microwave reactor for 5.5 minutes. The reaction was then cooled to room temperature and the product precipitated out by the addition of water. The product was isolated by filtration then purified by preparatory HPLC to give compound 254 as a white powder.

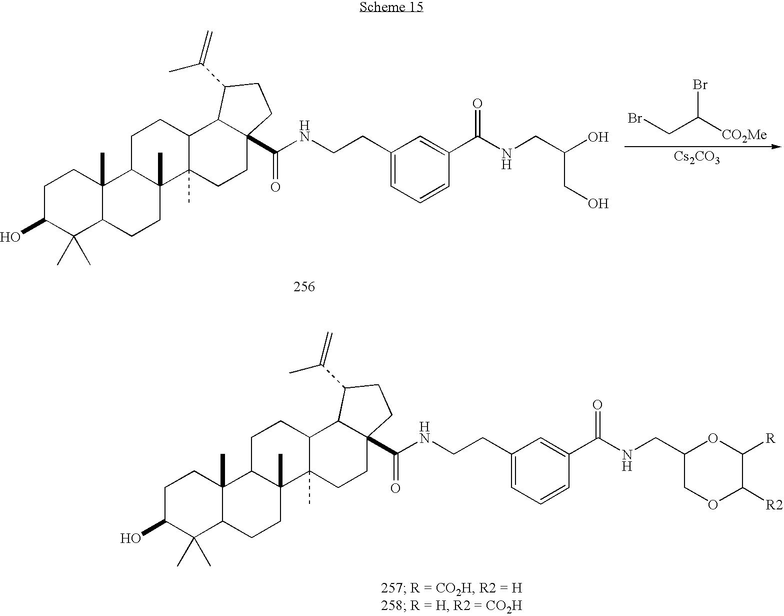

A solution of 256 (0.415 mmol, 1.0 equiv.) in DMF (3 mL) was treated with a 2,3-dibromo-propionic acid methyl ester (0.415 mmol, 1.0 equiv.) and Cs₂CO₃ (1.66 mmol, 4.0 equiv.). The reaction was then heated at 60° C. overnight. The product was precipitated from solution by the addition of an excess of water. The precipitate was isolated via filtration and treated to typical hydrolysis conditions. The resulting product was determined to be a mixture of compounds 257 and 258 with compound 257 being the major isomer. The mixture was purified by preparatory HPLC giving compounds 257 and 258 as white powders.

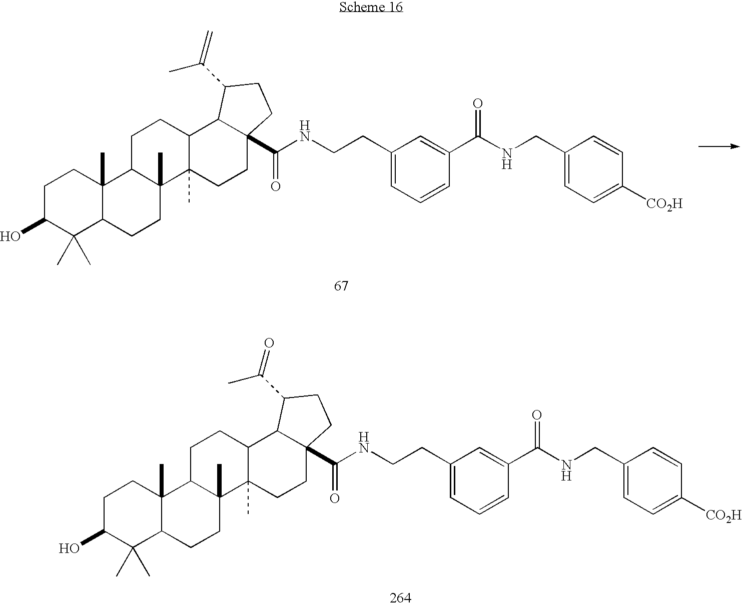

Synthesis of 4-[(3-{2-[((1R,3aS,5aR,5bR,9S,1aR)-1-Acetyl-9-hydroxy-5a,5b,8,8,11a-pentamethyl-icosahydro-cyclopenta[a]chrysene-3a-carbonyl)-amino]-ethyl}-benzoylamino)-methyl]-benzoic acid (264): A solution of 67 (0.339 mmol, 1.0 equiv.) in a mixture of CCl₄ and AcCN (3 mL, 1:1) was treated with a solution of NaIO₄ (1.02 mmol, 3.0 equiv.). To this was then added RUCl₃ (0.017 mmol, 0.05 equiv.) and the resulting biphasic mixture was stirred vigorously for 3 h at ambient temperatures. The aqueous layer was separated and the organic layer extracted with 1 N HCl (1×). The organic layer was then concentrated in vacuo providing 264 as an off-white powder that was purified by preparative layer chromatography.

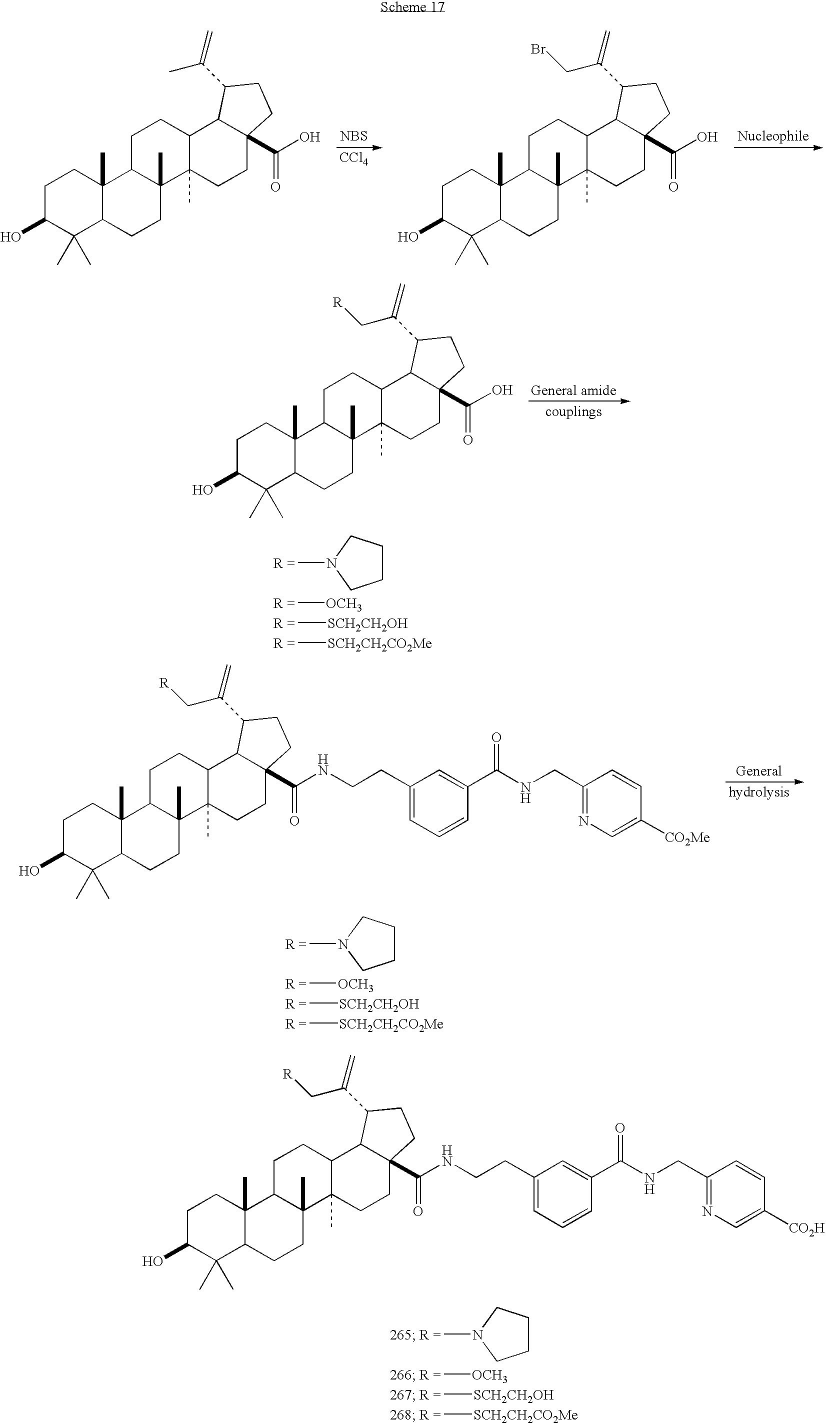

A solution of betulinic acid (6.02 mmol, 1.0 equiv.) and NBS (12.04 mmol, 2.0 equiv.) in CCl₄ was allowed to stir at room temperature overnight. The solids were removed via filtration and the solvent removed in vacuo to give (1R,3aS,5aR,5bR,9S,11aR)-1-(1-bromomethyl-vinyl)-9-hydroxy-5a,5b,8,8,11a-pentamethyl-icosahydro-cyclopenta[a]chrysene-3a-carboxylic acid which was purified by MPLC (SiO₂, EtOAc-hexanes).

Synthesis of 6-{[3-(2-{[(1R,3aS,5aR,5bR,9S,11aR)-9-Hydroxy-5a,5b,8,8,11a-pentamethyl-1-(1-pyrrolidin-1-ylmethyl-vinyl)-icosahydro-cyclopenta[a]chrysene-3a-carbonyl]-amino}-ethyl)-benzoylamino]-methyl}-nicotinic acid (265): A solution of (1R,3aS,5aR,5bR,9S,11aR)-1-(1-bromomethyl-vinyl)-9-hydroxy-5a,5b,8,8,11a-pentamethyl-icosahydro-cyclopenta[a]chrysene-3a-carboxylic acid (0.254 mmol) in neat pyrrolidine was heated at 150° C. in a microwave reactor for 1 h. The intermediate (1R,3aS,5aR,5bR,9S,11aR)-9-Hydroxy-5a,5b,8,8,11a-pentamethyl-1-(1-pyrrolidin-1-ylmethyl-vinyl)-icosahydro-cyclopenta[a]chrysene-3a-carboxylic acid was then precipitated from solution by the addition of an excess of 25% MeOH in water. Compound 3 was then taken in its crude form and subjected to typical EDC coupling conditions followed by hydrolysis to give compound 265, which was purified by. RP-HPLC.

Synthesis of 6-{[3-(2-{[(1R,3aS,5aR,5bR,9S,11aR)-9-hydroxy-1-(1-methoxymethyl-vinyl)-5a,5b,8,8,11a-pentamethyl-icosahydro-cyclopenta[a]chrysene-3a-carbonyl]-amino}-ethyl)-benzoylamino]-methyl}-nicotinic acid (266): A solution of (1R,3aS,5aR,5bR,9S,1aR)-1-(1-bromomethyl-vinyl)-9-hydroxy-5a,5b,8,8,11a-pentamethyl-icosahydro-cyclopenta[a]chrysene-3a-carboxylic acid (0.260 mmol, 1.0 equiv.) in 50% MeOH/THF was treated with a 4 N aqueous NaOH (0.800 mmol, 3.1 equiv.). The resulting solution was heated at 60° C. overnight. Intermediate (1R,3aS,5aR,5bR,9S,11aR)-9-hydroxy-1-(1-methoxymethyl-vinyl)-5a,5b,8,8,11a-pentamethyl-icosahydro-cyclopenta[a]chrysene-3a-carboxylic acid was then precipitated from solution by the addition of an excess of water, and then taken in its crude form and subjected to typical EDC coupling conditions followed by hydrolysis to give crude compound 266 which was purified by RP-HPLC.

Synthesis of 6-({3-[2-({(1R,3aS,5aR,5bR,9S,11aR)-9-Hydroxy-1-[1-(2-hydroxy-ethylsulfanylmethyl)-vinyl]-5a,5b,8,8,11a-pentamethyl-icosahydro-cyclopenta[a]chrysene-3a-carbonyl}-amino)-ethyl]-benzoylamino}-methyl)-nicotinic acid (267): To a solution of (1R,3aS,5aR,5bR,9S,11aR)-1-(1-bromomethyl-vinyl)-9-hydroxy-5a,5b,8,8,11a-pentamethyl-icosahydro-cyclopenta[a]chrysene-3a-carboxylic acid (0.260 mmol, 1.0 equiv.) in DMF (1 mL) was added HOCH₂CH₂SH (0.286 mmol, 1.1 equiv.) and K₂CO₃ (0.800 mmol, 3.0 equiv.). The resulting solution was heated at 60° C. overnight. The mixture was treated with H₂O and the precipitated thiol adduct was collected by filtration. This material was converted to 267 via the typical EDCI coupling and hydrolysis conditions detailed above. Similarly, 3-mercaptopropionic acid methyl ester provided 6-({3-[2-({(1R,3aS,5aR,5bR,9S,11aR)-1-[1-(2-Carboxy-ethylsulfanylmethyl)-vinyl]-9-hydroxy-5a,5b,8,8,11a-pentamethyl-icosahydro-cyclopenta[a]chrysene-3a-carbonyl}-amino)-ethyl]-benzoylamino}-methyl)-nicotinic acid (268) using the reaction conditions and sequences outlined above.

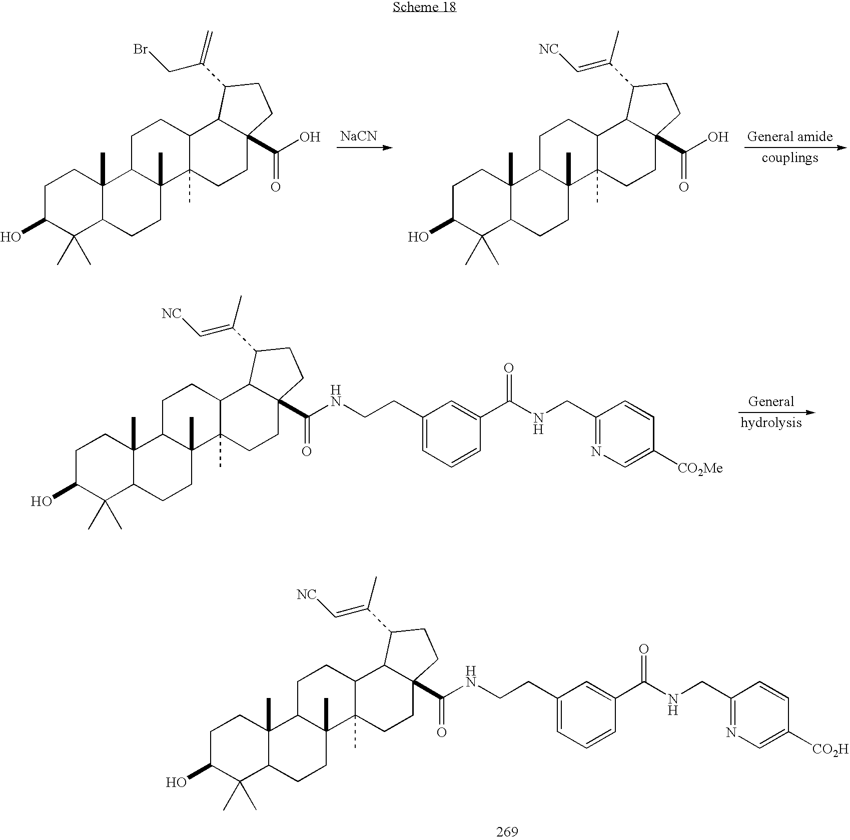

Synthesis of 6-{[3-(2-{[(1R,3aS,5aR,5bR,9S,11aR)-1-((E)-2-Cyano-1-methyl-vinyl)-9-hydroxy-5a,5b,8,8,11a-pentamethyl-icosahydro-cyclopenta[a]chrysene-3a-carbonyl]-amino}-ethyl)-benzoylamino]-methyl}-nicotinic acid (269): A solution of (1R,3aS,5aR,5bR,9S,11aR)-1-(1-bromomethyl-vinyl)-9-hydroxy-5a,5b,8,8,11a-pentamethyl-icosahydro-cyclopenta[a]chrysene-3a-carboxylic acid (0.563 mmol, 1.0 equiv.) in DMF and treated with aqueous NaCN (1.689 mmol, 3.0 equiv., 0.5 mL H₂O). The resulting solution was heated at 60° C. overnight. Intermediate (1R,3aS,5aR,5bR,9S,11aR)-1-((Z)-2-cyano-1-methyl-vinyl)-9-hydroxy-5a,5b,8,8,11a-pentamethyl-icosahydro-cyclopenta[a]chrysene-3a-carboxylic acid was precipitated from solution by the addition of an excess of H₂O, collected by filtration, and then taken in its crude form and subjected to typical EDC coupling conditions followed by hydrolysis to give crude compound 269 which was then purified by preparative layer chromatography.

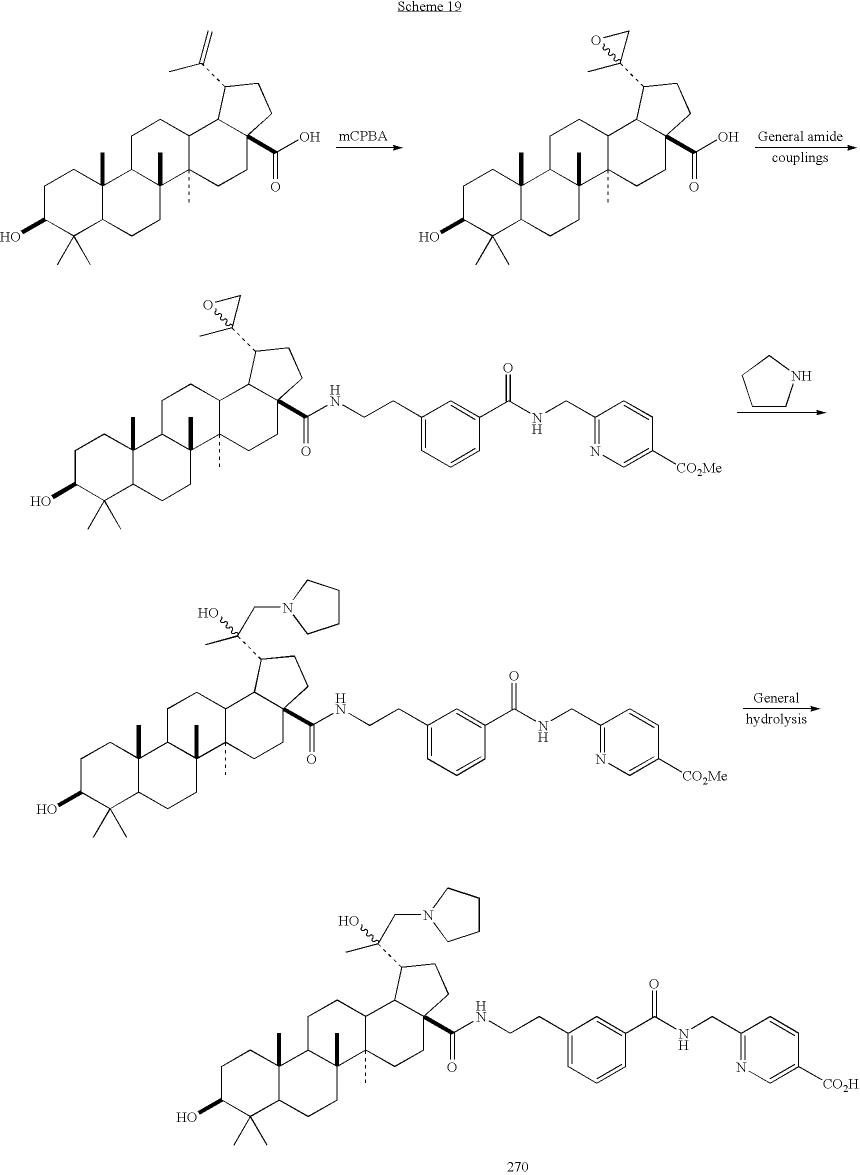

Synthesis of 6-{[3-(2-{[(1R,3aS,5aR,5bR,9S,11aR)-9-Hydroxy-1-(1-hydroxy-1-methyl-2-pyrrolidin-1-yl-ethyl)-5a,5b,8,8,11a-pentamethyl-icosahydro-cyclopenta[a]chrysene-3a-carbonyl]-amino}-ethyl)-benzoylamino]-methyl}-nicotinic acid (270): A solution of betulinic acid (2.19 mmol, 1.0 equiv.) a mixture of CH₂Cl₂ (60 mL) and THF (20 mL) was cooled to 0° C. (ice bath). In a separate vial, a solution of mCPBA (4.38 mmol, 2.0 equiv.) in CH₂Cl₂ (40 mL) was prepared and also cooled to 0° C. The mCPBA solution was then added to the betulinic acid solution and the reaction was warmed to room temperature and stirred for 3 h. The mixture was then extracted with a saturated aqueous Na₂CO₃; the organic layer was separated and concentrated to dryness to give crude epoxide. Epoxide (0.254 mmol) was then subjected to the series of typical EDC amide couplings, dissolved in neat pyrrolidine and heated to 150° C. in the microwave for 5 h. The residual pyrrolidine was concentrated in vacuo and the crude solid hydrolyzed in the usual manner and purified by RP-HPLC to give 270 as a white powder.

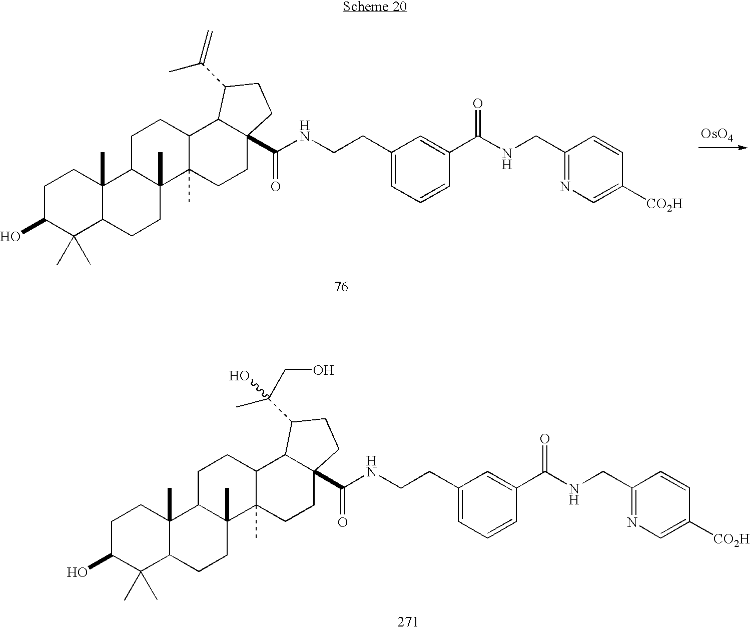

Synthesis of 6-{[3-(2-{[(1R,3aS,5aR,5bR,9S,11aR)-1-(1,2-Dihydroxy-1-methyl-ethyl)-9-hydroxy-5a,5b,8,8,11a-pentamethyl-icosahydro-cyclopenta[a]-chrysene-3a-carbonyl]-amino}-ethyl)-benzoylamino]-methyl}-nicotinic acid (271): A solution of 76 (0.266 mmol, 1.0 equiv.) in pyridine (4 mL) was treated with OsO₄ (0.266 mmol, 1.0 equiv.). The mixture was stirred at room temperature for 4 h, the product precipitated by the addition of excess water and the crude product purified by RP-HPLC giving compound 271 as a white powder.

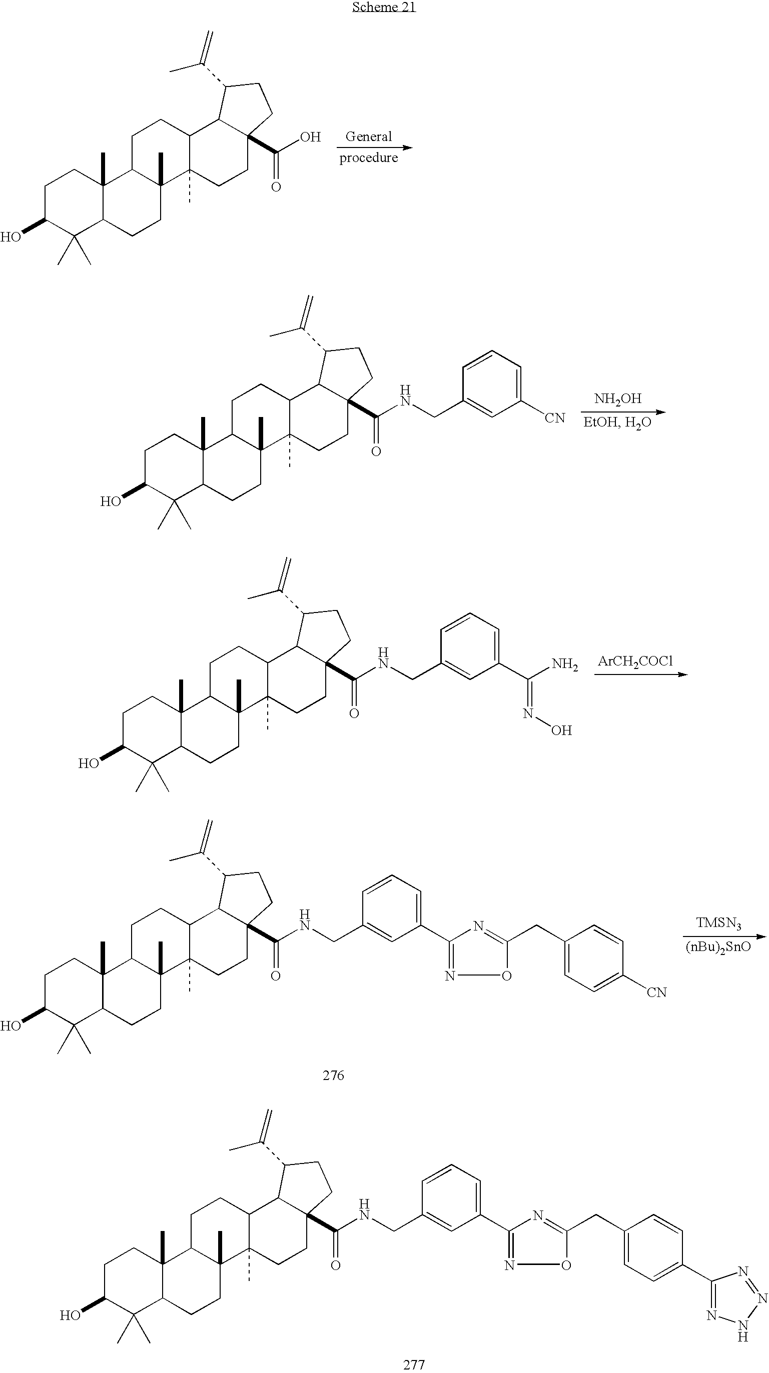

Synthesis of (1R,3aS,5aR,5bR,9S,11aR)-9-hydroxy-1-isopropenyl-5a,5b,8,8,11a-pentamethyl-icosahydro-cyclopenta[a]chrysene-3a-carboxylic acid 3-[5-(4-cyano-benzyl)-[1,2,4]oxadiazol-3-yl]-benzylamide (276): Betulinic acid (2.29 g, 5 mmol) was coupled to 3-aminomethyl-benzonitrile (900 mg, 7 mmol) in DMF (10 mL) under the standard conditions outlined above using EDCI (1.2 g, 6 mmol), HOBT (340 mg, 2.5 mmol) and N-methyl morpholine (1.5 mL) to provide amide (2.0 g, 70% yield) after column chromatography (SiO₂). This material (350 mg, 0.61 mmol) was dissolved in EtOH (25 mL) and heated at reflux with 50% aqueous NH₂OH solution (0.5 mL). After 1 h the mixture was concentrated, dried, and was used as such without further purification. To a solution of hydroxyamidine (100 mg, 0.165 mmol) in THF (10 mL) in a microwave reaction vial was added (iPr)₂NEt (64 mg, 0.48 mmol) and (4-cyanophenyl)-acetyl chloride (29 mg, 0.165 mmol, generated from corresponding acid and oxalyl chloride in CH₂Cl₂). The reaction vial was sealed, heated in a microwave reactor at 180° C. for 15 minutes and then stirred at room temperature for 30 minutes. The mixture was concentrated and the residue purified by column chromatography (SiO₂, EtOAc-hexane eluent) providing 276 (40 mg, 33% yield).

Synthesis of (1R,3aS,5aR,5bR,9S,11aR)-9-Hydroxy-1-isopropenyl-5a,5b,8,8,11a-pentamethyl-icosahydro-cyclopenta[a]chrysene-3a-carboxylic acid 3-{5-[4-(2H-tetrazol-5-yl)-benzyl]-[1,2,4]oxadiazol-3-yl}-benzylamide (277): To a solution of 276 (40 mg, 0.06 mmol) in toluene (2 mL) contained in a microwave reaction vial was added (nBu)₂SnO (2 mg, 0.006 mmol) and TMSN₃ (5.76 μL, 0.12 mmol) and the mixture was heated at 110° C. After 1 h, the mixture was concentrated and purified by reversed phase HPLC to provide 277 (10 mg).

### Example 2

### Determination of Antiviral Activity

The compounds of the invention can be tested in the following MT-4 assay to detect antiviral activity.

### MT-4 Cytoprotection Assay

The HTLV-1 transformed T cell line, MT-4, is highly susceptible to HIV-1 infection. Anti-HIV-1 agents were evaluated in this target cell line by protection from the HIV-induced cytopathic effect. In this assay, viability of both HIV-1 and mock-infected cells was assessed in a calorimetric assay that monitors the ability of metabolically-active cells to reduce the tetrazolium salt WST-1. Cytoprotection by antiviral compounds is indicated by the positive readout of increased WST-1 cleavage.

Briefly, exponentially growing MT-4 cells were mock-infected or batch-infected with the HIV-1 laboratory strain, NL4-3, at a multiplicity of infection of 0.0005. Following a two hour infection, the cells were washed to remove unbound virus and plated in the presence of increasing concentrations of compound. After four days incubation, cytoprotection in the infected cells and compound toxicity in mock-infected cells were analyzed using the WST-1 assay.

It was found that compounds of the invention have antiviral activity according to this assay. Representative compounds of the invention include those with an MT-4 assay EC50 (concentration of compound that reduces the virus induced cytopathic effect by 50%) of less than about 100 nm, such as compounds 6, 7, 9, 17, 19, 22, 25, 27, 29, 33-43, 45, 47, 48, 58, 59, 63, 65, 67, 68, 70, 73-76, 93, 95-103, 105, 108, 109, 121, 123-125, 133, 136, and 137.

### P4-MAGI Assay

P4 cells were used to test the effects of compounds on virus fusion activity. P4 cells are HIV-1 infectable CD4⁺ HeLa cells that bear the bacterial lacZ gene under the control of a minimal HIV-1 LTR. Charneau, et. al., J. Mol. Biol. 241:651-662 (1994). When these cells are infected with HIV-1, β-galactosidase is expressed due to Tat activation of the viral LTR. In this assay, P4 cells were infected with the laboratory adapted strain HIV-1_{NL4-3} at a multiplicity of infection of 1 in the presence of DEAE-Dextran. Immediately after addition of virus to the cells, increasing concentrations of compound were added to the cells. Following a 24 hour incubation, the concentration of compound at which 50% of the maximal reduction in viral replication was observed (IC₅₀) was determined by quantitating β-galactosidase expression with the Gal-Screen chemiluminescent reporter gene assay system (Applied Biosystems). The effect on cell viability (TC₅₀, concentration of compound that inhibits growth by 50%) was determined in mock-infected cells with the WST-1 Cell Proliferation Reagent (Roche Diagnostics).

It was found that compounds of the invention have antiviral activity according to this assay. Representative compounds of the invention include those with a P4-MAGI assay IC₅₀ of less than about 100 nm, such as compounds 1, 2, 6, 7, 9, 11, 13, 17, 19, 21-23, 25-27, 29, 32, 34-37, 39-48, 56-63, 67-70, 73-76, 93-103, 105, 111, 114, 117, 121, 123-126, 130, and 133-137.

TABLE 1

| | | | P4- | |
|---|---|---|---|---|
| | | MT4 | MAGI | |
| Example | | EC₅₀ | IC₅₀ | |
| No. | Structure | (nM) | (nM) | Analytical Data |
| 1 | 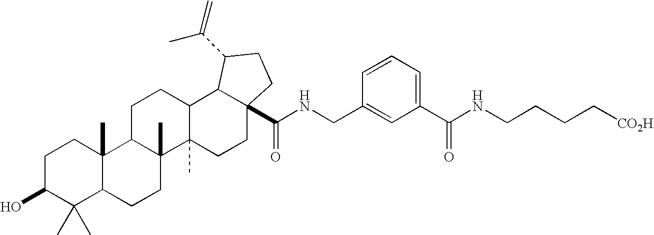 | 615 | 49.3 | ¹H NMR (DMSO-d6, 400 MHz) δ 8.41 (t, J = 5.6 Hz, 1H), 8.22 (t, J = 6.0 Hz, 1H), 7.73 (s, 1H), 7.65 (t, J = 5.6 Hz, 1H), 7.36 (d, J = 5.6 Hz, 2H), 4.64 (s, 1H), 4.53 (s, 1H), 4.34 (m, 1H), 4.20 (m, 1H), 3.23 (m, 1H), 3.01 (m, 2H), 2.25-1.05 (m, 34H), 0.90 (s, 3H), 0.86 (s, 3H), 0.73 (s, 3H), 0.70 (s, 3H), 0.64 (s, 3H). TOF-MS m/z 689 (M + H)⁺ |
| 2 | 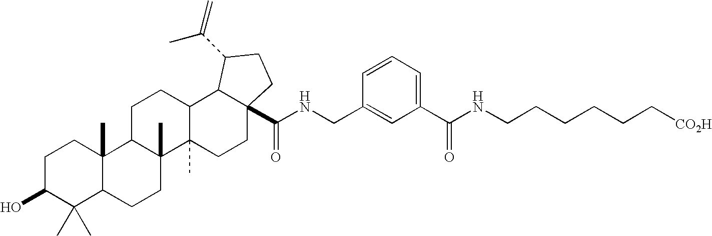 | 940 | 46.7 | ¹H NMR (DMSO-d6, 400 MHz) δ 8.38 (t, J = 5.6 Hz, 1H), 8.22 (t, J = 6.0 Hz, 1H), 7.72 (s, 1H), 7.65 (t, J = 5.6 Hz, 1H), 7.36 (d, J = 5.6 Hz, 2H), 4.64 (s, 1H), 4.53 (s, 1H), 4.31 (m, 1H), 4.19 (m, 1H), 3.23 (m, 1H), 3.01 (m, 2H), 2.25-1.05 (m, 37H), 0.89 (s, 3H), 0.86 (s, 3H), 0.73 (s, 3H), 0.69 (s, 3H), 0.64 (s, 3H). TOF-MS m/z 717 (M + H)⁺ |
| 3 | 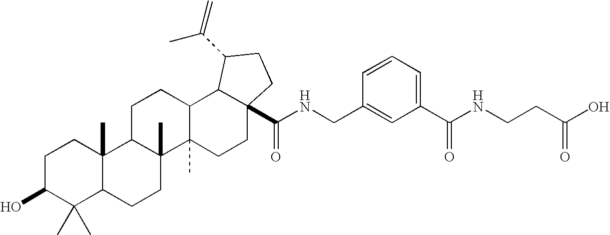 | 10000 | N/A | ¹H NMR (DMSO-d6, 400 MHz) δ 8.48 (t, J = 5.6 Hz, 1H), 8.32 (t, J = 6.0 Hz, 1H), 7.73 (s, 1H), 7.65 (t, J = 5.6 Hz, 1H), 7.37 (d, J = 5.6 Hz, 2H), 4.64 (s, 1H), 4.53 (s, 1H), 4.31 (m, 2H), 4.19 (m, 1H), 3.23 (m, 1H), 3.01 (m, 2H), 2.25-1.05 (m, 29H), 0.90 (s, 3H), 0.86 (s, 3H), 0.74 (s, 3H), 0.71 (s, 3H), 0.64 (s, 3H). TOF-MS m/z 661 (M + H)⁺ |
| 4 | 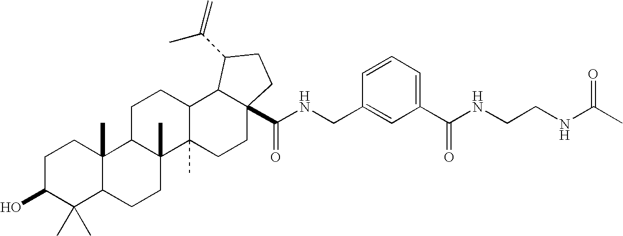 | 2300 | 155 | ¹H NMR (DMSO-d6, 400 MHz) δ 8.48 (t, J = 5.6 Hz, 1H), 8.23 (t, J = 6.0 Hz, 1H), 7.99 (t, J = 5.6 Hz, 1H), 7.74 (s, 1H), 7.66 (m, 1H), 7.37 (d, J = 4.8 Hz, 2H), 4.64 (s, 1H), 4.53 (s, 1H), 4.28 (m, 2H), 3.28 (m, 2H), 3.20 (m, 2H), 3.00 (m, 1H), 2.19 (m, 1H), 1.90-0.95 (m, 30H), 0.90 (s, 3H), 0.86 (s, 3H), 0.74 (s, 3H), 0.71 (s, 3H), 0.64 (s, 3H). TOF-MS m/z 674 (M + H)⁺ |
| 5 | 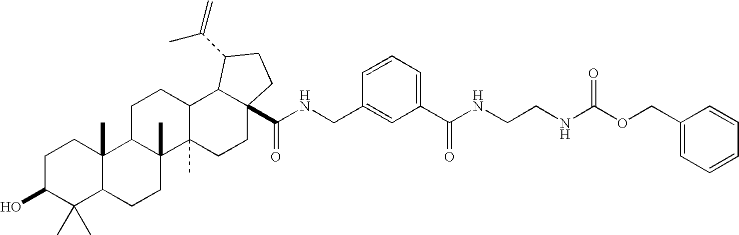 | 2500 | 107 | ¹H NMR (DMSO-d6, 400 MHz) δ 8.48 (t, J = 5.6 Hz, 1H), 8.23 (t, J = 6.0 Hz, 1H), 7.74 (s, 1H), 7.66 (m, 1H), 7.38-7.30 (m, 7H), 5.02 (s, 2H), 4.64 (s, 1H), 4.53 (s, 1H), 4.26 (m, 2H), 3.17 (m, 3H), 3.00 (m, 1H), 2.47 (m, 1H), 2.21 (m, 1H), 1.90-0.95 (m, 28H), 0.90 (s, 3H), 0.86 (s, 3H), 0.73 (s, 3H), 0.71 (s, 3H), 0.64 (s, 3H). TOF-MS m/z 766 (M + H)⁺ |
| 6 | 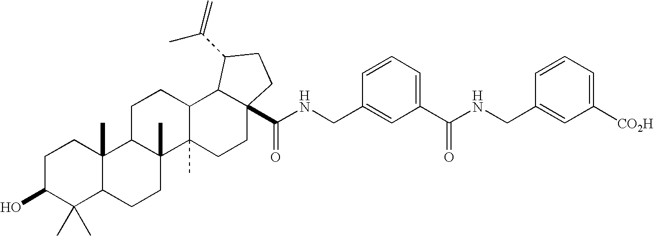 | 71 | 15.6 | ¹H NMR (400 MHz, DMSO) δ 12.95 (s, 1H), 9.08 (bt, J = 6 Hz, 1H), 8.22 (bt, J = 6 Hz, 1H), 7.91 (s, 1H), 7.82 (d, J = 6 Hz, 1H), 7.79 (s, 1H), 7.74 (m, 1H), 7.56 (d, J = 8 Hz, 1H), 7.45 (t, J = 8 Hz, 1H), 7.395 (d, J = 5 Hz, 2H), 4.64 (s, 1H), 4.53 (s, 3H), 4.27 (m, 3H), 3.60 (m, 2H), 2.98 (m, 2H), 2.3-0.4 (m, 40H) [M + H]+ Ion mass = 723.4713, [M + Na]+ Ion mass = 745.4535 |
| 7 | 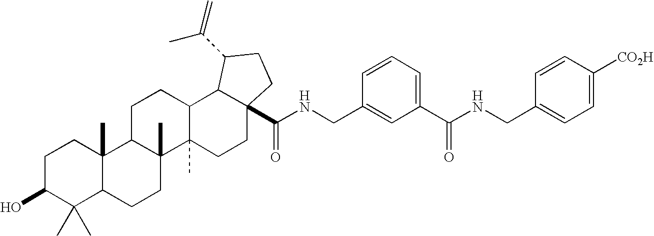 | 7.2 | 21.4 | ¹H NMR (400 MHz, DMSO) δ 12.88 (s, 1H), 9.07 (bt, J = 6 Hz, 1H), 8.23 (bt, J = 6 Hz, 1H), 7.89 (d, H = 9 Hz, 2H), 7.80 (s, 1H), 7.75 (m, 1H), 7.45-7.38 (m, 4H), 4.64 (s, 1H), 4.53 (m, 3H), 4.27 (m, 3H), 2.98 (m, 2H), 2.3-0.4 (m, 42H) [M + H]+ Ion mass = 723.4729, [M + Na]+ Ion mass = 745.4546 |
| 8 | 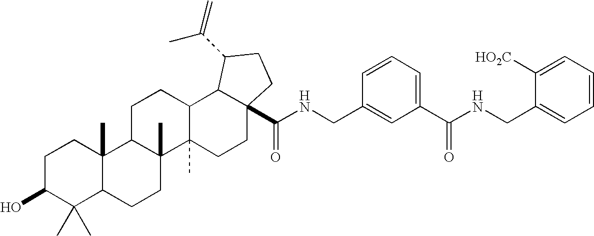 | N/A | 875 | ¹H NMR (400 MHz, DMSO) δ 8.30 (bt, J = 6 Hz, 1H), 8.00-7.30 (m, 8H), 4.81 (d, J = 6 Hz, 1H), 4.64 (s, 1H), 4.53 (s, 1H), 4.28 (m, 2H), 2.98 (m, 2H), 2.3-0.4 (m, 44H) [M + H]+ Ion mass = 723.4716, [M + Na]+ Ion mass = 745.4538 |
| 9 | 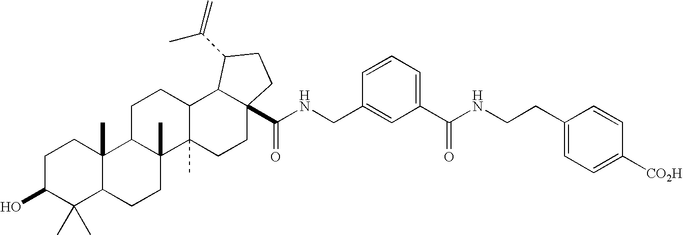 | 5.5 | 22.3 | ¹H NMR (400 MHz, DMSO) δ 12.83 (s, 1H), 8.55 (bt, J = 6 Hz, 1H), 8.22 (bt, J = 6 Hz, 1H), 7.87 (d, J = 9 Hz, 2H), 7.73 (s, 1H), 7.62 (m, 1H), 7.36 (m, 4H), 4.64 (s, 1H), 4.53 (s, 1H), 4.26 (m, 3H), 3.49 (m, 2H), 2.96 (m, 4H), 2.19 (d, J = 15 Hz, 1H), 1.9-0.5 (m, 41H) [M + H]+ Ion mass = 737.4893, [M + Na]+ Ion mass = 759.4712 |
| 10 | 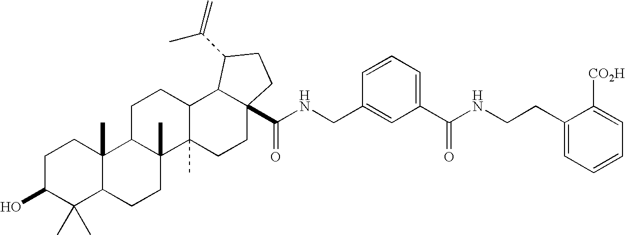 | 1300 | 128 | ¹H NMR (400 MHz, DMSO) δ 12.94 (s, 1H), 8.51 (br, J = 6 Hz, 1H), 8.21 (bt, J = 6 Hz, 1H), 7.82 (dd, J = 2, 8 Hz, 1H), 7.72 (s, 1H), 7.62 (m, 1H), 7.45 (dt, J = 2, 8 Hz, 1H), 7.32 (m, 4H), 4.64 (s, 1H), 4.53 (s, 1H), 4.26 (m, 3H), 3.49 (q, J = 7 Hz, 2H), 3.2 (t, J = 7 Hz, 2H), 2.98 (m, 2H), 2.20 (d, J = 13 Hz, 1H), 1.9-0.5 (m, 41H) [M + H]+ Ion mass = 737.4878, [M + Na]+ Ion mass = 759.4694 |
| 11 | 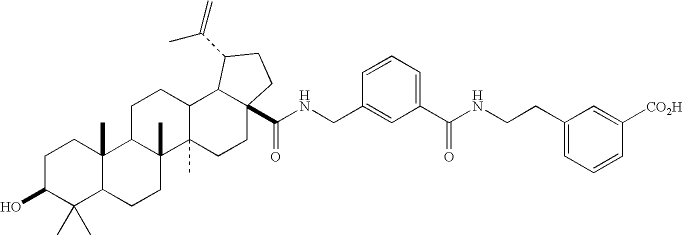 | N/A | 55 | ¹H NMR (400 MHz, DMSO) δ 12.91 (s, 1H), 8.55 (bs, 1H), 8.22 (bs, 1H), 7.89-7.28 (m, 8H), 4.64 (s, 1H), 4.53 (m, 3H), 3.50 (m, 3H), 2.49 (m, 4H), 2.3-0.4 (m, 42H) [M + H]+ Ion mass = 737.4854, [M + H]+ Ion mass = 759.4669 |
| 12 | 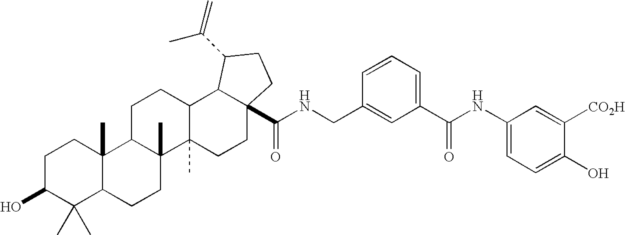 | N/A | 7200 | ¹H NMR (400 MHz, DMSO) δ 10.16 (s, 1H), 8.26 (bt, J = 6 Hz, 1H), 8.15 (m, 1H), 7.83 (m, 3H), 7.43 (m, 2H), 6.82 (d, J = 9 Hz, 1H), 4.64 (s, 1H), 4.53 (s, 1H), 4.34 (m, 2H), 2.98 (m, 2H), 2.22 (d, J = 13 Hz, 1H), 1.9-0.5 (m, 44H) [M + H]+ Ion mass = 725.4530, [M + Na]+ Ion mass = 747.4349 |
| 13 | 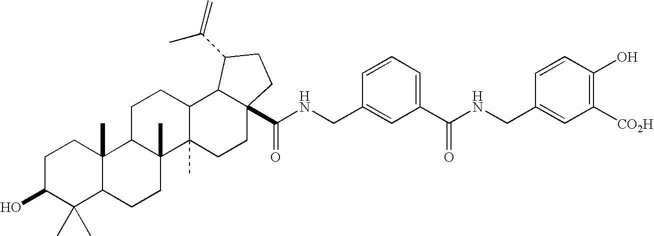 | 873 | 28 | ¹H NMR (400 MHz, DMSO) δ 9.05 (bt, J = 6 Hz, 1H), 8.26 (bt, J = 6 Hz, 1H), 7.75 (m, 3H), 7.46 (d, J = 8 Hz, 1H), 7.38 (m, 2H), 6.89 (d, J = 8 Hz, 1H), 4.64 (s, 1H), 4.52 (s, 1H), 4.39 (d, J = 6 Hz, 2H), 4.25 (m, 3H), 2.97 (m, 2H), 2.21 (d, J = 13 Hz, 1H), 1.9-0.5 (m, 42H) [M + H]+ Ion mass = 739.4677, [M + Na]+ Ion mass = 761.4498 |
| 14 | 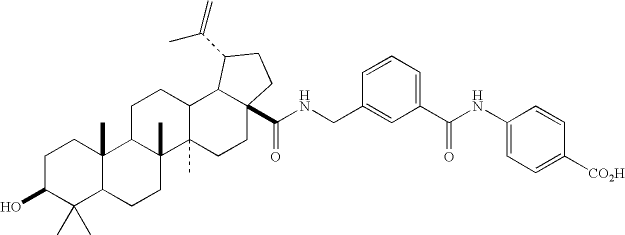 | N/A | 1800 | [M + H]+ Ion mass = 709.4575, [M + Na]+ Ion mass = 731.4394 |
| 15 | 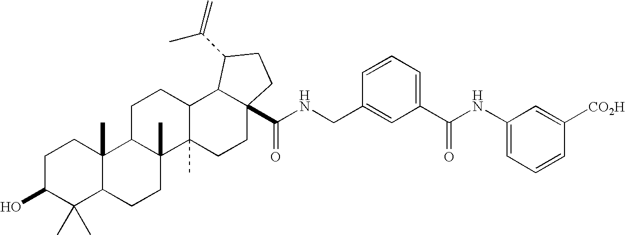 | N/A | 320 | ¹H NMR (400 MHz, DMSO) δ 12.94 (bs, 1H), 10.43 (s, 1H), 8.45 (s, 1H), 8.27 (bt, J = 6 Hz, 1H), 8.06 (d, J = 9 Hz, 1H), 7.84 (s, 1H), 7.79 (m, 1H), 7.66 (d, J = 8 Hz, 1H), 4.50 (m, 2H), 4.12 (m, 1H), 2.96 (m, 3H), 2.21 (d, J = 13 Hz, 1H), 1.8-0.5 (m, 41H) [M + H]+ Ion mass = 709.4574, [M + Na]+ Ion mass = 731.4431 |
| 16 | 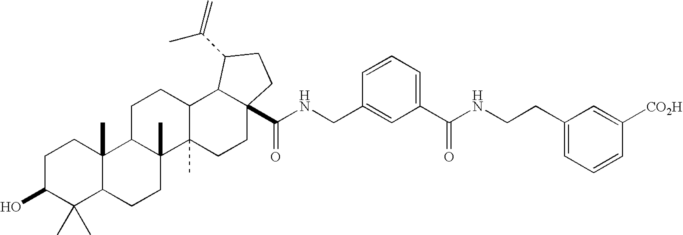 | 210 | N/A | ¹H NMR (400 MHz, DMSO) δ 12.91 (s, 1H), 8.55 (bs, 1H), 8.22 (bs, 1H), 7.89-7.28 (m, 8H), 4.64 (s, 1H), 4.53 (m, 3H), 3.50 (m, 3H), 2.94 (m, 4H), 2.3-0.4 (m, 42H) [M + H]+ Ion mass = 737.4854, [M + Na]+ Ion mass = 759.4669 |
| 17 | 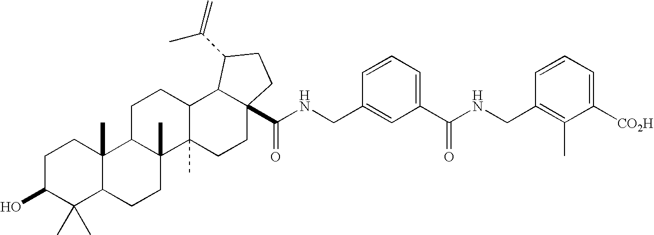 | 87 | 32 | ¹H NMR (DMSO-d6, 400 MHz) δ 8.86 (m, 1H), 8.23 (m, 1H), 7.79 (s, 1H), 7.73 (m, 1H), 7.39 (m, 3H), 7.23 (m, 1H), 7.12 (d, J = 7.6 Hz, 1H), 4.64 (s, 1H), 4.53 (s, 1H)4.46 (br s, 2H), 4.26 (m, 2H), 2.97 (m, 2H), 2.41 (s, 3H), 2.20-0.80 (m, 31H), 0.74 (s, 3H), 0.72 (s, 3H), 0.64 (s, 3H). TOF-MS m/z 737 (M + H)⁺ |
| 18 | 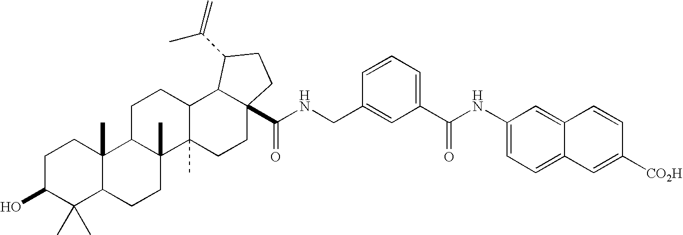 | 6100 | 108 | ¹H NMR (400 MHz, DMSO) δ 12.95 (bs, 1H), 10.59 (s, 1H), 8.56 (m, 2H), 8.23 (bt, J = 6 Hz, 1H), 8.18-7.75 (m, 6H), 7.45 (m, 2H), 4.63 (s, 1H), 4.54 (m, 2H), 4.27 (m, 1H), 4.10 (m, 1H), 3.10-0.20 (m, 44H) [M + H]+ Ion mass = 759.4730, [M + Na]+ Ion mass = 781.4561 |
| 19 | 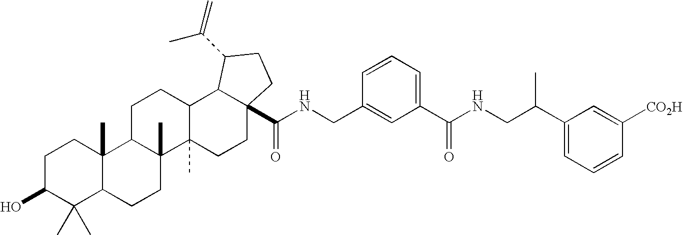 | 19 | 6.2 | ¹H NMR (DMSO-d6, 400 MHz) δ 8.48 (m, 1H), 8.24 (m, 1H), 7.83 (s, 1H), 7.76 (d, J = 7.2 Hz, 1H), 7.70 (s, 1H), 7.60 (s, 1H), 7.44 (d, J = 6.8 Hz, 1H), 7.35 (m, 3H), 4.64 (s, 1H), 4.53 (s, 1H), 4.31 (m, 2H), 4.17 (m, 1H), 2.99 (m, 2H), 2.50-0.71 (m, 41H), 0.61 (s, 3H), 0.59 (s, 3H). TOF-MS m/z 751 (M + H)⁺ |
| 20 | 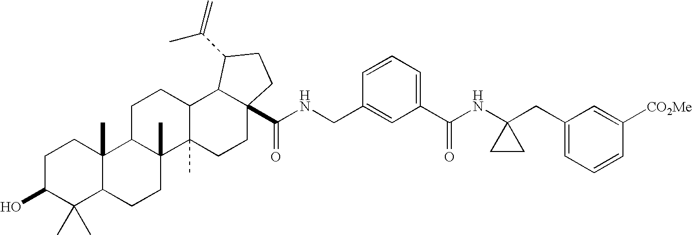 | N/A | 357 | ¹H NMR (DMSO-d6, 400 MHz) δ 8.02 (t, 1H, J = 5.1 Hz, NH), 7.79 (d, 2H, J = 2.0 Hz, CH Arom), 7.69 (bs, 1H, CH Arom), 7.52 (d, 1H, J = 7.6 Hz, CH Arom), 7.40 (bs, 2H, CH Arom), 7.32-7.29 (m, 2H, CH Arom), 4.64 (bs, 1H, CH═), 4.52 (bs, 1H, CH═), 4.34-4.29 (dd, 1H, J = 6.0, J = 5.6, CH2), 4.27 (d, 1H, J = 5.3 Hz, H-3), 4.17-4.12 (dd, 1H, J = 6.0, J = 5.8, CH2), 3.00-0.64 (m, 52H). 763.5046 (M + H)+. |
| 21 | 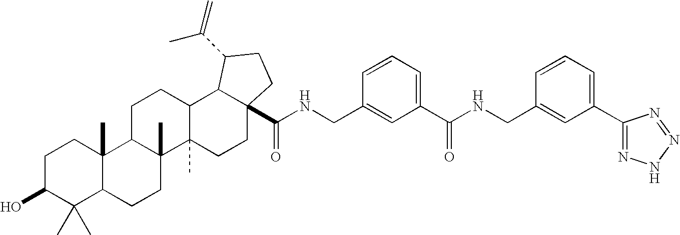 | 180 | 24.8 | ¹H NMR (DMSO-d6, 400 MHz) 9.11 (m, 1H), 8.23 (m, 1H), 8.03 (s, 1H), 7.91 (d, H = 6.8 Hz, 1H), 7.81 (s, 1H), 7.76 (m, 1H), 7.54 (m, 2H), 7.41 (m, 2H), 4.64 (s, 1H), 4.57 (br s, 2H), 4.52 (s, 1H), 4.26 (m, 2H), 2.97 (m, 2H), 2.20-0.80 (m, 33H), 0.71 (s, 6H), 0.62 (s, 3H). TOF-MS m/z 747 (M + H)⁺ |
| 22 | 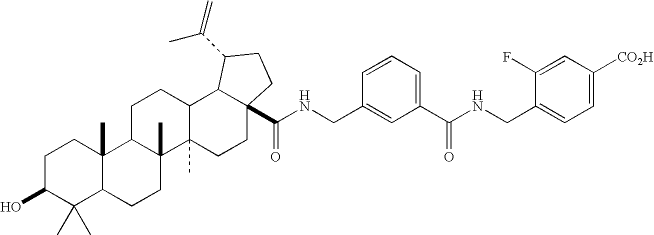 | 9.7 | 15.4 | ¹H NMR (400 MHz, d6-DMSO) δ 9.04-9.07 (1H, m), 8.20-8.25 (1H, m), 7.78-7.80 (1H, m), 7.70-7.75 (2H, m), 7.64 (1H, dd, J = 10 & 1.2 Hz), 7.38-7.48 (3H, m), 4.30-35 (1H, m), 4.15-4.22 (1H, m), 2.90-3.00 (1H, m), 0.5-3.3 (45H, m). Mass Spec (m/z): 742 (M + 1). |
| 23 | 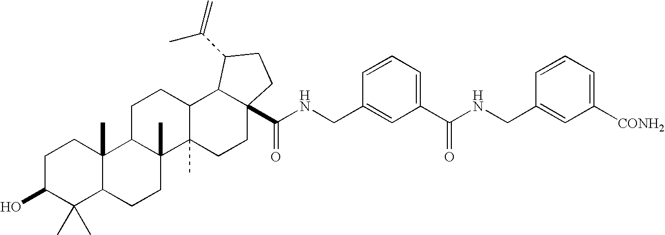 | 329 | 52.7 | ¹H NMR (400 MHz, d6-DMSO) δ 9.04 (bt, J = 6 Hz, 1H), 8.22 (bt, J = 6 Hz, 1H), 7.97 (s, 1H), 7.83 (s, 1H), 7.79 (s, 1H), 7.74 (m, 2H), 7.39 (m, 5H), 4.64 (s, 1H), 4.51 (m, 3H), 4.27 (m, 3H), 2.98 (m, 2H), 2.19 (d, J = 13, 1H), 1.90-0.55 (m, 41H) [M + H]+ Ion mass = 722.4891, [M + Na]+ Ion mass = 744.4706 |
| 24 | 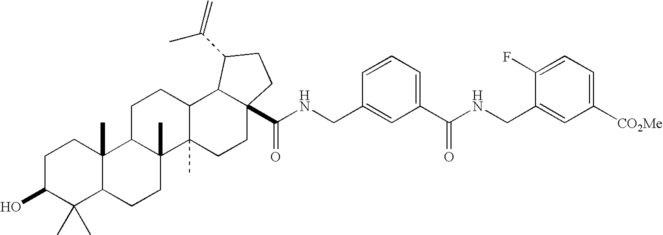 | >10000 | 186 | ¹H NMR (400 MHz, d6-DMSO) δ 9.08 (t, J = 5.8 Hz, 1H), 8.24 (t, J = 6.0 Hz, 1H), 7.99 (dd, J = 2, 7.4 Hz, 1H), 7.92 (m, 1H), 7.78 (s, 1H), 7.74 (m, 1H), 7.40 (d, J = 5 Hz, 2H), 7.35 (dd, J = 9, 11 Hz, 1H), 4.63 (d, J = 2.1 Hz, 1H), 4.54 (m, 3H), 4.28 (m, 2H), 3.87 (s, 3H), 2.96 (m, 2H), 2.50-0.63 (m, 43H); Mass Spec (m/z): 755.5 (M + 1). |
| 25 | 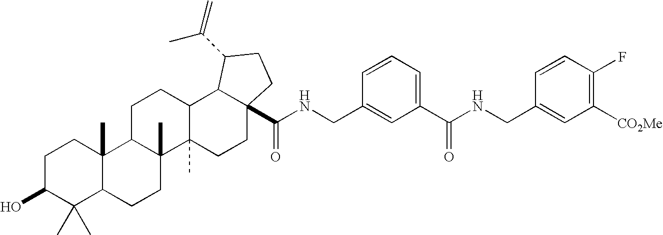 | 100 | 40.4 | ¹H NMR (400 MHz, d6-DMSO) δ 9.08 (t, J = 6.2 Hz, 1H), 8.24 (t, J = 5.7 Hz, 1H), 7.85 (dd, J = 2.3, 7 Hz, 1H), 7.79 (s, 1H), 7.72 (m, 1H), 7.61 (m, 1H), 7.39 (d, J = 5 Hz, 2H), 7.30 (dd, J = 9, 11 Hz, 1H), 4.63 (d, J = 1 Hz, 1H), 4.52 (s, 1H), 4.48 (d, J = 6 Hz, 1H), 4.52 (s, 1H), 4.48 (d, J = 6 Hz, 1H), 4.25 (m, 2H), 3.00 (m, 2H), 2.50-0.64 (m, 44H); Mass Spec (m/z): 755.4 (M + 1). |
| 26 | 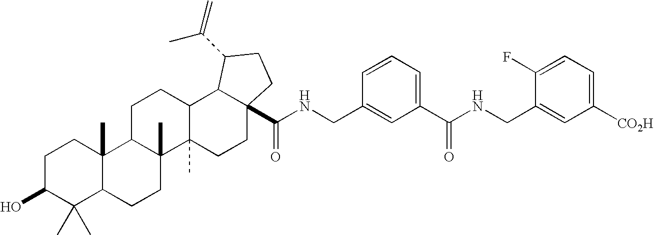 | 200 | 78.6 | ¹H NMR (400 MHz, d6-DMSO) δ 13.09 (s, 1H), 9.08 (t, J = 5.8 Hz, 1H), 8.24 (t, J = 6.0 Hz, 1H), 7.96 (dd, J = 2.1, 7.4 Hz, 1H), 7.89 (m, 1H), 7.41 (d, J = 5 Hz, 2H), 7.31 (dd, J = 9, 10.3 Hz, 1H), 4.64 (d, J = 2 Hz, 1H), 4.53 (m, 3H), 4.26 (m, 2H), 2.97 (m, 2H), 2.47-0.58 (m, 43H); Mass Spec (m/z): 741.5 (M + 1). |
| 27 | 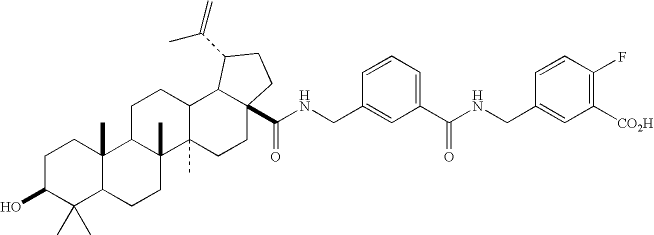 | 73 | 53.5 | ¹H NMR (400 MHz, d6-DMSO) δ 9.10 (t, J = 6.2 Hz, 1H), 8.24 (t, J = 5.7 Hz, 1H), 7.82 (dd, J = 2.5, 7 Hz, 1H), 7.78 (s, 1H), 7.73 (m, 1H), 7.56 (m, 1H), 7.39 (d, J = 5 Hz, 2H), 7.26 (dd, J = 9, 11 Hz, 1H), 4.63 (d, J = 2 Hz, 1H), 4.52 (s, 1H), 4.47 (d, J = 5.9 Hz, 2H), 4.26 (m, 2H), 2.96 (m, 2H), 2.50-0.65 (m, 43H); Mass Spec (m/z): 741.5 (M + 1). |
| 28 | 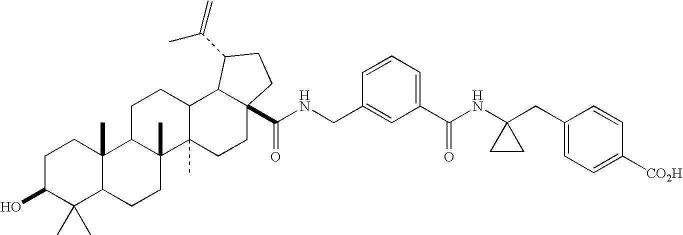 | N/A | 1200 | ¹H NMR (400 MHz, d6-DMSO) δ 8.20 (t, 1H, J = 5.1 Hz, NH), 7.85 (d, 2H, J = 8.3 Hz, CH Arom), 7.69 (bs, 1H, J = 7.6 Hz, CH Arom), 7.33-7.28 (m, 3H, CH Arom), 4.64 (bs, 1H, CH═), 4.52 (bs, 1H, CH═), 4.34-4.29 (dd, 1H, J = 6.0, J = 5.6, CH2), 4.27 (d, 1H, J = 5.3 Hz, H-3), 4.17-4.12 (dd, 1H, J = 6.0, J = 5.8, CH2), 3.00-0.64 (m, 53H). 763.5040 (M + H)+. |
| 29 | 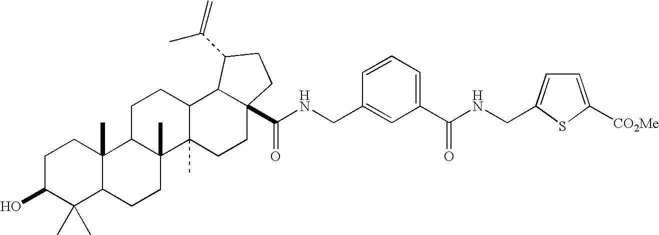 | 18 | 37.9 | ¹H NMR (400 MHz, d6-DMSO) δ 9.24-9.20 (1H, m), 8.25-8.20 (1H, m), 7.78 (1H, s), 7.72-7.69 (1H, m), 7.63-7.66 (1H, m), 7.38-7.40 (2H, m), 7.09-7.1 (1H, m), 4.66-4.63 (2H, m), 4.5 (1H, s), 4.17-4.22 (2H, m), 4.5 (1H, s), 4.17-4.22 (2H, m), 3.77 (3H, s), 3.5-0.55 (m, 46H). Mass Spec: (m/z): 743 (M + 1). |
| 30 | 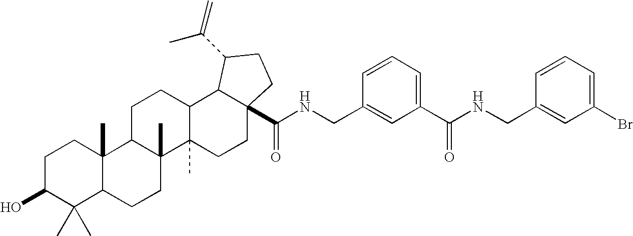 | N/A | 410 | ¹H NMR (400 MHz, d6-DMSO) δ 9.04 (bt, J = 6 Hz, 1H), 8.23 (bt, J = 6 Hz, 1H), 7.78 (s, 1H), 7.83 (s, 1H), 7.73 (m, 1H), 7.50 (s, 1H), 7.42 (m, 3H), 7.29 (m, 1H), 4.64 (s, 1H), 4.53 (s, 1H), 4.46 (d, J = 6 Hz, 2H), 4.26 (m, 3H), 2.98 (m, 2H), 2.19 (d, J = 13, 1H), 2.00-0.50 (m, 41H) [M + H]+ Ion mass = 757.3945, [M + Na]+ Ion mass = 779.3760 |
| 31 | 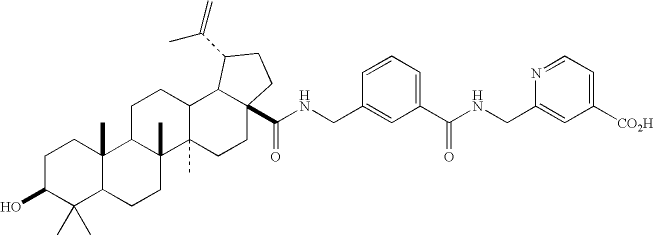 | N/A | 573 | ¹H NMR (400 MHz, d6-DMSO) δ 13.65 (bs, 1H), 9.15 (bt, J = 6 Hz, 1H), 8.70 (d, J = 5 Hz, 1H), 8.23 (bt, J = 6 Hz, 1H), 7.82 (s, 1H), 7.78 (bt, J = 6 Hz, 1H), 7.74 (s, 1H), 7.69 (d, J = 5 Hz, 1H), 7.20 (d, J = 5 Hz, 2H), 4.64 (m, 2H), 4.52 (s, 1H), 4.28 (m, 2H), 2.98 (m, 2H), 2.19 (d, J = 13, 1H), 1.90-0.50 (m, 43H) [M + H]+ Ion mass = 724.4678, [M + Na]+ Ion mass = 746.4503 |
| 32 | 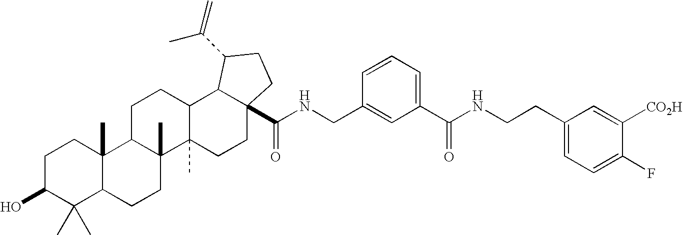 | 3000 | 15 | ¹H NMR (400 MHz, d6-DMSO) δ 13.19 (bs, 1H), 8.53 (bt, J = 6 Hz, 1H), 8.22 (bt, J = 6 Hz, 1H), 7.73 (m, 2H), 7.61 (m, 1H), 7.48 (m, 1H), 7.37 (s, 2H), 7.23 (t, J = 10 Hz, 1H), 4.64 (s, 1H), 4.53 (s, 1H), 4.40-4.10 (m, 2H), 3.60 (m, 2H), 2.92 (m, 5H), 2.19 (d, J = 13 Hz, 1H), 1.90-0.50 (m, 41H) [M + H]+ Ion mass = 755.4790, [M + Na]+ Ion mass = 777.4618 |
| 33 | 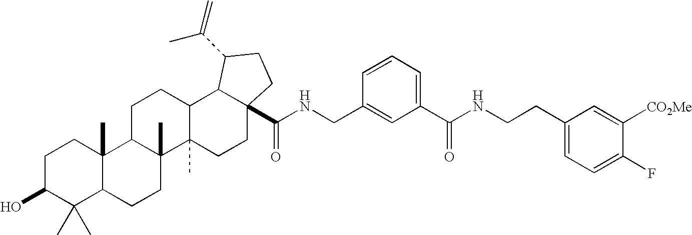 | 18 | 135 | [M + H]+ Ion mass = 769.4943, [M + Na]+ Ion mass = 791.4767 |
| 34 | 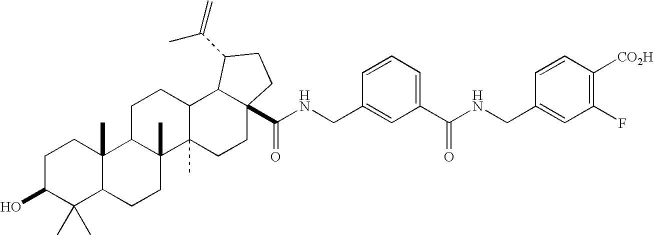 | 19 | 24.3 | ¹H NMR (DMSO-d6, 400 MHz) δ 9.04 (m, 1H), 8.23 (m, 1H), 7.79 (s, 1H), 7.73 (m, 1H), 7.63 (m, 1H), 7.40 (m, 2H), 7.05 (m, 2H), 4.64 (s, 1H), 4.53 (s, 1H), 4.46 (br s, 2H), 4.26 (m, 3H), 2.97 (m, 2H), 2.41 (s, 3H), 2.30-0.80 (m, 29H), 0.73 (s, 3H), 0.72 (s, 3H), 0.63 (s, 3H). |
| 35 | 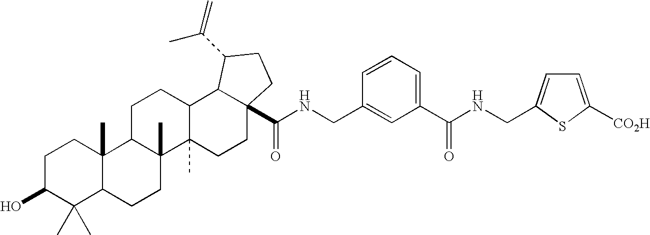 | 85 | 53.2 | ¹H NMR (400 MHz, d6-DMSO) δ 9.17-9.22 (1H, m), 8.20-8.25 (1H, m), 7.78 (1H, br s), 7.68-7.73 (1H, m), 7.56 (1H, d, J = 4 Hz), 7.37-7.41 (2H, m), 7.055 (1H, d, J = 4 Hz), 4.60-4.64 (3H, m), 4.52 (1H, br s), 4.17-4.35 (2H, m), 2.93-3.03 (1H, m), 0.55-2.5 (45H, m). Mass Spec (m/z): 729 (M + 1). |
| 36 | 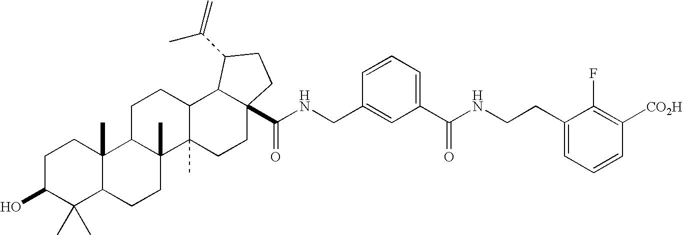 | 8.5 | 16.4 | ¹H NMR (400 MHz, d6-DMSO) δ 8.56-8.59 (1H, m), 8.21-8.24 (1H, m), 7.20-7.78 (7H, m), 4.64 (1H, br s), 4.53 (1H, br s), 4.17-4.4 (2H, m), 0.55-3.5 (50H, m). Mass Spec (m/z): 755 (M + 1). |
| 37 | 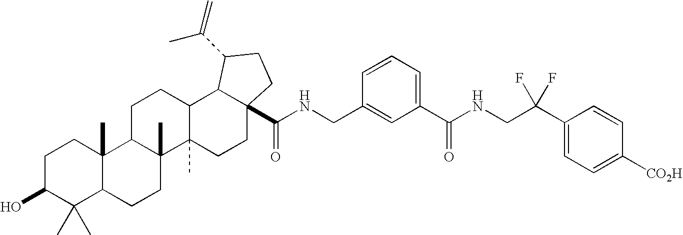 | 7.3 | 56.7 | ¹H NMR (DMSO-d6, 400 MHz) δ 8.85 (t, 1H, J = 6.4 Hz, NH), 8.21 (t, 1H, J = 6.4 Hz, NH), 8.03 (d, 1H, CH Arom), 7.39 (m, 3H, CH Arom), 6.51 (bs, 3H, CH Arom), 4.64 (bs, 1H, CH═), 4.52 (bs, 1H, CH═), 4.28-4.03 (m, 5H, H-3, 2 × CH2), 3.00-0.64 (m, 43H). 771.6 (M − H). |
| 38 | 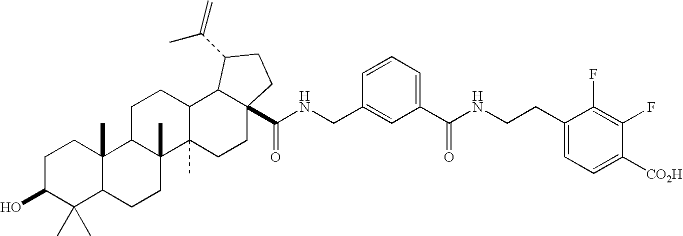 | 4.4 | 160 | ¹H NMR (DMSO-d6, 400 MHz) δ 8.57 (t, 1H, J = 6.0 Hz, NH), 8.21 (t, 1H, J = 6.0 Hz, NH), 7.69 (s, 1H, CH Arom), 7.63-7.59 (m, 2H, CH Arom), 7.37 (m, 2H, CH Arom), 7.37 (m, 2H, CH Arom), 7.23 (t, 1H, J = 8.2 Hz, CH Arom), 4.64 (bs, 1H, CH═), 4.52 (bs, 1H, CH═), 4.35-4.15 (m, 4H, 2 × CH2), 3.00-0.64 (m, 50H). 787.4866 (M + H)+. |
| 39 | 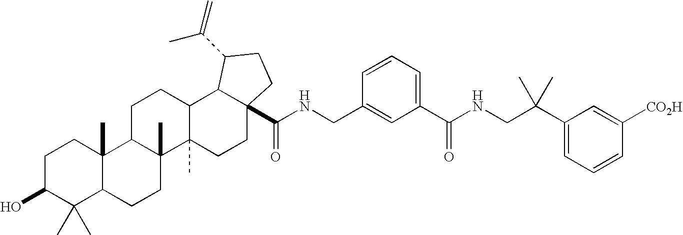 | 5.3 | 40.2 | ¹H NMR (DMSO-d6, 400 MHz) δ 8.26 (m, 2H), 8.00 (s, 1H), 7.77 (d, J = 7.6 Hz, 1H), 7.68 (s, 1H), 7.60 (m, 2H), 7.36 (m, 3H), 4.64 (s, 1H), 4.53 (s, 1H), 4.28 (m, 3H), 3.45 (m, 2H), 2.99 (m, 2H), 2.50-0.80 (m, 38H), 0.73 (s, 3H), 0.71 (s, 3H), 0.62 (s, 3H). TOF-MS m/z 765 (M + H)⁺ |
| 40 | 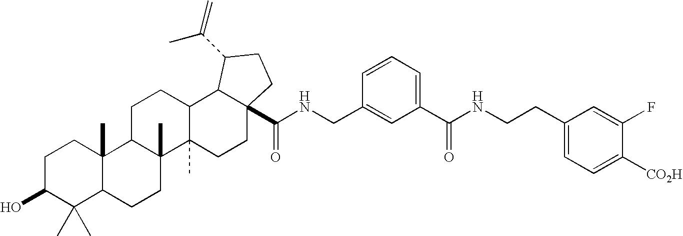 | 6.3 | 19.9 | ¹H NMR (DMSO-d6, 400 MHz) δ 8.53 (m, 1H), 8.23 (m, 1H), 7.72 (s, 1H), 7.62 (m, 2H), 7.36 (m, 2H), 7.03 (m, 2H), 4.64 (s, 1H), 4.53 (s, 1H), 4.28 (m, 3H), 3.48 (m, 2H), 2.99 (m, 2H), 2.50-0.72 (m, 40H), 0.61 (s, 3H). TOF-MS m/z 755 (M + H)⁺ |
| 41 | 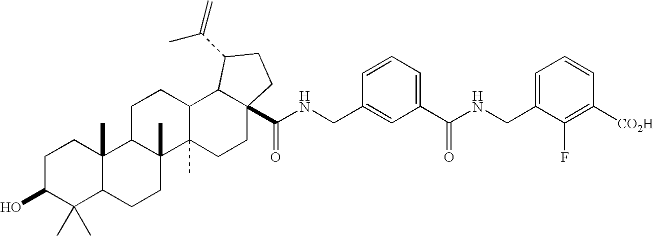 | 1.9 | 36.7 | ¹H NMR (400 MHz, d6-DMSO) δ 13.25 (bs, 1H), 9.02 (bt, J = 6 Hz, 1H), 8.22 (bt, J = 6 Hz, 1H), 7.75 (m, 3H), 7.55 (m, 1H), 7.39 (m, 2H), 7.24 (t, J = 9 Hz, 1H), 4.64 (s, 1H), 4.53 (m, 3H), 4.38-4.13 (m, 3H), 3.60 (m, 2H), 2.97 (m, 3H), 2.19 (d, J = 13 Hz, 1H), 1.90-0.50 (m, 38H) [M + H]+ Ion mass = 741.4636, [M + Na]+ Ion mass = 763.4462 |
| 42 | 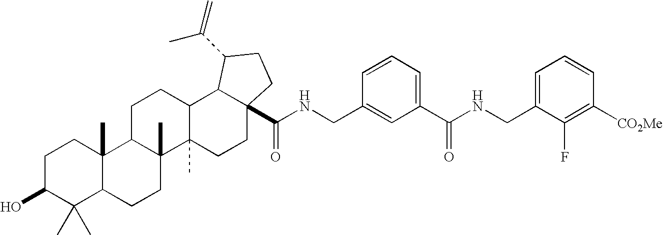 | 42 | 58.3 | ¹H NMR (400 MHz, d6-DMSO) δ 9.03 (bt, J = 6 Hz, 1H), 8.23 (bt, J = 6 Hz, 1H), 7.75 (m, 3H), 7.59 (m, 1H), 7.40 (m, 2H), 7.28 (t, J = 8 Hz, 1H), 4.64 (s, 1H), 4.53 (m, 3H), 4.38-4.15 (m, 3H), 3.86 (s, 3H), 2.97 (m, 3H), 2.19 (d, J = 13 Hz, 1H), 1.90-0.50 (m, 40H) [M + H]+ Ion mass = 755.4802, [M + Na]+ Ion mass = 777.4618 |
| 43 | 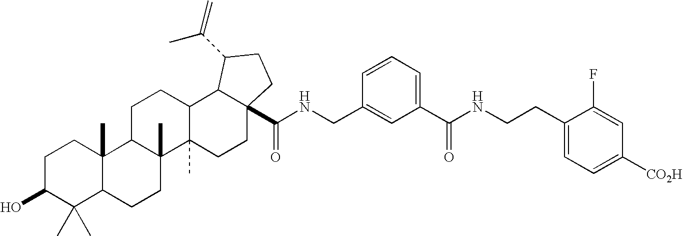 | 9.3 | 16.8 | ¹H NMR (400 MHz, d6-DMSO) δ 13.17 (bs, 1H), 8.58 (bt, J = 6 Hz, 1H), 8.22 (bt, J = 6 Hz, 1H), 7.71 (m, 2H), 7.61 (m, 2H), 7.44 (t, J = 8 Hz, 1H), 7.36 (m, 2H), 4.64 (s, 1H), 4.53 (s, 1H), 4.38-4.12 (m, 2H), 3.50 (m, 2H), 2.94 (m, 3H), 2.19 (d, J = 13 Hz, 1H), 1.90-0.50 (m, 43H) [M + H]+ Ion mass = 755.4794, [M + Na]+ Ion mass = 777.4610 |
| 44 | 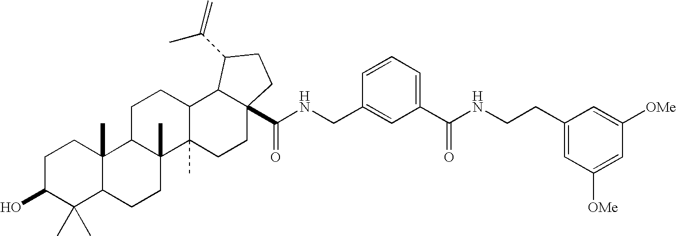 | N/A | 52 | ¹H NMR (400 MHz, d6-DMSO) δ 8.52 (bt, J = 6 Hz, 1H), 8.23 (bt, J = 6 Hz, 1H), 7.74 (s, 1H), 7.64 (m, 1H), 7.36 (d, J = 5 Hz, 1H), 6.39 (d, J = 2 Hz, 2H), 6.33 (t, J = 2 Hz, 1H), 4.64 (s, 1H), 4.53 (s, 1H), 4.38-4.12 (m, 3H), 3.70 (s, 6H), 3.45 (m, 2H), 2.98 (m, 2H), 2.77 (m, 2H), 2.20 (d, J = 13 Hz, 1H), 1.90-0.50 (m, 42H) [M + H]+ Ion mass = 753.5197, [M + Na]+ Ion mass = 775.5016 |
| 45 | 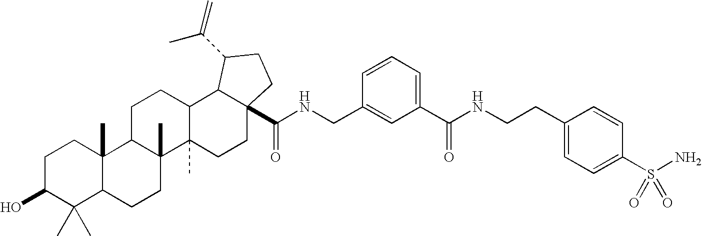 | 94 | 15.9 | ¹H NMR (400 MHz, d6-DMSO) δ 8.56 (bt, J = 6 Hz, 1H), 8.23 (bt, J = 6 Hz, 1H), 7.74 (m, 3H), 7.63 (m, 1H), 7.42 (d, J = 8 Hz, 2H), 7.36 (d, J = 5 Hz, 2H), 7.30 (s, 2H), 4.64 (s, 1H), 4.53 (s, 1H), 4.39-4.13 (m, 3H), 3.49 (m, 2H), 2.96 (m, 4H), 2.19 (d, J = 13 Hz, 1H), 2.09 (s, 1H), 1.9-0.5 (m, 40H) [M + H]+ Ion mass = 772.4709, [M + Na]+ Ion mass = 793.4419 |
| 46 | 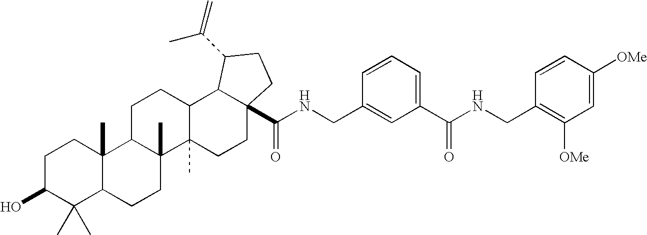 | N/A | 59 | ¹H NMR (400 MHz, d6-DMSO) δ 8.68 (bt, J = 6 Hz, 1H), 8.22 (bt, J = 6 Hz, 1H), 7.77 (s, 1H), 7.72 (m, 1H), 7.39 (m, 2H), 7.06 (d, J = 8 Hz, 1H), 6.56 (d, J = 2 Hz, 1H), 6.45 (dd, J = 8.2 Hz, 1H), 4.64 (s, 1H), 4.53 (s, 1H), 4.41-4.15 (m, 5H), 3.80 (s, 3H), 3.73 (s, 3H), 2.98 (m, 2H), 2.19 (d, J = 18 Hz, 1H), 1.90-0.50 (m, 40H) [M + H]+ Ion mass = 739.5041, [M + Na]+ Ion mass = 761.4864 |
| 47 | 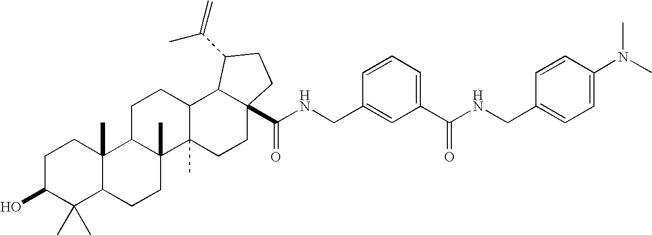 | 41.3 | 36.3 | ¹H NMR (400 MHz, d6-DMSO) δ 8.82 (bt, J = 6 Hz, 1H), 8.22 (bt, J = 6 Hz, 1H), 7.77 (s, 1H), 7.70 (m, 1H), 7.37 (m, 2H), 7.13 (d, J = 9 Hz, 2H), 6.67 (d, J = 9 Hz, 2H), 4.64 (s, 1H), 4.53 (s, 1H), 4.38-4.15 (m, 5H), 2.98 (m, 2H), 2.85 (s, 6H), 2.19 (d, J = 13 Hz, 1H), 1.9-0.5 (m, 41H) [M + H]+ Ion mass = 722.5255, [M + Na]+ Ion mass = 744.5080 |
| 48 | 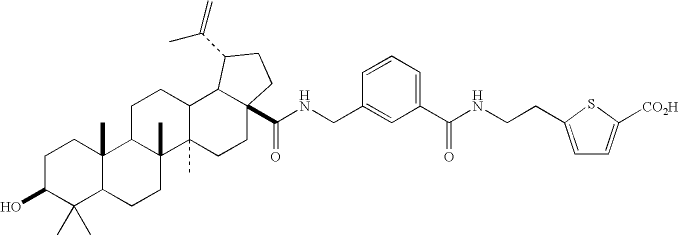 | 63 | 29 | ¹H NMR (400 MHz, d6-DMSO) δ 12.91 (bs, 1H), 8.63 (bt, J = 6 Hz, 1H), 8.23 (bt, J = 6 Hz, 1H), 7.75 (s, 1H), 7.65 (m, 1H), 7.57 (d, J = 4 Hz, 1H), 7.38 (d, J = Hz, 2H), 6.98 (d, J = 4 Hz, 1H), 4.64 (s, 1H), 4.53 (s, 1H), 4.39-4.12 (m, 3H), 3.60-0.50 (m, 48H) [M + H]+ Ion mass = 743.4444, [M + Na]+ Ion mass = 765.4280 |
| 49 | 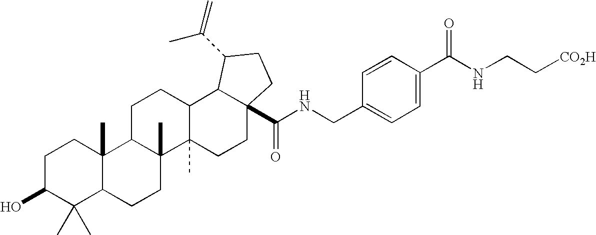 | 10000 | N/A | ¹H NMR (DMSO-d6, 400 MHz) δ 8.48 (m, 1H), 8.23 (m, 1H), 7.76 (d, J = 8.4 Hz, 2H), 7.29 (d, J = 8.4 Hz, 2H), 4.64 (s, 1H), 4.53 (s, 1H), 4.40-4.20 (m, 3H), 3.44 (m, 2H), 3.01 (m, 1H), 2.25-0.95 (m, 29H), 0.91 (s, 3H), 0.87 (s, 3H), 0.79 (s, 3H), 0.76 (s, 3H), 0.65 (s, 3H). TOF-MS m/z 661 (M + H)⁺ |
| 50 | 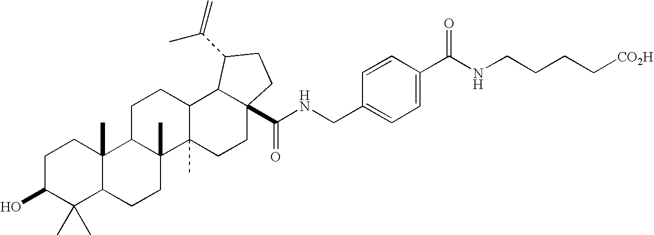 | 10000 | N/A | ¹H NMR (DMSO-d6, 400 MHz) δ 8.41 (m, 1H), 8.23 (m, 1H), 7.76 (d, J = 8.4 Hz, 2H), 7.29 (d, J = 8.4 Hz, 2H), 4.65 (s, 1H), 4.53 (s, 1H), 4.36-4.17 (m, 2H), 3.24 (m, 2H), 3.01 (m, 1H), 2.25-0.95 (m, 32H), 0.91 (s, 3H), 0,87 (s, 3H), 0.79 (s, 3H), 0.76 (s, 3H), 0.65 (s, 3H). TOF-MS m/z 689 (M + H)⁺ |
| 51 | 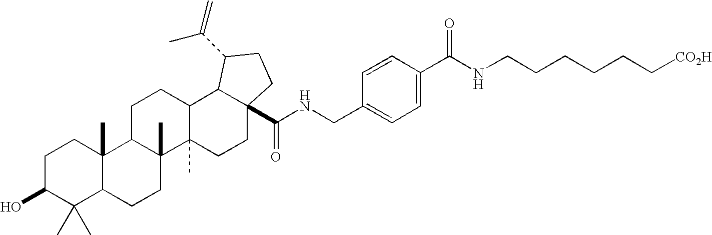 | 10000 | N/A | ¹H NMR (DMSO-d6, 400 MHz) δ 8.38 (m, 1H), 8.23 (m, 1H), 7.76 (d, J = 8.4 Hz, 2H), 7.29 (d, J = 8.4 Hz, 2H), 4.65 (s, 1H), 4.53 (s, 1H), 4.36-4.17 (m, 2H), 3.22 (m, 2H), 3.01 (m, 1H), 2.25-0.95 (m, 36H), 0.91 (s, 3H), 0.86 (s, 3H), 0.77 (s, 3H), 0.75 (s, 3H), 0.65 (s, 3H). TOF-MS m/z 717 (M + H)⁺ |
| 52 | 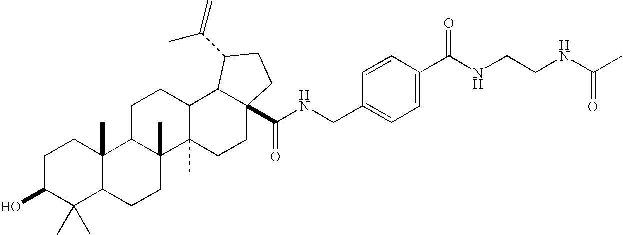 | 10000 | N/A | ¹H NMR (DMSO-d6, 400 MHz) δ 8.47 (m, 1H), 8.24 (m, 1H), 7.99 (m, 1H), 7.76 (d, J = 8.4 Hz, 2H), 7.29 (d, J = 8.4 Hz, 2H), 4.64 (s, 1H), 4.53 (s, 1H), 4.36-4.17 (m, 2H), 3.28 (m, 2H), 3.19 (m, 2H), 3.00 (m, 1H), 2.25-0.95 (m, 31H), 0.91 (s, 3H), 0.86 (s, 3H), 0.80 (s, 3H), 0.76 (s, 3H), 0.65 (s, 3H). |
| 53 | 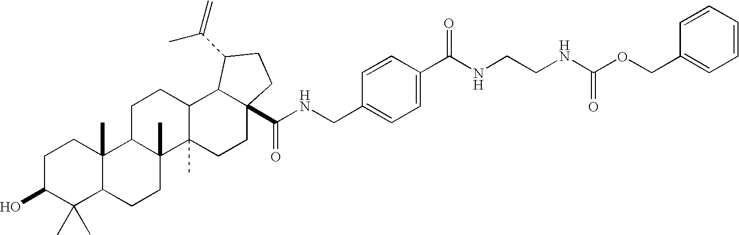 | 10000 | N/A | ¹H NMR (DMSO-d6, 400 MHz) δ 8.46 (m, 1H), 8.24 (m, 1H), 7.76 (d, J = 8.4 Hz, 2H), 7.40-7.28 (m, 8H), 5.02 (s, 2H), 4.64 (s, 1H), 4.53 (s, 1H), 4.36-4.17 (m, 2H), 3.17 (m, 3H), 3.00 (m, 1H), 2.25-0.95 (m, 29H), 0.91 (s, 3H), 0.86 (s, 3H), 0.80 (s, 3H), 0.75 (s, 3H), 0.64 (s, 3H). TOF-MS m/z 766 (M + H)⁺ |
| 54 | 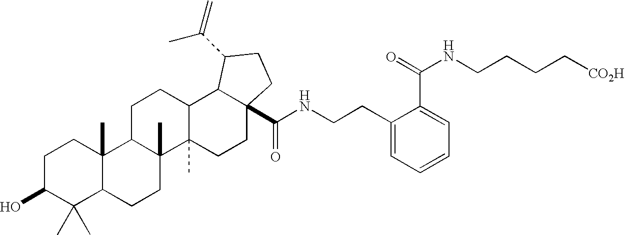 | N/A | 3400 | ¹H NMR (DMSO-d6, 400 MHz) δ 8.43 (m, 1H), 7.92 (m, 1H), 7.36 (t, J = 7.2 Hz, 1H), 7.30 (d, J = 6.8 Hz, 2H), 7.24 (t, J = 7.2 Hz, 1H), 4.63 (s, 1H), 4.51 (s, 1H), 4.28 (s, 1H), 3.24 (m, 2H), 3.00 (m, 1H), 2.80 (m, 2H), 2.48-0.95 (m, 35H), 0.86 (s, 3H), 0.85 (s, 3H), 0.74 (s, 3H), 0.69 (s, 3H), 0.64 (s, 3H). TOF-MS m/z 703 (M + H)⁺ |
| 55 | 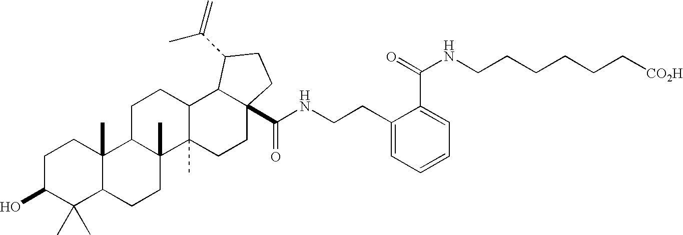 | N/A | 4700 | ¹H NMR (DMSO-d6, 400 MHz) δ 8.39 (m, 1H), 7.93 (m, 1H), 7.36 (t, J = 7.2 Hz, 1H), 7.30 (d, J = 6.8 Hz, 2H), 7.24 (t, J = 7.2 Hz, 1H), 4.63 (s, 1H), 4.51 (s, 1H), 4.27 (s, 1H), 3.24 (m, 2H), 3.00 (m, 1H), 2.80 (m, 2H), 2.48-0.95 (m, 39H), 0.86 (s, 3H), 0.85 (s, 3H), 0.74 (s, 3H), 0.69 (s, 3H), 0.64 (s, 3H). TOF-MS m/z 731 (M + H)⁺ |
| 56 | 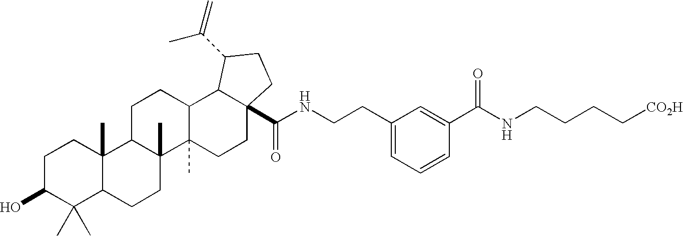 | 310 | 46.7 | ¹H NMR (DMSO-d6, 400 MHz) δ 12.02 (bs, 1H, CO2H), 8.42 (t, 1H, J = 5.7 Hz, NH), 7.69 (bs, 1H, NH), 7.65 (m, 2H, CH Arom), 7.33 (m, 2H, CH Arom), 4.64 (bs, 1H, CH═), 4.52 (bs, 1H, CH═), 4.27 (d, 1H, J = 5.1 Hz, H-3), 3.50-0.64 (m, 56H). 703.7 (M + H)+. |
| 57 | 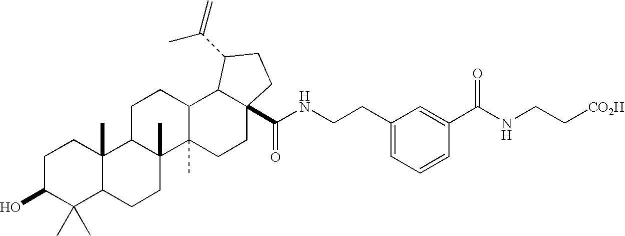 | 1200 | 98.2 | ¹H NMR (DMSO-d6, 400 MHz) δ 12.02 (bs, 1H, CO2H), 8.49 (t, 1H, J = 5.5 Hz, NH), 7.69 (bs, 1H, NH), 7.65 (m, 2H, CH Arom), 7.34 (m, 2H, CH Arom), 4.64 (bs, 1H, CH═), 4.52 (bs, 1H, CH═), 4.27 (d, 1H, J = 5.1 Hz, H-3), 3.50-0.64 (m, 52H). 675.6 (M + H)+. |
| 58 | 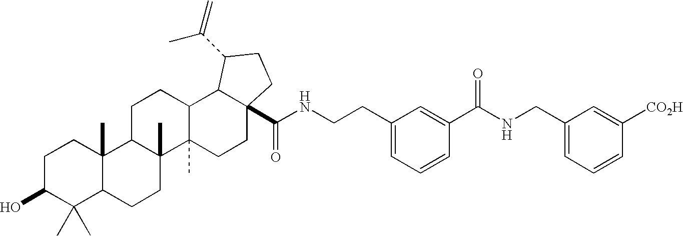 | 18 | 18.6 | ¹H NMR (400 MHz, DMSO) δ 12.95 (s, 1H), 9.07 (bt, J = 6 Hz, 1H), 7.91 (s, 1H), 7.82 (d, J = 8 Hz, 1H), 7.77-7.70 (m, 2H), 7.62 (bt, J = 6 Hz, 1H), 7.59-7.54 (m, 1H), 7.45 (t, J = 8 Hz, 1H), 7.37 (m, 2H), 4.63 (s, 1H), 4.52 (m, 3H), 4.26 (bs, 1H), 3.25 (m, 1H), 2.95 (m, 2H), 2.76 (m, 2H), 2.04 (bd, J = 13 Hz, 1H), 1.9-0.5 (m, 42H) [M + H]+ Ion mass = 737.4929, [M + Na]+ Ion mass = 759.4747 |
| 59 | 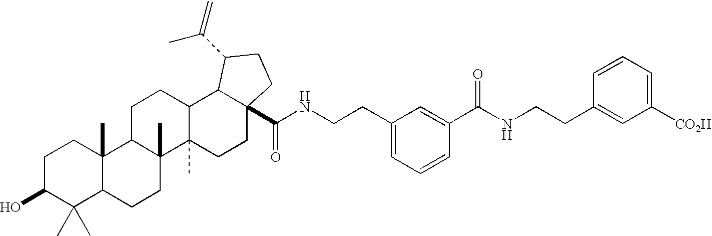 | 68 | 66.1 | ¹H NMR (400 MHz, DMSO) δ 12.94 (bs, 1H), 8.57 (bt, J = 6 Hz, 1H), 7.84 (s, 1H), 7.79 (d, J = 8 Hz, 1H), 7.66 (m, 3H), 7.49 (d, J = 8 Hz, 1H), 7.43 (t, J = 8 Hz, 1H), 7.33 (d, H = 5 Hz, 2H), 4.64 (s, 1H), 4.52 (s, 1H), 3.30 (m, 3H), 2.94 (m, 4H), 2.75 (m, 2H), 2.04 (d, J = 13 Hz, 1H), 1.8-0.5 (m, 43H) [M + H]+ Ion mass = 751.5083, [M + Na]+ Ion mass = 773.4903 |
| 60 | 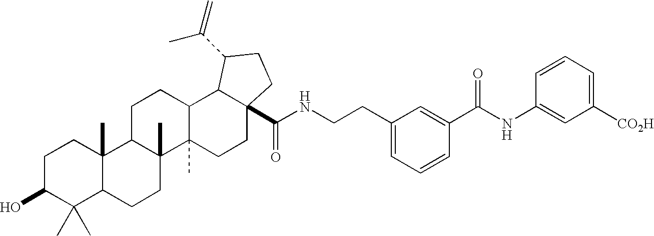 | 248 | 31 | ¹H NMR (400 MHz, DMSO) δ 12.97 (bs, 1H), 10.39 (s, 1H), 8.44 (s, 1H), 8.09 (d, J = 8 Hz, 1H), 7.85 (bs, 3H), 7.66 (m, 2H), 7.45 (m, 3H), 4.61 (s, 1H), 4.50 (s, 1H), 4.26 (d, J = 5 Hz, 1H), 3.60 (m, 1H), 2.88 (m, 4H), 1.99 (d, J = 13 Hz, 1H), 1.8-0.5 (m, 41H) [M + H]+ Ion mass = 723.4767, [M + Na]+ Ion mass = 745.4599 |
| 61 | 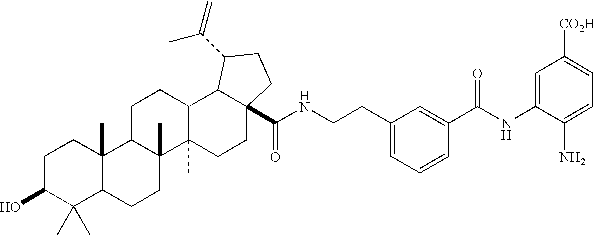 | 2300 | 65 | [M + H]+ Ion mass = 738.4832, [M + Na]+ Ion mass = 760.4564 |
| 62 | 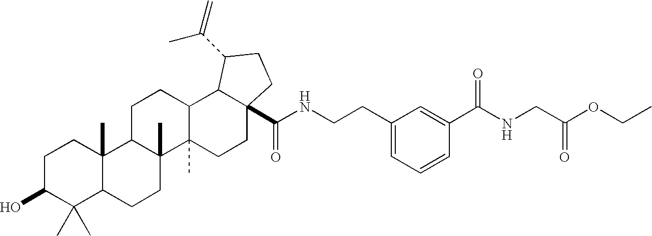 | 230 | 40.4 | ¹H NMR (DMSO-d6, 400 MHz) δ 8.90 (t, 1H, J = 5.1 Hz, NH), 7.23 (bs, 1H, NCH), 7.72-7.68 (m, 2H, CH Arom), 7.72-7.68 (m, 2H, CH Arom), 7.37 (m, 2H, CH Arom), 4.64 (bs, 1H, CH═), 4.52 (bs, 1H, CH═), 4.27 (bs, 1H, Hz, H-3), 4.11 (q, 2H, J = 7.1 Hz, OCH2), 3.98 (d, 2H, J = 5.9 Hz, CH2), 3.00-0.64 (m, 51H). 689.4892 (M + H)+. |
| 63 | 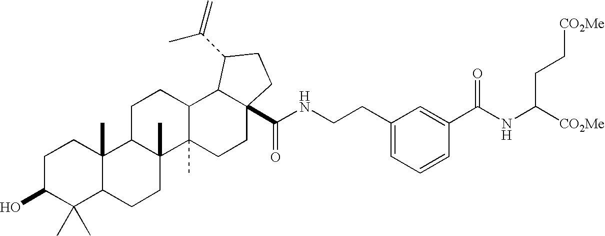 | 90 | 35.9 | ¹H NMR (DMSO-d6, 400 MHz) δ 8.72 (d, 1H, J = 7.4 Hz, NH), 7.90-7.01 (m, 2H, Arom), 7.80 (t, 1H, J = 5.1 Hz, NH), 7.39 (d, 2H, CH Arom), 4.64 (bs, 1H, CH═), 4.52 (bs, 1H, CH═), 4.49-4.20 (m, 1H, CH2), 4.27 (d, 1H, J = 5.0 Hz, H-3), 3.65 (s, 3H, OCH3), 3.59 (s, 3H, OCH3), 3.00-0.64 (m, 52H). 761.5094 (M + H)+. |
| 64 | 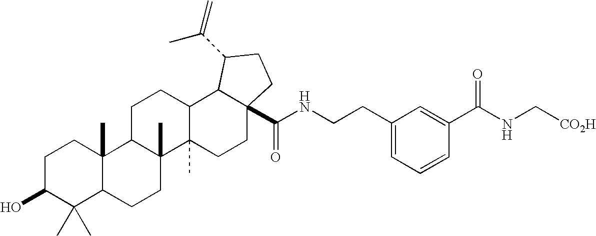 | N/A | 220 | ¹H NMR (DMSO-d6, 400 MHz) δ 8.82 (t, 1H, J = 5.7 Hz, NH), 7.74 (bs, 1H, NH), 7.69 (m, 2H, CH Arom), 7.36 (m, 2H, CH Arom), 4.64 (bs, 1H, CH═), 4.52 (bs, 1H, CH═), 3.91 (d, 2H, J = 5.8 Hz, CH2), 3.00-0.64 (m, 50H). 661.4594 (M + H)+. |
| 65 | 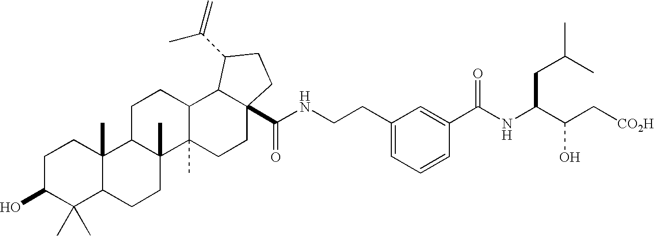 | 83 | 156 | ¹H NMR (DMSO-d6, 400 MHz) δ 7.78 (d, 1H, J = 9.1 Hz, NCH), 7.71 (bs, 1H, NH), 7.69 (m, 2H, CH Arom), 7.36 (m, 2H, CH Arom), 4.64 (bs, 1H, CH═), 4.52 (bs, 1H, CH═), 4.01 (bs, 1H, H-3), 3.00-0.64 (m, 63H). 761.5482 (M + H)+. |
| 66 | 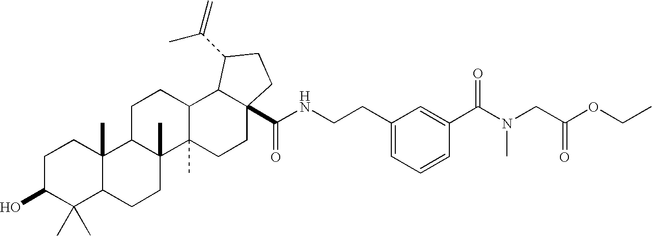 | N/A | 420 | ¹H NMR (DMSO-d6, 400 MHz) δ 7.95 (bs, 1H, CH Arom), 7.64 (bs, 1H, NH), 7.39-7.20 (m, 3H, CH Arom), 4.64 (bs, 1H, CH═), 4.52 (bs, 1H, CH═), 4.27 (d, 1H, J = 5.2 Hz, H-3), 4.23 (s, 1H, CH) 3.00-0.64 (m, 55H). 689.4896 (M + H)+. |
| 67 | 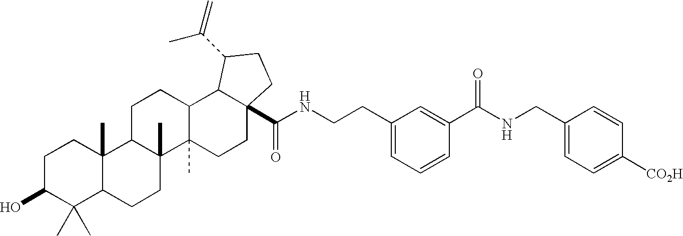 | 23 | 7.5 | ¹H NMR (400 MHz, d6-DMSO) δ 12.87 (s, 1H), 9.07 (bt, J = 6 Hz, 1H), 7.90 (d, J = 8 Hz, 2H), 7.77 (s, 1H), 7.74 (m, 1H), 7.62 (bt, J = 6 Hz, 1H), 7.42 (d, J = 8 Hz, 2H), 4.63 (s, 1H), 4.53 (m, 3H), 3.60 (m, 1H), 3.26 (m, 2H), 2.95 (m, 2H), 2.76 (m, 2H), 2.04 (d, J = 13 Hz, 1H), 1.80-0.55 (m, 43H) [M + H]+ Ion mass = 737.4884, [M + Na]+ Ion mass = 759.4702 |
| 68 | 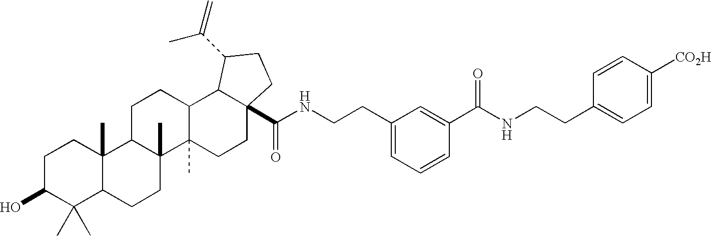 | 64 | 15.2 | ¹H NMR (400 MHz, d6-DMSO) δ 12.83 (s, 1H), 8.55 (bt, J = 6 Hz, 1H), 7.87 (d, J = 8 Hz, 2H), 7.68 (s, 1H), 7.63 (m, 2H), 7.35 (m, 4H), 4.63 (s, 1H), 7.35 (m, 4H), 4.63 (s, 1H), 4.52 (s, 1H), 3.60 (m, 1H), 3.25 (m, 2H), 2.92 (m, 4H), 2.76 (m, 2H), 2.03 (d, J = 13 Hz, 1H), 1.80-0.55 (m, 43H) [M + H]+ Ion mass = 751.5045, [M + Na]+ Ion mass = 773.4863 |
| 69 | 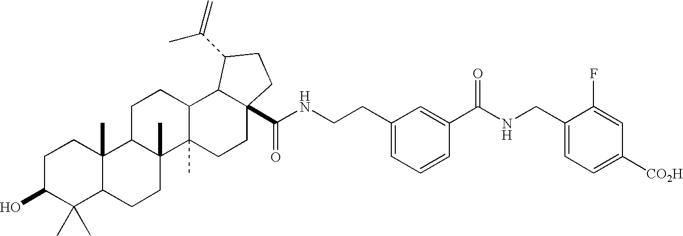 | 670 | 11 | ¹H NMR (400 MHz, d6-DMSO) δ 9.02-9.07 (1H, m), 7.72-7.77 (4H, m), 7.77-7.66 (2H, m), 7.40-7.49 (1H, m), 7.36-7.39 (2H, m), 6.55 (1H, s), 4.50-4.63 (4H, m), 4.25-4.28 (1H, m), 0.5-3.5 (47H, m). Mass Spec (m/z): 755 (M + 1). |
| 70 | 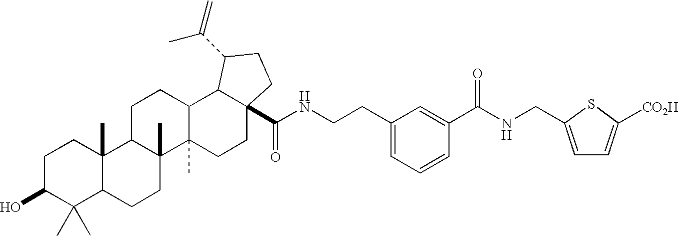 | 1.6 | 19.7 | ¹H NMR (400 MHz, d6-DMSO) δ 9.17-9.22 (1H, m), 8.20-8.25 (1H, m), 7.78 (1H, br s), 7.68-7.73 (1H, m), 7.56 (1H, d, J = 4 Hz), 7.37-7.41 (2H, m), 7.055 (1H, d, J = 4 Hz), 4.60-4.64 (3H, m), 4.52 (1H, br s), 2.93-3.03 (1H, m), 0.55-2.5 (47H, m). Mass Spec (m/z): 743 (M + 1). |
| 71 | 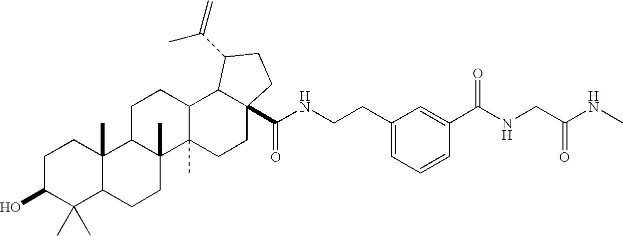 | N/A | 2700 | ¹H NMR (DMSO-d6, 400 MHz) δ 8.70 (t, 1H, J = 6.0 Hz, NH), 7.84 (d, 1H, J = 4.8 Hz, CH Arom), 7.75 (s, 1H, CH Arom), 7.72-7.65 (m, 2H, CH Arom), 7.37 (m, 2H, CH Arom), 4.64 (bs, 1H, CH═), 4.52 (bs, 1H, CH═), 3.00-0.64 (m, 54H). 674.5 (M + H)+. |
| 72 | 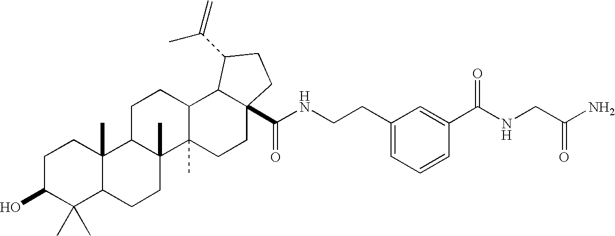 | 10000 | 194 | ¹H NMR (DMSO-d6, 400 MHz) δ 8.62 (t, 1H, J = 6.0 Hz, NH), 7.74 (s, 1H, CH Arom), 7.72-7.65 (m, 2H, CH Arom), 7.37 (m, 3H, CH Arom), 7.05 (bs, 1H, NH), 4.64 (bs, 1H, CH═), 4.52 (bs, 1H, CH═), 4.27 (d, 1H, J = 5.0 Hz, CH), 3.81 (d, 2H, J = 5.8 Hz, CH2), 3.00-0.64 (m, 48H). 660.5 (M + H)+. |
| 73 | 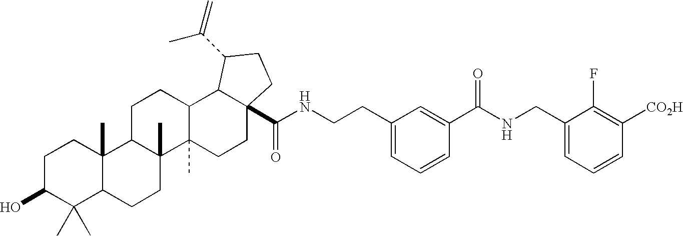 | 1.5 | 16.2 | ¹H NMR (400 MHz, d6-DMSO) δ 9.05 (bt, J = 6 Hz, 1H), 7.75 (m, 3H), 7.64 (m, 1H), 7.56 (m, 1H), 7.37 (m, 2H), 7.24 (t, J = 9 Hz, 1H), 4.63 (s, 1H), 4.52 (m, 3H), 3.37 (m, 2H), 3.25 (m, 1H), 2.96 (m, 3H), 2.76 (m, 2H), 2.04 (d, J = 14 Hz, 1H), 1.90-0.50 (m, 40H) [M + H]+ Ion mass = 755.4794, [M + Na]+ Ion mass = 777.4608 |
| 74 | 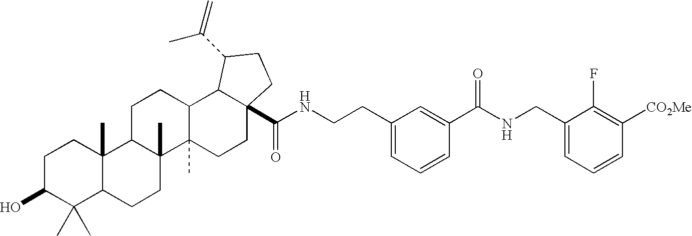 | 0.8 | 27.6 | ¹H NMR (400 MHz, d6-DMSO) δ 9.02 (bt, J = 6 Hz, 1H), 7.76 (m, 3H), 7.62 (m, 2H), 7.37 (m, 2H), 7.28 (t, J = 9 Hz, 1H), 4.63 (s, 1H), 4.52 (m, 3H), 4.27 (d, J = 5 Hz, 1H), 3.86 (s, 3H), 3.25 (m, 1H), 3.00-2.64 (m, 5H), 2.04 (d, J = 13 Hz, 1H), 1.90-0.50 (m, 41H) [M + H]+ Ion mass = 769.4947, [M + Na]+ Ion mass = 791.4760 |
| 75 | 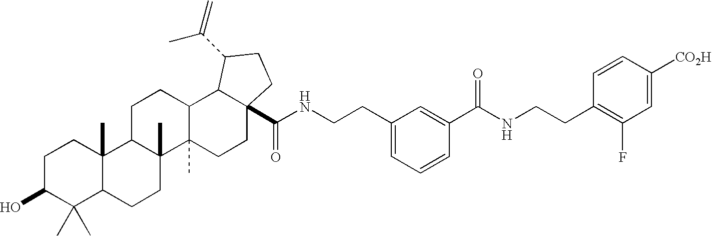 | 1.4 | 36.3 | ¹H NMR (400 MHz, d6-DMSO) δ 13.17 (bs, 1H), 8.57 (bt, J = 6 Hz, 1H), 7.76-7.57 (m, 5H), 7.45 (t, J = 8 Hz, 1H), 7.33 (m, 2H), 4.63 (s, 1H), 4.52 (s, 1H), 3.65-0.50 (m, 53H) [M + H]+ Ion mass = 769.4961, [M + Na]+ Ion mass = 791.4773 |
| 76 | 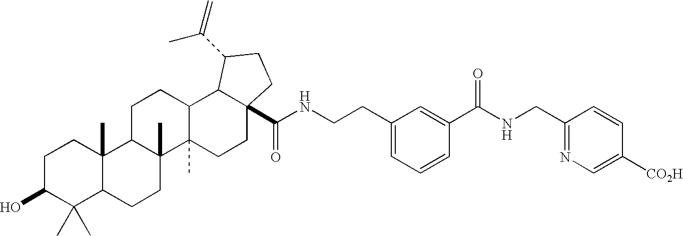 | 89 | 10.4 | ¹H NMR (DMSO-d6, 400 MHz) δ 9.09 (m, 1H), 8.91 (s, 1H), 8.09 (d, J = 8.0 Hz, 1H), 7.80 (s, 1H), 7.77 (m, 1H), 7.64 (m, 1H), 7.37 (m, 2H), 7.23 (d, J = 7.2 Hz, 1H), 4.63 (s, 1H), 4.58 (d, J = 5.6 Hz, 2H), 4.52 (s, 1H), 4.27 (s, 1H), 2.99 (m, 2H), 2.82-0.85 (m, 36H), 0.75 (s, 3H), 0.74 (s, 3H), 0.64 (s, 3H). TOF-MS m/z 738 (M + H)⁺ |
| 77 | 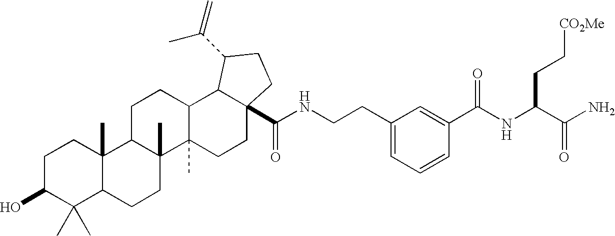 | N/A | 105.5 | ¹H NMR (DMSO-d6, 400 MHz) δ 8.31 (d, 1H, J = 8.0 Hz, NH), 7.74 (s, 1H, CH Arom), 7.72-7.65 (m, 2H, CH Arom), 7.42 (s, 1H, CH arom), 7.37 (m, 2H, CH Arom), 7.10 (bs, 1H, NH), 6.56 (m, 1H, NCH), 4.64 (bs, 1H, CH═), 4.52 (bs, 1H, CH═), 4.27 (d, 1H, J = 5.0 Hz, CH), 3.61 (s, 3H, OCH3), 3.00-0.64 (m, 52H). 746.5114 (M + H)+. |
| 78 | 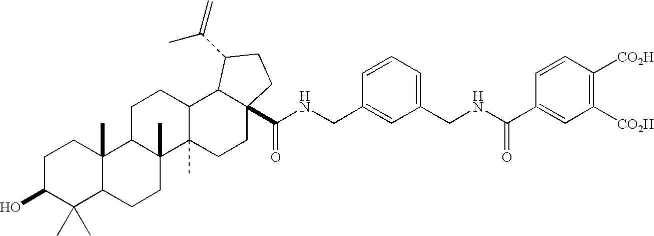 | N/A | 7000 | ¹H NMR (400 MHz, DMSO) δ 13.30 (bs, 1H), 9.05 (bs, 1H), 8.16 (bt, J = 6 Hz, 1H), 8.05 (dd, J = 2.8 Hz, 1H), 7.99 (d, J = 2 Hz, 1H), 7.83 (d, J = 8 Hz, 1H), 7.24 (m, 3H), 7.12 (bd, J = 7 Hz, 1H), 4.64 (s, 1H), 4.53 (s, 1H), 4.40 (d, J = 6 Hz, 2H), 4.24 (m, 3H), 3.00 (m, 2H), 2.19 (d, J = 11 Hz, 1H), 1.8-0.5 (m, 41H) [M + H]+ Ion mass = 768, [M + Na]+ Ion mass = 789.7 |
| 79 | 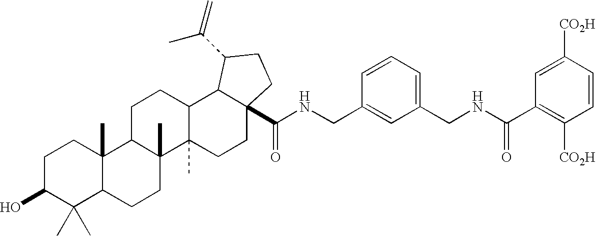 | N/A | 412 | ¹H NMR (400 MHz, DMSO) δ 13.32 (bs, 1H), 9.02 (bs, 1H), 8.29 (s, 1H), 8.16 (bt, J = 6 Hz, 1H), 8.09 (d, J = 8 Hz, 1H), 7.59 (d, J = 8 Hz, 1H), 7.24 (m, 3H), 7.12 (m, 1H), 4.64 (s, 1H), 4.53 (s, 1H), 4.40 (d, J = 6 Hz, 2H), 4.24 (m, 3H), 2.99 (m, 2H), 2.19 (d, J = 12 Hz, 1H), 1.8-0.5 (m, 41H) [M + H]+ Ion mass = 768, [M + Na]+ Ion mass = 789.8 |
| 80 | 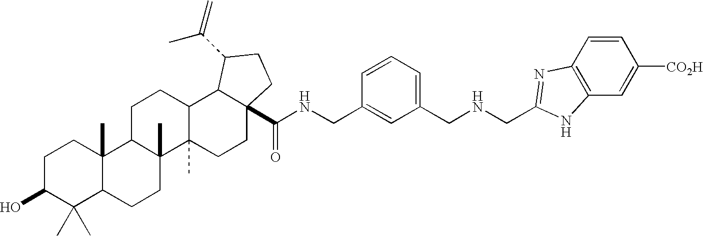 | 1000 | 242 | ¹H NMR (400 MHz, DMSO) δ 12.88 (bs, 1H), 9.84 (bs, 1H), 8.19 (m, 3H), 7.33 (m, 4H), 4.64 (s, 1H), 4.53 (s, 1H), 4.45 (s, 1H), 4.24 (m, 3H), 3.99 (m, 1H), 2.97 (m, 2H), 2.19 (d, J = 13 Hz, 1H), 1.8-0.5 (m, 45H) [M + H]+ Ion mass = 749.5000 |
| 81 | 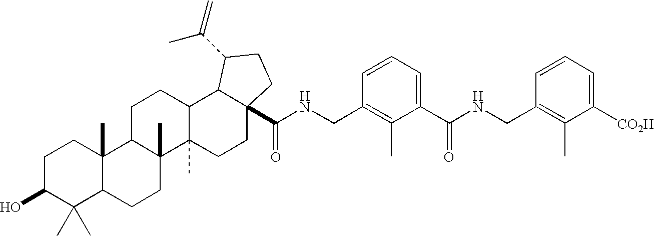 | N/A | 6200 | ¹H NMR (DMSO-d6, 400 MHz) δ 8.70 (m, 1H), 8.10 (m, 1H), 7.39 (d, J = 7.6 Hz, 1H), 7.30-7.11 (m, 5H), 4.65 (s, 1H), 4.53 (s, 1H), 4.43 (d, J = 6.0 Hz, 1H), 4.20 (m, 2H), 2.97 (m, 2H), 2.41 (s, 3H), 2.21 (s, 3H), 2.20-0.80 (m, 37H), 0.74 (s, 3H), 0.64 (s, 3H). TOF-MS m/z 751 (M + H)⁺ |
| 82 | 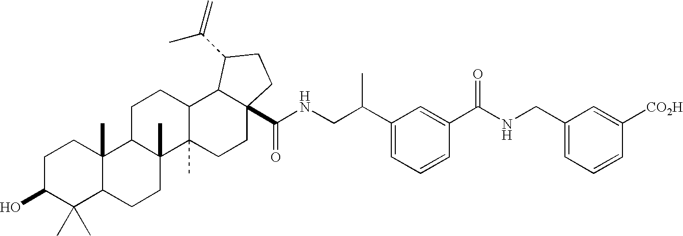 | N/A | 150 | ¹H NMR (DMSO-d6, 400 MHz) δ 9.06 (m, 1H), 7.89 (m, 1H), 7.78 (m, 3H), 7.59 (m, 1H), 7.48 (d, J = 7.2 Hz, 1H), 7.40 (m, 3H), 4.63 (s, 1H), 4.50 (s, 3H), 4.27 (s, 1H), 2.99 (m, 2H), 2.50-0.71 (m, 45H), 0.64 (s, 3H). TOF-MS m/z 751 (M + H)⁺ |
| 83 | 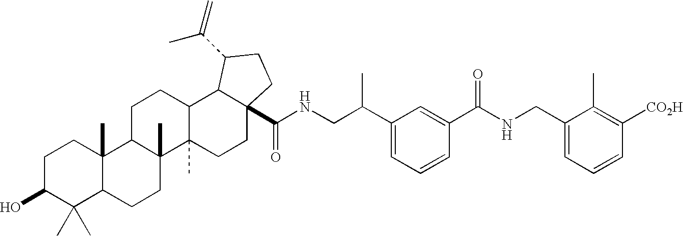 | N/A | 270 | ¹H NMR (DMSO-d6, 400 MHz) δ 8.87 (m, 1H), 7.74 (m, 2H), 7.56 (m, 1H), 7.38 (m, 3H), 7.22 (m, 1H), 7.11 (m, 1H), 4.63 (s, 1H), 4.50 (m, 2H), 4.27 (s, 1H), 2.99 (m, 2H), 2.50-0.71 (m, 48H), 0.64 (s, 3H). TOF-MS m/z 765 (M + H)⁺ |
| 84 | 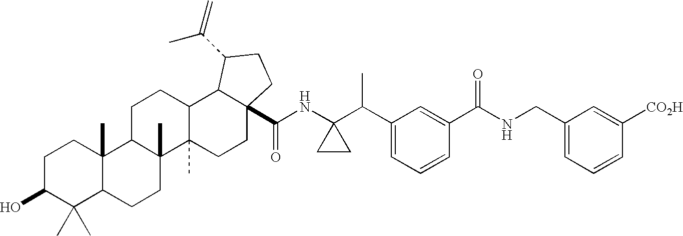 | N/A | 2500 | 763.5045 (M + H)+ |
| 85 | 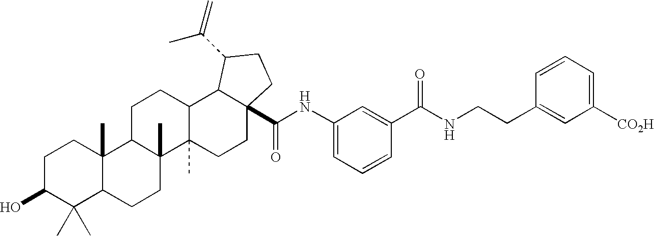 | N/A | 10000 | Mass Spec (m/z): 723 (M + 1). |
| 86 | 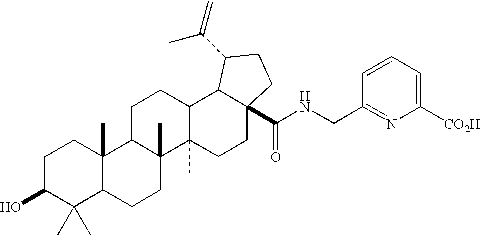 | N/A | 3600 | ¹H NMR (400 MHz, d6-DMSO) δ 8.34 (t, J = 5.8 Hz, 1H), 7.85 (t, J = 3.9 Hz, 2H), 7.36 (t, J = 3.9 Hz, 1H), 4.62 (d, J = 2 Hz, 1H), 4.51 (s, 1H), 4.37-4.27 (m, 2H), 2.96 (m, 2H), 2.20 (m, 1H), 1.87 (m, 1H), 1.72-0.62 (m, 41H); Mass Spec (m/z): 591 (M + 1). |
| 87 | 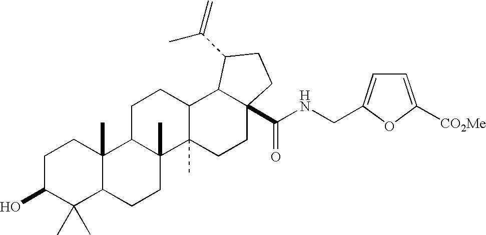 | N/A | 1400 | ¹H NMR (400 MHz, d6-DMSO) δ 8.24 (t, J = 5.8 Hz, 1H), 7.25 (d, J = 3.4 Hz, 1H), 6.33 (d, J = 3.4 Hz, 1H), 4.64 (d, J = 2.2 Hz, 1H), 4.53 (s, 1H), 4.25 (m, 2H), 3.77 (s, 3H), 3.00 (m, 2H), 2.90-0.64 (m, 43H); Mass Spec (m/z): 594.4 (M + 1). |
| 88 | 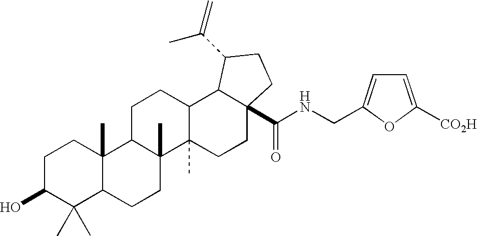 | N/A | 6900 | ¹H NMR (400 MHz, d6-DMSO) δ 12.94 (s, 1H), 8.23 (t, J = 5.8 Hz, 1H), 7.13 (d, J = 3.4 Hz, 1H), 6.29 (d, J = 3.4 Hz, 1H), 4.65 (t, J = 1 Hz, 1H), 4.53 (s, 1H), 4.27 (m, 3H), 2.98 (m, 2H), 2.47-0.63 (m, 43H); Mass Spec (m/z): 580.4 (M + 1). |
| 89 | 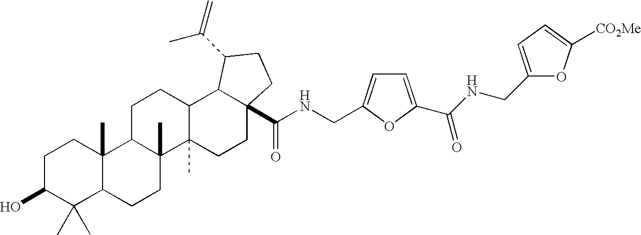 | N/A | 2300 | ¹H NMR (400 MHz, d6-DMSO) δ 8.82 (t, J = 5.9 Hz, 1H), 8.20 (t, J = 5.9 Hz, 1H), 7.23 (d, J = 5.6 Hz, 1H), 7.08 (d, J = 3.9 Hz, 1H), 6.46 (d, J = 3.4 Hz, 1H), 6.24 (d, J = 3.4 Hz, 1H), 4.65 (d, J = 1 Hz, 1H), 4.53 (s, 1H), 4.46 (d, J = 5.8 Hz, 1H), 4.28 (m, 2H), 2.97 (m, 2H), 2.46-0.63 (m, 44H); Mass Spec (m/z): 717.4 (M + 1). |
| 90 | 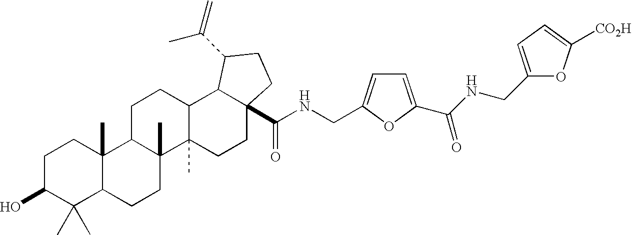 | N/A | 6500 | ¹H NMR (400 MHz, d6-DMSO) δ 8.24 (t, J = 5.9 Hz, 1H), 8.20 (t, J = 5.9 Hz, 1H), 7.12 (d, J = 3.4 Hz, 1H), 7.10 (d, J = 3.4 Hz, 1H), 6.41 (d, J = 3.4 Hz, 1H), 6.23 (d, J = 3.4 Hz, 1H), 4.65 (d, J = 3.4 Hz, 1H), 4.65 (d, J = 2 Hz, 1H), 4.53 (s, 1H), 4.45 (d, J = 5.8 Hz, 1H), 4.25 (m, 2H), 2.96 (m, 2H), 2.45-0.64 (m, 44H); Mass Spec (m/z): 703.4 (M + 1). |
| 91 | 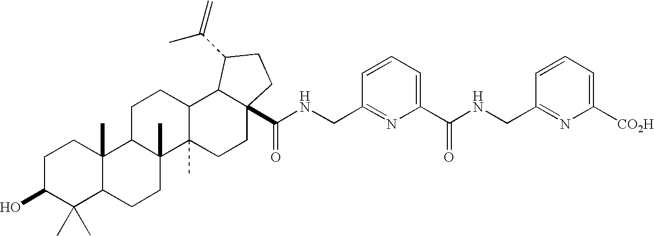 | N/A | 3900 | ¹H NMR (400 MHz, d6-DMSO) δ 9.38 (t, J = 6 Hz, 1H), 8.35 (t, J = 3.2 Hz, 1H), 7.94 (m, 4H), 7.54 (t, J = 4.5 Hz, 1H), 7.45 (m, 1H), 4.69 (d, J = 2 Hz, 1H), 4.63 (s, 1H), 4.40 (m, 3H), 2.95 (m, 2H), 2.54-0.63 (m, 44H); Mass Spec (m/z): 725 (M + 1). |
| 92 | 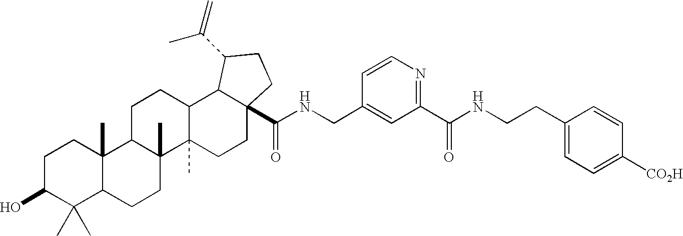 | 4867 | 121 | ¹H NMR (DMSO-d6, 400 MHz) δ 8.85 (m, 1H), 8.52 (d, J = 6.4 Hz, 1H), 8.38 (m, 1H), 7.95 (s, 1H), 7.83 (d, J = 8.4 Hz, 2H), 7.42 (d, J = 6.4 Hz, 1H), 7.29 (d, J = 8.4 Hz, 2H), 4.63 (s, 1H), 4.52 (s, 1H), 4.26 (m, 3H), 3.55 (m, 2H), 2.92 (m, 2H), 2.50-0.80 (m, 34H), 0.70 (s, 3H), 0.62 (s, 3H), 0.58 (s, 3H). TOF-MS m/z 738 (M + H)⁺ |
| 93 | 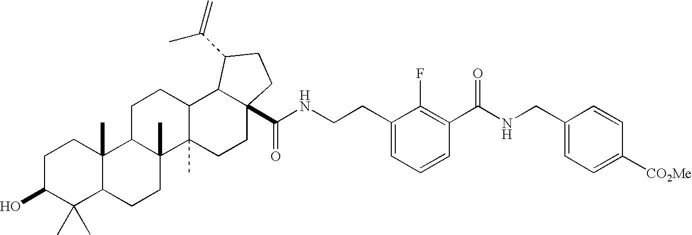 | 40 | 33.9 | ¹H NMR (400 MHz, d6-DMSO) δ 8.86-8.91 (1H m), 7.93 (1H, d, J = 8.4 Hz), 7.66-7.70 (1H, m), 7.44-7.52 (3H, m), 7.36-7.42 (1H, m), 7.18 (1H, t, J = 7.6 Hz), 4.62-4.65 (1H, m), 4.51-4.55 (3H, m), 4.68 (1H, d, J = 5.2 Hz), 3.84 (3H, s), 0.5-3.5 (49H, m). Mass Spec (m/z): 769 (M + 1). |
| 94 | 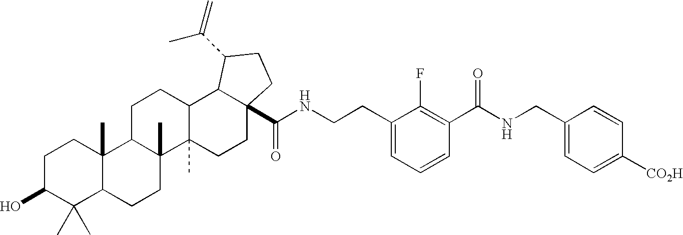 | 290 | 70.5 | ¹H NMR (400 MHz, d6-DMSO) δ 8.86-8.95 (1H, m), 7.85-7.93 (2H, m), 7.70-7.75 (1H, m), 7.30-7.55 (4H, m), 7.00-7.2 (1H, m), 6.56 (1H, br s), 4.64 (1H, br s), 4.50-4.55 (2H, m), 4.29 (1H, br s), 0.55-3.6 (46H, m). Mass Spec (m/z): 756 (M + 1). |
| 95 | 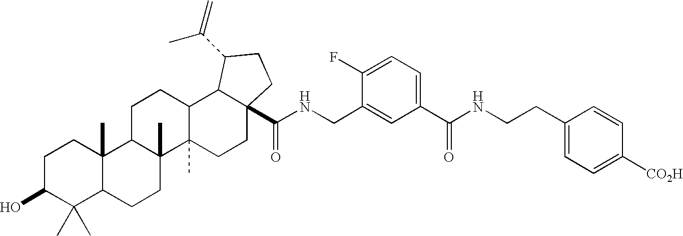 | 1.5 | 11.2 | ¹H NMR (400 MHz, d6-DMSO) δ 12.83 (bs, 1H), 8.37 (m, 1H), 8.23 (bt, J = 6 Hz, 1H), 7.87 (d, J = 8 Hz, 2H), 7.49 (d, J = 8 Hz, 1H), 7.36 (m, 3H), 7.20 (t, J = 8 Hz, 1H), 4.64 (s, 1H), 4.53 (s, 1H), 4.38-4.04 (m, 2H), 3.48 (m, 2H), 2.95 (m, 4H), 2.17 (d, J = 13 Hz, 1H), 1.90-0.50 (m, 42H) [M + H]+ Ion mass = 755.4803, [M + Na]+ Ion mass = 777.4613 |
| 96 | 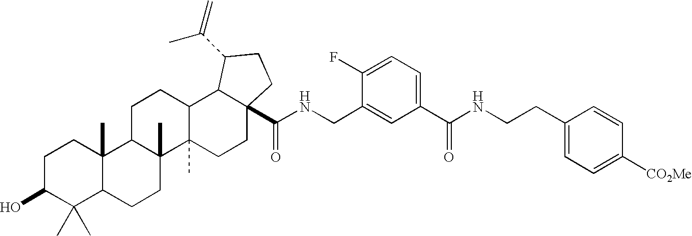 | 3.5 | 33 | ¹H NMR (400 MHz, d6-DMSO) δ 8.37 (m, 1H), 8.23 (bt, J = 6 Hz, 1H), 7.89 (d, J = 8 Hz, 2H), 7.49 (d, J = 8 Hz, 1H), 7.40 (d, J = 8 Hz, 2H), 7.34 (m, 1H), 7.20 (t, J = 10 Hz, 1H), 4.64 (s, 1H), 4.53 (s, 1H), 4.36-4.06 (m, 3H), 3.84 (s, 3H), 3.49 (m, 2H), 2.95 (m, 4H), 2.17 (d, J = 13 Hz, 1H), 1.90-0.50 (m, 41H) [M + H]+ Ion mass = 769.4937, [M + Na]+ Ion mass = 791.4751 |
| 97 | 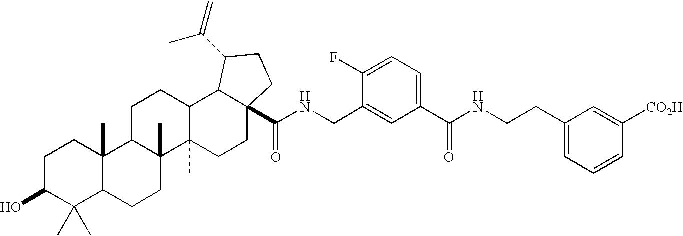 | 1 | 23.7 | ¹H NMR (400 MHz, d6-DMSO) δ 12.91 (bs, 1H), 8.37 (m, 1H), 8.23 (bt, J = 6 Hz, 1H), 7.83 (s, 1H), 7.79 (d, J = 8 Hz, 1H), 7.49 (m, 2H), 7.43 (t, J = 8 Hz, 1H), 7.34 (m, 1H), 7.20 (t, J = 10 Hz, 1H), 4.64 (s, 1H), 4.53 (s, 1H), 4.37-4.06 (m, 2H), 3.47 (m, 2H), 2.94 (m, 4H), 2.17 (d, J = 13 Hz, 1H), 1.90-0.50 (m, 42H) [M + H]+ Ion mass = 755.4779, [M + Na]+ Ion mass = 777.4601 |
| 98 | 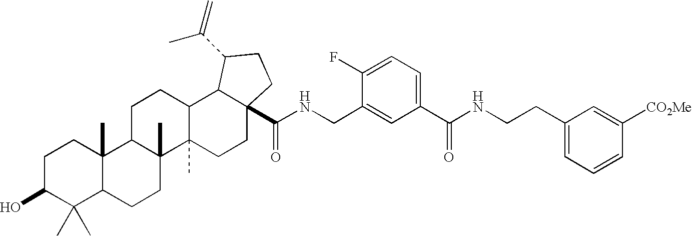 | 13 | 57.7 | [M + H]+ Ion mass = 769.4939, [M + Na]+ Ion mass = 791.4755 |
| 99 | 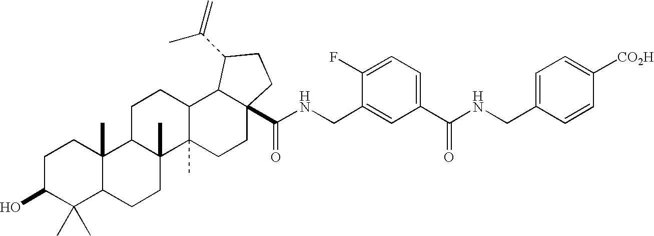 | 2.8 | 20.1 | ¹H NMR (400 MHz, d6-DMSO) δ 12.89 (bs, 1H), 8.89 (m, 1H), 8.24 (bt, J = 6 Hz, 1H), 7.90 (d, J = 8 Hz, 2H), 7.50 (d, J = 8 Hz, 1H), 7.41 (m, 3H), 7.24 (t, J = 10 Hz, 1H), 4.64 (s, 1H), 4.53 (m, 3H), 4.32-4.13 (m, 2H), 3.60 (m, 3H), 2.97 (m, 2H), 2.17 (d, J = 13 Hz, 1H), 1.90-0.50 (m, 39H) [M + H]+ Ion mass = 741.4622, [M + Na]+ Ion mass = 763.4446 |
| 100 | 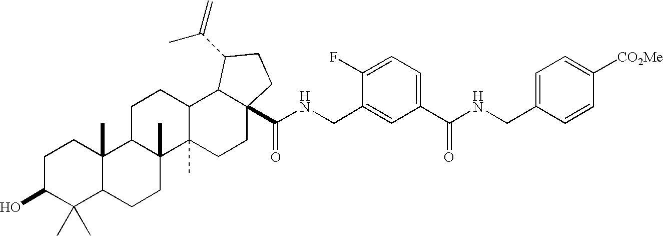 | 1.3 | 21.5 | ¹H NMR (400 MHz, d6-DMSO) δ 8.90 (m, 1H), 8.24 (bt, J = 6 Hz, 1H), 7.93 (d, J = 8 Hz, 2H), 7.55 (d, J = 8 Hz, 1H), 7.45 (d, J = 8 Hz, 2H), 7.38 (m, 1H), 7.24 (t, J = 10 Hz, 1H), 4.64 (s, 1H), 4.53 (m, 3H), 4.32-4.13 (m, 3H), 3.84 (s, 3H), 2.97 (m, 2H), 2.17 (d, J = 13 Hz, 1H), 1.90-0.50 (m, 41H) [M + H]+ Ion mass = 755.4778, [M + Na]+ Ion mass = 777.4597 |
| 101 | 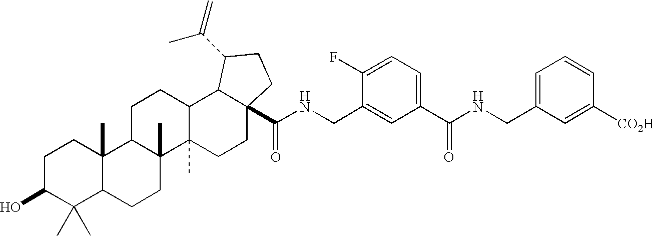 | 3.2 | 28.3 | ¹H NMR (400 MHz, d6-DMSO) δ 12.94 (bs, 1H), 8l.90 (m, 1H), 8.23 (bt, J = 6 Hz, 1H), 8.90 (m, 1H), 8.23 (bt, J = 6 Hz, 1H), 7.94 (s, 1H), 7.83 (d, J = 8 Hz, 1H), 7.55 (m, 2H), 7.46 (t, J = 8 Hz, 1H), 7.38 (m, 1H), 7.24 (t, H = 10 Hz, 1H), 4.64 (s, 1H), 4.52 (m, 3H), 4.32-4.12 (m, 2H), 2.97 (m, 2H), 2.17 (d, J = 13 Hz, 1H), 1.90-0.50 (m, 42H) [M + H]+ Ion mass = 741.4631, [M + Na]+ Ion mass = 763.4438 |
| 102 | 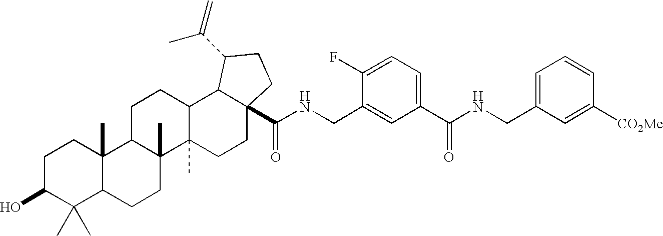 | 0.6 | 22 | [M + H]+ Ion mass 755.4794, [M + Na]+ Ion mass = 777.4613 |
| 103 | 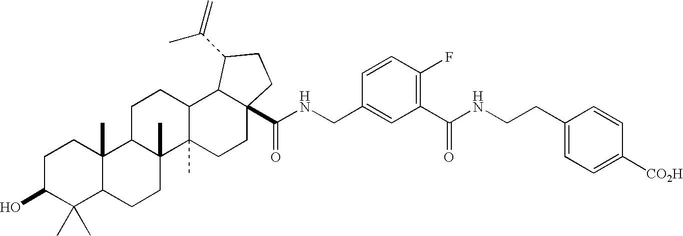 | 7.3 | 69.3 | ¹H NMR (400 MHz, d6-DMSO) δ 12.83 (bs, 1H), 8.57 (bt, J = 6 Hz, 1H), 8.24 (bt, J = 6 Hz, 1H), 7.86 (m, 3H), 7.70 (m, 1H), 7.35 (d, J = 8 Hz, 2H), 7.23 (t, J = 9 Hz, 1H), 4.64 (s, 1H), 4.52 (s, 1H), 4.43-4.08 (m, 2H), 3.60 (m, 1H), 2.93 (m, 4H), 2.45 (m, 1H), 2.22 (d, J = 13 Hz, 1H), 1.90-0.50 (m, 42H) [M + H]+ Ion mass = 755.4793, [M + Na]+ Ion mass = 777.4623 |
| 104 | 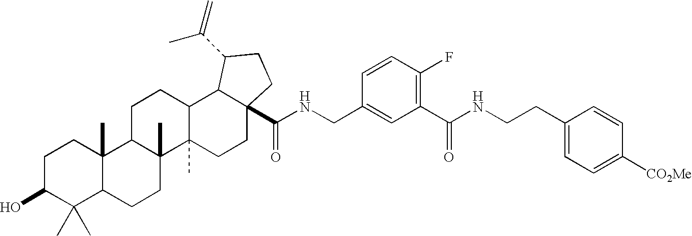 | N/A | 350 | ¹H NMR (400 MHz, d6-DMSO) δ 8.57 (bt, J = 6 Hz, 1H), 8.24 (bt, J = 6 Hz, 1H), 7.89 (d, J = 8 Hz, 2H), 7.85 (m, 1H), 7.70 (m, 1H), 7.38 (d, J = 8 Hz, 2H), 7.23 (t, J = 10 Hz, 1H), 4.63 (s, 1H), 4.52 (s, 1H), 4.41-4.09 (m, 3H), 3.83 (s, 3H), 3.47 (m, 2H), 2.94 (m, 4H), 2.45 (m, 1H), 2.22 (d, J = 13 Hz, 1H), 1.90-0.50 (m, 40H) [M + H]+ Ion mass = 769.4652, [M + Na]+ Ion mass = 769.4652, [M + Na]+ 791.4768 |
| 105 | 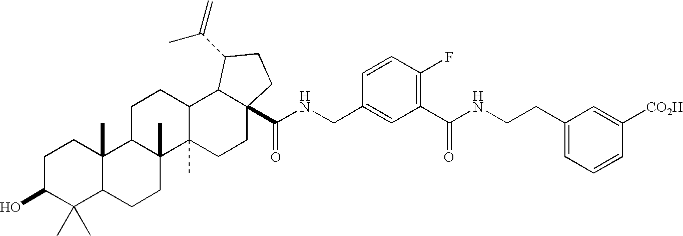 | 8.5 | 62.1 | ¹H NMR (400 MHz, d6-DMSO) δ 12.91 (bs, 1H), 8.58 (bt, J = 6 Hz, 1H), 8.25 (bt, J = 6 Hz, 1H), 7.83 (m, 3H), 7.70 (m, 1H), 7.48 (d, J = 8 Hz, 1H), 7.42 (t, J = 8 Hz, 1H), 7.22 (t, J = 9 Hz, 1H), 4.63 (s, 1H), 4.52 (s, 1H), 4.43-4.06 (m, 3H), 3.46 (m, 3H), 2.93 (m, 4H), 2.45 (m, 1H), 2.23 (d, J = 13 Hz, 1H), 1.90-0.50 (m, 39H) [M + H]+ Ion mass = 755.4785, [M + Na]+ Ion mass = 777.4608 |
| 106 | 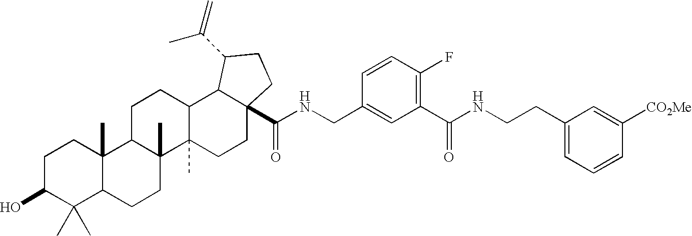 | N/A | 1300 | ¹H NMR (400 MHz, d6-DMSO) δ 8.58 (bt, J = 6 Hz, 1H), 8.25 (bt, J = 6 Hz, 1H), 8.25 (bt, J = 6 Hz, 1H), 7.85 (m, 2H), 7.81 (d, J = 8 Hz, 1H), 7.70 (m, 1H), 7.52 (d, J = 8 Hz, 1H), 7.45 (t, J = 8 Hz, 1H), 7.23 (t, J = 9 Hz, 1H), 4.63 (s, 1H), 4.52 (s, 1H), 4.44-4.07 (m, 3H), 3.43 (m, 3H), 2.93 (m, 4H), 2.22 (d, J = 13 Hz, 1H), 1.90-0.50 (m, 39H) [M + H]+ Ion mass = 769.4954, [M + Na]+ Ion mass = 791.4770 |
| 107 | 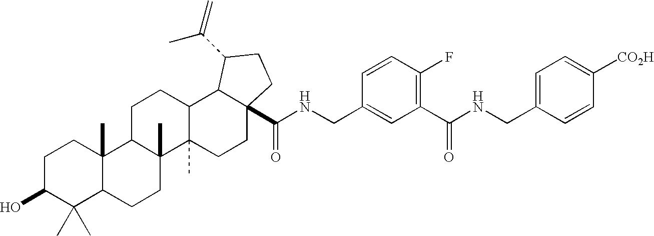 | 130 | 103 | ¹H NMR (400 MHz, d6-DMSO) δ 12.85 (bs, 1H), 9.07 (bt, J = 6 Hz, 1H), 8.24 (bt, J = 6 Hz, 1H), 7.90 (m, 3H), 7.80 (m, 1H), 7.40 (d, J = 8 Hz, 2H), 7.26 (t, J = 9 Hz, 1H), 4.68-4.12 (m, 6H), 3.60 (m, 1H), 2.93 (m, 2H), 2.20 (d, J = 13 Hz, 1H), 1.90-0.50 (m, 41H) [M + H]+ Ion mass = 741.4635, [M + Na]+ Ion mass = 763.4457 |
| 108 | 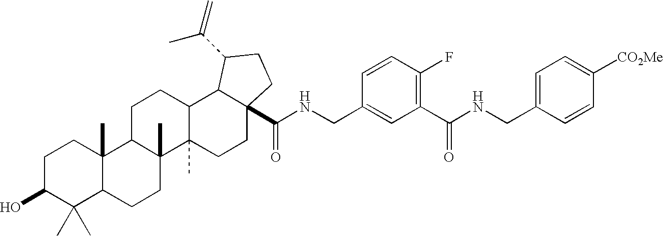 | 100 | 109 | ¹H NMR (400 MHz, d6-DMSO) δ 9.08 (bt, J = 6 Hz, 1H), 8.25 (bt, J = 6 Hz, 1H), 7.9 (d, J = 6 Hz, 2H), 7.26 (t, J = 10 Hz, 1H), 4.68-4.45 (m, 4H), 4.41-4.13 (m, 3H), 3.83 (s, 3H), 2.96 (m, 2H), 2.21 (d, J = 13 Hz, 1H),]1.90-0.50 (m, 41H) [M + H]+ Ion mass = 755.4786, [M + Na]+ Ion mass = 777.4602 |
| 109 | 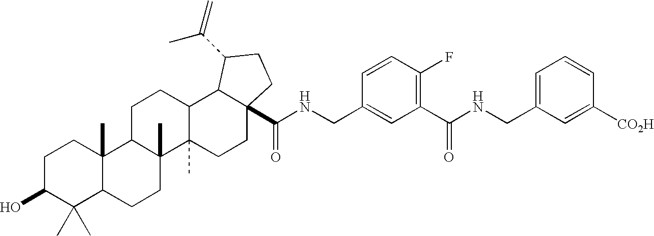 | 50 | 149 | ¹H NMR (400 MHz, d6-DMSO) δ 12.93 (bs, 1H), 9.07 (bt, J = 6 Hz, 1H), 8.22 (bt, J = 6 Hz, 1H), 7.90 (m, 2H), 7.82 (m, 2H), 7.54 (d, J = 8 Hz, 1H), 7.43 (t, J = 8 Hz, 1H), 7.26 (t, J = 10 Hz, 1H), 4.63 (s, 1H), 4.50 (m, 3H), 4.38-4.16 (m, 2H), 3.60 (m, 1H), 2.96 (m, 2H), 2.20 (d, J = 13 Hz, 1H), 1.90-0.50 (m, 41H) [M + H]+ Ion mass = 741.4639, [M + Na]+ Ion mass = 763.4461 |
| 110 | 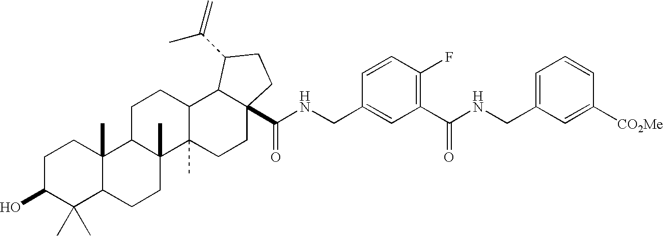 | N/A | 856 | ¹H NMR (400 MHz, d6-DMSO) δ 9.07 (bt, J = 6 Hz, 1H), 8.23 (bt, J = 6 Hz, 1H), 7.90 (m, 2H), 7.83 (m, 2H), 7.58 (d, J = 8 Hz, 1H), 7.47 (t, J = 8 Hz, 1H), 7.26 (t, J = 9 Hz, 1H), 4.62 (s, 1H), 4.52 (m, 3H), 4.38-4.15 (m, 3H), 3.84 (s, 3H), 2.96 (m, 2H), 2.20 (d, J = 13 Hz, 1H), 1.90-0.50 (m, 41H) [M + H]+ Ion mass = 755.4793, [M + Na]+ Ion mass = 777.4609 |
| 111 | 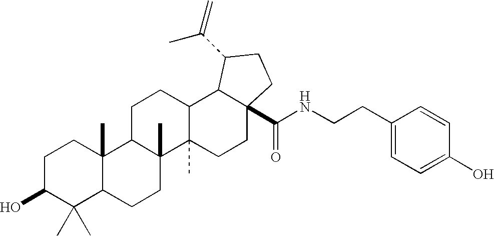 | 3800 | 72.2 | ¹H NMR (DMSO-d6, 400 MHz) δ 9.13 (s, 1H, PhOH), 7.56 (t, 1H, J = 5.4 Hz, NH), 7.57 (d, 2H, J = 8,6 Hz, CH Arom), 6.96 (d, 2H, J = 8,6 Hz, CH Arom), 4.64 (bs, 1H, CH═), 4.52 (bs, 1H, CH═), 4.27 (bs, 1H, H-3), 3.50-0.64 (m, 48H). 576.6 (M + H)+. |
| 112 | 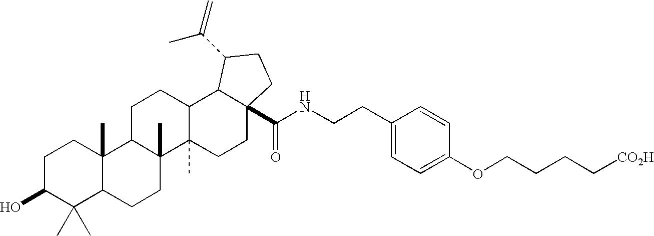 | N/A | 317 | ¹H NMR (DMSO-d6, 400 MHz) δ 12.05 (s, 1H, CO2H), 7.56 (t, 1H, J = 5.4 Hz, NH), 7.07 (d, 2H, J = 8.7 Hz, CH Arom), 6.80 (d, 2H, J = 8,7 Hz, CH Arom), 4.63 (bs, 1H, CH═), 4.52 (bs, 1H, CH═), 4.27 (d, 1H, J = 5.1 Hz, H-3), 3.90 (t, 2H, J = 5.9 Hz, CH2), 3.50-0.64 (m, 54H). 676.4967 (M + H)+. |
| 113 | 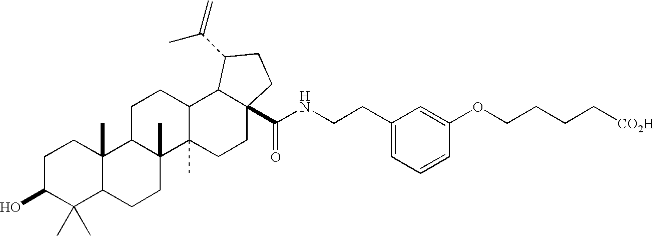 | N/A | 721 | ¹H NMR (DMSO-d6, 400 MHz) δ 12.05 (s, 1H, CO2H), 7.57 (t, 1H, J = 5.0 Hz, NH), 7.15 (m, 1H, CH Arom), 6.74 (m, 3H, CH Arom), 4.64 (bs, 1H, CH═), 4.52 (bs, 1H, CH═), 4.27 (d, 1H, J = 5.1 Hz, H-3), 3.92 (t, 2H, J = 5.9 Hz, CH2), 3.50-0.64 (m, 54H). 676.4919 (M + H)+. |
| 114 | 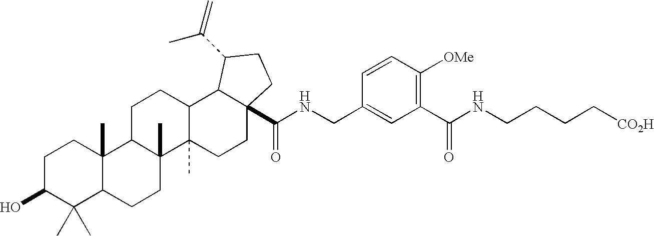 | 211 | 48.4 | ¹H NMR (DMSO-d6, 400 MHz) δ 8.16-8.12 (m, 2H, 2 × NH), 7.63 (bs, 1H, CH Arom), 7.29 (d, 1H, J = 2.3 Hz, CH Arom), 7.04 (d, 1H, J = 8.5 Hz, CH Arom), 4.64 (bs, 1H, CH═), 4.52 (bs, 1H, CH═), 4.27 (m, 2H, CH2), 3.80 (s, 3H, OCH2), 3.25-0.64 (m, 54H). 719.4987 (M + H)+. |
| 115 | 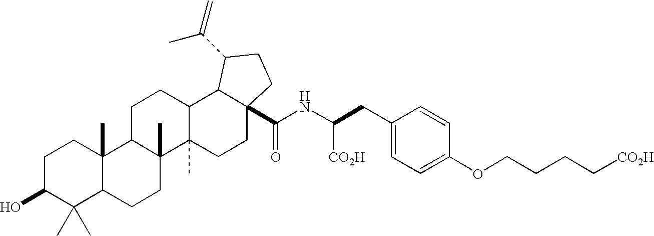 | N/A | 10000 | ¹H NMR (DMSO-d6, 400 MHz) δ 7.70 (d, 1H, J = 9.3 NH), 7.13 (d, 2H, J = 8.6 Hz, CH Arom), 6.75 (d, 2H, J = 8.7 Hz, CH Arom), 4.59 (bs, 1H, CH═), 4.49 (bs, 1H, CH═), 4.27 (d, 1H, J = 5.2 Hz, H-3), 3.85 (t, 2H, J = 5.2 Hz, CH2), 3.31 (s, 2H, CH2), 3.30-0.64 (m, 53H). 720.4945 (M + H)+. |
| 116 | 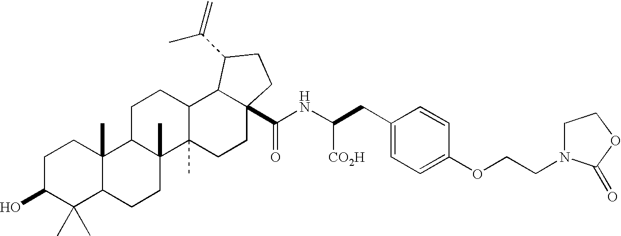 | N/A | 177 | ¹H NMR (DMSO-d6, 400 MHz) δ 12.46 (s, 1H, CO2H), 9.08 (bs, 1H, NH), 7.65 (bs, 1H, NCH), 6.99 (bs, 2H, CH Arom), 6.58 (bs, 2H, CH Arom), 4.59 (bs, 1H, CH═), 4.49 (bs, 1H, CH═), 4.27 (d, 1H, J = 5.2 Hz, H-3), 3.30-0.64 (m, 54H). 733.4907 (M + H)+. |
| 117 | 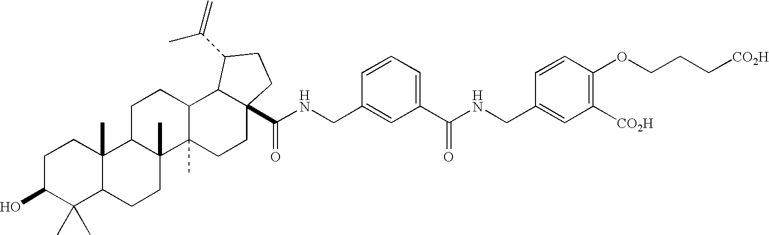 | 384 | 76 | ¹H NMR (400 MHz, d6-DMSO) δ 8.95 (bt, J = 6 Hz, 1H), 8.22 (bt, J = 6 Hz, 1H), 7.83 (s, 1H), 7.72 (m, 1H), 7.37 (m, 2H), 7.31 (s, 1H), 7.20 (d, J = 9 Hz, 1H), 6.90 (d, J = 9 Hz, 1H), 4.64 (s, 1H), 4.53 (s, 1H), 4.38 (d, J = 6 Hz, 2H), 4.23 (m, 2H), 3.93 (m, 2H), 2.98 (m, 2H), 2.60-0.54 (m, 47H) [M + H]+ Ion mass = 825.5047, [M + Na]+ Ion mass = 847.4853 |
| 118 | 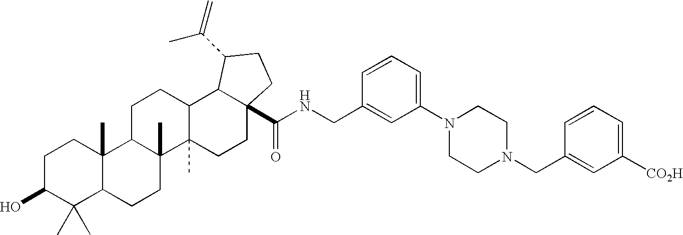 | N/A | 458 | ¹H NMR (400 MHz, d6-DMSO) δ 8.15 (bt, J = 6 Hz, 1H), 7.84 (s, 1H), 7.77 (d, J = 7 Hz, 1H), 7.24 (m, 2H), 7.10 (t, J = 8 Hz, 1H), 6.76 (m, 2H), 6.65 (d, J = 8 Hz, 1H), 4.64 (s, 1H), 4.52 (s, 1H), 4.35-4.00 (m, 3H), 3.5-0.5 (m, 55H) [M + H]+ Ion mass = 764.5349, [M + Na]+ Ion mass = 786.5164 |
| 119 | 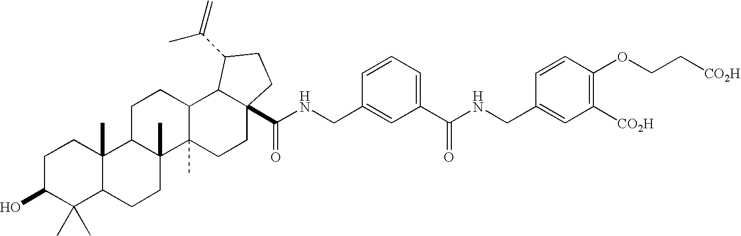 | N/A | 1100 | [M + H]+ Ion mass = 811.4889, [M + Na]+ Ion mass = 833.4713 |
| 120 | 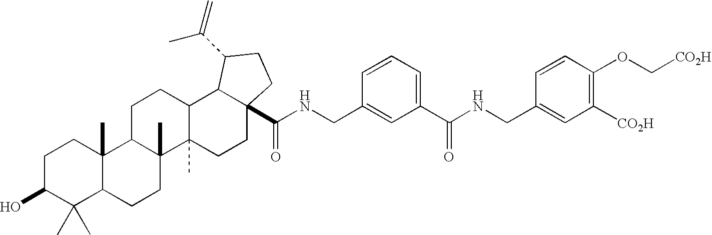 | N/A | 620 | ¹H NMR (400 MHz, d6-DMSO) δ 8.95 (bt, J = 6 Hz, 1H), 8.22 (bt, J = 6 Hz, 1H), 7.78 (s, 1H), 7.72 (m, 1H), 7.40 (m, 3H), 7.18 (m, 1H), 6.97 (m, 1H), 4.64 (s, 1H), 4.53 (s, 1H), 4.38 (d, J = 6 Hz, 2H), 4.25 (m, 4H), 2.98 (m, 2H), 3.10-0.54 (m, 44H) [M + H]+ Ion mass = 797.4742, [M + Na]+ Ion mass = 819.4564 |
| 121 | 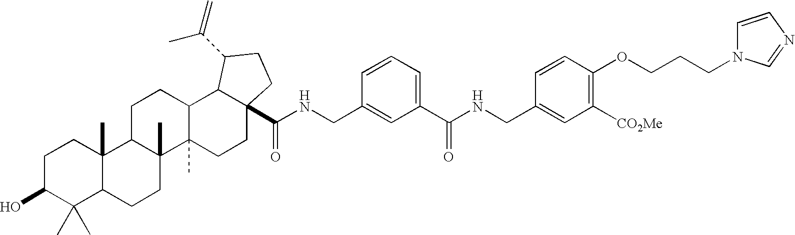 | 17 | 21.3 | ¹H NMR (400 MHz, d6-DMSO) δ 9.06 (s, 1H), 9.01 (bt, J = 6 Hz, 1H), 8.23 (bt, J = 6 Hz, 1H), 7.76 (m, 2H), 7.70 (m, 1H), 7.66 (m, 2H), 7.48 (dd, J = 8, 2 Hz, 1H), 7.39 (m, 2H), 7.10 (d, J = 8 Hz, 1H), 4.64 (s, 1H), 4.52 (s, 1H), 4.39 (m, 4H), 4.26 (m, 3H), 3.81 (s, 3H), 2.98 (m, 3H), 2.23 (m, 2H), 2.19 (bd, J = 13 Hz, 1H), 1.9-0.5 (m, 42H) [M + H]+ Ion mass = 861.5522, [M + Na]+ Ion mass = 883.5355 |
| 122 | 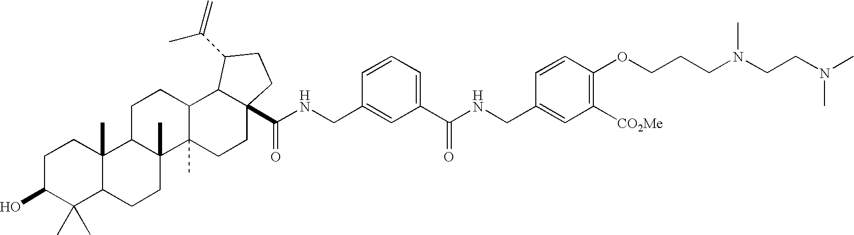 | N/A | 265 | ¹H NMR (400 MHz, d6-DMSO) δ 9.03 (bt, J = 6 Hz, 1H), 8.23 (bt, J = 6 Hz, 1H), 7.76 (s, 1H), 7.74 (m, 1H), 7.69 (m, 2H), 7.50 (dd, J = 9, 2 Hz, 1H), 7.39 (m, 2H), 7.13 (d, J = 9 Hz, 1H), 4.64 (s, 1H), 4.53 (s, 1H), 4.41 (d, J = 5 Hz, 2H), 4.26 (m, 2H), 4.11 (m, 2H), 3.81 (s, 3H), 3.61-0.5 (m, 61H) [M + H]+ Ion mass = 896.1 |
| 123 | 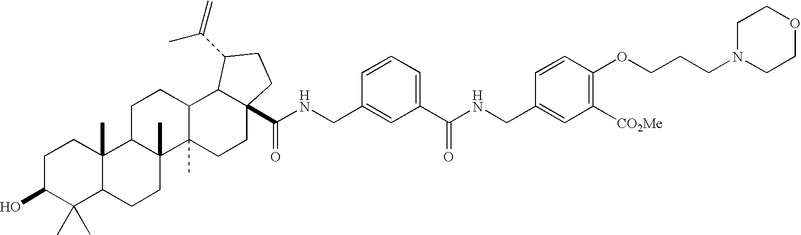 | 13 | 18.9 | ¹H NMR (400 MHz, d6-DMSO) δ 9.02 (bt, J = 6 Hz, 1H), 8.23 (bt, J = 6 Hz, 1H), 7.76 (s, 1H), 7.70 (m, 2H), 7.51 (dd, J = 9, 2 Hz, 1H), 7.39 (d, J = 5 Hz, 2H), 7.14 (d, J = 9 Hz, 1H), 4.64 (s, 1H), 4.53 (s, 1H), 4.42 (d, J = 6 Hz, 1H), 4.34-4.18 (m, 3H), 4.12 (bt, J = 6 Hz, 2H), 4.00 (m, 2H), 3.81 (s, 3H), 3.68 (t, J = 12 Hz, 2H), 351 (d, J = 12 Hz, 2H), 3.12 (m, 2H), 2.96 (m, 2H), 2.16 (m, 3H), 1.9-0.5 (m, 44H) [M + H]+ Ion mass = 880.5829, [M + Na]+ Ion mass = 902.5666 |
| 124 | 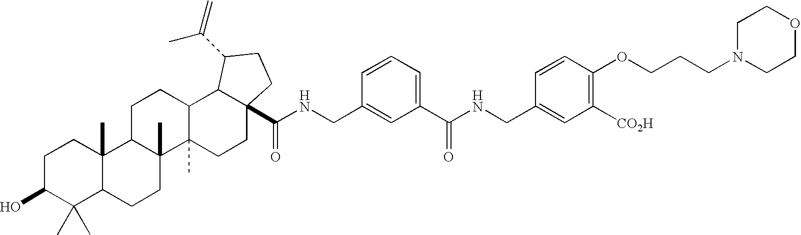 | 8 | 27.9 | [M + H]+ Ion mass = 866.5670, [M + Na]+ Ion mass = 888.5497 |
| 125 | 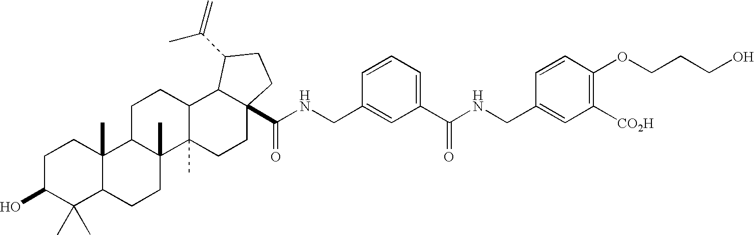 | 56 | 42 | [M + H]+ Ion mass = 798.0 |
| 126 | 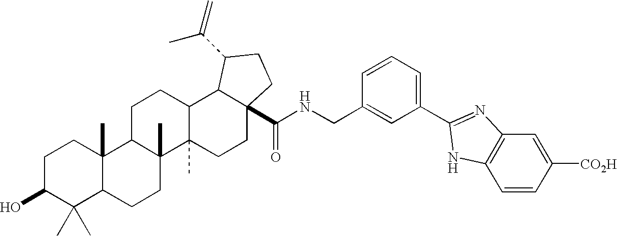 | N/A | 66 | ¹H NMR (400 MHz, d6-DMSO) δ 13.21 (bs, 1H), 8.31 (bt, J = 6 Hz, 1H), 8.15 (m, 2H), 7.97 (d, J = 7 Hz, 1H), 7.83 (m, 1H), 7.62 (m, 1H), 7.49 (t, J = 8 Hz, 1H), 7.39 (d, J = 8 Hz, 1H), 4.63 (s, 1H), 4.48 (m, 2H), 4.17 (m, 3H), 3.23-0.35 (m, 43H) [M + H]+ Ion mass = 706.8 |
| 127 | 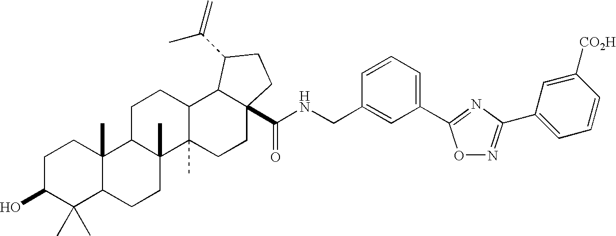 | N/A | 768 | ¹H NMR (400 MHz, d6-DMSO) δ 8.50-8.67 (m, 1H), 8.40-8.46 (1H, m), 8.30-8.34 (1H, m), 8.10-8.18 (2H, m), 8.04-8.07 (1H, m), 7.72-7.76 (1H, m), 7.60-7.61 (2H, m), 4.63 (1H, br s), 4.45-4.54 (2H, m), 4.15-4.20 (1H, m), 0.55-3.6 (46H, m). Mass Spec (m/z): 734 (M + 1). |
| 128 | 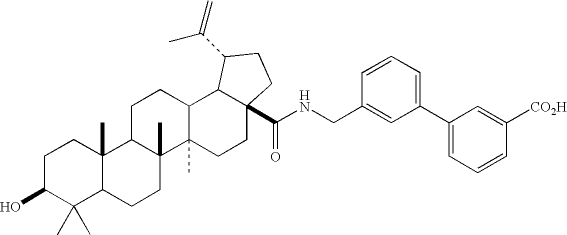 | N/A | 1000 | ¹H NMR (400 MHz, d6-DMSO) δ 8.24-8.28 (1H, m), 8.13-8.14 (1H, m), 7.92-7.94 (1H, m), 7.82-7.85 (1H, m), 7.52-7.61 (3H, m), 7.39-7.43 (1H, m), 7.26-7.28 (1H, m), 4.629-4.63 (1H, br m), 4.49-4.51 (1H, br m), 4.43-4.49 (1H, m), 4.13-4.18 (1H, m), 0.5-3.2 (46H, m). Mass Spec (m/z): 666 (M + 1). |
| 129 | 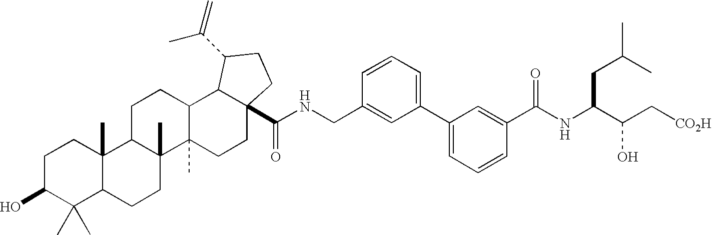 | N/A | 3600 | ¹H NMR (400 MHz, d6-DMSO) δ 8.20-8.30 (1H, m), 8.1-8.11 (1H, br s), 8.02-8.04 (1H, m), 7.86-7.88 (1H, m), 7.69-7.74 (1H, m), 7.50-7.60 (3H, m), 7.35-7.45 (1H, m), 7.25-7.27 (1H, m), 4.63 (1H, br s), 4.52 (1H, br s), 4.40-4.43 (1H, m), 4.17-4.23 (1H, m), 4.17-4.23 (1H, m), 0.5-3.33 (60H, m). Mass Spec (m/z): 823 (M + 1). |
| 130 | 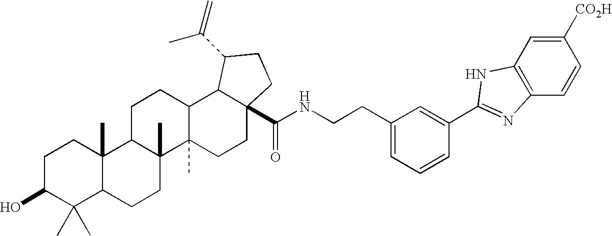 | 230 | 39 | ¹H NMR (400 MHz, d6-DMSO) δ 8.25 (m, 3H), 7.96 (d, J = 8 Hz, 1H), 7.78 (d, J = 8 Hz, 1H), 7.69 (m, 1H), 7.52 (m, 2H), 4.61 (s, 1H), 4.50 (s, 1H), 4.00-0.40 (m, 50H) [M + H]+ Ion mass = 720.4720 |
| 131 | 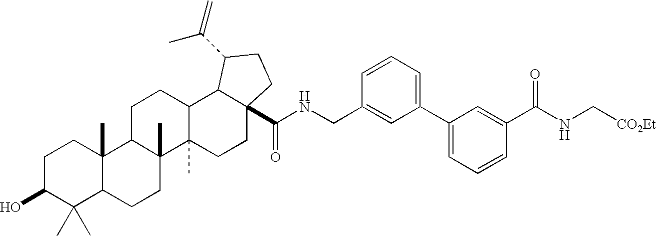 | N/A | 6500 | ¹H NMR (DMSO-d6, 400 MHz) δ 9.07 (t, 1H, J = 6.1 Hz, NH), 8.25 (t, 1H, J = 6.0 Hz, NH), 8.13 (s, 1H, CH Arom), 7.86 (d, 1H, J = 7.6 Hz, CH Arom), 7.76 (d, 1H, J = 7.5 Hz, CH Arom), 7.57 (m, 3H, CH Arom), 7.43 (t, 1H, J = 7.7 Hz, CH Arom), 7.27 (d, 1H, J = 7.8 Hz, CH Arom), 4.64 (bs, 1H, CH═), 4.52 (bs, 1H, CH═), 4.45 (dd, 1H, J = 5.8, J = 6.4, CH2), 4.27-4.12 (m, 3H, H-3, OCH2), 4.02 (d, 2H, J = 5.7, CH2), 3.00-0.64 (m, 48H). 751.5048 (M + H)+. |
| 132 | 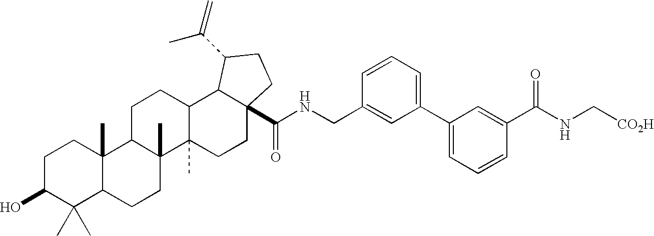 | N/A | 1100 | ¹H NMR (DMSO-d6, 400 MHz) δ 8.96 (t, 1H, J = 6.4 Hz, NH), 8.26 (t, 1H, J = 6.3 Hz, NH), 8.13 (s, 1H, CH Arom), 7.87 (d, 1H, J = 8.0 Hz, CH Arom), 7.75 (d, 1H, J = 7.8 Hz, CH Arom), 7.57 (m, 3H, CH Arom), 7.43 (t, 1H, J = 7.8 Hz, CH Arom), 7.27 (d, 1H, J = 8.0 Hz, CH Arom), 4.64 (bs, 1H, CH═), 4.52 (bs, 1H, CH═), 4.46 (dd, 1H, J = 5.8, J = 6.0, CH), 4.27-4.12 (m, 2H, H-3, CH2), 3.94 (d, 2H, J = 5.8, CH2), 3.00-0.64 (m, 45H). 751.5048 (M + H)+. |
| 133 | 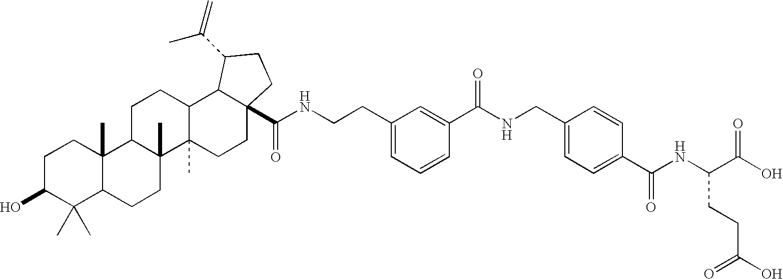 | 84 | 34 | ¹H NMR (400 MHz, d6-DMSO) δ 12.60 (bs, 1.5H), 9.06 (bt, J = 6 Hz, 1H), 8.52 (bs, 1H), 7.84 (d, J = 8 Hz, 2H), 7.76 (s, 1H), 7.76 (s, 1H), 7.73 (m, 1H), 7.63 (bt, J = 5 Hz, 1H), 7.39 (m, 4H), 4.63 (s, 1H), 4.53 (m, 3H), 4.38 (m, 1H), 4.27 (d, J = 5 Hz, 1H), 3.26 (m, 2H), 2.96 (m, 2H), 2.76 (m, 2H), 2.33 (m, 3H), 2.14-0.5 (m, 43H) [M + H]+ Ion mass = 866.5306, [M + Na]+ Ion mass = 888.5130 |
| 134 | 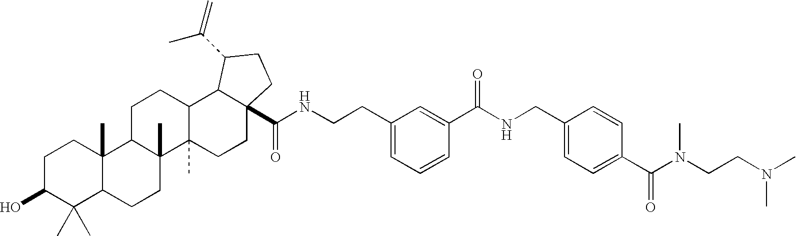 | 240 | 68.7 | ¹H NMR (400 MHz, d6-DMSO) δ 9.04 (bt, J = 6 Hz, 1H), 7.95 (s, 0.5H), 7.77 (s, 1H), 7.74 (m, 1H), 7.63 (m, 1H), 7.33 (m, 6H), 4.63 (s, 1H), 4.51 (m, 3H), 4.27 (d, J = 5 Hz, 1H), 3.61-0.5 (m, 61H) [M + H]+ Ion mass = 821.5950, [M + Na]+ Ion mass = 843.5761 |
| 135 | 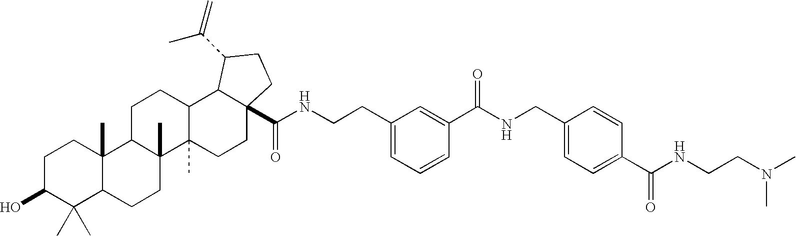 | 533 | 70 | ¹H NMR (400 MHz, d6-DMSO) δ 9.04 (bt, J = 6 Hz, 1H), 8.35 (bt, J = 6 Hz, 1H), 7.95 (s, 0.5H), 7.76 (m, 4H), 7.63 (bt, J = 6 Hz, 1H), 7.37 (m, 4H), 4.63 (s, 1H), 4.52 (m, 3H), 4.28 (d, J = 5 Hz, 1H), 3.61-0.5 (m, 58H) [M + H]+ Ion mass = 807.5785, [M + Na]+ Ion mass = 829.5604 |
| 136 | 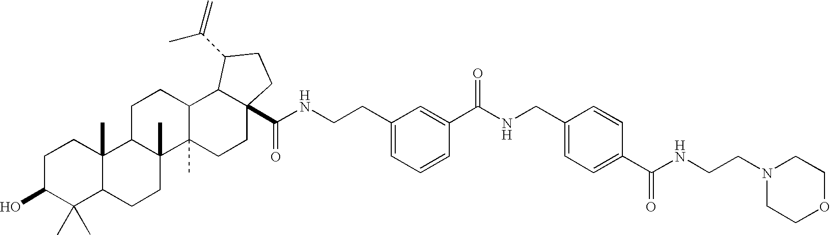 | 14.1 | 11.6 | ¹H NMR (400 MHz, d6-DMSO) δ 9.04 (bt, J = 6 Hz, 1H), 8.36 (bt, J = 6 Hz, 1H), 7.76 (m, 4H), 7.63 (bt, J = 6 Hz, 1H), 7.37 (m, 4H), 4.63 (s, 1H), 4.51 (m, 3H), 4.26 (d, J = 5 Hz, 1H), 3.56 (m, 4H), 3.26 (m, 2H), 2.96 (m, 2H), 2.76 (m, 2H), 2.43 (m, 6H), 2.04 (d, J = 13 Hz, 1H), 1.9-0.5 (m, 43H) [M + H]+ Ion mass = 849.5888, [M + Na]+ Ion mass = 871.5670 |
| 137 | 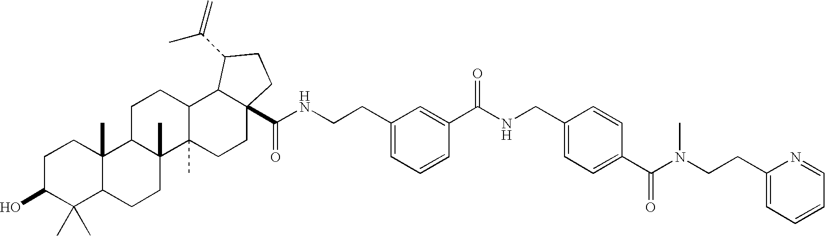 | 10.5 | 10.8 | ¹H NMR (400 MHz, d6-DMSO) δ 9.03 (bs, 1H), 8.45 (bd, J = 50 Hz, 1H), 7.84-7.55 (m, 4H), 7.45-7.00 (m, 8H), 4.63 (s, 1H), 4.50 (m, 3H), 4.27 (d, J = 5 Hz, 1H), 3.77 (bs, 1H), 3.56 (bs, 1H), 3.46-0.53 (m, 53H) [M + H]+ Ion mass = 855.5792, [M + Na]+ Ion mass = 877.5604 |
| 138 | 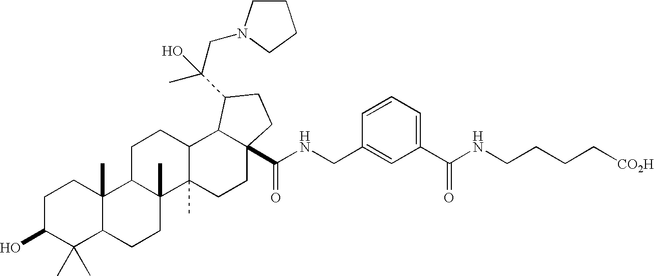 | N/A | 550 | [M + H]+ Ion mass = 776.5581, [M + Na]+ Ion mass 798.5401 |
| 139 | 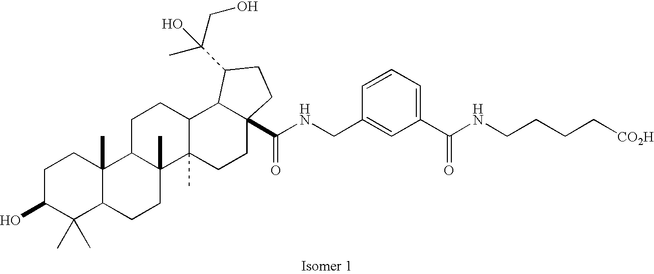 | N/A | 1500 | ¹H NMR (DMSO-d6, 400 MHz) δ 8.55 (m, 1H), 8.10 (m, 1H), 7.75 (s, 1H), 7.68 (d, J = 6.8 Hz, 2H), 7.35 (m, 2H), 4.30-4.18 (m, 4H), 3.17 (m, 3H), 3.00 (m, 1H), 2.25-0.95 (m, 29H), 0.99 (s, 3H), 0.92 (s, 3H), 0.78 (s, 3H), 0.74 (s, 3H), 0.67 (s, 3H), 0.64 (s, 3H). TOF-MS m/z 745 (M + Na)⁺ |
| 140 | 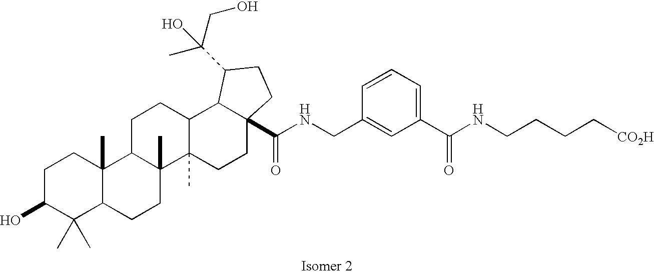 | N/A | 1600 | ¹H NMR (DMSO-d6, 400 MHz) δ 8.52 (m, 1H), 8.08 (m, 1H), 7.74 (s, 1H), 7.66 (d, J = 6.8 Hz, 2H), 7.34 (m, 2H), 4.30-4.18 (m, 4H), 3.17 (m, 3H), 3.00 (m, 1H), 2.25-0.95 (m, 29H), 0.99 (s, 3H), 0.92 (s, 3H), 0.78 (s, 3H), 0.74 (s, 3H), 0.67 (s, 3H), 0.64 (s, 3H). TOF-MS m/z 745 (M + Na)⁺ |
| 141 | 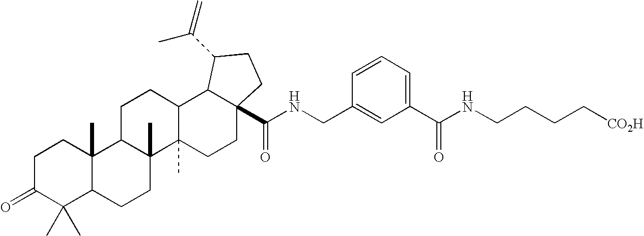 | 1500 | 157 | ¹H NMR (DMSO-d6, 400 MHz) δ 8.51 (m, 1H), 8.23 (m, 1H), 7.74 (s, 1H), 7.67 (m, 1H), 7.36 (d, J = 5.6 Hz, 2H), 4.64 (s, 1H), 4.53 (s, 1H), 4.32 (m, 1H), 4.19 (m, 1H), 3.23 (m, 1H), 3.01 (m, 1H), 2.50-1.00 (m, 34H), 0.98 (s, 3H), 0.92 (s, 6H), 0.82 (s, 3H), 0.74 (s, 3H). TOF-MS m/z 687 (M + H)⁺ |
| 142 | 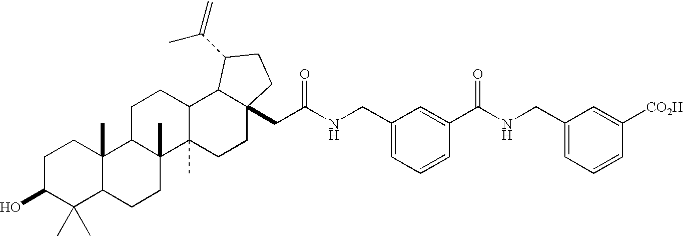 | N/A | 3400 | ¹H NMR (DMSO-d6, 400 MHz) δ 9.10 (m, 1H), 8.37 (m, 1H), 7.88 (s, 1H), 7.78 (m, 3H), 7.48-7.36 (m, 4H), 4.67 (s, 1H), 4.54 (s, 1H), 4.51 (d, J = 6.0 Hz, 2H), 4.29 (d, J = 6.0 Hz, 2H), 2.97 (m, 1H), 2.50-0.80 (m, 39H), 0.77 (s, 3H), 0.65 (s, 3H). TOF-MS m/z 737 (M + H)⁺ |
| 143 | 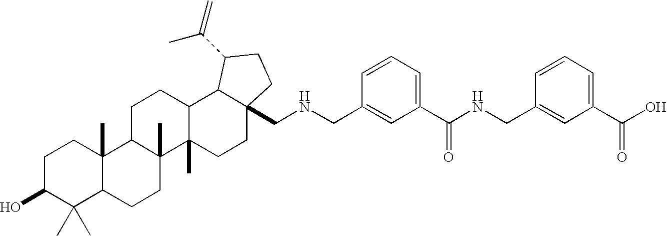 | 1300 | 120 | ¹H NMR (400 MHz, d6-DMSO) δ 8.15 (1H, br s), 7.90-7.93 (2H, m), 7.80-7.84 (1H, m), 7.50-7.58 (4H, m), 7.40-7.46 (1H, m), 4.50-4.70 (3H, m), 4.1-4.3 (1H, m), 0.55-3.5 (51H, m). Mass Spec (m/z): 709 (M + 1). |
| 144 | 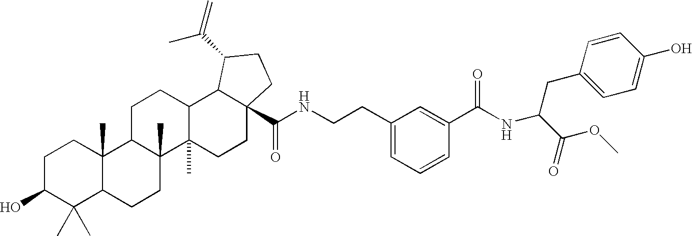 | N/A | 204 | 1H NMR (DMSO-d6, 400 MHz) 9.21 (s, 1H, PhOH), 8.75 (d, J = 7.8 Hz, 1H, NH), 7.64-7.77 (m, 1H, NH, 2H, CH arom), 7.36 (m, 2H, CH Arom), 7.07 (d, J = 8.6 Hz, 2H, CH Arom), 6.66 (d, 2H, CH arom), 4.64 (bs, 1H, CH═), 4.52 (bs, 1H, CH═), 4.27 (d, 1H), J = 5.0 Hz, CH), 3.61 (s, 3H, OCH3), 3.00-0.64 (m, 51H); Mass Spec (m/z): 780.0 (M + 1). |
| 145 | 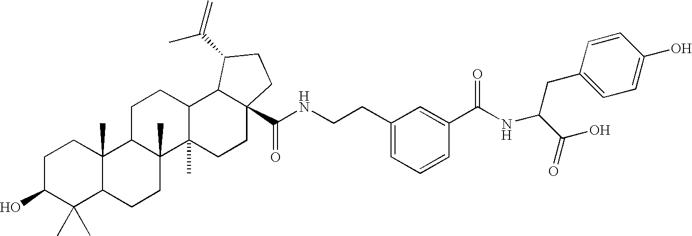 | N/A | 1100 | 1H NMR (DMSO-d6, 400 MHz) δ 8.60 (d, J = 7.82 Hz, 1H, NH), 7.64-7.77 (m, 1H, NH, 2H, CH arom), 7.36 (m, 2H, CH Arom), 7.07 (d, 2H, J = 8.4, CH Arom), 6.66 (d, 2H, J = 8.4 Hz, CH arom), 4.64 (bs, 1H, CH═), 4.52 (bs, 1H, CH═), 4.27 (d, J = 5.0 Hz, 1H, CH), 3.00-0.64 (m, 53H); Mass Spec (m/z): 767.07 (M + 1). |
| 146 | 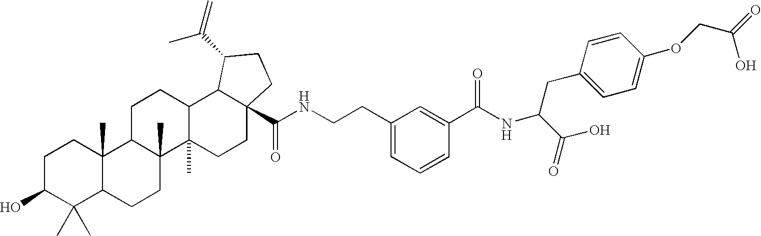 | 3300 | 197 | 1H NMR (DMSO-d6, 400 MHz) δ 8.60 (d, J = 7.6 Hz, 1H, NH), 7.64-7.67 (m, 1H, NH, 2H, CH arom), 7.3 (d, J = 6 Hz, 2H, CH Arom), 7.20 (d, , J = 8.8 Hz, 2HCH Arom), 6.79 (d, J = 8.8 Hz, 2H, CH arom), 4.64 (bs, 1H, CH═), 4.52 (bs, 1H, CH═), 4.27 (d, J = 5.0 Hz, 1H, CH), 3.00-0.64 (m, 55H); Mass Spec (m/z): 825.11 (M + 1). |
| 147 | 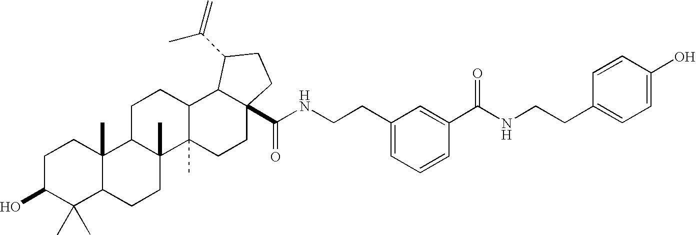 | 30 | 26.6 | 1H NMR (DMSO-d6, 400 MHz) δ 9.18 (s, 1H, PhOH), 8.49 (t, J = 5.6 Hz, 1H, NH), 7.61-7.68 (m, 1H, NH, 2H, CH arom), 7.35 (m, 2H, CH Arom), 7.03 (d, J = 8.4 Hz, 2H, CH Arom), 6.66 (d, J = 6.4 Hz, 2H, CH arom), 4.64 (bs, 1H, CH═), 4.52 (bs, 1H, CH═), 4.27 (d, J = 5.0 Hz, 1H, CH), 3.00-0.64 (m, 52H); Mass Spec (m/z): 723.06 (M + 1). |
| 148 | 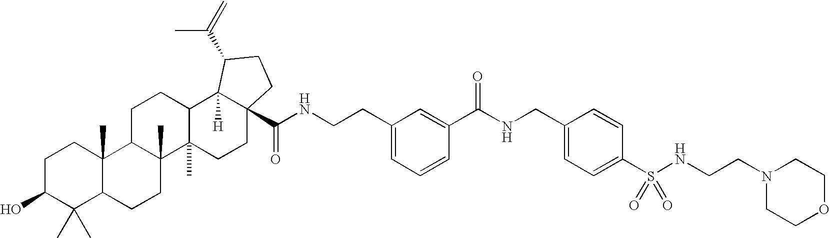 | 53 | 6 | 1H NMR (DMSO-d6, 400 MHz) δ 9.11 (m, 1H), 7.75 (m, 4H), 7.64 (m, 1H), 7.54 (m, 1H), 7.50 (d, J = 8.8 Hz, 2H), 7.38 (m, 2H), 4.63 (s, 1H), 4.53 (m, 3H), 4.29 (d, J = 4.8 Hz, 1H), 4.13 (m, 2H), 3.48 (m, 3H), 3.17 (d, J = 4.8 Hz, 4H), 2.99 (m, 2H), 2.50-0.90 (m, 39H), 0.86 (s, 3H), 0.75 (s, 3H), 0.64 (s, 3H). TOF-MS m/z 885 (M + H)+ |
| 149 | 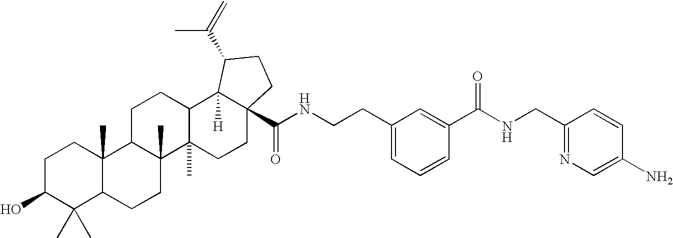 | 88 | 23 | 1H NMR (DMSO-d6, 400 MHz) δ 8.87 (m, 1H), 7.86 (s, 1H), 7.76 (s, 1H), 7.72 (m, 1H), 7.63 (m, 1H), 7.35 (m, 2H), 6.98 (d, J = 8.4 Hz, 1H), 6.87 (d, J = 8.4 Hz, 1H), 4.63 (s, 1H), 4.52 (s, 1H), 4.38 (d, J = 6.0 Hz, 2H), 4.27 (d, J = 5.6 Hz, 1H), 2.99 (m, 2H), 2.80-0.72 (m, 42H), 0.64 (s, 3H). TOF-MS m/z 709 (M + H)+ |
| 150 | 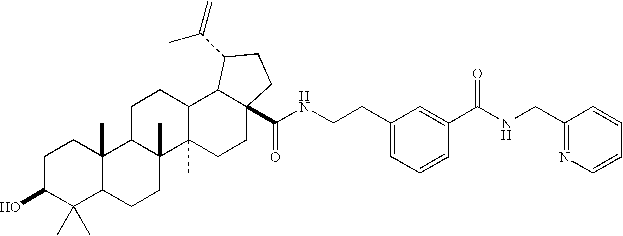 | 122 | 24 | 1H NMR (DMSO-d6, 400 MHz) δ 9.07 (m, 1H), 8.51 (m, 1H), 7.78 (s, 1H), 7.75 (m, 2H), 7.64 (m, 1H), 7.38 (m, 2H), 7.30 (d, J = 7.6 Hz, 1H), 7.26 (m, 1H), 4.63 (s, 1H), 4.56 (d, J = 6.0 Hz, 2H), 4.52 (s, 1H), 4.27 (d, J = 5.2 Hz, 1H), 2.99 (m, 2H), 2.80-0.72 (m, 42H), 0.64 (s, 3H). TOF-MS m/z 694 (M + H)+ |
| 151 | 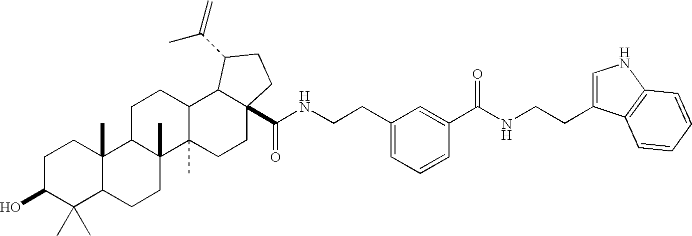 | 325 | 85 | 1H NMR (400 MHz, d6-DMSO) δ 10.83 (s, 1H), 8.59 (t, J = 5.6 Hz, 1H), 7.72 (s, 1H), 7.66 (m, 2H), 7.59 (d, J = 8.9 Hz, 1H), 7.35 (m, 2H), 7.18 (m, 1H), 7.07 (t, J = 7.3 Hz, 1H), 6.98 (t, J = 7.3 Hz, 1H), 4.64 (s, 1H), 4.52 (s, 1H), 4.28 (m, 1H), 3.53 (m, 1H), 3.35 (m, 2H), 2.94 (m, 3H), 2.05 (m, 1H), 1.71 (m, 1H), 1.5-0.60 (m, 45 H); Mass Spec (m/z): 746.5 (M + 1). |
| 152 | 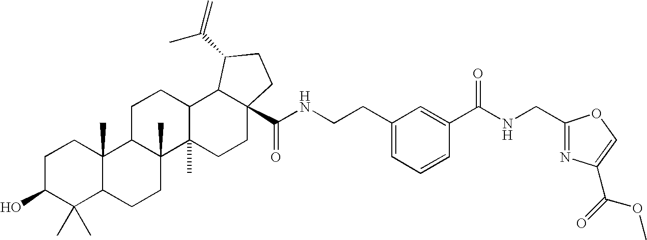 | 14 | 36 | 1H NMR (400 MHz, CDCl3) δ 8.19 (s, 1H), 7.68 (s, 1H), 7.60 (m, 1H), 7.48 (t, J = 5.8 Hz, 1H), 7.31 (m, 2H), 5.83 (t, J = 5.2 Hz, 1H), 4.80 (m, 2H), 4.69 (s, 1H), 4.55 (s, 1H), 3.87 (s, 3H), 3.44 (q, J = 6.4 Hz, 2H), 3.15 (m, 2H), 2.80 (m, 2H), 2.42 (dt, J = 12.8, 3.6 Hz, 1H), 1.84 (m, 3H), 1.64-0.63 (m, 41H); Mass Spec (m/z): 742.5 (M + 1). |
| 153 | 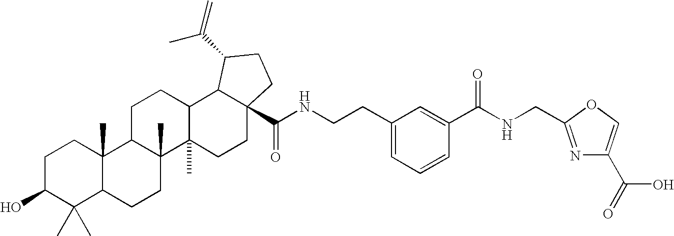 | 14 | 44.7 | 1H NMR (400 MHz, d6-DMSO) δ 9.18 (t, J = 5.4 H, 1H), 8.69 (s, 1H), 7.74 (m, 2H), 7.65 (t, J = 5.4 Hz, 1H), 7.38 (m, 2H), 4.63 (m, 4H), 3.29 (m, 3H), 2.96 (m, 2H), 2.76 (m, 2H), 2.46 (m, 2H), 2.05 (m, 1H), 1.48-1.09 (m, 12H), 0.96-0.59 (m 19H); Mass Spec (m/z): 728.5 (M + 1). |
| 154 | 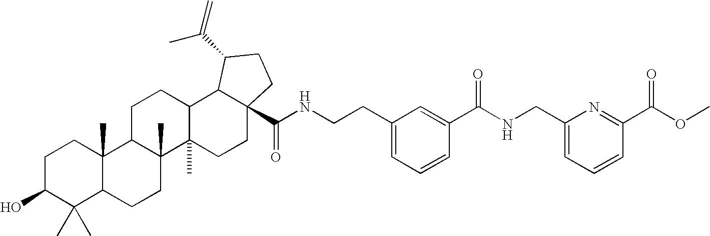 | 8 | 34 | 1H NMR (400 MHz, d6-DMSO) δ 9.20 (bt, 1H), 7.95 (s, 2H), 7.78 (m, 2H), 7.60 (m, 2H), 7.39 (m, 2H), 4.56 (m, 4H), 4.27 (m, 1H), 3.89 (s, 3H), 3.26 (m, 3H), 2.86 (m, 5H), 2.50 (m, 6H), 2.03 (m, 2H), 1.70-1.0 (m, 15H), 0.96-0.58 (m, 17H); Mass Spec (m/z): 753.5 (M + 1). |
| 155 | 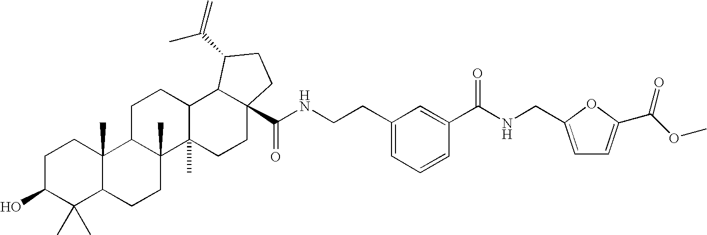 | 17 | 25.6 | 1H NMR (400 MHz, d6-DMSO) δ 9.06 (t, J = 5.8 Hz, 1H), 7.75 (s, 1H), 7.71 (m, 1H), 7.63 (t, J = 5.0 Hz, 1H), 7.37 (d, J = 4.9 Hz, 1H), 7.25 (d, J = 3.6 Hz, 1H), 6.48 (d, J = 3.5 Hz, 1H), 5.76 (s, 1H), 4.63 (s, 1H), 4.51 (m, 3H), 4.27 (d, J = 5.3 Hz, 1H), 3.40-3.20 (m, 3H), 2.97 (m, 2H), 2.74 (m, 3H), 2.04 (m, 1H), 1.77-0.59 (m, 42H); Mass Spec (m/z): 742.5 (M + 1). |
| 156 | 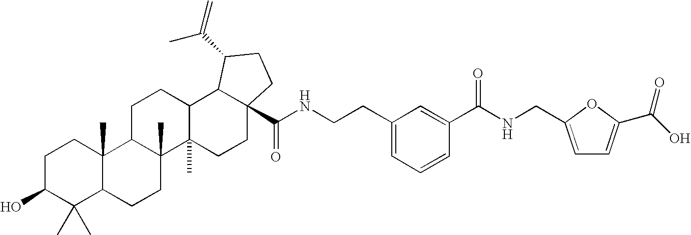 | 8 | 51 | 1H NMR (400 MHz, d6-DMSO) δ 9.05 (t, J = 5.0 Hz, 1H), 7.73 (m, 2H), 7.63 (t, J = 5.0 Hz, 1H), 7.14 (d, J = 3.8 Hz, 1H), 6.44 (d, J = 4.5 Hz, 1H), 4.63 (s, 1H), 4.51 (m, 4H), 3.30 (m, 3H), 2.96 (m, 2H), 2.75 (m, 2H), 2.04 (m, 2H), 1.69-0.61 (m, 39H); Mass Spec (m/z): 727.5 (M + 1). |
| 157 | 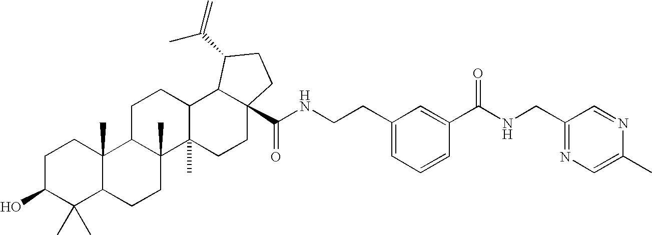 | 111 | 32 | 1H NMR (400 MHz, d6-DMSO) δ 9.10 (t, J = 5.0 Hz, 1H), 8.48 (m, 2H), 7.74 (m, 2H), 7.62 (t, J = 3.7 Hz, 1H), 7.37 (m, 2H), 4.63 (s, 1H), 4.56 (m, 2H), 4.51 (s, 1H), 4.27 (d, J = 5.0 Hz, 1H), 3.34 (m, 4H), 2.96 (m, 2H), 2.75 (m, 2H), 2.47 (m, 4H), 2.04 (m, 1H), 1.59 (m, 7H), 1.40-1.10 (m, 18H); Mass Spec (m/z): 710.1 (M + 1). |
| 158 | 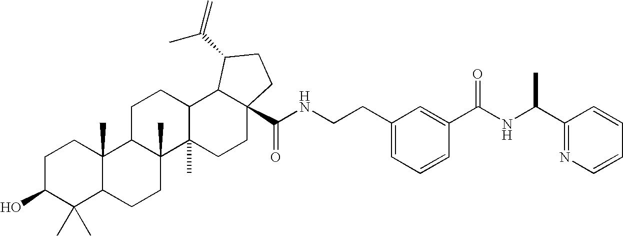 | >10000 | 249 | 1H NMR (400 MHz, d6-DMSO) δ 8.76 (d, J = 8.5 Hz, 1H), 8.53 (d, J = 5.3 Hz, 1H), 7.75 (m, 3H), 7.64 (bt, 1H), 7.38 (m, 3H), 7.26 (m, 1H), 5.20 (p, J = 7.6 Hz, 1H), 4.63 (s, 1H), 4.52 (s, 1H), 4.27 (d, J = 5.4 Hz, 1H), 3.34 (m, 4H), 2.97 (m, 2H), 2.77 (m, 2H), 2.05 (m, 1H), 1.78-1.49 (m, 13H), 1.46-1.00 (m, 14H), 0.93-58 (m, 15H); Mass Spec (m/z): 709.1 (M + 1). |
| 159 | 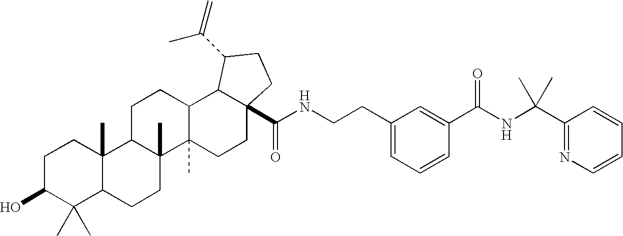 | >10000 | 250 | 1H NMR (400 MHz, d6-DMSO) δ 8.59 (s, 1H), 8.50 (d, J = 6.4 Hz, 1H), 7.75-7.60 (m, 4H), 7.43 (d, J = 8.5 Hz, 1H), 7.36 (d, J = 4.3 Hz, 2H), 7.22 (m, 1H), 4.63 (s, 1H), 4.51 (s, 1H), 4.27 (d, J = 6.4 Hz, 1H), 3.40-3.22 (m, 2H), 2.95 (m, 2H), 2.77 (m, 2H), 2.54 (m, 1H), 2.06 (m, 1H), 1.76-1.48 (m, 13H), 1.45-1.03 (m, 12H), 0.92-57 (m, 21H); Mass Spec (m/z): 723.1 (M + 1). |
| 160 | 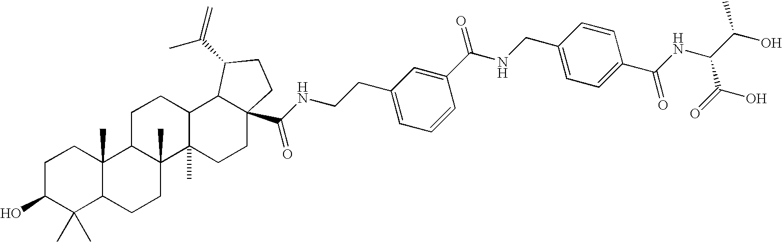 | 39 | 15 | 1H NMR (400 MHz, d6-DMSO) δ 9.07 (bt, J = 6 Hz, 1H), 7.99 (d, J = 8 Hz, 1H), 7.86 (d, J = 8 Hz, 2H) 7.75 (m, 2H), 7.64 (bt, J = 6 Hz, 1H), 7.42 (d, J = 8 Hz, 2H), 7.37 (m, 2H), 4.63 (s, 1H), 4.53 (m, 3H), 4.42 (dd, J = 4.9 Hz, 1H), 4.19 (m, 1H), 3.60 (m, 2H), 2.77 (m, 2H), 2.05 (bd, J = 13 Hz, 1H), 1.80-0.50 (m, 45H) [M + H]+ Ion mass = 838.5378, [M + Na]+ Ion mass = 860.5201 |
| 161 | 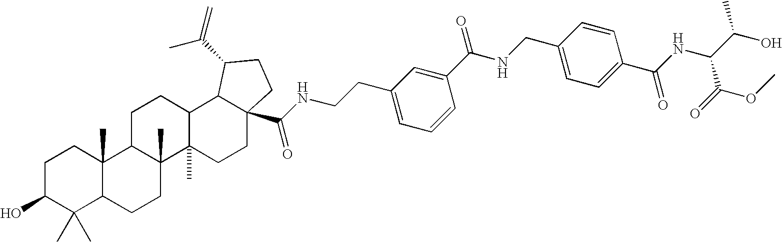 | 15 | 15 | 1H NMR (400 MHz, d6-DMSO) δ 9.07 (bt, J = 6 Hz, 1H), 8.17 (d, J = 8 Hz, 1H), 7.86 (d, J = 8 Hz, 2H), 7.75 (m, 2H), 7.63 (bt, J = 6 Hz, 1H), 7.42 (d, J = 8 Hz, 2H), 7.37 (m, 2H), 4.98 (d, J = 8 Hz, 1H), 4.63 (s, 1H), 4.51 (m, 4H), 4.27 (d, J = 6 Hz, 1H), 4.18 (m, 1H), 3.65 (s, 3H), 3.26 (m, 1H), 2.96 (m, 2H), 2.76 (m, 2H), 2.05 (bd, J = 13 Hz, 1H), 1.80-0.50 (m, 45H) [M + H]+ Ion mass = 853.5521, [M + Na]+ Ion mass = 874.5347 |
| 162 | 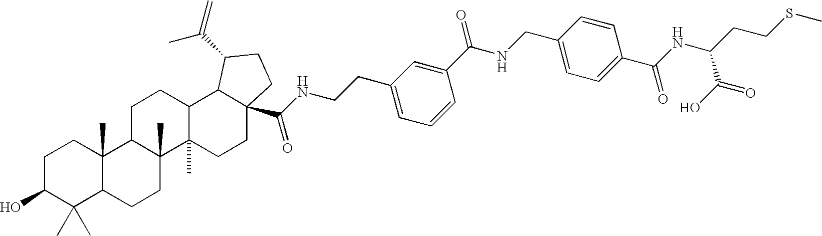 | 12 | 9 | 1H NMR (400 MHz, d6-DMSO) δ 9.07 (bt, J = 6 Hz, 1H), 8.60 (d, J = 7 Hz, 1H), 7.85 (d, J = 8 Hz, 2H), 7.75 (m, 2H), 7.64 (bt, J = 6 Hz, 1H), 7.39 (m, 4H), 4.63 (s, 1H), 4.52 (m, 4H), 4.28 (m, 1H), 3.25 (m, 2H), 2.95 (m, 2H), 2.77 (m, 2H), 2.56 (m, 2H), 2.05 (m, 6H), 1.80-0.50 (m, 42H) [M + H]+ Ion mass = 882.5395 |
| 163 | 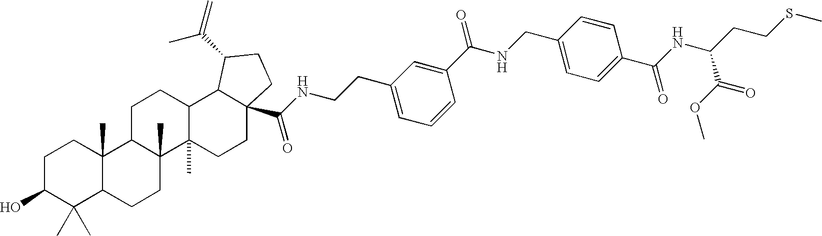 | 12 | 11 | [M + H]+ Ion mass = 868.5248 |
| 164 | 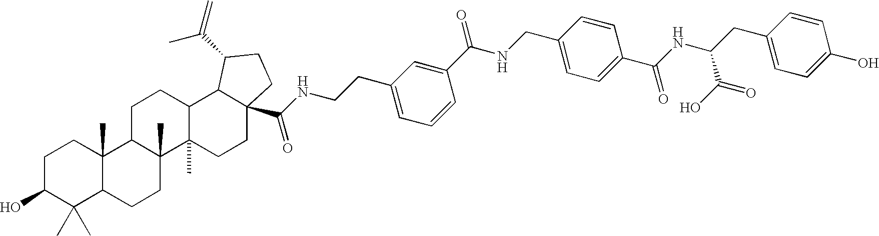 | 7 | 7 | 1H NMR (400 MHz, d6-DMSO) δ 9.20 (s, 1H), 9.07 (bt, J = 6 Hz, 1H), 8.60 (d, J = 8 Hz, 1H), 7.75 (m, 4H), 7.65 (bt, J = 6 Hz, 1H), 7.37 (d, J = 7 Hz, 4H), 7.08 (d, J = 8 Hz, 2H), 6.63 (d, J = 8 Hz, 2H), 4.63 (s, 1H), 4.51 (m, 4H), 4.29 (d, J = 5 Hz, 1H), 3.26 (m, 1H), 3.09-2.88 (m, 4H), 2.77 (m, 2H), 2.05 (bd, J = 13 Hz, 1H), 1.80-0.50 (m, 43H) [M + H]+ Ion mass = 900.5516, [M + Na]+ Ion mass = 922.5333 |
| 165 | 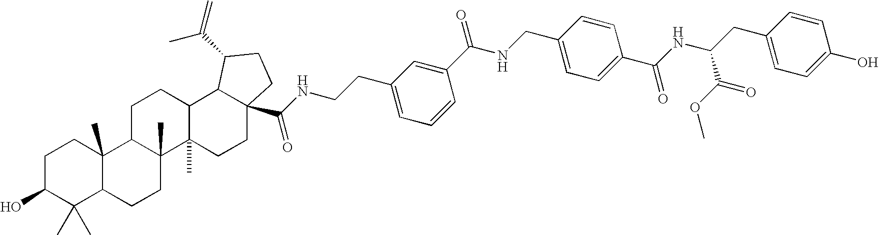 | 20 | 13 | [M + H]+ Ion mass = 914.5673, [M + Na]+ Ion mass = 936.5499 |
| 166 | 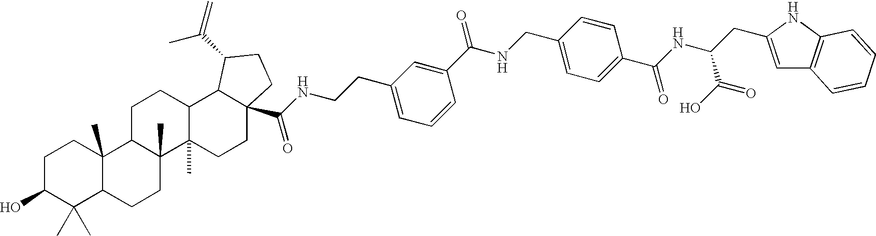 | 13 | 22 | 1H NMR (400 MHz, d6-DMSO) δ 10.77 (s, 1H), 9.08 (bt, J = 6 Hz, 1H), 8.28 (bs, 1H), 7.73 (m, 3H), 7.67 (bt, J = 6 Hz, 1H), 7.55 (d, J = 8 Hz, 1H), 7.36 (m, 4H), 7.28 (d, J = 8 Hz, 2H), 7.12 (bs, 1H), 7.01 (t, J = 8 Hz, 1H), 6.92 (t, J = 8 Hz, 1H), 4.63 (s, 1H), 4.50 (m, 4H), 4.30 (s, 1H), 3.18 (m, 1H), 3.03-2.64 (m, 5H), 2.05 (bd, J = 13 Hz, 1H), 1.80-0.50 (m, 43H) [M + H]+ Ion mass = 923.5679, [M + Na]+ Ion mass = 959.5671 |
| 167 | 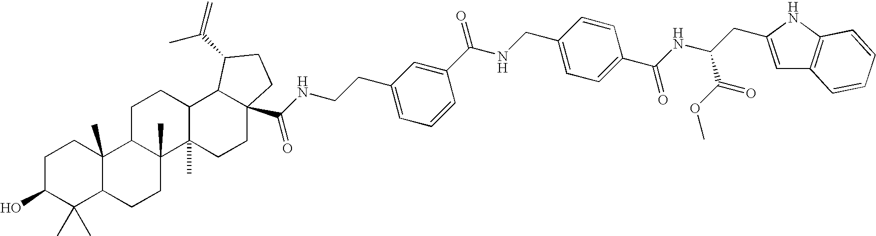 | 19 | 22 | [M + H]+ Ion mass = 937.5834, [M + Na]+ Ion mass = 959.5671 |
| 168 | 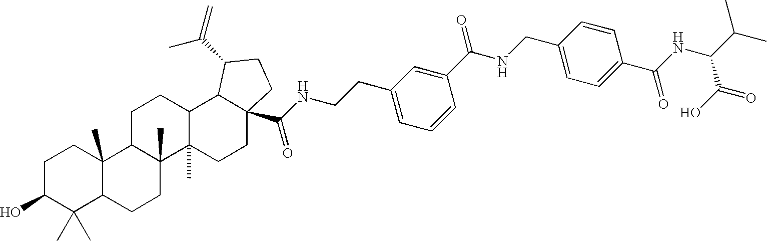 | 6 | 14 | 1H NMR (400 MHz, d6-DMSO) δ 12.63 (bs, 1H), 9.06 (bt, J = 6 Hz, 1H), 8.36 (d, J = 8 Hz, 1H), 7.85 (d, J = 8 Hz, 2H) 7.76 (s, 2H), 7.73 (m, 1H), 7.63 (bt, J = 6 Hz, 1H), 7.38 (m, 4H), 4.63 (s, 1H), 4.52 (m, 3H), 4.27 (t, J = 7.5 Hz, 1H), 3.25 (m, 1H), 2.95 (m, 2H), 2.77 (m, 2H), 2.18 (m, 1H), 2.05 (bd, J = 13 Hz, 1H), 1.80-0.50 (m, 49H) [M + H]+ Ion mass = 836.5561, [M + Na]+ Ion mass = 858.5384 |
| 169 | 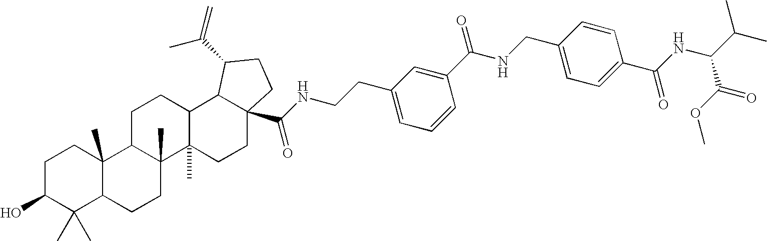 | 9 | 20 | [M + H]+ Ion mass = 850.5724, [M + Na]+ Ion mass = 872.5552 |
| 170 | 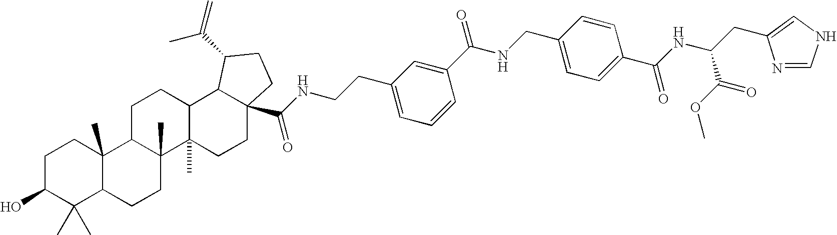 | 5 | 10 | [M + H]+ Ion mass = 888.5626, [M + Na]+ Ion mass = 910.5462 |
| 171 | 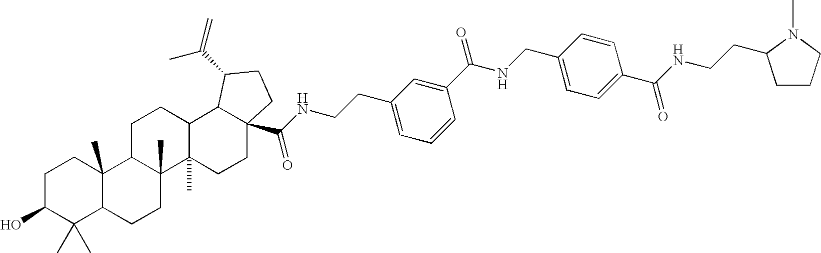 | 11 | 50 | 1H NMR (400 MHz, d6-DMSO) δ 9.08 (bt, J = 6 Hz, 1H), 8.55 (bs, 1H), 7.78 (m, 4H), 7.65 (bt, J = 6 Hz, 1H), 7.38 (m, 3H), 4.63 (s, 1H), 4.52 (m, 3H), 4.29 (bd, J = 5 Hz, 1H), 3.50-0.50 (m, 63H) [M + H]+ Ion mass = 847.6080, [M + Na]+ Ion mass = 869.5909 |
| 172 | 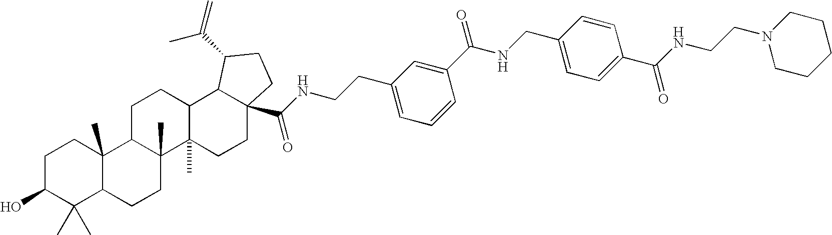 | 14 | 93 | 1H NMR (400 MHz, d6-DMSO) δ 9.07 (bt, J = 6 Hz, 1H), 7.80 (m, 4H), 7.64 (bt, J = 6 Hz, 1H), 7.38 (m, 4H), 4.63 (s, 1H), 4.52 (m, 3H), 4.28 (m, 1H), 3.49 (m, 1H), 3.25 (m, 2H), 2.96 (m, 2H), 2.76 (m, 2H), 2.05 (bd, J = 13 Hz, 1H), 1.80-0.50 (m, 55H) [M + H]+ Ion mass = 847.6079, [M + Na]+ Ion mass = 869.5907 |
| 173 | 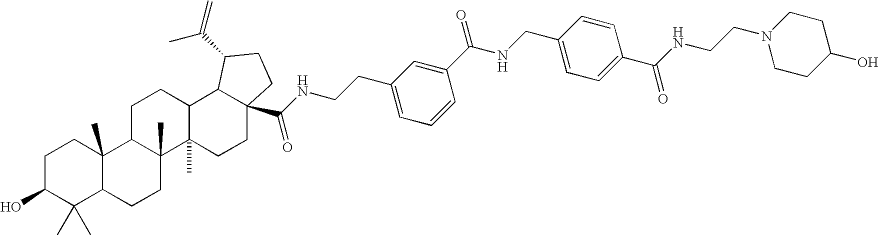 | 2 | 28 | 1H NMR (400 MHz, d6-DMSO) δ 9.06 (bt, J = 6 Hz, 1H), 8.44 (bs, 1H), 7.77 (m, 4H), 7.64 (bt, J = 6 Hz, 1H), 7.37 (m, 4H), 4.63 (s, 1H), 4.51 (m, 3H), 4.27 (d, J = 5 Hz, 1H), 3.60-0.50 (m, 62H) [M + H]+ Ion mass = 863.6022, [M + Na]+ Ion mass = 885.5835 |
| 174 | 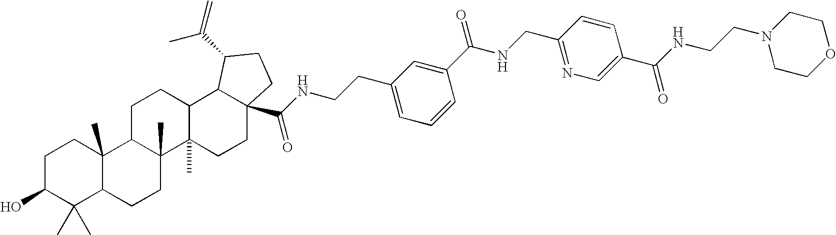 | 7 | 6 | 1H NMR (400 MHz, d6-DMSO) δ 9.15 (bt, J = 6 Hz, 1H), 8.93 (s, 1H), 8.60 (bt, J = 6 Hz, 1H), 8.13 (dd, J = 2, 8 Hz, 1H), 7.77 (m, 2H), 7.65 (bt, J = 6 Hz, 1H), 7.39 (m, 3H), 4.61 (m, 3H), 4.51 (s, 1H), 4.28 (d, J = 5 Hz, 1H), 3.56 (m, 4H), 3.39 (m, 3H), 3.26 (m, 1H), 3.02-2.68 (m, 4H), 2.43 (m, 7H), 2.05 (bd, J = 13 Hz, 1H), 1.80-0.50 (m, 40H) [M + H]+ Ion mass = 850.5826, [M + Na]+ Ion mass = 872.5657 |
| 175 | 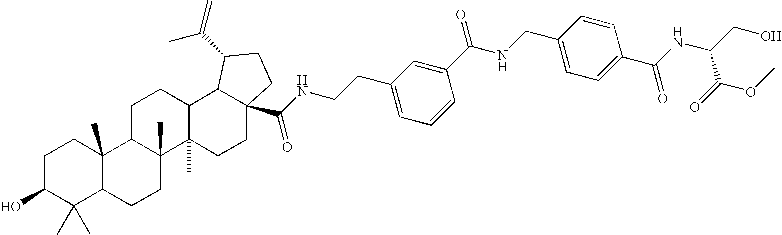 | 4 | 13 | [M + H]+ Ion mass = 838.5365, [M + Na]+ Ion mass = 860.5186 |
| 176 | 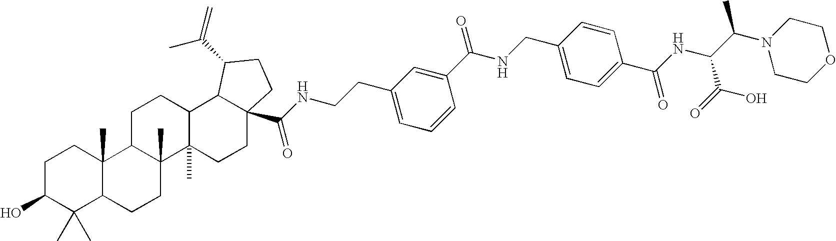 | 57 | 72 | [M + H]+ Ion mass = 907.5936 |
| 177 | 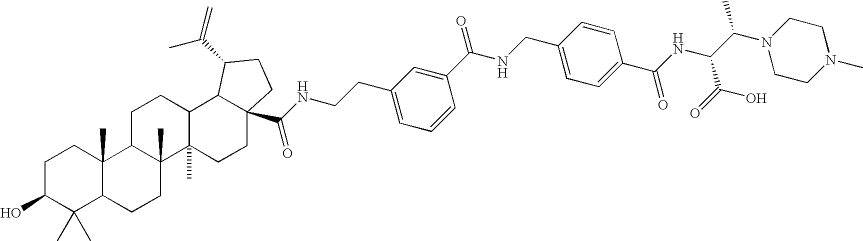 | 92 | 8 | 1H NMR (400 MHz, d6-DMSO) δ 12.76 (bs, 1H), 9.29 (bs, 1H), 9.11 (bt, J = 6 Hz, 1H), 8.34 (d, J = 7 Hz, 1H), 7.84 (d, J = 8 Hz, 2H), 7.75 (m, 2H), 7.67 (bt, J = 6 Hz, 1H), 7.40 (m, 4H), 4.64 (s, 1H), 4.53 (m, 3H), 4.35 (m, 1H), 3.45-2.55 (m, 17H), 2.06 (bd, J = 13 Hz, 1H), 1.80-0.50 (m, 47H) [M + H]+ Ion mass = 920.6277 |
| 178 | 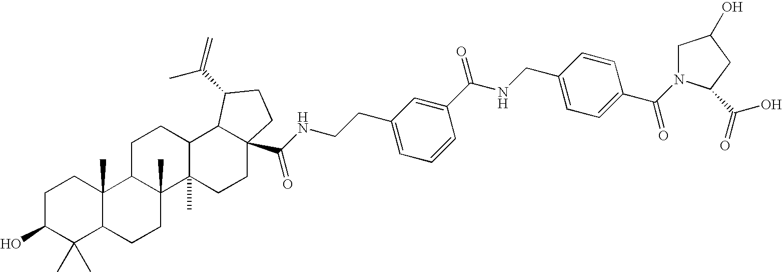 | 35 | 21 | 1H NMR (400 MHz, d6-DMSO) δ 12.54 (bs, 1H), 9.05 (bt, J = 6 Hz, 1H), 7.73 (m, 2H), 7.61 (bt, J = 6 Hz, 1H), 7.47 (d, J = 8 Hz, 3H), 7.35 (m, 5H), 5.04 (s, 1H), 4.60 (s, 1H), 4.47 (m, 4H), 4.24 (m, 2H), 3.69 (m, 1H), 3.25 (m, 2H), 2.94 (m, 2H), 2.74 (m, 2H), 2.16 (m, 1H), 2.02 (m, 1H), 1.91 (m, 1H), 1.80-0.50 (m, 43H) [M + H]+ Ion mass = 850.5365, [M + Na]+ Ion mass = 872.5193 |
| 179 | 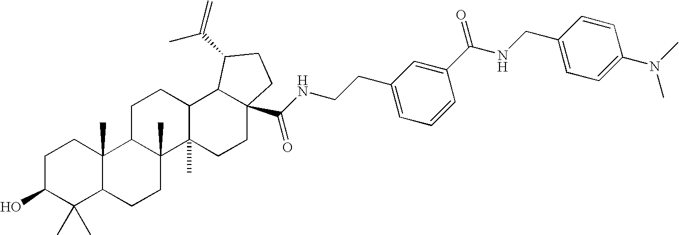 | 98 | 72 | 1H NMR (400 MHz, d6-DMSO) δ 8.83 (bt, J = 6 Hz, 1H), 7.72 (m, 2H), 7.63 (bt, J = 6 Hz, 1H), 7.34 (m, 2H), 7.14 (d, J = 9 Hz, 2H), 6.67 (d, J = 9 Hz, 2H), 4.63 (s, 1H), 4.52 (s, 1H), 4.35 (d, J = 5 Hz, 2H), 4.28 (d, J = 5 Hz, 1H), 3.24 (m, 1H), 2.96 (m, 2H), 2.04 (bd, J = 13 Hz, 1H), 1.80-0.50 (m, 42H) [M + H]+ Ion mass = 736.5403, [M + Na]+ Ion mass = 758.5234 |
| 180 | 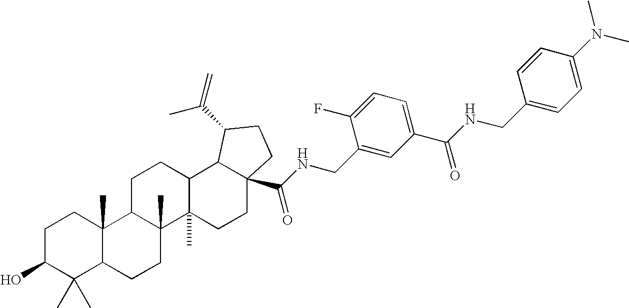 | N/A | 1000 | 1H NMR (400 MHz, d6-DMSO) δ 8.82 (bt, J = 6 Hz, 1H), 8.24 (bt, J = 6 Hz, 1H), 7.89 (d, J = 8 Hz, 2H), 7.77 (m, 1H), 7.22 (t, J = 9 Hz, 1H), 7.12 (d, J = 8 Hz, 2H), 6.66 (d, , J = 9 Hz, 2H), 4.63 (s, 1H), 4.52 (s, 1H), 4.31 (m, 4H), 4.19 (m, 1H), 2.97 (m, 2H), 2.84 (s, 6H), 2.21 (bd, J = 13 Hz, 1H), 1.80-0.50 (m, 41H) [M + H]+ Ion mass = 740.5161, [M + Na]+ Ion mass = 762.49801 |
| 181 | 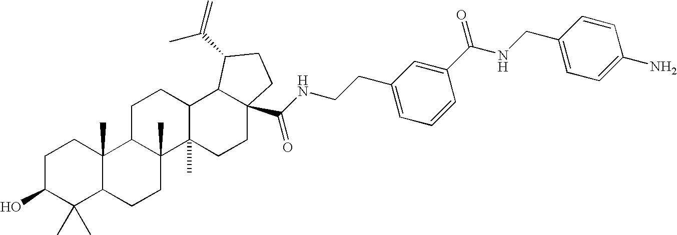 | 28 | 19 | 1H NMR (400 MHz, d6-DMSO) δ 8.79 (bt, J = 6 Hz, 1H), 7.72 (m, 2H), 7.64 (bt, J = 6 Hz, 1H), 7.34 (m, 2H), 6.97 (d, J = 9 Hz, 2H), 6.50 (d, J = 9 Hz, 2H), 4.96 (s, 2H), 4.64 (s, 1H), 4.52 (s, 1H), 4.29 (m, 3H), 3.34 (m, 1H), 3.24 (m, 1H), 2.96 (m, 2H), 2.75 (m, 2H), 2.05 (m, 2H), 1.80-0.50 (m, 40H) [M + H]+ Ion mass = 708.5159, [M + Na]+ Ion mass = 730.4987 |
| 182 | 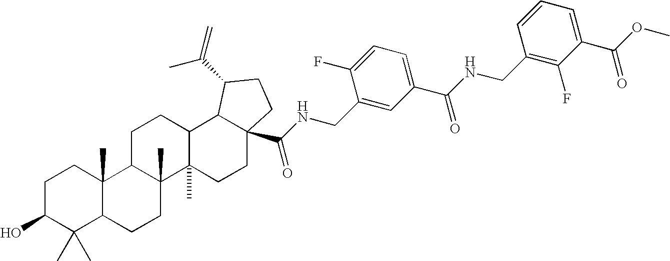 | N/A | 600 | [M + H]+ Ion mass = 773.4693, [M + Na]+ Ion mass = 795.4517 |
| 183 | 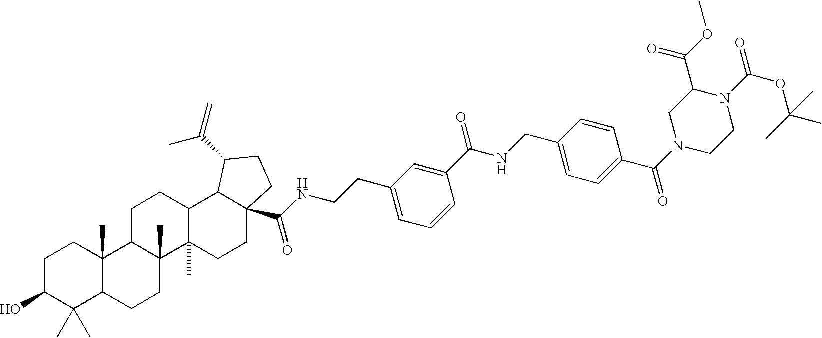 | 108 | 44 | [M + H]+ Ion mass = 963.6212, [M + Na]+ Ion mass = 985.6042 |
| 184 | 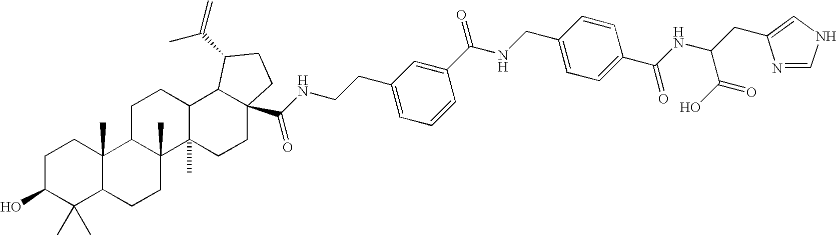 | 830 | 104 | 1H NMR (400 MHz, d6-DMSO) δ 12.97 (s, 1H), 9.11 (bt, J = 6 Hz, 1H), 8.98 (bs, 1H), 8.81 (d, J = 8 Hz, 1H), 7.78 (m, 4H), 7.66 (bt, J = 6 Hz, 1H), 7.38 (m, 5H), 4.76 (m, 1H), 4.64 (s, 1H), 4.52 (m, 3H), 3.42-3.12 (m, 5H), 2.97 (m, 2H), 2.76 (m, 2H), 2.06 (bd, J = 13 Hz, 1H), 1.80-0.50 (m, 42H) [M + H]+ Ion mass = 874.5443, [M + Na]+ Ion mass = 896.5254 |
| 185 | 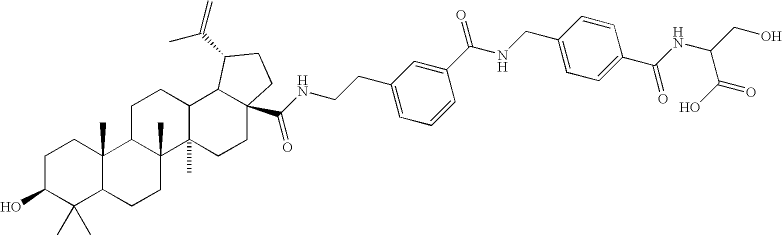 | 36 | 14 | 1H NMR (400 MHz, d6-DMSO) δ 9.08 (bt, J = 6 Hz, 1H), 8.35 (d, J = 8 Hz, 1H), 7.83 (m, 4H), 7.40 (m, 4H), 5.00 (bs, 1H), 4.50 (m, 3H), 4.30 (t, J = 7.5 Hz, 1H), 3.79 (m, 2H), 2.98 (m, 2H), 3.50-0.50 (m, 50H) [M + H]+ Ion mass = 824.5218, [M + Na]+ Ion mass = 846.5052 |
| 186 | 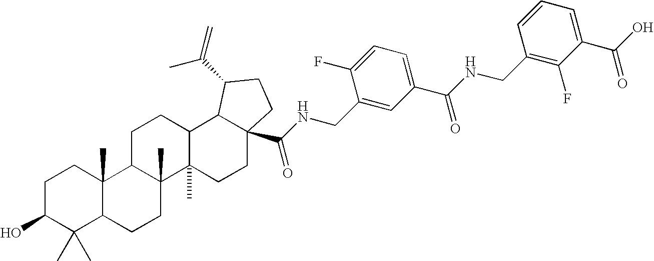 | 830 | 120 | 1H NMR (400 MHz, d6-DMSO) δ 9.01 (bt, J = 6 Hz, 1H), 8.23 (bt, J = 6 Hz, 1H), 7.90 (d, J = 8 Hz, 2H), 7.82 (m, 1H), 7.75 (t, J = 9 Hz, 1H), 7.54 (t, J = 7 Hz, 1H), 7.24 (m, 2H), 4.63 (s, 1H), 4.52 (m, 3H), 4.40-4.14 (m, 2H), 2.96 (m, 2H), 2.45 (m, 1H), 2.20 (bd, J = 13 Hz, 1H), 1,80-0.50 (m, 41H) [M + H]+ Ion mass = 759.4591 |
| 187 | 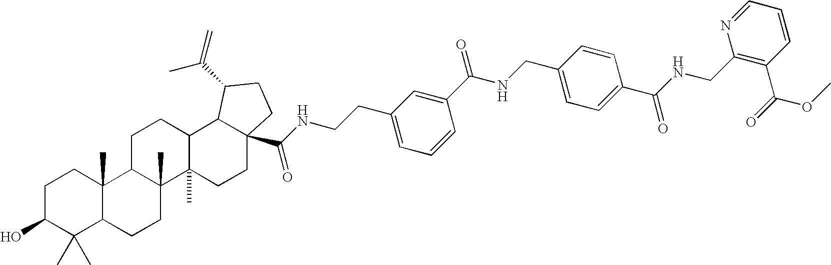 | 40 | 8 | 1H NMR (400 MHz, d6-DMSO) δ 9.06 (bt, J = 6 Hz, 1H), 8.81 (bt, J = 6 Hz, 1H), 8.70 (m, 1H), 8.22 (m, 1H), 7.84 (d, J = 8 Hz, 2H), 7.76 (m, 2H), 7.63 (bt, J = 6 Hz, 1H), 7.45 (m, 1H), 7.38 (m, 4H), 4.88 (d, J = 6 Hz, 2H), 4.63 (s, 1H), 4.52 (m, 2H), 4.27 (d, J = 5 Hz, 1H), 3.88 (s, 3H), 3.42-3.19 (m, 2H), 3.05-2.64 (m, 8H), 2.05 (bd, J = 13 Hz, 1H), 1.80-0.50 (m, 38H) [M + H]+ Ion mass = 885.5500, [M + Na]+ Ion mass = 907.5325 |
| 188 | 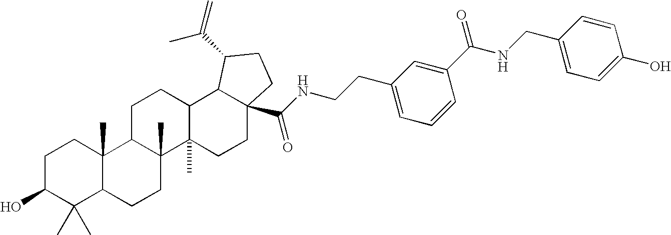 | 37 | 12 | 1H NMR (400 MHz, d6-DMSO) δ 9.27 (s, 1H), 8.87 (bt, J = 6 Hz, 1H), 7.72 (m, 2H), 7.63 (bt, J = 6 Hz, 1H), 7.34 (m, 2H), 7.11 (d, J = 9 Hz, 2H), 6.70 (d, J = 9 Hz, 2H), 4.63 (s, 1H), 4.52 (s, 1H), 4.35 (d, J = 6 Hz, 2H), 4.27 (d, J = 5 Hz, 1H), 3.25 (m, 1H), 2.95 (m, 2H), 2.75 (m, 2H), 2.05 (bd J = 13 Hz, 1H), 1.80-0.50 (m, 41H) [M + H]+ Ion mass = 709.4920, [M + Na]+ Ion mass = 731.4737 |
| 189 | 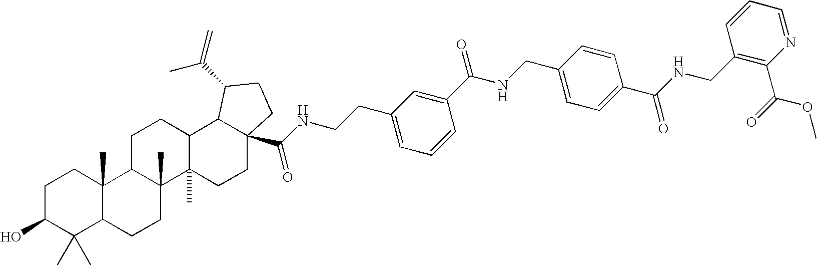 | 25 | 4 | 1H NMR (400 MHz, d6-DMSO) δ 9.06 (bt, J = 6 Hz, 1H), 9.00 (bt, J = 6 Hz, 1H), 8.54 (dd, J = 5, 2 Hz, 1H), 7.84 (m, 3H), 7.74 (m, 2H), 7.63 (bt, J = 6 Hz, 1H), 7.57 (m, 1H), 7.39 (m, 4H), 4.71 (d, J = 6 Hz, 2H), 4.63 (s, 1H), 4.52 (m, 3H), 4.27 (d, J = 5 Hz, 1H), 3.88 (s, 3H), 3.24 (m, 1H), 2.96 (m, 2H), 2.77 (m, 2H), 2.05 (bd, J = 13 Hz, 1H), 1.80-0.50 (m, 42H) [M + H]+ Ion mass = 885.5528, [M + Na]+ Ion mass = 907.5357 |
| 190 | 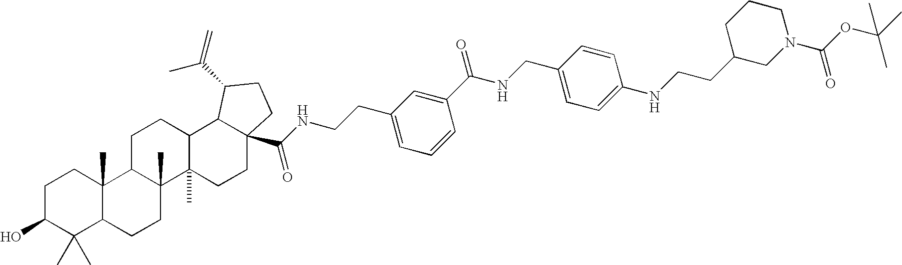 | N/A | 1000 | [M + H]+ Ion mass = 947.6957, [M + Na]+ Ion mass = 969.6776 |
| 191 | 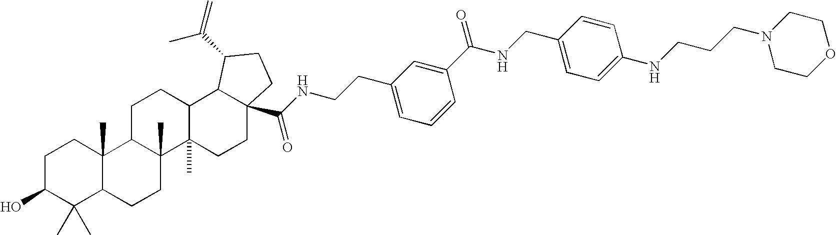 | 16 | 29 | 1H NMR (400 MHz, d6-DMSO) δ 8.84 (bt, J = 6 Hz, 1H), 7.68 (m, 3H), 7.36 (m, 3H), 7.07 (d, J = 8 Hz, 2H), 6.54 (d, J = 6 Hz, 2H), 4.63 (s, 1H), 4.52 (s, 1H), 4.31 (m, 2H), 4.00-0.50 (m, 63H) [M + H]+ Ion mass = 835.6121 |
| 192 | 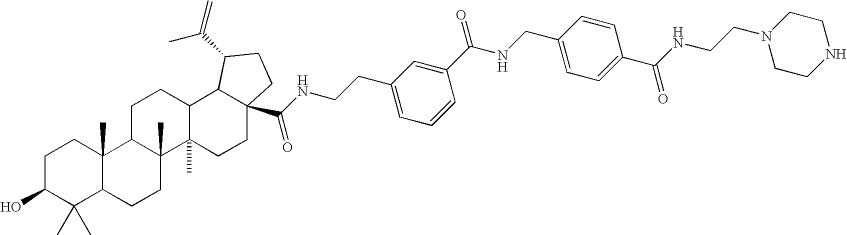 | 7 | 7 | 1H NMR (400 MHz, d6-DMSO) δ 9.07 (bt, J = 6 Hz, 1H), 8.65 (bs, 1H), 8.47 (bs, 1H), 7.76 (m, 4H), 7.64 (s, 1H), 4.52 (m, 3H), 3.31-2.65 (m, 12H), 2.06 (bd, J = 13 Hz, 1H), 1.80-0.50 (m, 48H) [M + H]+ Ion mass = 848.5899 |
| 193 | 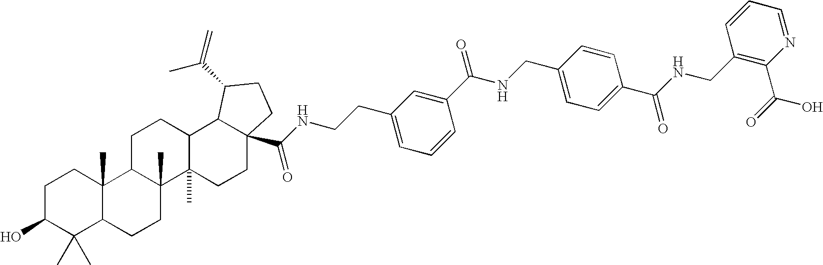 | 2 | 9 | 1H NMR (400 MHz, d6-DMSO) δ 9.07 (bt, J = 6 Hz, 1H), 9.01 (bt, J = 6 Hz, 1H), 8.54 (m, 1H), 7.87 (d, J = 8 Hz, 2H), 7.81 (d, J = 8 Hz, 1H), 7.75 (m, 2H), 7.63 (bt, J = 6 Hz, 1H), 7.42 (d, J = 8 Hz, 2H), 7.37 (m, 2H), 4.76 (d, J = 6 Hz, 2H), 4.63 (s, 1H), 4.53 (m, 3H), 3.37 (m, 1H), 3.25 (, 1H), 2.96 (m, 2H), 2.77 (m, 2H), 2.05 (bd, J = 13 Hz, 1H), 1.80-0.50 (m, 42H) [M + H]+ Ion mass = 871.5222 |
| 194 | 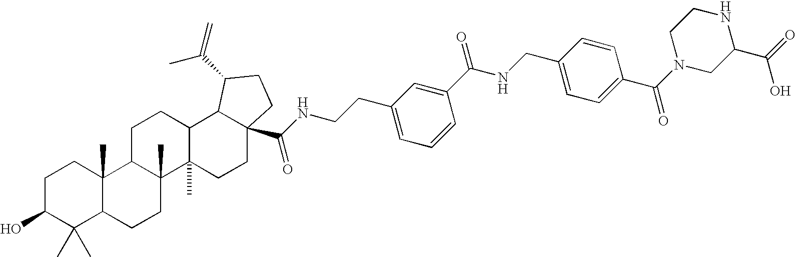 | 16 | 18 | 1H NMR (400 MHz, d6-DMSO) δ 9.12 (bt, J = 6 Hz, 1H), 7.77 (m, 2H), 7.67 (bt, J = 6 Hz, 1H), 7.40 (m, 6H), 4.63 (s, 1H), 4.52 (m, 3H), 4.28 (m, 1H), 3.89-0.45 (m, 56H) [M + H]+ Ion mass = 849.5501, [M + Na]+ Ion mass = 871.5336 |
| 195 | 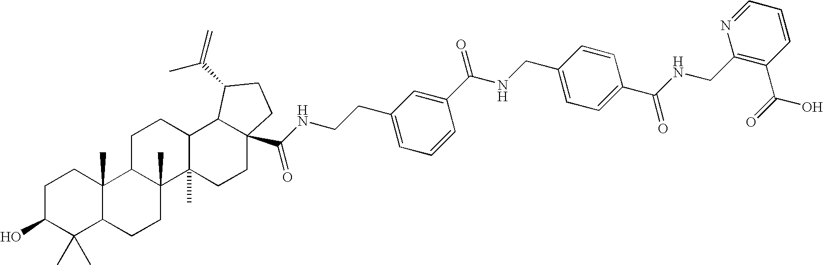 | 5 | 6 | 1H NMR (400 MHz, d6-DMSO) δ 9.06 (bt, J = 6 Hz, 1H), 8.79 (bt, J = 6 Hz, 1H), 8.68 (m, 1H), 8.24 (d, J = 8 Hz, 1H), 7.86 (d, J = 8 Hz, 2H), 7.76 (m, 2H), 7.63 (bt, J = 6 Hz, 1H), 7.40 (m, 5H), 4.92 (m, 2H), 4.63 (s, 1H), 4.53 (m, 3H), 3.31 (m, 2H), 2.95 (m, 2H), 2.76 (m, 2H), 2.05 (bd, J = 13 Hz, 1H), 1.80-0.50 (m, 42H) [M + H]+ Ion mass = 871.5207 |
| 196 | 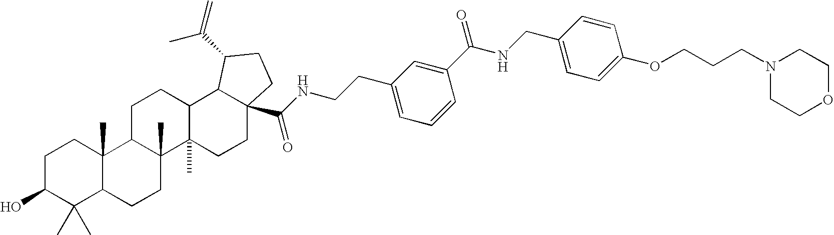 | 12.5 | 13 | 1H NMR (400 MHz, d6-DMSO) δ 8.91 (bt, J = 6 Hz, 1H), 7.72 (m, 2H), 7.62 (bt, J = 6 Hz, 1H), 7.35 (m, 2H), 7.22 (d, J = 9 Hz, 2H), 6.86 (d, J = 9 Hz, 2H), 4.63 (s, 1H), 4.52 (s, 1H), 4.39 (d, J = 6 Hz, 2H), 4.27 (d, J = 5 Hz, 1H), 3.97 (t, J = 6 Hz, 2H) 3.56 (m, 5H), 3.43-0.50 (m, 43H) [M + H]+ Ion mass = 836.5935, [M + Na]+ Ion mass = 858.5753 |
| 197 | 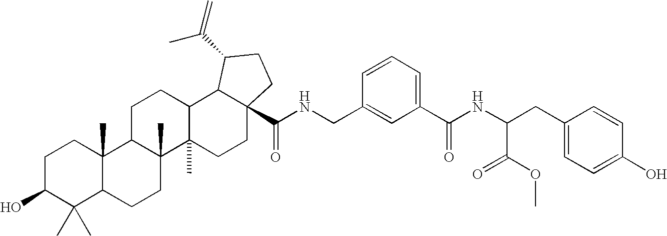 | N/A | 5750 | 1H NMR (400 MHz, d6-DMSO) δ 9.23 (s, 1H), 8.74 (d, J = 8 Hz, 1H), 8.20 (t, J = 4 Hz, 1H), 7.67-7.53 (2H, m), 7.37 (bs, 2H), 7.06 (d, J = 8.4 Hz, 2H), 6.64 (d, J = 8 Hz, 2H), 4.64 (bs, 1H), 4.52 (bs, 2H), 4.35-4.19 (m, 3H), 3.61 (s, 3H), 3.1-0.64 (m, 48H). MS (m/z) observed: 765.9 (M + 1). |
| 198 | 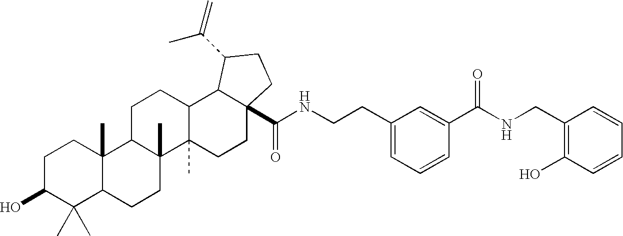 | 468 | 57.6 | 1H NMR (400 MHz, d6-DMSO) δ 9.62 (s, 1H), 8.91 (t, J = 6.4 Hz, 1H), 7.95 (s, 1H), 7.78 (s, 1H), 7.74 (m, 1H), 7.62 (t, J = 6.2 Hz, 1H), 7.38 (m, 2H), 7.09 (m, 2H), 6.81 (d, J = 8.8 Hz, 1H), 6.74 (t, J = 6.2 Hz, 1H), 4.63 (s, 1H), 4.52 (s, 1H), 4.41 (d, J = 5.8 Hz, 1H), 4.27 (d, J = 5.1 Hz, 1H), 3.38 (m, 1H), 3.25 (m, 1H), 2.97 (m, 2H), 2.04 (m, 1H), 1.70-0.58 (m, 40H); Mass Spec (m/z): 709.5 (M + 1). |
| 199 | 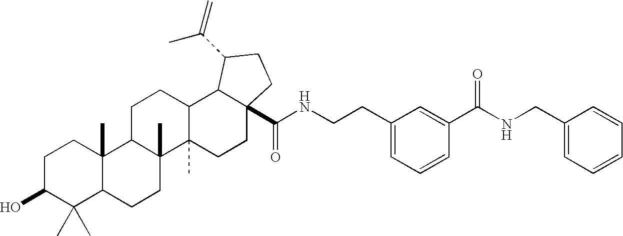 | 297 | 53.2 | 1H NMR (400 MHz, d6-DMSO) δ 8.99 (bt, 1H), 7.95 (s, 1H), 7.75 (m, 2H), 7.62 (s, 1H), 7.40-7.20 (m, 6H), 4.63 (s, 1H), 4.52 (s, 1H), 4.48 (d, J = 4.5 Hz, 1H), 4.27 (d, J = 3.5 Hz, 1H), 3.40-3.25 (m, 2H), 2.96 (m, 2H), 2.89 (m, 2H), 2.73 (m, 4H), 2.05 (m, 1H), 1.81-0.60 (m, 38H); Mass Spec (m/z): 693.5 (M + 1). |
| 200 | 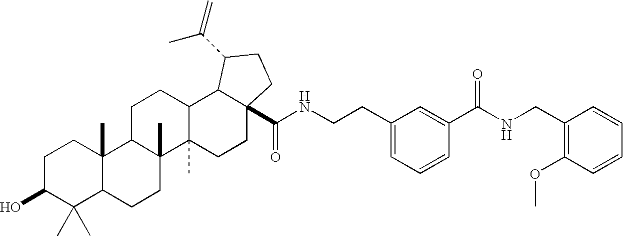 | 759 | 76.3 | 1H NMR (400 MHz, d6-DMSO) δ 8.80 (t, J = 6.1 Hz, 1H), 7.77 (s, 1H), 7.75 (m, 1H), 7.62 (t, J = 6.1 Hz, 1H), 7.37 (m, 2H), 7.23 (t, J = 7.6 Hz, 1H), 7.15 (d, J = 6.8 Hz, 1H), 6.99 (d, J = 8.0 Hz, 1H), 6.89 (t, J = 7.2 Hz, 1H), 4.63 (s, 1H), 4.52 (s, 1H), 4.44 (d, J = 6.2 Hz, 1H), 4.27 (d, J = 5.2 Hz, 1H), 3.83 (s, 3H). 3.38 (m, 1H), 3.26 (m, 1H), 2.96 (m, 2H), 2.77 (m, 2H), 2.04 (m, 1H), 1.71 (m, 1H), 1.60-0.58 (m, 41H); Mass Spec (m/z): 723.5 (M + 1). |
| 201 | 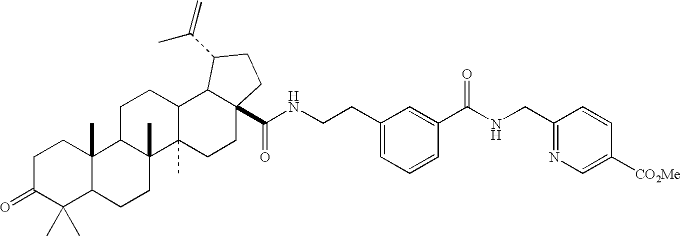 | 15.5 | 28.8 | 1H NMR (400 MHz, CDCl₃) δ 9.15 (d, J = 2.1 Hz, 1H), 8.34 (dd, J = 2.1, 7.9 Hz, 1H), 7.81 (t, J = 4.6 Hz, 1H), 7.75 (s, 2H), 7.72 (m, 1H), 7.50 (d, J = 9.2 Hz, 1H), 7.36 (m, 2H), 5.73 (t, J = 5.9 Hz, 1H), 4.83 (d, J = 5.1 Hz, 2H), 4.69 (s, 1H), 4.55 (s, 1H), 3.95 (s, 3H), 3.53 (o, J = 6.7 Hz, 2H), 3.05 (td, J = 10.7, 4.1 Hz, 1H), 2.87 (m, 2H), 2.44 (m, 4H), 1.83 (m, 4H), 1.75-0.84 (m, 33H); Mass Spec (m/z): 750.5 (M + 1). |
| 202 | 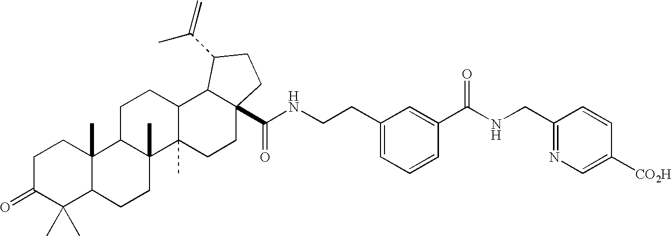 | 20.5 | 22.2 | 1H NMR (400 MHz, d6-DMSO) δ 9.17 (t, J = 5.9 Hz, 1H), 9.01 (d, J = 2.2 Hz, 1H), 8.24 (dd, J = 2.4 Hz, 8.2 Hz, 1H), 7.80 (s, 1H), 7.77 (t, J = 4.30 Hz, 1H), 7.65 (t, J = 4.30 Hz, 1H), 7.44 (d, J = 9.1 Hz, 1H), 7.39 (m, 2H), 4.63 (m, 3H), 4.52 (s, 1H), 3.32 (m, 2H), 2.97 (m, 1H), 2.78 (m, 2H), 2.57 (m, 1H), 2.38 (m, 3H), 2.05 (m, 1H), 1.82-0.76 (m, 38H); Mass Spec (m/z): 736.47 (M + 1). |
| 203 | 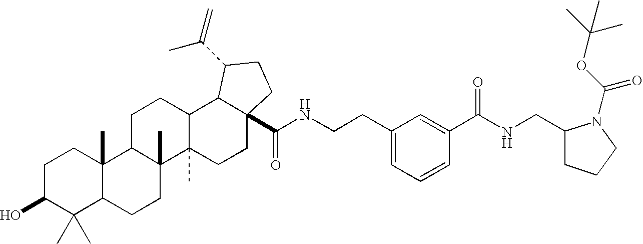 | N/A | 856 | 1H NMR (400 MHz, d6-DMSO) δ 8.45 (bs, 1H), NH), 7.70-7.60 (m, 1H), NH, 2H, Ar), 7.61 (bs, 1H), Ar), 4.62 (s, 1H), CH═), 4.52 (s, 1H), CH═), 2.89-0.64 (m, 68H, CH); Mass Spec (m/z): 786.6 (M + 1). |
| 204 | 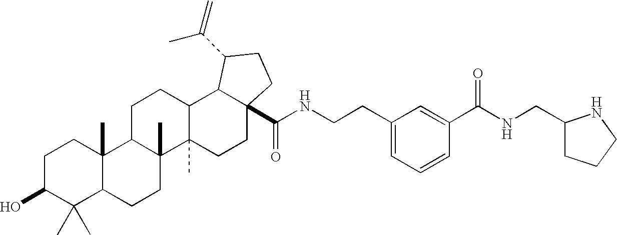 | N/A | 1940 | 1H NMR (400 MHz, d6-DMSO) δ 9.09 (bs, 1H, NH), 8.80 (bs, 1H, NH), 8.49 (bs, 1H), NH), 7.81 (s, 1H, Ar), 7.75 (d, J = 7.43 Hz, 1H, Ar), 7.50-7.42 (m, 3H, Ar), 3.90-0.64 (m, 59H, CH); Mass Spec (m/z): 686.53 (M + 1). |
| 205 | 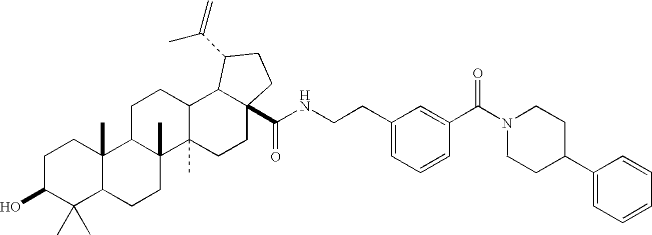 | N/A | 1000 | 1H NMR (400 MHz, d6-DMSO) δ 7.63 (t, J = 5.08 Hz, 1H, NH), 7.36-7.18 (m, 9H, Ar), 4.62 (s, 1H, CH═), 4.51 (s, 1H, CH═), 4.28 (d, J = 5.47 Hz, 1H, CH), 3.0-0.63 (m, 57H, CH); Mass Spec (m/z): 747.53 (M + 1). |
| 206 | 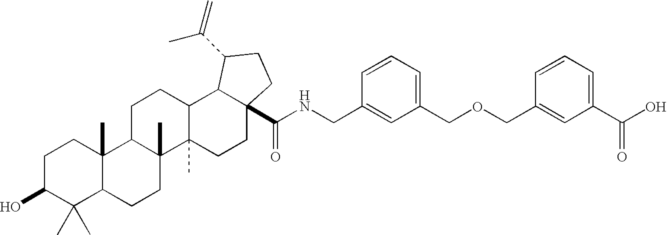 | N/A | 444 | 1H NMR (400 MHz, d6-DMSO) δ 12.91 (s, 1H), CO₂H), 8.19 (t, J = 11.2 Hz J = 5.6 Hz, 1H, NH), 7.93 (dd, J = 6.4 Hz, J = 2 Hz, J = 2 Hz, 2H, Ar), 7.47 (d, J = 8.8 Hz, 2H, Ar), 7.31-7.12 (m, 4H, Ar), 4.63 (s, 1H, CH═), 4.59 (s, 2H, OCH2), 4.52 (s, 1H, CH═, 2H, OCH2). 4.35 (dd, J = 15.2 Hz, J = 6 Hz, J = 5.6 Hz, 1H, NCH) 4.27 (bs, 1H, CH), 4.15 (dd, J = 14.8 Hz, J = 6 Hz, J = 5.6 Hz, 1H, NCH); 3.0-0.61 (m, 47H, CH); Mass Spec (m/z): 710.50 (M + 1). |
| 207 | 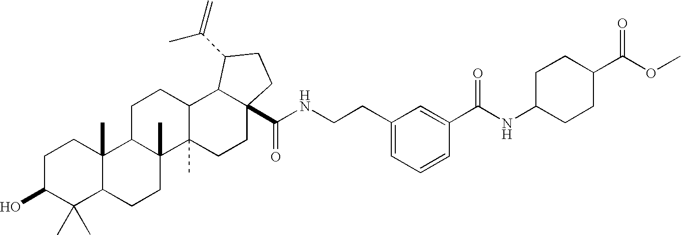 | N/A | 193 | 1H NMR (400 MHz, d6-DMSO) δ 8.12 (d, J = 7.6 Hz, 1H Ar), 7.69-7.61 (m, 1H, NH, 2H, Ar), 7.33 (d, J = 5.2 Hz, 2H, Ar), 4.63 (s, 1H, CH═), 4.52 (s, 1H, CH═), 3.63 (s, 3H, OCH3), 3.0-0.64 (m, 60H, CH); Mass Spec (m/z): 743.53 (M + 1). |
| 208 | 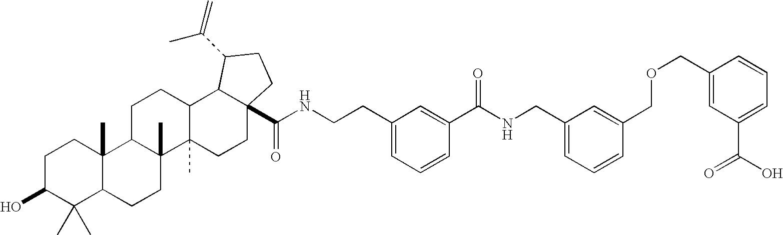 | 42.5 | 28.9 | 1H NMR (400 MHz, d6-DMSO) δ 7.90-7.25 (m, 2H, NH, 12H, Ar), 4.61-4.49 (m, 4H, OCH2, 2H, NCH2, 2H, CH2), 3.0-0.64 (m, 50H, CH); Mass Spec (m/z): 857.55 (M + 1). |
| 209 | 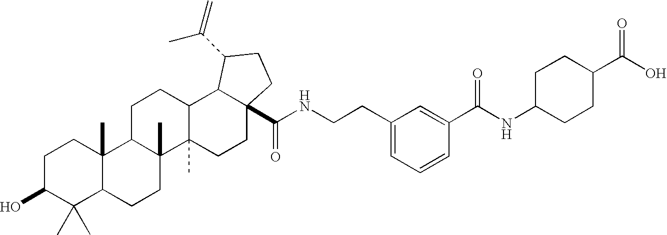 | N/A | 356 | 1H NMR (400 MHz, d6-DMSO) δ 12.16 (s, 1H, CO₂H), 8.13 (d, J = 8.0 Hz, 1H, NH), 7.70-7.66 (m, 2H, Ar), 7.62 (t, J = 10.8 Hz, J = 5.6 Hz, J = 6 Hz, 1H, NH), 7.33 (d, J = 5.2 Hz, 2H, Ar), 4.63 (s, 1H, CH═), 4.52 (s, 1H, CH═), 3.84 (s, 1H, CH), 3.94-0.61 (m, 58H, CH); Mass Spec (m/z): 729.52 (M + 1). |
| 210 | 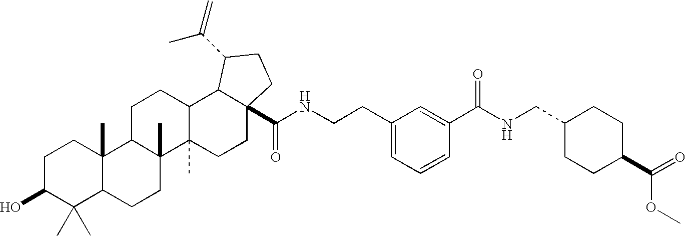 | 33 | 30.8 | 1H NMR (400 MHz, d6-DMSO) δ 8.38 (t, J = 11.2 Hz, J = 5.2 Hz, J = 6 Hz, 1H, NH), 7.70-7.65 (m, 2H, Ar), 7.61 (t, J = 11.2 Hz, J = 5.6 Hz, 1H, NH), 7.34 (d, J = 5.2 Hz, 2H, Ar), 4.63 (s, 1H, CH═), 4.52 (s, 1H, CH═), 3.59 (s, 3H, OCH3), 3.37-0.64 (m, 61H, CH); Mass Spec (m/z): 757.60 (M + 1). |
| 211 | 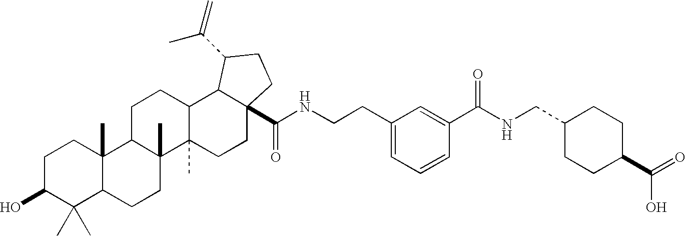 | 19 | 11.2 | 1H NMR (400 MHz, d6-DMSO) δ 8.38 (t, J = 11.2 Hz, J = 5.2 Hz, J = 6 Hz, 1H, NH), 7.70-7.66 (m, 2H, Ar), 7.61 (t, J = 11.2 Hz, J = 5.6 Hz, 1H, NH), 7.34 (d, J = 5.2 Hz, 2H, Ar), 4.63 (s, 1H, CH═), 4.52 (s, 1H, CH═), 3.37-0.64 (m, 61H, CH); Mass Spec (m/z): 743.55 (M + 1). |
| 212 | 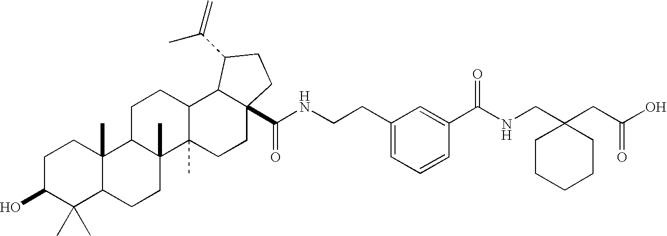 | 96.5 | 85 | 1H NMR (400 MHz, d6-DMSO) δ 8.37 (bs, 1H, NH), 7.71-7.64 (m, 1H, 2H, Ar), 7.35 (d, J = 5.08 Hz, 2H Ar), 4.63 (s, 1H, CH═), 4.62 (s, 1H, CH═), 3.38-0.64 (m, 64H, CH); Mass Spec (m/z): 757.55 (M + 1). |
| 213 | 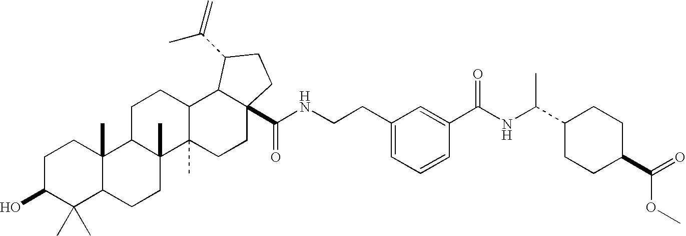 | 22 | 38.9 | 1H NMR (400 MHz, d6-DMSO) δ 8.06 (d, J = 8.4 Hz, 1H, Ar) 7.69-7.67 (m, 2H, Ar), 7.61 (t, J = 11.2 Hz, J = 5.6, 1H, NH), 7.34 (d, J = 4.8 Hz, 2H, Ar), 4.63 (s, 1H, CH═), 4.51 (s, 1H, CH═), 2.97-0.64 (m, 66H, CH); Mass Spec (m/z): 771.57 (M + 1). |
| 214 | 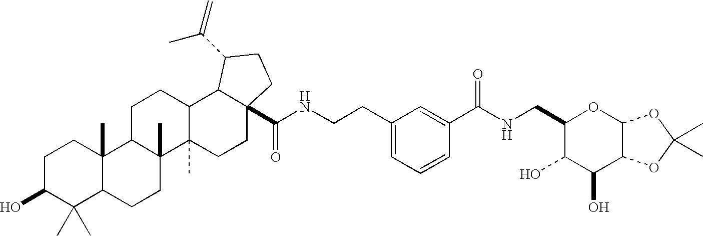 | 214 | 200 | 1H NMR (400 MHz, d6-DMSO) δ 8.33 (t, J = 11.2 Hz, J = 5.6 Hz, 1H, NH), 7.71-7.65 (m, 1H, NH, 2H, Ar), 7.37 (t, 2H, Ar), 5.83 (d, J = 3.6 Hz, 1H, CH), 5.22 (d, J = 4.8 Hz, 1H, CH), 5.02 (d, J = 5.6 Hz, 1H, CH), 4.65 (s, 1H, CH═), 4.53 (s, 1H, CH═), 4.40 (d, J = 3.6 Hz, 3.6 Hz, 1H, CH), 4.27 (d, J = 5.2 Hz, 1H, CH), 4.08 (dd, J = 4.8 Hz, J = 2.4 Hz, 1H, CH), 3.91 (m, 1H, CH), 3.81 (d, J = 2.8 Hz, 1H, CH), 3.79 (d, J = 2.4 Hz, 1H, CH), 3.36-0.64 (m, 57H, CH); Mass Spec (m/z): 805.54 (M + 1). |
| 215 | 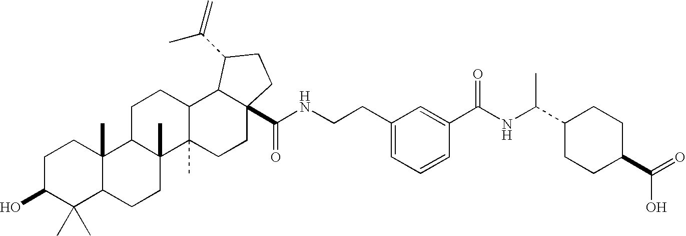 | 11.5 | 22 | 1H NMR (400 MHz, d6-DMSO) δ 8.05 (d, J = 8.8 Hz, 1H, Ar) 7.69-7.67 (m, 2H, Ar), 7.61 (t, J = 11.2 Hz, J = 5.6 Hz, 1H, NH), 7.34 (d, J = 4.8 Hz, 2H, Ar), 4.63 (s, 1H, CH═), 4.52 (s, 1H, CH═), 3.84 (m, 1H, NCH), 3.37-3.25 (m, 2H, CH2), 2.98-0.64 (m, 61H, CH); Mass Spec (m/z): 757.56 (M + 1). |
| 216 | 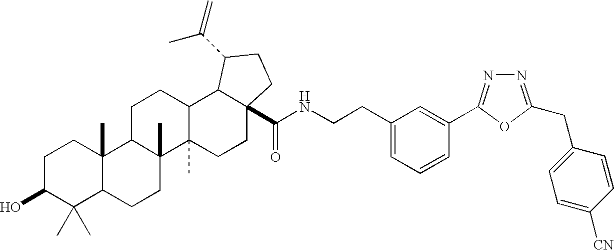 | 2700 | 66.5 | 1H NMR (400 MHz, d6-DMSO) δ 7.88-7.47 (m, 1H, NH, 8H, Ar), 4.60 (s, 1H, CH═), 4.50 (s, 1H, CH═), 4.46 (m, 2H, CH2), 3.0-0.60 (m, 49H, CH); Mass Spec (m/z): 743.50 (M + 1). |
| 217 | 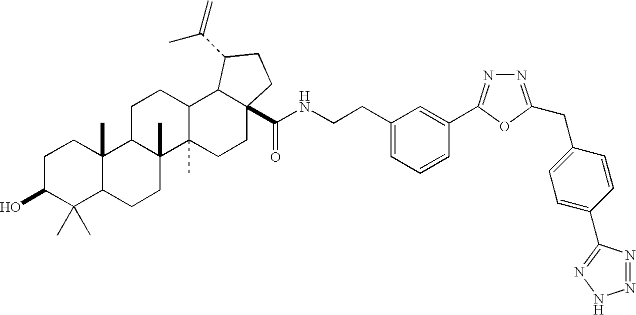 | 49 | 87 | Mass Spec (m/z): 786.50 (M + 1). |
| 218 | 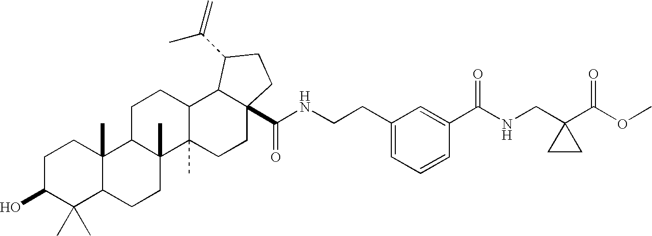 | 12.5 | 19 | 1H NMR (400 MHz, d6-DMSO) δ 8.58 (t, J = 6.0 Hz, 1H, NH) 7.70-7.61 (m, 1H, NH, 2H, Ar), 7.36 (d, J = 4.8 Hz, 2H), 4.64 (s, 1H, CH═), 4.52 (s, 1H, CH═), 4.04 (m, 2H, CH2), 2.98-0.64 (m, 58H, CH); Mass Spec (m/z): 729.53 (M + 1). |
| 219 | 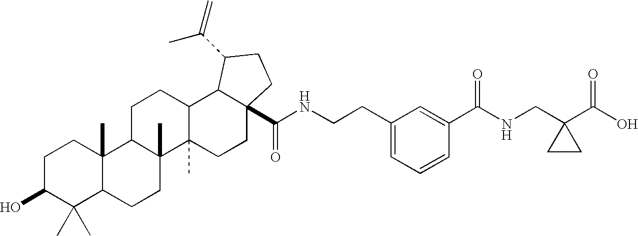 | N/A | 396 | Mass Spec (m/z): 701.50 (M + 1). |
| 220 | 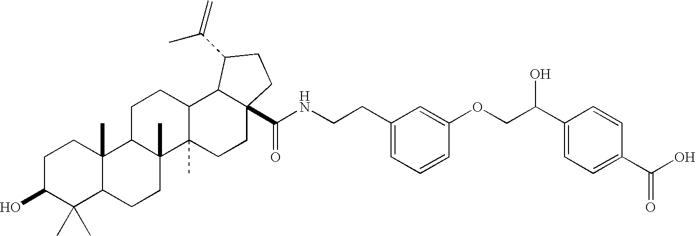 | N/A | 259 | 1H NMR (400 MHz, d6-DMSO) δ 8.03 (d, J = 8 Hz, 2H), 7.58 (d, J = 8 Hz, 2H), 7.50 (bt, J = 6 Hz, 1H), 7.17 (t, J = 8 Hz, 1H), 6.79 (m, 3H), 5.09 (m, 1H), 4.66(s, 1H), 4.55 (s, 1H), 4.07 (d, J = 6 Hz, 2H), 3.51 (m, 1H), 3.11 (m, 1H), 3.00 (m, 1H), 2.74 (m, 2H), 2.49 (m, 1H), 1.98 (bd, J = 13 Hz, 1H), 1.90-0.60 (m, 40H) [M + H]+ Ion mass = 740.48847, [M + Na]+ Ion mass = 762.47041 |
| 221 | 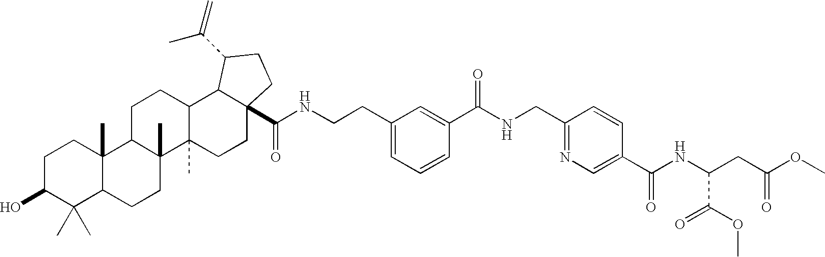 | 35 | 9.0 | 1H NMR (400 MHz, d6-DMSO) δ 9.06 (bt, J = 6 Hz, 1H), 8.88 (d, J = 8 Hz, 1H), 7.80 (d, J = 8 Hz, 2H), 7.76 (s, 1H), 7.73 (m, 1H), 7.63 (bt, J = 6 Hz, 1H), 7.41 (d, J = 8 Hz, 2H), 7.37 (m, 2H), 4.83 (m, 1H), 4.63 (s, 1H), 4.52 (m, 3H), 4.27 (d, J = 6 Hz, 1H), 3.64 (s, 3H), 3.61 (s, 3H), 3.41-3.18 (m, 2H), 3.02-2.65 (m, 6H), 2.05 (bd, J = 13 Hz, 1H), 1.80-0.50 (m, 42H) [M + H]+ Ion mass = 881.55487, [M + Na]+ Ion mass = 903.53681 |
| 222 | 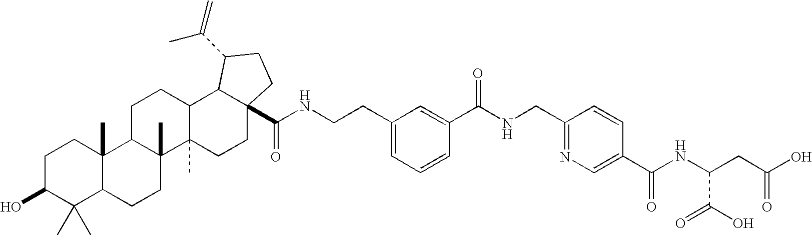 | 727 | 65.7 | [M + H]+ Ion mass = 853.52357, [M + Na]+ Ion mass = 875.50551 |
| 223 | 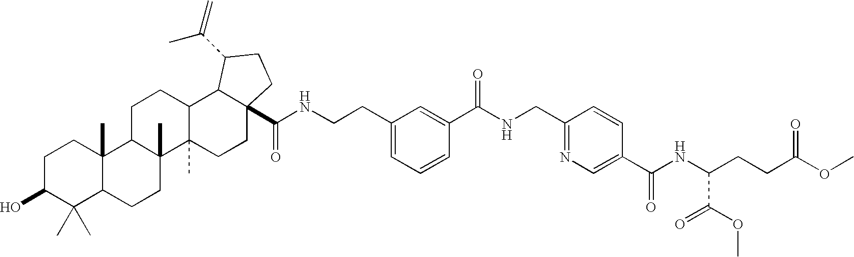 | 21 | 21.7 | 1H NMR (400 MHz, d4-MeOH) δ 9.15 (bt, J = 6 Hz, 1H), 8.97 (d, J = 2 Hz, 1H), 8.94 (d, J = 8 Hz, 1H), 8.17 (dd, J = 8, 2 Hz, 2H), 7.79 (s, 1H), 7.75 (m, 1H), 7.64 (bt, J = 6 Hz, 1H), 7.44-7.36 (m, 3H), 4.62 (m, 3H), 4.49 (m, 2H), 4.27 (d, J = 6 Hz, 1H), 3.65 (s, 3H), 3.58 (s, 3H), 3.41-3.18 (m, 2H), 2.96 (m, 2H), 2.77 (m, 2H), 2.59-0.50 (m, 56H) [M + H]+ Ion mass = 896.56577, [M + Na]+ Ion mass = 918.54771 |
| 224 | 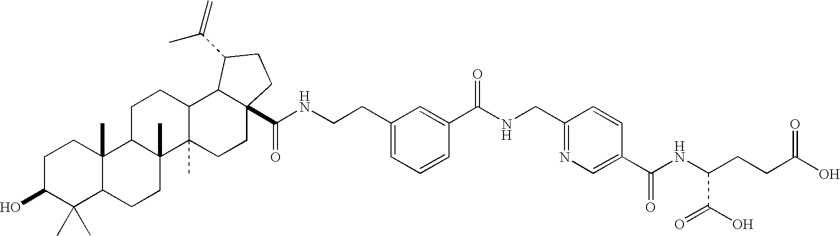 | 257.5 | 35.5 | 1H NMR (400 MHz, d6-DMSO) δ 12.74 (bs, 1H), 12.18 (bs, 1H), 9.15 (bt, J = 6 Hz, 1H), 8.97 (d, J = 2 Hz, 1H), 8.80 (d, J = 8 Hz, 1H), 8.18 (dd, J = 8, 2 Hz, 1H), 7.79 (s, 1H), 7.76 (m, 1H), 7.64 (bt, J = 6 Hz, 1H), 7.39 (m, 3H), 4.62 (m, 3H), 4.52 (s, 1H), 4.42 (m, 1H), 3.47-3.18 (m, 2H), 2.96 (m, 2H), 2.77 (m, 2H), 2.55-0.50 (m, 46H) [M + H]+ Ion mass = 867.5272 |
| 225 | 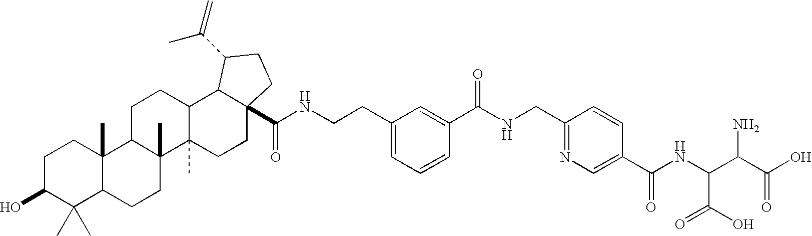 | 53 | 38.5 | 1H NMR (400 MHz, d4-MeOH) δ 9.06 (bt, J = 6 Hz, 1H), 7.89 (d, J = 8 Hz, 2H), 7.77 (s, 1H), 7.72 (m, 1H), 7.63 (bt, J = 6 Hz, 1H), 7.49 (d, J = 8 Hz, 2H), 7.46-7.36 (m, 2H), 5.07 (s, 1), 4.65 (m, 3H), 4.56 (s, 1H), 4.45 (s, 1H), 3.80-0.50 (m, 46H) [M + H]+ Ion mass = 867.52664 |
| 226 | 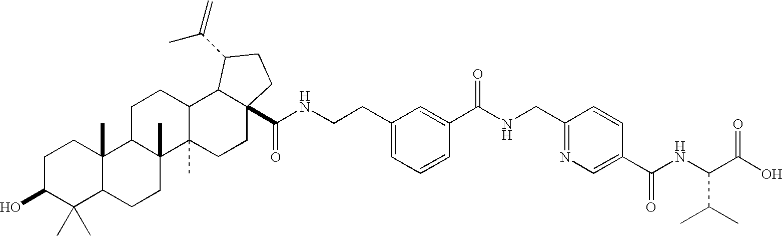 | 102 | 11.8 | 1H NMR (400 MHz, d6-DMSO) δ 9.16 (bt, J = 6 Hz, 1H), 8.96 (bs, 1H), 8.17 (m, 1H), 7.80 (s, 1H), 7.77 (m, 2H), 7.65 (bt, J = 6 Hz, 1H), 7.40 (m, 3H), 4.62 (m, 3H), 4.52 (s, 1H), 4.29 (m, 1H), 2.96 (m ,2H), 2.77 (m, 2H), 2.59-0.56 (m, 54H) [M + H]+ Ion mass = 837.5 |
| 227 | 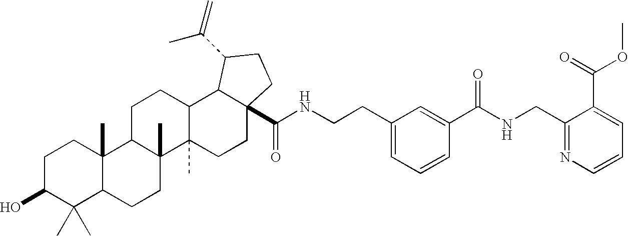 | 761 | 67.5 | 1H NMR (400 MHz, d6-DMSO) δ 8.79 (bt, J = 6 Hz, 1H), 8.72 (d, J = 4 Hz, 1H), 8.24 (d, J = 8 Hz, 1H), 7.74 (s, 1H), 7.71 (m, 1H), 7.64 (bt, J = 6 Hz, 1H), 7.46 (dd, J = 8 4 Hz, 1H), 7.37 (m, 2H), 4.89 (d, J = 6 Hz, 2H), 4.63 (s, 1H), 4.52 (s, 1H), 4.27 (d, J = 6 Hz, 1H), 3.88 (s, 3H), 3.43-3.18 (m, 2H), 2.96 (m, 2H), 2.77 (m, 2H) 2.04 (bd, J = 13 Hz, 1H), 1.80-0.50 (m, 40H) [M + H]+ Ion mass = 752.49970, [M + Na]+ Ion mass = 774.48164 |
| 228 | 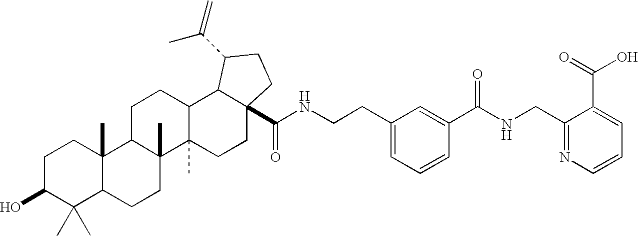 | N/A | 135 | 1H NMR (400 MHz, d6-DMSO) δ 13.48 (bs, 1H), 8.77 (bt, J = 6 Hz, 1H), 8.70 (d, J = 4 Hz, 1H), 8.25 (d, J = 8 Hz, 1H), 7.75 (s, 1H), 7.72 (m, 1H), 7.64 (bt, J = 6 Hz, 1H), 7.45 (dd, J = 8 4 Hz, 1H), 7.37 (m, 2H), 4.92 (m, 2H), 4.63 (s, 1H), 4.52 (s, 1H), 3.48-3.18 (m, 2H), 2.95 (m, 2H), 2.77 (m, 2H), 2.04 (bd, J = 13 Hz, 1H), 1.80-0.50 (m, 42H) [M + H]+ Ion mass = 738.4791 |
| 229 | 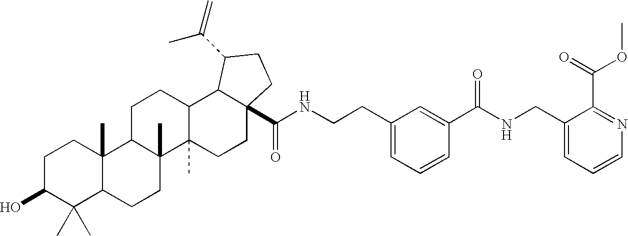 | 44 | 7.0 | 1H NMR (400 MHz, d6-DMSO) δ 9.00 (bt, J = 6 Hz, 1H), 8.55 (d, J = 4 Hz, 1H), 7.83 (d, J = 8 Hz, 1H), 7.75 (s, 1H), 7.73 (m, 1H), 7.63 (bt, J = 6 Hz, 1H), 7.57 (dd, J = 8 4 Hz, 1H), 7.38 (m, 2H), 4.72 (d, J = 6 Hz, 2H), 4.63 (s, 1H), 4.52 (s, 1H), 4.27 (d, J = 6 Hz, 1H), 3.89 (s, 3H), 3.43-3.18 (m, 2H), 2.96 (m, 2H), 2.77 (m, 2H) 2.04 (bd, J = 13 Hz, 1H), 1.80-0.52 (m, 40H) [M + H]+ Ion mass = 752.49970, [M + Na]+ Ion mass = 774.48164 |
| 230 | 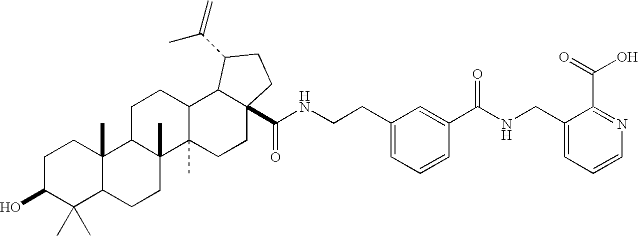 | 300 | 56 | 1H NMR (400 MHz, d6-DMSO) δ 9.02 (bs, 1H), 8.55 (s, 1H), 7.78 (m, 4H), 7.64 (bs, 1H), 7.56 (bs, 1H), 7.39 (bs, 2H), 4.76 (m, 2H), 4.63 (s, 1H), 4.51 (s, 1H), 3.74-0.52 (m, 40H) [M + H]+ Ion mass = 738.48405, [M + Na]+ Ion mass = 760.46599 |
| 231 | 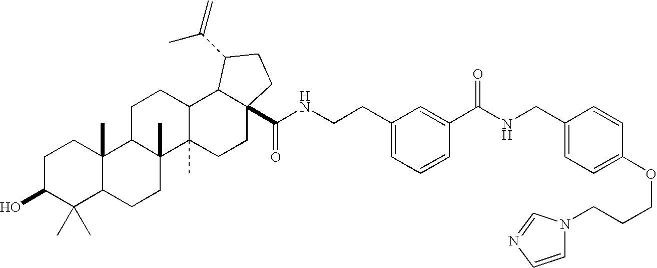 | 18.5 | 14.2 | 1H NMR (400 MHz, d6-DMSO) δ 9.06 (s, 1H), 8.95 (bt, J = 6 Hz, 1H), 7.78 (s, 1H), 7.74 (s, 1H), 7.71 (m, 1H), 7.65 (m, 2H), 7.35 (m, 2H), 7.23 (d, J = 8 Hz, 2H), 6.84 (d, J = 8 Hz, 2H), 6.57 (bs, 1H), 4.63 (s, 1H), 4.52 (s, 1H), 4.39 (d, J = 6 Hz, 2H), 3.97 (t, J = 6 Hz, 2H), 2.77 (m, 2H), 2.27 (t, J = 6 Hz, 1H), 2.05 (bd, J = 13 Hz, 1H), 1.80-0.50 (bd, J = 13 Hz, 1H), 1.80-0.50 (m, 40H) [M + H]+ Ion mass = 817.5697, [M + Na]+ Ion mass = 839.5513 |
| 232 | 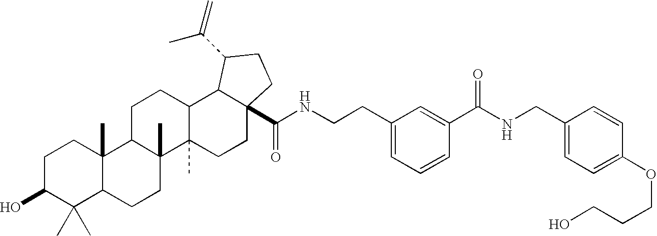 | 71.5 | 38.5 | 1H NMR (400 MHz, d6-DMSO) δ 8.91 (bt, J = 6 Hz, 1H), 7.74 (s, 1H), 7.71 (m, 1H), 7.62 (m, 2H), 7.35 (m, 2H), 7.26-7.15 (m, 3H), 6.87 (m, 3H), 4.63 (s, 1H), 4.52 (s, 1H), 4.40 (d, 6 Hz, 2H), 4.27 (d, 6 Hz, 2H), 4.12 (t, 6 Hz, 2H), 4.01 (m, 1H), 3.86 (t, 6 Hz, 2H) 3.42-3.18 (m, 2H), 2.96 (m, 2H), 2.75 (m, 2H) 2.60-0.50 (m, 40H) [M + H]+ Ion mass = 767.53575, [M + Na]+ Ion mass = 789.51769 |
| 233 | 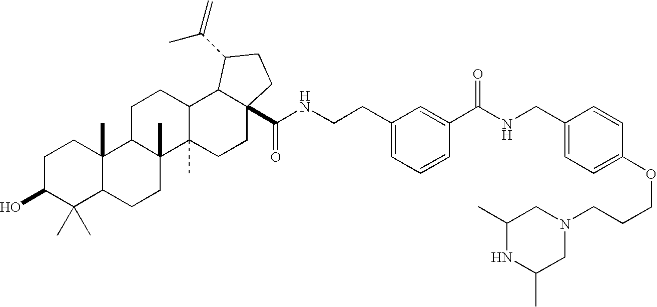 | N/A | 70 | 1H NMR (400 MHz, d4-MeOH) δ 7.73 (bs, 1H), 7.69 (m, 1H), 7.44-7.34 (m, 2H), 7.30 (d, J = 9 Hz, 2H), 6.90 (d, J = 9 Hz, 2H), 4.66 (s, 1H), 4.55 (s, 1H), 4.50 (s, 2H), 4.07 (m, 2H), 3.52 (m, 1H), 3.38 (m, 1H), 3.11 (m, 2H), 3.00 (m, 2H), 3.86 (t, J = 6 Hz, 2H), 2.51 (m, 1H), 2.00 (bd, J = 13 Hz, 1H), 1.84-0.63 (m, 54H) [M + H]+ Ion mass = 863.6447 |
| 234 | 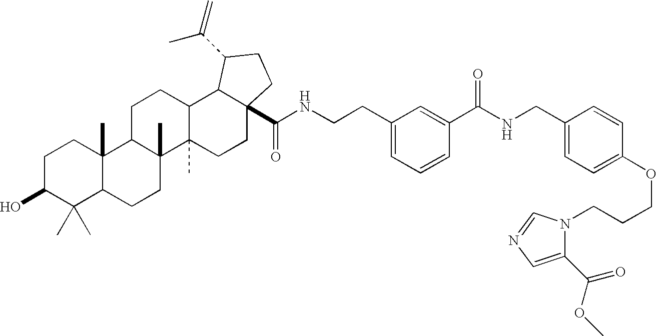 | 27 | 18.5 | 1H NMR (400 MHz, d6-DMSO) δ 8.92 (bt, J = 6 Hz, 1H), 8.17 (bs, 1H), 7.80 (bs, 1H), 7.74 (s, 1H), 7.71 (m, 1H), 7.63 (bt, J = 6 Hz, 1H), 7.34 (m, 2H), 7.23 (d, J = 8 Hz, 2H), 6.84 (d, J = 8 Hz, 2H), 4.63 (s, 1H), 4.52 (s, 1H), 4.47 (t, J = 6 Hz, 2H), 4.39 (d, J = 6 Hz, 2H), 3.90 (t, J = 6 Hz, 2H), 3.77 (s, 3H), 3.43-3.18 (m, 2H), 2.97 (m, 2H), 2.74 (m, 2H) 2.17 (t, J = 6 Hz, 1H), 2.05 (bd, J = 13 Hz, 1H), 1.80-0.53 (m, 42H) [M + H]+ Ion mass = 875.5789, [M + Na]+ Ion mass = 897.5660 |
| 235 | 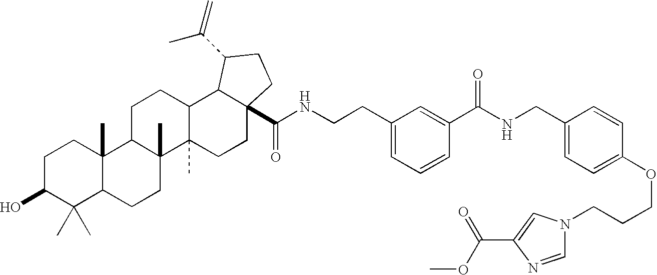 | 66.5 | 18 | 1H NMR (400 MHz, d6-DMSO) δ 8.92 (bt, J = 6 Hz, 1H), 7.99 (bs, 1H), 7.80 (bs, 1H), 7.74 (s, 1H), 7.71 (m, 1H), 7.63 (t, J = 6 Hz, 1H), 7.36 (m, 2H), 7.23 (d, J = 8 Hz, 2H), 6.87 (d, J = 8 Hz, 2H), 4.63 (s, 1H), 4.52 (s, 1H), 4.40 (d, J = 6 Hz, 2H), 4.19 (t, J = 6 Hz, 2H), 3.88 (t, J = 6 Hz, 2H), 3.76 (s, 1H), 3.72 (s, 3H), 3.48-3.18 (m, 2H), 2.97 (m, 2H), 2.76 (m, 2H), 2.20 (m, 1H), 2.05 (bd, J = 13 Hz, 1H), 1.80-0.54 (m, 41H) [M + H]+ Ion mass = 875.5789, [M + Na]+ Ion mass = 897.5659 |
| 236 | 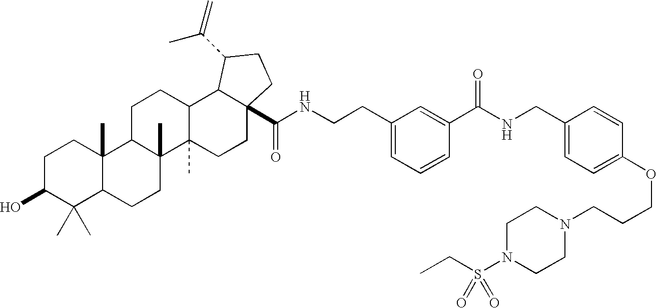 | 30 | 19.8 | 1H NMR (400 MHz, d6-DMSO) δ 8.94 (bs, 1H), 7.74 (s, 1H), 7.71 (m, 1H), 7.63 (m, 1H), 7.36 (m, 2H), 7.24 (m, 2H), 6.88 (m, 2H), 4.63 (s, 1H), 4.52 (s, 1H), 4.40 (m, 2H), 4.28 (m, 2H), 4.00 (m, 2H), 3.80-0.55 (m, 65H) [M + H]+ Ion mass = 927.5999, [M + Na]+ Ion mass = 949.5906 |
| 237 | 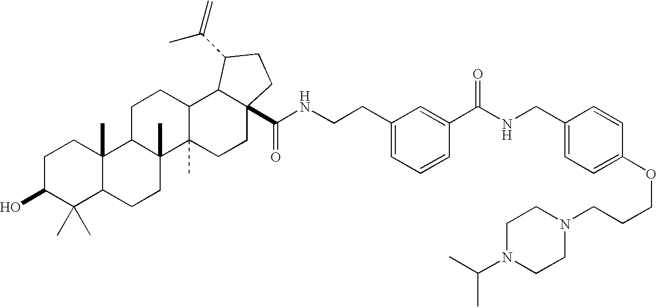 | N/A | 764 | 1H NMR (400 MHz, d6-DMSO) δ 8.94 (bt, J = 6 Hz, 1H), 7.74 (s, 1H), 7.71 (m, 1H), 7.64 (bt, J = 6 Hz, 1H), 7.36 (m, 2H), 7.24 (d, J = 8 Hz, 2H), 6.81 (d, J = 8 Hz, 2H), 4.63 (s, 1H), 4.52 (s, 1H), 4.40 (d, J = 6 Hz, 2H), 4.00 (t, J = 6 Hz, 2H), 3.42-3.18 (m, 2H), 2.97 (m, 2H), 2.75 (m, 2H), 2.06 (bd, J = 13 Hz, 1H), 2.02-0.46 (m, 59H) [M + H]+ Ion mass = 921.6363 |
| 238 | 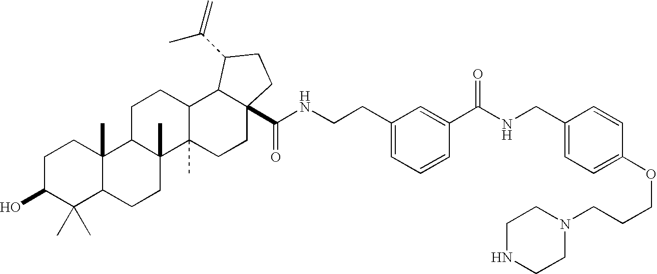 | N/A | 678 | 1H NMR (400 MHz, d6-DMSO) δ 8.94 (bt, J = 6 Hz, 1H), 7.74 (s, 1H), 7.71 (m, 1H), 7.64 (bt, J = 6 Hz, 1H), 7.36 (m, 2H), 7.24 (d, J = 8 Hz, 2H), 6.88 (d, J = 8 Hz, 2H), 4.63 (s, 1H), 4.52 (s, 1H), 4.40 (d, J = 6 Hz, 2H), 3.99 (t, J = 6 Hz, 2H), 3.51-0.50 (m, 61H) [M + H]+ Ion mass = 835.6013 |
| 239 | 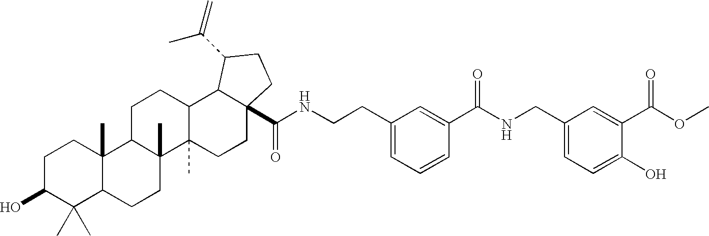 | 105 | 32.1 | [M + H]+ Ion mass = 768.50719, [M + Na]+ Ion mass = 790.48913 |
| 240 | 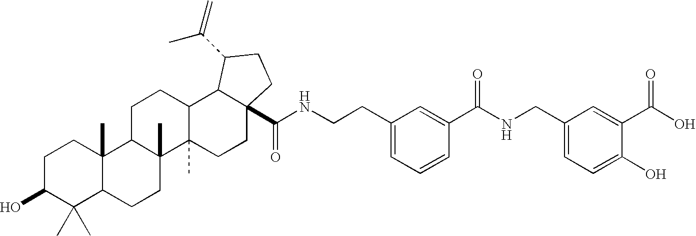 | 33 | 19 | 1H NMR (400 MHz, d6-DMSO) δ 11.19 (bs, 1H), 8.97 (bt, J = 6 Hz, 1H), 7.76 (d, J = 8 Hz, 1H), 7.73 (s, 1H), 7.71 (m, 1H), 7.61 (bt, J = 6 Hz, 1H), 7.47 (dd, J = 8 4 Hz, 1H), 7.36 (m, 2H), 6.91 (d, J = 8 Hz, 1H), 7.36 (m, 2H), 6.91 (d, J = 8 Hz, 1H), 4.62 (s, 1H), 4.51 (s, 1H), 4.40 (d, J = 6 Hz, 2H), 3.60 (m, 1H), 3.43-3.18 (m, 2H), 2.95 (m, 2H), 2.76 (m, 2H) 2.03 (bd, J = 13 Hz, 1H), 1.86-0.51 (m, 40H) [M + H]+ Ion mass = 753.48371, [M + Na]+ Ion mass = 775.46566 |
| 241 | 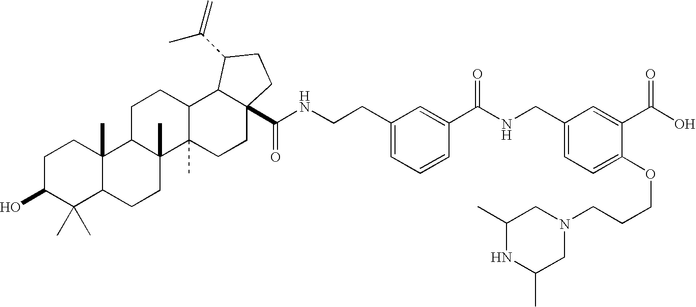 | 22.5 | 5 | 1H NMR (400 MHz, d6-DMSO) δ 9.00 (bt, J = 6 Hz, 1H), 7.74 (s, 1H), 7.71 (m, 1H), 7.63 (m, 2H), 7.44 (d, J = 8 Hz, 1H), 6.56 (bs, 3H), 4.63 (s, 1H), 4.52 (s, 1H), 4.41 (d, J = 6 Hz, 2H), 4.28 (bs, 1H), 4.04 (bs, 2H), 3.43-3.18 (m, 2H), 2.97 (m, 2H), 2.75 (m, 2H), 2.05 (bd, J = 13 Hz, 1H), 1.95-0.50 (m, 54H) [M + H]+ Ion mass = 907.6280 |
| 242 | 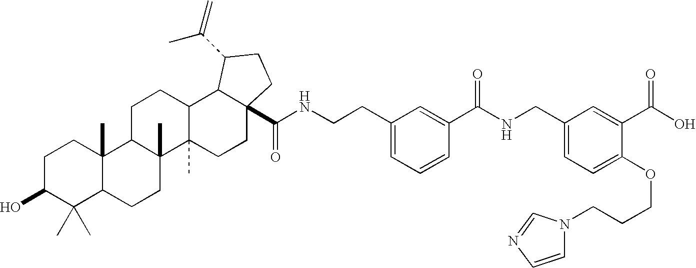 | 91 | 41.3 | 1H NMR (400 MHz, d6-DMSO) δ 9.00 (bt, J = 6 Hz, 1H), 7.74 (m, 2H), 7.65 (bs, 1H), 7.50 (bs, 1H), 7.44 (d, J = 8 Hz, 1H), 7.35 (m, 2H), 7.06 (d, J = 8 Hz, 1H), 6.57 (bs, 3H), 5.76 (s, 1H), 4.63 (s, 1H), 4.52 (s, 1H), 4.42 (m, 3H), 4.30 (m, 3H), 3.94 (m, 2H), 3.46-0.46 (m, 48H) [M + H]+ Ion mass = 861.5590 |
| 243 | 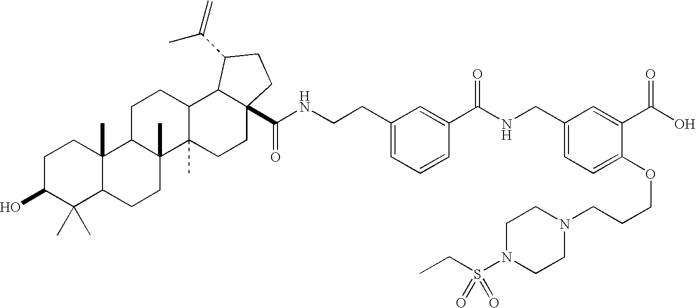 | 26 | 17.5 | 1H NMR (400 MHz, d6-DMSO) δ 12.90 (bs, 1H), 9.03 (bt, J = 6 Hz, 1H), 7.73 (m, 3H), 7.65 (m, 1H), 7.49 (d, J = 8 Hz, 1H), 7.37 (m, 2H), 7.12 (d, J = 8 Hz, 1H), 6.57 (bs, 1H), 4.64 (s, 1H), 4.52 (s, 1H), 4.42 (m, 2H), 4.13 (m, 2H), 4.00-0.47 (m, 65H) [M + H]+ Ion mass = 971.5888, [M + H]+ Ion mass = 993.5858 |
| 244 | 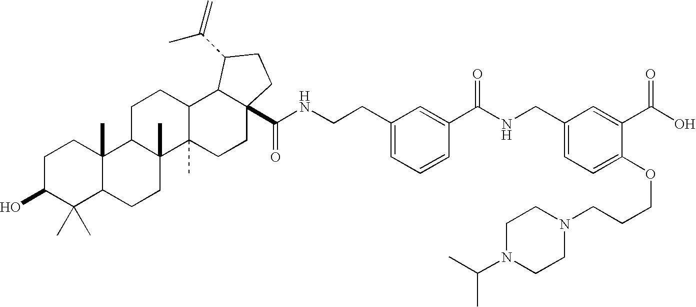 | 20 | 18.8 | 1H NMR (400 MHz, d6-DMSO) δ 9.02 (bt, J = 6 Hz, 1H), 7.69 (m, 4H), 7.46 (d, J = 8 Hz, 1H), 7.37 (m, 2H), 7.09 (d, J = 8 Hz, 1H), 4.63 (s, 1H), 4.52 (s, 1H), 4.42 (m, 2H), 4.09 (m, 2H), 3.97-0.47 (m, 68H) [M + H]+ Ion mass = 877.6538 |
| 245 | 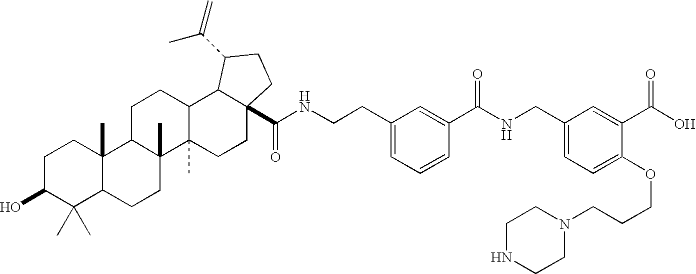 | 35 | 27.4 | 1H NMR (400 MHz, d6-DMSO) δ 9.01 (bt, J = 6 Hz, 1H), 7.69 (m, 4H), 7.46 (d, J = 8 Hz, 1H), 7.37 (m, 2H), 7.09 (d, J = 8 Hz, 1H), 4.63 (s, 1H), 4.52 (s, 1H), 4.42 (m, 2H), 4.09 (m, 2H), 3.97-0.47 (m, 60H) [M + H]+ Ion mass = 879.5898 |
| 246 | 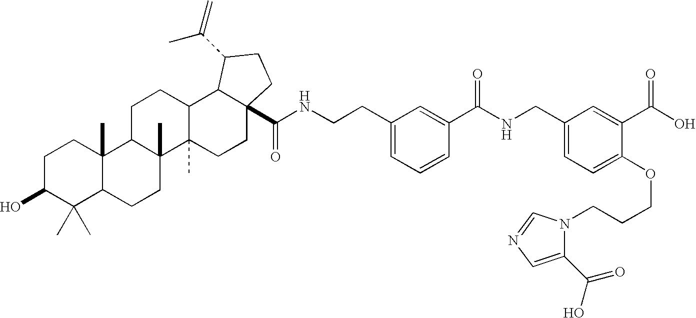 | 233 | 57.3 | 1H NMR (400 MHz, d4-MeOH) δ 8.95 (bt, J = 6 Hz, 1H), 8.38 (s, 1H), 7.85 (d, J = 2 Hz, 1H), 7.77 (s, 1H), 7.73 (s, 1H), 7.70 (d, J = 8 Hz, 1H), 7.61 (t, J = 6 Hz, 1H), 7.51 (dd, J = 8, 2 Hz, 1H), 7.40 (m, 2H), 7.04 (d, J = 8 Hz, 1H), 4.73 (t, J = 6 Hz, 2H), 4.66 (s, 1H), 4.54 (m, 3H), 3.99 (t, J = 6 Hz, 2H), 3.59-3.27 (m, 2H), 3.11 (m, 1H), 3.00 (m, 1H), 2.86 (t, J = 6 Hz, 2H), 2.51 (bt, J = 12 Hz, 1H), 2.36 (t, J = 6 Hz, 2H), 1.99 (bd, J = 13 Hz, 1H), 1.87-0.55 (m, 40H) [M + H]+ Ion mass = 905.5517 |
| 247 | 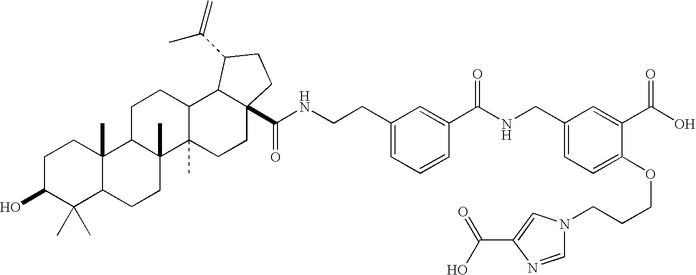 | N/A | 417 | 1H NMR (400 MHz, d4-MeOH) δ 8.95 (bt, J = 6 Hz, 1H), 8.02 (s, 1H), 7.88 (s, 1H), 7.85 (d, J = 2 Hz, 1H), 7.74 (s, 1H), 7.70 (d, J = 8 Hz, 1H), 7.61 (t, J = 6 Hz, 1H), 7.51 (dd, J = 8, 2 Hz, 1H), 7.45-7.35 (m, 2H), 7.04 (d, J = 8 Hz, 1H), 4.66 (s, 1H), 4.54 (m, 3H), 4.42 (t, J = 6 Hz, 2H), 4.01 (t, J = 6 Hz, 2H), 3.60-3.28 (m, 2H), 3.10 (m, 1H), 3.00 (m, 1H), 2.86 (t, J = 6 Hz, 2H), 2.51 (bt, J = 12 Hz, 1H), 2.32 (t, J = 6 Hz, 2H), 1.99 (bd, J = 13 Hz, 1H) 1.85-0.61 (m, 40H) [M + H]+ Ion mass = 905.5541, [M + Na]+ Ion mass = 9927.5279 |
| 248 | 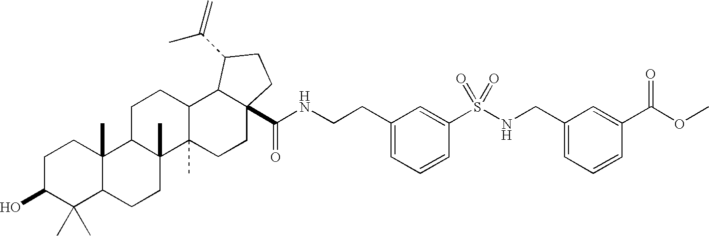 | N/A | 208 | 1H NMR (400 MHz, d6-DMSO) δ 8.22 (bs, 1H), 7.84 (s, 1H), 7.81 (d, J = 8 Hz, 1H), 7.65 (bt, J = 6 Hz, 2H), 7.61 (bs, 2H), 7.54-7.39 (m, 4H), 4.62 (s, 1H), 4.51 (s, 1H), 4.26 (d, J = 5 Hz, 1H), 4.05 (s, 2H), 3.85 (s, 3H), 3.39-3.20 (m, 2H), 2.96 (m, 2H), 3.39-3.20 (m, 2H), 2.96 (m, 2H), 2.76 (m, 2H), 2.05 (bd, J = 12 Hz, 1H), 1.82-0.54 (m, 40H) [M + H]+ Ion mass = 787.47078, [M + Na]+ Ion mass = 809.45480 |
| 249 | 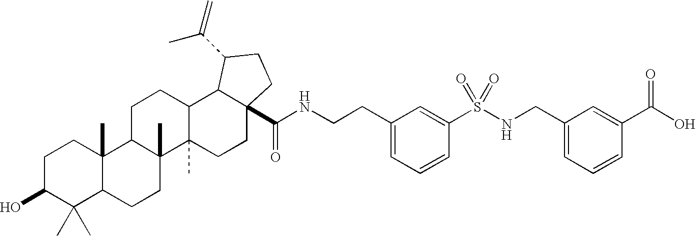 | N/A | 143 | [M + H]+ Ion mass = 773.45578, [M + Na]+ Ion mass = 795.43773 |
| 250 | 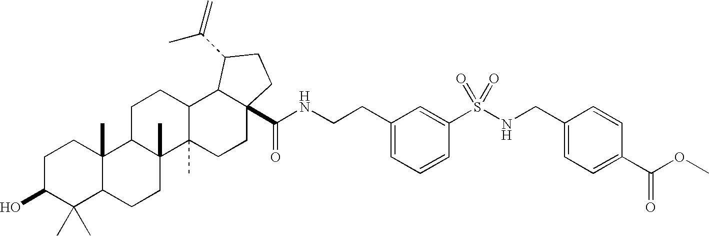 | 594 | 106 | 1H NMR (400 MHz, d6-DMSO) δ 8.23 (bt, J = 6 Hz, 1H), 7.87 (d, J = 8 Hz, 1H), 7.67 (bt, J = 6 Hz,2H), 7.63 (m, 2H), 7.48 (m, 2H), 7.40 (d, J = 8 Hz, 2H), 4.61 (s, 1H), 4.51 (s, 1H), 4.27 (d, J = 5 Hz, 1H), 4.04 (m, 2H), 3.84 (s, 3H), 3.38-3.21 (m, 2H), 2.95 (m, 2H), 2.77 (m, 2H), 2.55 (m, 1H), 2.05 (bd, J = 12 Hz, 1H), 1.81-0.54 (m, 40H) [M + H]+ Ion mass = 787.47078, [M + Na]+ Ion mass = 809.45480 |
| 251 | 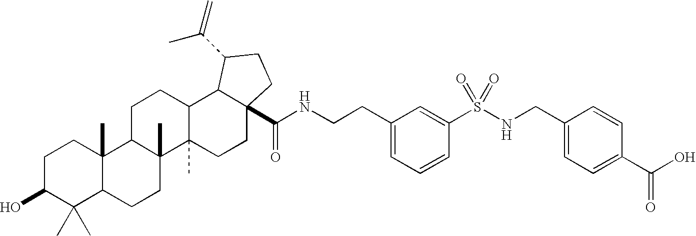 | 257.5 | 68 | 1H NMR (400 MHz, d6-DMSO) δ 12.92 (bs, 1H), 8.21 (bt, J = 6 Hz, 1H), 7.85 (d, J = 8 Hz, 1H), 7.69-7.61 (m, 3H), 7.51-7.43 (m, 2H), 7.37 (d, J = 8 Hz, 2H), 4.61 (s, 1H), 4.51 (s, 1H), 4.03 (m, 2H), 3.43-3.23 (m, 2H), 2.95 (m, 2H), 2.77 (m, 2H), 2.55 (m, 1H), 2.05 (bd, J = 12 Hz, 1H), 1.81-0.54 (m, 40H) [M + H]+ Ion mass = 773.5 |
| 252 | 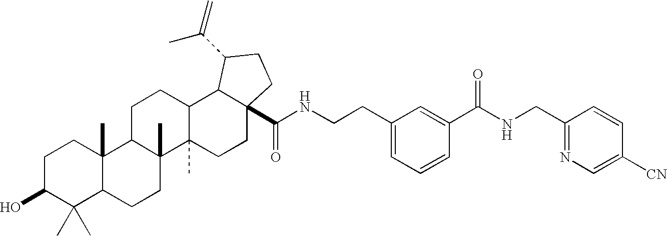 | 14 | 25 | 1H NMR (400 MHz, d6-DMSO) δ 9.18 (bt, J = 6 Hz, 1H), 8.98 (d, J = 2 Hz, 1H), 8.26 (dd, J = 8, 2 Hz, 1H), 8.26 (dd, J = 8, 2 Hz, 1H), 7.78 (s, 1H), 7.75 (m, 1H), 7.64 (bt, J = 6 Hz, 1H), 7.51 (d, J = 8 Hz, 1H), 7.39 (m, 2H), 4.63 (m, 3H), 4.27 (bs, 1H), 3.42-3.18 (m, 2H), 2.96 (m, 2H), 2.78 (m, 2H), 2.05 (bd, J = 12 Hz, 1H), 1.80-0.50 (m, 40H) [M + H]+ Ion mass = 719.48947, [M + Na]+ Ion mass = 741.47141 |
| 253 | 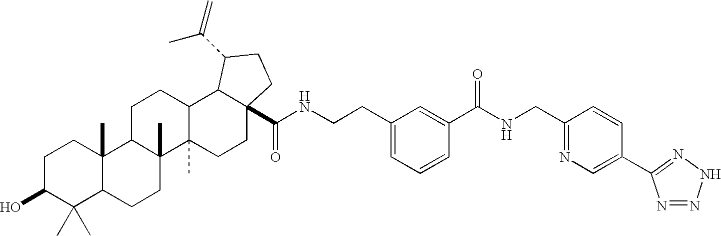 | 3.5 | 10.8 | 1H NMR (400 MHz, d6-DMSO) δ 9.19 (bt, J = 6 Hz, 1H), 9.15 (d, J = 2 Hz, 1H), 8.37 (dd, J = 8, 2 Hz, 1H), 7.80 (s, 1H), 7.77 (m, 1H), 7.64 (bt, J = 6 Hz, 1H), 7.55 (d, J = 8 Hz, 1H), 7.40 (m, 2H), 4.64 (m, 3H), 4.51 (s, 1H), 3.42-3.18 (m, 2H), 2.96 (m, 2H), 2.78 (m, 2H), 2.05 (bd, J = 12 Hz, 1H), 1.82-0.52 (m, 42H) [M + H]+ Ion mass = 762.50652, [M + Na]+ Ion mass = 784.48846 |
| 254 | 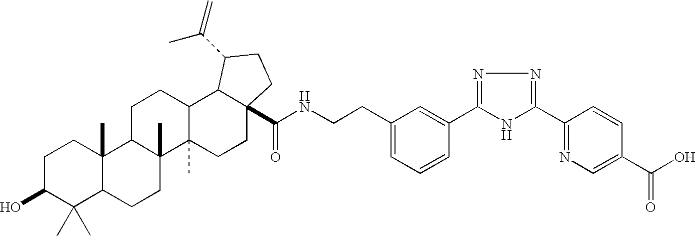 | 351 | 69 | 1H NMR (400 MHz, d6-DMSO) δ 13.59 (bs, 1H), 9.17 (s, 1H), 8.45 (d, J = 8 Hz, 1H), 8.31 (d, J = 8 Hz, 1H), 7.95 (m, 2H), 7.56 (bs, 1H), 7.41 (m, 1H), 7.32 (m, 1H), 4.61 (s, 1H), 4.50 (s, 1H), 3.55 (m, 1H), 3.23 (m, 1H), 3.00-2.79 (m, 4H), 1.84-0.36 (m, 42H) [M + H]+ Ion mass = 748.4694 |
| 255 | 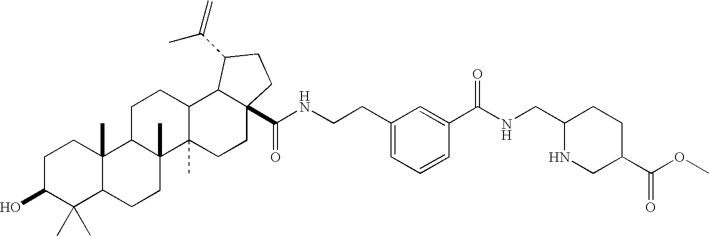 | N/A | 7330 | 1H NMR (400 MHz, d6-DMSO) δ 10.03 (bs, 1H), 8.87 (bt, J = 6 Hz, 1H), 7.78 (m, 2H), 7.65 (bt, J = 6 Hz, 1H), 7.78 (m, 2H), 7.65 (bt, J = 6 Hz, 1H), 7.37 (m, 2H), 4.31 (bs, 1H), 3.74-0.56 (m, 65H) [M + H]+ Ion mass = 760.5 |
| 256 | 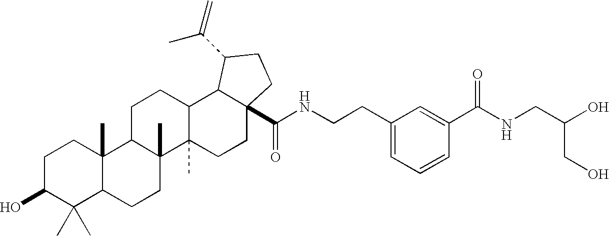 | 204 | 50.5 | 1H NMR (400 MHz, d6-DMSO) δ 8.35 (bt, J = 6 Hz, 1H), 7.71 (s, 1H), 7.68 (m, 1H), 7.64 (bt, J = 6 Hz, 1H), 7.35 (m, 2H), 4.81 (d, J = 5 Hz, 1H), 4.64 (s, 1H) 4.57 (t, J = 6 Hz, 1H), 4.64 (s, 1H) 4.57 (t, J = 6 Hz, 1H), 4.59 (bs, 1H), 4.26 (d, J = 5 Hz, 1H), 3.62 (m, 1H), 3.23 (m, 2H), 2.97 (m, 2H), 2.75 (m, 2H), 2.06 (bd, J = 13 Hz, 1H), 1.81-0.53 (m, 42H) [M + H]+ Ion mass = 677.48880, [M + Na]+ Ion mass = 699.47074 |
| 257 | 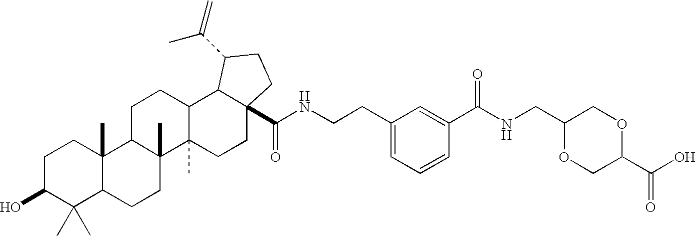 | 328 | 54 | 1H NMR (400 MHz, d6-DMSO) δ 8.54 (bt, J = 6 Hz, 1H), 7.71 (s, 1H), 7.68 (m, 1H), 7.61 (bt, J = 6 Hz, 1H), 7.36 (m, 2H), 6.58 (s, 1H), 4.64 (s, 1H), 4.52 (bs, 1H), 4.26 (d, J = 6 Hz, 1H), 4.18 (m, 1H), 3.88 (bd, 12 Hz, 1H), 3.76 (m, 2H), 3.54-3.13 (m, 2H), 2.96 (m, 2H), 2.76 (m, 2H), 2.04 (bd, J = 13 Hz, 1H), 1.81-0.53 (m, 44H) [M + H]+ Ion mass = 747.4746 |
| 258 | 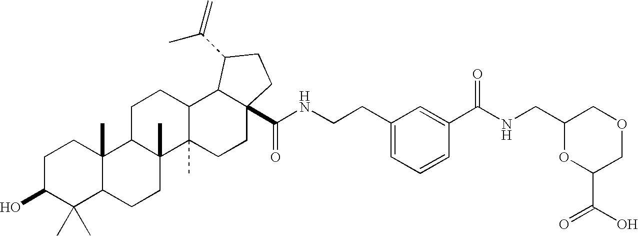 | 984 | 112 | 1H NMR (400 MHz, d6-DMSO) δ 12.91 (bs, 1H), 8.49 (bt, J = 6 Hz, 1H), 7.71 (s, 1H), 7.67 (m, 1H), 7.62 (bt, J = 6 Hz, 1H), 7.35 (m, 2H), 6.53 (s, 1H), 4.64 (s, 1H), 4.52 (bs, 1H), 4.35 (m, 1H), 4.25 (d, J = 6 Hz, 1H), 4.12 (m, 1H), 3.97 (bd, 12 Hz, 1H), 3.76 (m, 2H), 3.80-3.60 (m, 2H), 3.40-3.19 (m, 2H), 2.96 (m, 2H), 2.76 (m, 2H), 2.05 (bd, J = 13 Hz, 1H), 1.81-0.53 (m, 42H) [M + H]+ Ion mass = 747.5055 |
| 259 | 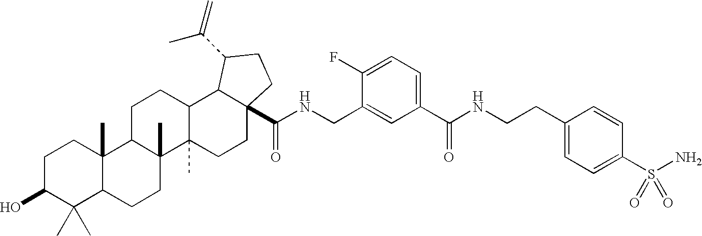 | N/A | 175 | 1H NMR (400 MHz, d6-DMSO) δ 8.58 (bt, J = 6 Hz, 1H), 8.24 (bt, J = 6 Hz, 1H), 7.86 (d, J = 8 Hz, 1H), 7.74 (d, J = 8 Hz, 2H) 7.69 (m, 1H), 7.41 (d, J = 8 Hz, 2H), 7.31 (s, 2H), 7.23 (t, J = 9 Hz, 1H), 4.63 (s, 1H), 4.52 (s, 1H), 4.40-4.12 (m, 3H), 3.47 (m, 2H), 3.04-2.86 (m, 4H), 2.46 (m, 1H), 2.21 (bd, J = 13 Hz, 1H), 1.93-0.52 (m, 40H) [M + H]+ Ion mass = 790.4415, [M + Na]+ Ion mass = 812.4455 |
| 260 | 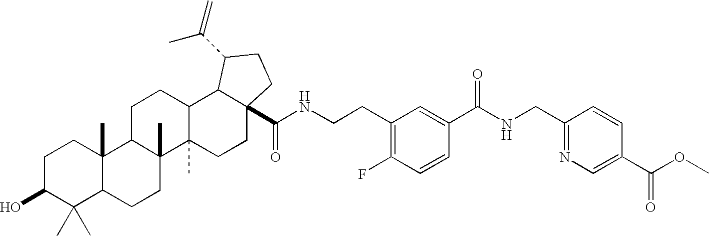 | N/A | 150 | 1H NMR (400 MHz, d6-DMSO) δ 9.18 (m, 1H), 9.03 (s, 1H), 8.25 (d, J = 8, Hz, 1H), 7.86 (m, 2H), 7.66 (m, 1H), 7.45 (d, J = 8 Hz, 1H), 7.26 (m, 1H), 4.62 (m, 3H), 4.50 (s, 1H), 4.26 (m, 1H), 3.88 (s, 3H), 3.33 (m, 2H), 2.94 (m, 2H), 2.78 (m, 2H), 2.03 (bd, J = 12 Hz, 1H), 1.80-0.50 (m, 40H) [M + H]+ Ion mass = 770.49028, [M + Na]+ Ion mass = 792.47222 |
| 261 | 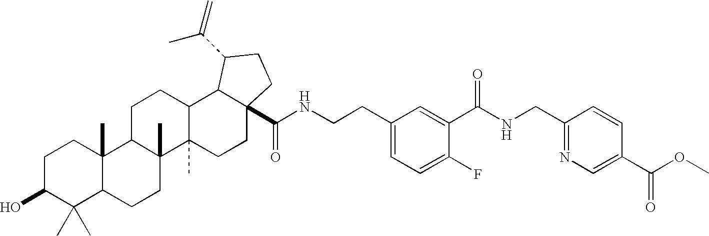 | 8.9 | 14.5 | 1H NMR (400 MHz, d6-DMSO) δ 9.04 (d, J = 2 Hz, 1H), 8.88 (m, 1H), 8.28 (dd, J = 8, 2 Hz, 1H), 7.59 (m, 2H), 7.50 (d, J = 8 Hz, 1H), 7.39 (m, 1H), 7.23 (m, 1H), 4.64 (m, 3H), 4.51 (s, 1H), 4.26 (d, J = 6 Hz, 1H), 3.89 (s, 3H), 3.39 (m, 1H), 3.31 (s, 1H), 3.20 (m, 1H), 2.94 (m, 2H), 2.74 (m, 2H), 2.02 (bd, J = 13 Hz, 1H), 1.80-0.50 (m, 42H) [M + H]+ Ion mass = 770.49028 |
| 262 | 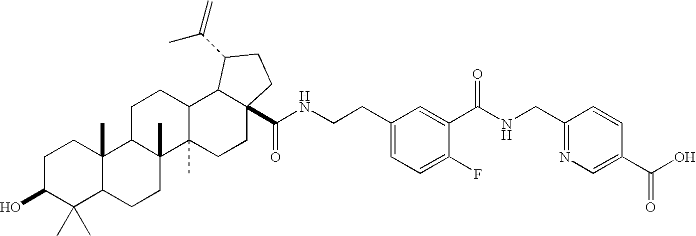 | 7.2 | 11.5 | 1H NMR (400 MHz, d6-DMSO) δ 8.98 (d, J = 2 Hz, 1H), 8.85 (m, 1H), 8.19 (dd, J = 8, 2 Hz, 1H), 7.60 (m, 2H), 7.38 (m, 2H), 7.23 (m, 2H), 4.62 (m, 3H), 4.51 (s, 1H), 3.38 (m, 2H), 3.20 (m, 1H), 2.96 (m, 2H), 2.75 (m, 2H), 2.03 (bd, J = 12 Hz, 1H), 2.00-0.52 (m, 42H) [M + H]+ Ion mass = 756.47463, [M + Na]+ Ion mass = 778.45657 |
| 263 | 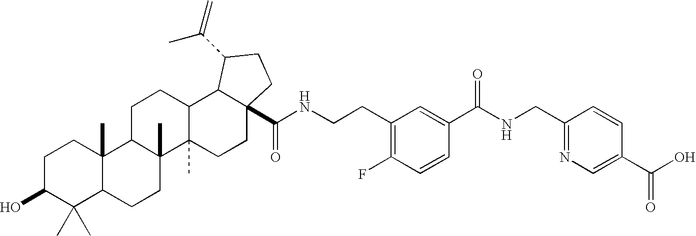 | N/A | 198 | 1H NMR (400 MHz, d6-DMSO) δ 9.21 (bt, J = 6 Hz, 1H), 8.94 (d, J = 2 Hz, 1H), 8.12 (dd, J = 8, 2 Hz, 1H), 7.94 (d, 8 Hz, 1H), 7.85 (m, 1H), 7.73 (bt, J = 6 Hz, 1H), 7.26 (m, 2H), 4.57 (m, 4H), 3.40 (m, 1H), 3.29 (m, 1H), 2.95 (m, 2H), 2.80 (m, 2H), 2.06 (bd, J = 12 Hz, 1H), 2.00-0.52 (m, 42H) [M + H]+ Ion mass = 756.47463, [M + Na]+ Ion mass = 778.45657 |
| 264 | 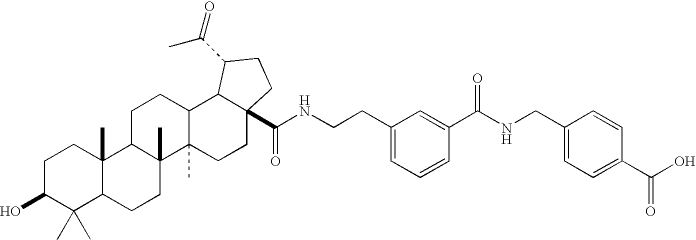 | 161 | 29.5 | 1H NMR (400 MHz, d6-DMSO) δ 8.98 (bt, J = 6 Hz, 1H), 7.82-7.66 (m, 4H), 7.36 (m, 2H), 7.19 (d, J = 8 Hz, 2H), 4.48 (d, J = 5 Hz, 2H), 3.42-3.22 (m, 2H), 3.14 (m, 1H), 2.96 (m, 2H), 2.76 (m, 2H), 2.05 (bd, J = 13 Hz, 1H), 1.82-0.54 (m, 40H) [M + H]+ Ion mass = 739.4647 |
| 265 | 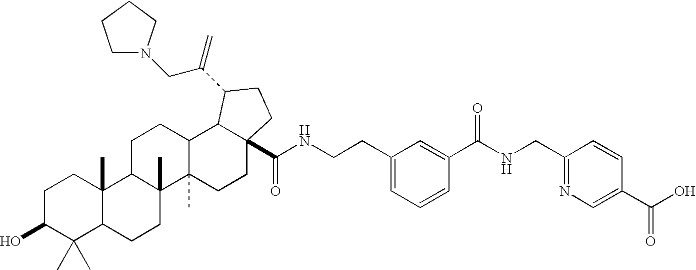 | 172.5 | 58 | 1H NMR (400 MHz, d2-DCM) δ 10.75 (bs, 1H), 9.15 (s, 1H), 8.52 (d, J = 8 Hz, 1H), 8.38 (bt, J = 6 Hz, 1H), 7.73 (m, 2H), 7.62 (s, 1H), 7.37 (m, 2H), 5.68 (bt, J = 6 Hz, 1H), 5.28 (s, 1H), 5.18 (d, J = 6 Hz, 1H), 4.94-4.71 (m, 2H), 3.92-0.52 (m, 55H) [M + H]+ Ion mass = 807.54190, [M + Na]+ Ion mass = 829.52384 |
| 266 | 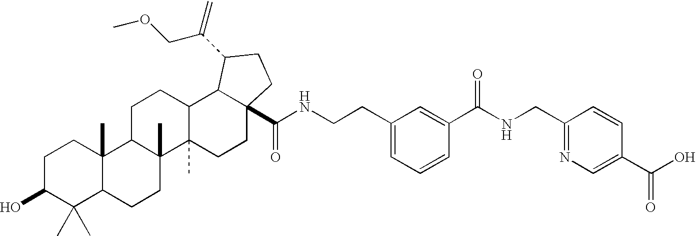 | 24 | 8.3 | 1H NMR (400 MHz, CDCl₃) δ 9.23 (s, 1H), 8.37 (d, J = 10 Hz, 1H), 7.76 (m, 3H), 7.53 (d, J = 8 Hz, 1H), 7.39 (m, 2H), 5.67 (bt, J = 6 Hz, 1H), 4.91 (d, J = 6 Hz, 2H), 4.86 (d, J = 4.5 Hz, 2H), 3.85 (s, 2H), 3.55 (m, 2H), 3.35 (s, 3H), 3.17 (m, 1H), 2.90 (m, 3H), 2.37 (bt, J = 13 Hz, 1H), 2.25-0.50 (m, 40H) [M + H]+ Ion mass = 768.5057 |
| 267 | 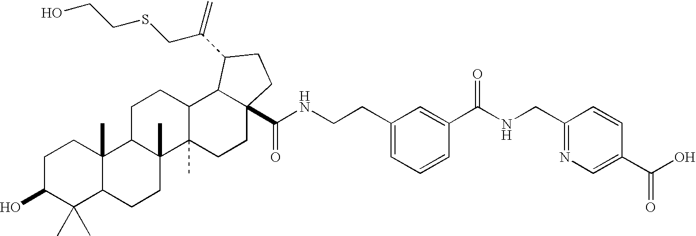 | 46 | 17.3 | 1H NMR (400 MHz, d6-DMSO) δ 13.32 (bs, 1H), 9.17 (bt, J = 6 Hz, 1H), 9.01 (d, J = 2 Hz, 1H), 8.23 (dd, J = 8, 2 Hz, 1H), 7.79 (s, 1H), 7.76 (m, 1H), 7.65 (bt, J = 6 Hz, 1H), 7.44 (d, J = 8 Hz, 1H), 7.39 (m, 2H), 4.84 (s, 1H), 4.78 (s, 1H), 4.63 (d, J = 6 Hz, 2H), 2.94 (m, 2H), 2.78 (m, 2H), 2.46 (m, 2H), 2.23-0.52 (m, 48H) [M + H]+ Ion mass = 814.5 |
| 268 | 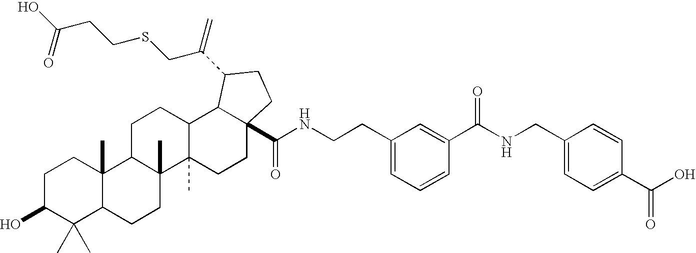 | 411 | 104 | 1H NMR (400 MHz, d6-Acetone) δ 8.40 (bt, J = 6 Hz, 1H), 8.01 (d, J = 8 Hz, 2H), 7.86 (s, 1H), 7.81 (d, J = 8 Hz, 1H), 7.52 (d, J = 8 Hz, 2H), 7.40 (m, 2H), 7.06 (bt, J = 6 Hz, 1H), 4.93 (s, 1H), 4.88 (s, 1H), 4.71 (d, J = 6 Hz, 2H), 3.59-3.40 (m, 2H), 3.28-3.06 (m, 4H), 2.87 (m, 2H), 2.73-2.56 (m, 6H), 2.19-0.55 (m, 38H) [M + H]+ Ion mass = 841.4808, [M + Na]+ Ion mass = 863.4620 |
| 269 | 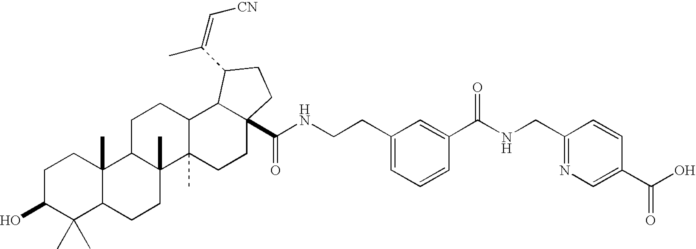 | 39 | 11.4 | 1H NMR (400 MHz, d6-Acetone) δ 9.11 (d, J = 2 Hz, 1H), 8.44 (bt, J = 6 Hz, 1H), 8.31 (dd, J = 8, 2 Hz, 1H), 7.89 (s, 1H), 7.83 (m, 1H), 7.55 (d, J = 8 Hz, 1H), 7.43 (m, 2H), 7.16 (bt, J = 6 Hz, 1H), 5.38 (s, 1H), 4.78 (d, J = 6 Hz, 2H), 3.51 (m, 2H), 3.30 (m, 1H), 3.11 (m, 2H), 2.90 (m, 1H), 2.65 (m, 1H), 2.19-0.55 (m, 40H) [M + H]+ Ion mass = 763.47930, [M + Na]+ Ion mass = 785.46124 |
| 270 | 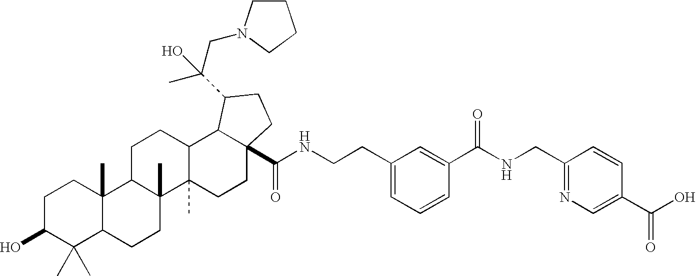 | 441 | 100 | 1H NMR (400 MHz, d6-DMSO) δ 9.16 (bt, J = 6 Hz, 1H), 9.01 (d, J = 2 Hz, 1H), 8.23 (dd, J = 8, 2 Hz, 1H), 7.77 (m, 2H), 7.58 (bt, J = 6 Hz, 1H), 7.44 (d, J = 8 Hz, 1H), 7.39 (m, 2H), 4.76 (s, 1H), 4.63 (d, J = 5 Hz, 2H), 3.72-0.52 (m, 59H) [M + H]+ Ion mass = 825.5552 |
| 271 | 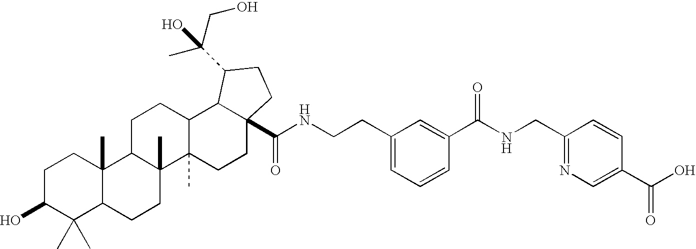 | 1810 | 118.8 | 1H NMR (400 MHz, d6-Acetone) δ 9.10 (s, 1H), 8.47 (bt, J = 6 Hz, 1H), 8.31 (d, J = 8 Hz, 1H), 7.57 (d, J = 8 Hz, 1H), 7.43 (m, 2H), 7.07 (bt, J = 6 Hz, 1H), 4.79 (d, J = 5 Hz, 2H), 4.02 (d, J = 9 Hz, 1H), 3.77 (d, 9 Hz, 1H), 3.50 (m, 2H), 3.20-0.63 (m, 48H) [M + H]+ Ion mass = 754.47896 |
| 272 | 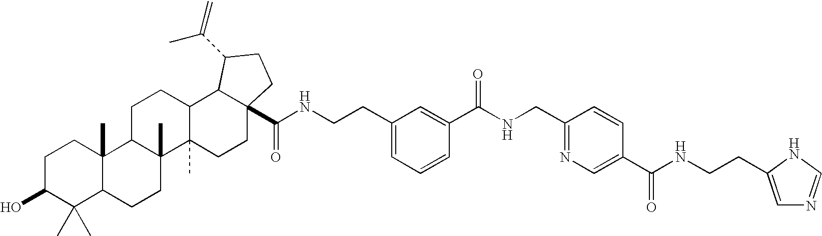 | 12.4 | 3.1 | 1H NMR (400 MHz, d6-DMSO) δ 9.17 (m, 1H), 8.93 (d, J = 1.6 Hz, 1H), 8.77 (m, 1H), 8.14 (d, J = 8.0 Hz, 1H), 7.79 (s, 1H), 7.75 (m, 1H), 7.67 (m, 1H), 7.54 (s, 1H), 7.39 (d, J = 6.4 Hz, 2H), 4.63 (s, 1H), 4.60 (d, J = 6.0 Hz, 2H), 4.52 (s, 1H), 4.29 (s, 1H), 3.50-0.80 (m, 44H), 0.75 (s, 3H), 0.74 (s, 3H), 0.64 (s, 3H); Mass Spec (m/z): 831 (M + 1). |
| 273 | 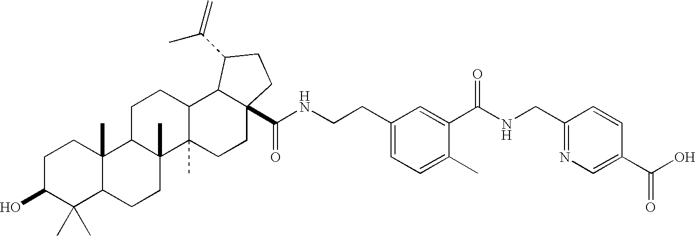 | 1860 | 127.3 | 1H NMR (400 MHz, d6-DMSO) δ 8.94 (s, 1H), 8.80 (m, 1H), 8.16 (d, J = 8.0 Hz, 1H), 7.68 (m, 1H), 7.34 (d, J = 8.4 Hz, 1H), 7.16 (m, 2H), 4.64 (s, 1H), 4.54 (d, J = 6.0 Hz, 2H), 4.52 (s, 1H), 4.29 (s, 1H), 3.50-0.80 (m, 42H), 0.77 (s, 3H), 0.74 (s, 3H), 0.64 (s, 3H); Mass Spec (m/z): 752 (M + 1). |
| 274 | 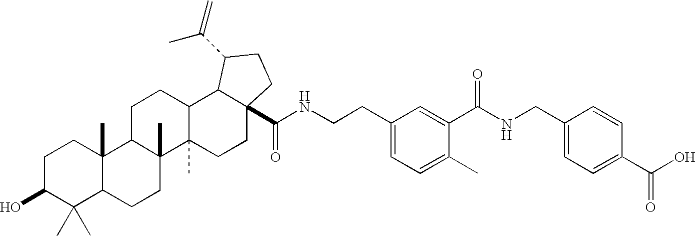 | 4320 | 84.5 | 1H NMR (400 MHz, d6-DMSO) δ 8.79 (s, 1H), 7.90 (d, J = 8.4 Hz, 1H), 7.66 (m, 1H), 7.41 (d, J = 8.8 Hz, 1H), 7.24 (s, 1H), 7.15 (s, 2H), 4.64 (s, 1H), 4.52 (s, 1H), 4.48 (d, J = 6.4 Hz, 2H), 4.29 (s, 1H), 3.50-0.80 (m, 43H), 0.77 (s, 3H), 0.74 (s, 3H), 0.64 (s, 3H); Mass Spec (m/z): 751 (M + 1). |
| 275 | 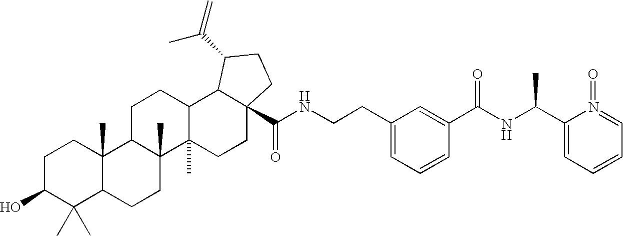 | 1000 | 180 | 1H NMR (400 MHz, d6-DMSO) δ 8.91 (d, J = 8 Hz, 1H), 8.27 (t, J = 3.2 Hz, 1H), 7.77-7.70 (br m, 2H), 7.645-7.60 (m, 1H), 7.47-7.3 (m, 5H), 5.55 (t, J = 7.2 Hz, 1H), 4.63 (br s, 1H), 4.51 (br s, 1H), 4.27 (m, 1H), 3.5-0.2 (m, 51H). |
| 276 | 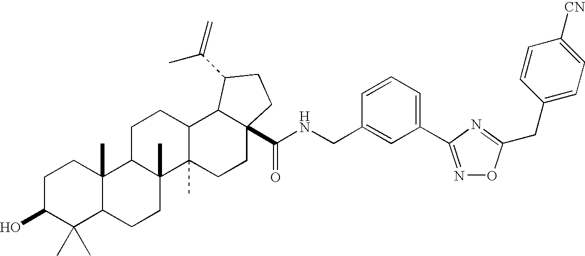 | N/A | 10000 | 1H NMR (400 MHz, d6-DMSO) δ 8.3 (t, J = 5.6 Hz, 1H), 7.9 (br s, 1H), 7.88 (br s, 1H), 7.83-7.76 (m, 3H), 7.62 (t, J = 8 Hz, 1H), 7.49-7.41 (m, 2H), 4.53 (br s, 1H), 4.53 (br s, 1H), 4.37-4.19 (m, 2H), 3.1-2.85 (m, 2H), 2.5-0.5 (m, 45H). MS (m/z): 729.46 (M + 1), 751.45 (M + 23). |
| 277 | 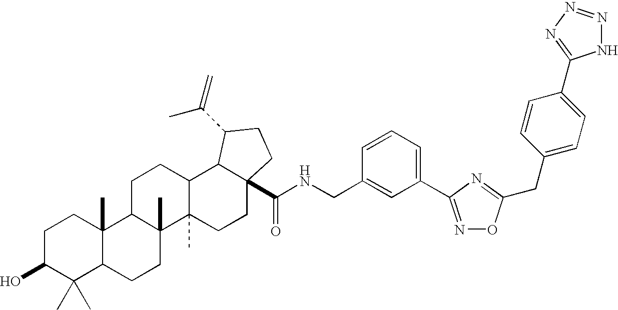 | N/A | 10000 | 1H NMR (400 MHz, CDCl3): δ 8.19 (br s, 1H), 8.1-7.9 (br m, 1H), 7.47-7.3 (m, 4H), 6.61 (br s, 1H), 4.66 (s, 1H), 4.56 (s, 1H), 4.46-4.33 (m, 2H), 4.25 (br s, 2H), 3.2-3.0 (m, 2H), 2.45-0.6 (m, 46H). MS (m/z): 771.5 (M + 1). |

N/A = Not available

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A compound having the structure: 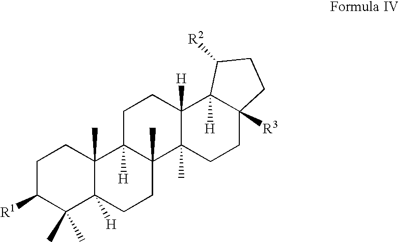
and pharmaceutically acceptably salts and stereoisomers thereof, wherein:
R¹ is —OH or ═O;
R² is optionally substituted carbonyl, isopropenyl or isopropyl, wherein each can be optionally substituted with one or two substituents independently selected from hydroxyl, halo, cyano, C₁₋₆ alkoxy (optionally substituted with hydroxyl, C-carboxyl or O-carboxyl), C₁₋₆ alkylthio (optionally substituted with hydroxyl, C-carboxyl or O-carboxyl) and —N(R²¹R²²) wherein R²¹ and R²² are independently H, C₁₋₆ alkyl, C₁₋₆ hydroxyalkyl, or R²¹ and R²² together with the nitrogen they are attached to form a 3 to 6-membered heterocycle;
R³ is represented by 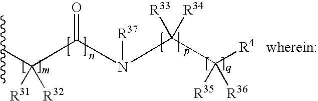
R³¹ and R³² are independently H or methyl or ethyl, or R³¹ and R³² can be taken together with the carbon they are attached to form a C₃₋₅ cycloalkyl;
R³³ is H, halo, —COOR^{33a} (R^{33a} is H or C₁₋₆ alkyl such as methyl, ethyl, propyl and isopropyl), methyl or ethyl, and R³⁴ is H, halo, methyl or ethyl, or R³³ and R³⁴ can be taken together with the carbon they are attached to form a C₃₋₅ cycloalkyl;
R³⁵ and R³⁶ are independently H, halo, methyl or ethyl, or R³⁵ and R³⁶ can be taken together with the carbon they are attached to form a C₃₋₅ cycloalkyl;
R³⁷ is H or methyl or ethyl;
m is an integer of 0 or 1;
n is an integer of 0 or 1;
p is an integer of 0 or 1 or 2; and
q is an integer of 0 or 1 or 2; and
R⁴ is an aryl or heteroaryl substituted with a first substitutent R¹¹, and optionally one or more other substituents, said one or more other substituents being independently chosen from the group consisting of halo, hydroxyl, optionally substituted C₁₋₁₀ alkyl, optionally substituted C₁₋₁₀ alkoxy, O-carboxy, and C-carboxy; wherein said first substituent R¹¹ is chosen from the group consisting of
(a) C-carboxy,
(b) O-carboxy,
(c) sulfonamido optionally substituted with a substituted or unsubstituted arylalkyl or heteroarylalkyl,
(d) one of the following groups (i)-(vii): 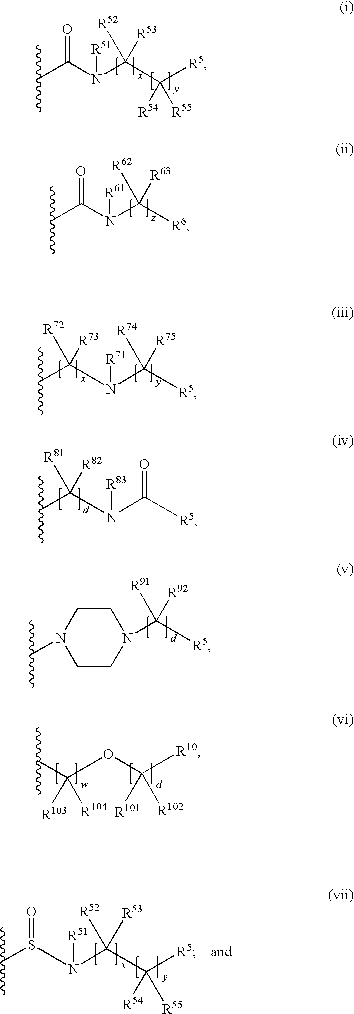
(e) R⁵, which is an aryl, arylalkyl, heteroaryl or heteroarylalkyl, each being optionally substituted with one or more substituents,
wherein in group (i), R⁵ is as defined above, R⁵¹ is H or methyl or ethyl; R⁵² and R⁵³ at each occurrence are independently chosen from the group consisting of H, F, hydroxyl, C₁₋₆ alkyl, C-carboxy, C-amido; and R⁵² and R⁵³ can be taken together with the carbon they are attached to form a cyclopryl; R⁵⁴ and R⁵⁵ at each occurrence are independently H, methyl, ethyl, F, or hydroxyl, or R⁵⁴ and R⁵⁵ can be taken together with the carbon they are attached to form a cyclopryl, or R⁵¹ and R⁵⁴ can be taken together with the nitrogen atom R⁵¹ is attached to, and the carbon atom(s) in the aliphatic chain between R⁵⁴ and the nitrogen atom, to form a 3, 4, 5 or 6-membered heterocycle; x is 0 or 1, and y is 0 or 1 or 2;
wherein in group (ii), R⁶ is chosen from the group consisting of:
(1) hydroxyl;
(2) C-carboxy;
(3) O-carboxy;
(4) optionally substituted carboxyalkyl (preferably having 4-10 carbon atoms);
(5) —N(R^{ca})C(═O)R^{cb}, N(R^{ca})C(═O)N(R^{cc})(R^{cd}), —C(═O)N(R^{cc})(R^{cd}) or —OC(═O)N(R^{cc})(R^{cd}), wherein R^{ca} is H or methyl or ethyl (preferably H), and R^{cb}, R^{cc} and R^{cd} are each independently H, OH (R^{cc} and R^{cd} are not both OH) or a chemical moiety chosen from the group of C₁₋₁₀ alkyl, C₂₋₁₀ alkenyl, C₂₋₁₀ alkynyl, C₁₋₁₀ hydroxyalkyl, C₁₋₁₀ alkoxy, C₁₋₁₀ alkylthiol, C₂₋₁₀ alkenyloxy, C₂₋₁₀ alkynyloxy, C₁₋₁₀ alkoxyalkyl, C₁₋₁₀ alkylthioalkyl, arylalkoxy, heteroarylalkoxy, carboxyalkyl, C₁₋₆ alkoxy-C₁₋₆ alkyl-, carbocycle, heterocycle, aryl and heteroaryl, or R^{cc} and R^{cd} together with the nitrogen atom to which they are both linked form a 3, 4, 5 or 6-membered optionally substituted heterocycle, wherein the chemical moiety is optionally substituted with one or more substituents;
(6) —N(R^{ab})(R^{ac}) or —SO₂N(R^{ab})(R^{ac}), wherein R^{ab} and R^{ac} are independently H, OH (R^{ab} and R^{ac} are not both OH) or optionally substituted C₁₋₆ alkyl (preferably C₁₋₃ alkyl), or R^{ab} and R^{ac} taken together with the nitrogen they are attached to form a 3, 4, 5 or 6-membered optionally substituted heterocycle;
(7) —CON(R^{ak})(R^{al}) wherein R^{ak} and R^{al} are independently H, or C₁₋₆ alkyl that is optionally substituted with 1, 2, or 3 substituents each being independently
(A) hydroxyl;
(B) halo;
(C) —N(R^{am})(R^{an}) where R^{am} and R^{an} are independently H, C₁₋₃ alkyl, hydroxyl, or C₁₋₃ hydroxylalkyl;
(D) heterocycle optionally substituted with 1, 2, or 3 substituents each being independently halo, C₁₋₆ alkyl, or C₁₋₃ haloalkyl; and
(E) aryl or heteroaryl, optionally substituted with 1, 2, or 3 substituents each being independent halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl (preferably C₁₋₃ alkyl), or R^{ao} and Rap taken together with the nitrogen they are attached to form a 3, 4, 5 or 6-membered heterocycle; and
(8) optionally substituted carbocycle;
wherein in group (ii), R⁶¹ is H or methyl or ethyl; R⁶² and R⁶³ at each occurrence are independently H, C₁₋₆ alkyl, F, hydroxyl, C-carboxy, carboxyalkyl, or C-amido, or R⁶² and R⁶³ can be taken together with the carbon they are attached to form a 3, 4, 5 or 6-membered cycloalkyl; and z is 0 to 10;
wherein in group (iii), R⁵ is as defined above, R⁷¹ is H or methyl or ethyl; R⁷², R⁷³, R⁷⁴, and R⁷⁴ at each occurrence are independently H, methyl, ethyl, or F; x and y are independently an integer of 0, 1 or 2;
wherein in group (iv), R⁵ is as defined above, R⁸³ is H or methyl or ethyl; R⁸¹ and R⁸² at each occurrence are independently H, methyl, ethyl, or F; d is an integer of 0, 1 or 2 or 3 or 4;
wherein in group (v), R⁵ is as defined above, R⁹¹ and R⁹² at each occurrence are independently H, methyl, ethyl, or F; d is an integer of 0, 1 or 2, 3 or 4;
wherein in group (vi), R¹⁰ is:
(1) heterocycle optionally substituted with 1, 2, or 3 substituents each being independently halo, C₁₋₆ alkyl, or C₁₋₃ haloalkyl;
(2) —CO₂R^{ad} where R^{ad} is H or C₁₋₃ alkyl; or
(3) optionally substituted aryl or heteroaryl;
R¹⁰¹ and R¹⁰² at each occurrence are independently H, methyl, ethyl, hydroxyl or F;
d is an integer of 0, 1 or 2, 3 or 4; and
w is an integer of 0, 1, 2, 3 or 4;
wherein in group (vii), R⁵, R⁵¹, R⁵², R⁵³, R⁵⁴, R⁵⁵, x and y are as defined above; and wherein when said first substitutent is C-carboxy, either R⁴ is heteroaryl, or R⁴ is additionally substituted with said one or more other substituents.

2. The compound of claim 1, wherein R⁵ is substituted with one or more substituents each being independently chosen from the group consisting of halo, and
(1) cyano;
(2) hydroxyl;
(3) C-carboxy, O-carboxy, or carboxyalkyl;
(4) alkyl or cycloalkyl each being optionally substituted with one or more substituents independently chosen from the group consisting of:
(A) hydroxyl; (B) halo; (C) amino;
(D) C₁₋₆ alkoxy; and
(E) arylalkoxy or heteroarylalkoxy optionally substituted with hydroxyl, halo, C-carboxy, amino, and C₁₋₆ alkoxy.
(5) —N(R^{ca})C(═O)R^{cb}, N(R^{ca})C(═O)N(R^{cc})(R^{cd}), or —OC(═O)N(R^{cc})(R^{cd}), wherein R^{ca} is H or methyl or ethyl, and R^{cb}, R^{cc} and R^{cd} are each independently H, OH (R^{cc} and R^{cd} are not both OH) or a chemical moiety chosen from the group of C₁₋₁₀ alkyl, C₂₋₁₀ alkenyl, C₂₋₁₀ alkynyl, C₁₀ hydroxyalkyl, C₁₀ alkoxy, C₁₀ alkylthiol, C₂₋₁₀alkenyloxy, C₂₋₁₀ alkynyloxy, C₁₋₁₀ alkoxyalkyl, C₁₋₁₀ alkylthioalkyl, carboxyalkyl, carbocycle, heterocycle, aryl and heteroaryl, or R^{cc} and R^{cd} together with the nitrogen atom to which they are both linked form a 3, 4, 5 or 6-membered optionally substituted heterocycle, wherein the chemical moiety is optionally substituted with one or more substituents;
(6) —N(R^{ab})(R^{ac}) or —SO₂N(R^{ab})(R^{ac}), wherein R^{ab} and R^{ac} are independently H, OH (R^{ab} and R^{ac} are not both OH) or optionally substituted C₁₋₆ alkyl, or R^{ab} and R^{ac} taken together with the nitrogen they are attached to form a 3, 4, 5 or 6-membered optionally substituted heterocycle;
(7) C₁₋₆ alkoxy optionally substituted with 1, 2 or 3 substituents each being independently chosen from the group consisting of:
(A) hydroxyl;
(B) halo;
(C) —CO₂R^{aa}, —O(C═O)R^{aa}, —C₁₋₆ alkyl-CO₂R^{aa}, or wherein R^{aa} is H or C₁₋₃ alkyl, preferably methyl or ethyl;
(D) —N(R^{ca})C(═O)R^{cb}, N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle;
(E) optionally substituted heterocycle;
(F) heteroaryl optionally substituted with 1, 2, or 3 substituents each being independent halo, hydroxyl, C₁₋₆ alkyl, C₁₋₆ alkoxy, C-carboxy, C₁₋₃ hydroxyalkyl, C₁₋₃ haloalkyl, or —N(R^{ae})(R^{af}) or —SO₂N(R^{ae})(R^{af}), wherein R^{ae} and R^{af} are independently H, OH (R^{ae} and R^{af} are not both OH), C₁₋₃ alkyl, C₁₋₆ hydroxyalkyl, or C₁₋₆ alkyl, or R^{ae} and R^{af} taken together with the nitrogen they are attached to form a 3, 4, 5 or 6-membered optionally substituted heterocycle; and
(G) —N(R^{ag})(R^{ah}) where R^{ag} and R^{ah} are independently H, hydroxyl, C₁₋₆ alkyl, optionally substituted C₁₋₆ hydroxyalkyl or aminoalkyl, or R^{ag} and R^{ah} can be taken together with the nitrogen they are attached to form a 3, 4, 5 or 6-membered optionally substituted heterocycle; and
(9) —CON(R^{ak})(R^{al}) wherein R^{ak} and R^{al} are independently H, OH (R^{ak} and R^{al} are not both OH) or a chemical moiety chosen from the group consisting of C₁₋₁₀ alkyl, C₂₋₁₀ alkenyl, C₂₋₁₀ alkynyl, C₁₋₁₀ hydroxyalkyl, C₁₋₁₀ alkoxy, C₁₋₁₀ alkylthiol, C₂₋₁₀ alkenyloxy, C₂₋₁₀ alkynyloxy, C₁₋₁₀ alkoxyalkyl, C₁₋₁₀ alkylthioalkyl, carboxyalkyl, aminoalkyl, carbocycle, heterocycle, aryl, arylakly, heteroaryl, heteroarylalkyl, or R^{ak} and R^{al} together with the nitrogen atom to which they are both linked form a 3, 4, 5 or 6-membered heterocycle, wherein the chemical moiety is optionally substituted with one or more substituents each being independently
(A) hydroxyl;
(B) halo;
(C) C₁₋₆ hydroxyalkyl;
(D) C₁₋₆ alkoxy;
(E) C₁₋₆ alkylthiol;
(F) C-carboxy, O-carboxy, or carboxyalkyl;
(G) —N(R^{am})(R^{an}) where R^{am} and R^{an} are independently H, hydroxyl, optionally substituted C₁₋₆ alkyl, optionally substituted C₁₋₆ hydroxyalkyl, or R^{am} and R^{an} together with the nitrogen atom they are attached to form a 3, 4, 5 or 6-membered optionally substituted heterocycle;
(H) heterocycle optionally substituted with one or more substituents; and
(I) aryl or heteroaryl, optionally substituted with 1, 2, or 3 substituents each being independent halo, hydroxyl, optionally substituted C₁₋₆ alkyl, C-carboxy, O-carboxy carboxyalkyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl, or R^{ao} and R^{ap} taken together with the nitrogen they are attached to form a 3, 4, 5 or 6-membered heterocycle; and
(10) R⁵ as defined herein.

3. The compound of claim 2, wherein R² is selected from the group consisting of: 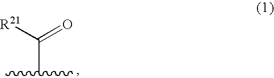
wherein R²¹ is methyl optionally substituted with halo, hydroxyl, cycloalkyl, aryl or heteroaryl; 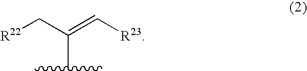
wherein R²² is (A) H, (B) C₁₋₆ alkoxy optionally substituted with 1, 2 or 3 substituents each being independently hydroxyl or C-carboxy; (C)C₁₋₆ alkylthiol optionally substituted with 1, 2 or 3 substituents each being independently hydroxyl or C-carboxy; or (D) —N(R²²¹)(R²²²) wherein R²²¹ and R²²² are independently H, C₁₋₆ alkyl, C₁₋₆ alkoxy, or R²²¹ and R²²² together with the nitrogen atom attached to them form a 3, 4, 5 or 6-membered heterocycle; and wherein R²³ is H or cyano; and 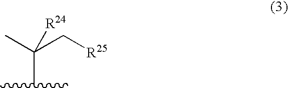
wherein R²⁴ is H or hydroxyl, R²⁵ is H, hydroxyl, or —N(R²²¹)(R²²²) wherein R²²¹ and R²²² are independently H, C₁₋₆ alkyl, C₁₋₆ alkoxy, or R²²¹ and R²²² together with the nitrogen atom attached to them form a 3, 4, 5 or 6-membered heterocycle.

4. The compound of claim 3, wherein R² is: 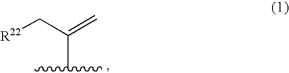
wherein R²² is (A) H, (B) C₁₋₆ alkoxy optionally substituted with 1, 2 or 3 substituents each being independently hydroxyl or C-carboxy; or (C) C₁₋₆ alkylthiol optionally substituted with 1, 2 or 3 substituents each being independently hydroxyl or C-carboxy; or 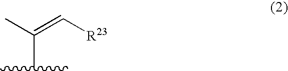
wherein R²³ is H or cyano.

5. The compound of claim 4, wherein R² is: 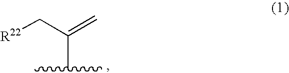
wherein R²² is H, methoxy, ethyoxy, hydroxymethoxy, hydroxyethoxy, methylthiol, ethylthiol, hydroxymethylthiol, or hydroxyethylthiol; or 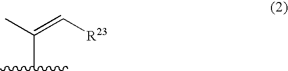
wherein R²³ is H or cyano.

6. The compound of claim 1, wherein m is 0 and n is 0.

7. The compound of claim 1, wherein m is 0 and n is 1.

8. The compound of claim 1, wherein p+q equals at least 1.

9. The compound of claim 1, wherein m is 0, n is 1, and p+q is 1 or 2.

10. The compound of claim 1, wherein R³³ and R³⁴ are independently H, halo, methyl, or R³³ and R³⁴ can be taken together with the carbon atom attached to them to form a cyclopropyl.

11. The compound of claim 1, wherein R³⁵ and R³⁶ are independently H, halo, methyl, or R³⁵ and R³⁶ can be taken together with the carbon atom attached to them to form a cyclopropyl.

12. The compound of claim 1, wherein R³ is represented by 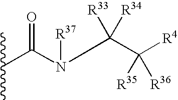
wherein R⁴, R³³, R³⁴, R³⁵, R³⁶, R³⁷ are as defined in claim 1.

13. The compound of claim 1, wherein R³ is represented by 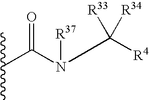
wherein R⁴, R³³, R³⁴, and R³⁷ are as defined in claim 1.

14. The compound of claim 1, R³ is according to Formula IV′ 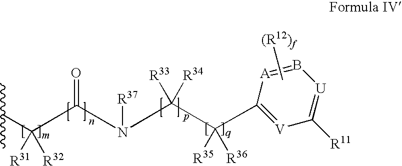
and pharmaceutically acceptably salts and stereoisomers thereof, wherein:
R³¹, R³², R³³, R³⁴, R³⁵, R³⁶, R³⁷, m, n, p, and q are as defined in claim 1;
A, B, U and V are each independently C or N;
f is an integer of 0, 1, 2, 3 or 4;
R¹² can be attached any of A, B, U and V, and at each occurance independently is H, halo, hydroxyl, optionally substituted C₁₋₁₀ alkyl, optionally substituted C₁₋₁₀ alkoxy, O-carboxy or C-carboxy; and
R¹¹ is as defined in claim 1.

15. The compound of claim 14, wherein R¹² is attached to A.

16. The compound of any one of claim 15, wherein R¹¹ is represented by

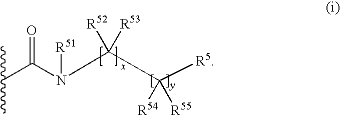

17. The compound of claim 1, wherein R³ is according to Formula IV″ 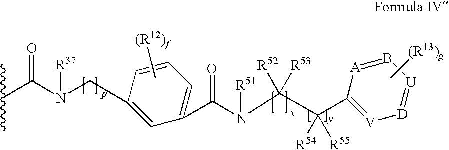
wherein
g is an integer of 0, 1, 2, 3, 4 or 5;
A, B, D, U and V are independently C or N;
R¹³ can be attached any of A, B, D, U and V, and at each occurance independently is H or
(1) halo (e.g., F, Cl, Br, I);
(2) hydroxyl; cyano;
(3) C-carboxy, O-carboxy, or carboxyalkyl;
(4) optionally substituted alkyl or cycloalkyl, for example, substituted with one or more substituents independently chosen from the group consisting of:
(A) hydroxyl; (B) halo; (C) amino;
(D) C₁₋₆ alkoxy; and
(E) arylalkoxy or heteroarylalkoxy optionally substituted with hydroxyl, halo, C-carboxy, amino, and C₁₋₆ alkoxy.
(5) —N(R^{ca})C(═O)R^{cb}, —N(R^{ca})C(═O)N(R^{cc})(R^{cd}), or —OC(═O)N(R^{cc})(R^{cd}), wherein R^{ca} is H or methyl or ethyl (preferably H), and R^{cb}, R^{cc} and R^{cd} are each independently H, OH(R^{cc} and R^{cd} are not both OH) or a chemical moiety chosen from the group of C₁₋₁₀ alkyl, C₂₋₁₀ alkenyl, C₂₋₁₀ alkynyl, C₁₋₁₀ hydroxyalkyl, C₁₋₁₀ alkoxy, C₁₋₁₀ alkylthiol, C₂₋₁₀ alkenyloxy, C₂₋₁₀ alkynyloxy, C₁₋₁₀ alkoxyalkyl, C₁₋₁₀ alkylthioalkyl, carboxyalkyl, carbocycle, heterocycle, aryl and heteroaryl, or R^{cc} and R^{cd} together with the nitrogen atom to which they are both linked form a 3, 4, 5 or 6-membered optionally substituted heterocycle, wherein the chemical moiety is optionally substituted with one or more substituents;
(6) —N(R^{ab})(R^{ac}) or —SO₂N(R^{ab})(R^{ac}), wherein R^{ab} and R^{ac} are independently H, OH (R^{ab} and R^{ac} are not both OH) or optionally substituted C₁₋₆ alkyl, or R^{ab} and R^{ac} taken together with the nitrogen they are attached to form a 3, 4, 5 or 6-membered optionally substituted heterocycle; for example, the optionally substituted C₁₋₆ alkyl may include one or more substituents each independently being hydroxyl, halo, C-carboxy, O-carboxy, amino, optionally substituted heterocycle or optionally substituted heteroaryl.
(7) optionally substituted C₁₋₆ alkoxy; for example, the C₁₋₆ alkoxy can be optionally substituted with 1, 2 or 3 substituents each being independently chosen from the group consisting of:
(A) hydroxyl;
(B) halo;
(C)—CO₂R^{aa}, —O(C═O)R^{aa}, —C₁₋₆ alkyl-CO₂R^{aa}, or wherein R^{aa} is H or C₁₋₃ alkyl, preferably methyl or ethyl;
(D) —N(R^{ca})C(═O)R^{cb}, N(R^{ca})C(═O)N(R^{cc})(R^{cd}), —C(═O)N(R^{cc})(R^{cd}) or —OC(═O)N(R^{cc})(R^{cd}), where R^{ca} is H or methyl or ethyl; R^{cb}, R^{cc} and R^{cd} are independently H, OH(R^{cc} and R^{cd} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, carbocycle, heterocycle, aryl, heteroaryl, or R^{cc} and R^{cd} together with the nitrogen atom to which they are both linked form a 3, 4, 5 or 6-membered heterocycle;
(E) optionally substituted heterocycle; for example with one or more substituents each being independently halo, hydroxyl, C₁₋₆ alkyl, C₁₋₃ haloalkyl, C-carboxyl, and sulfonyl;
(F) heteroaryl (e.g., imidazolyl) optionally substituted with 1, 2, or 3 substituents each being independent halo, hydroxyl, C₁₋₆ alkyl, C₁₋₆ alkoxy, C-carboxy, C₁₋₃ hydroxyalkyl, C₁₋₃ haloalkyl, or —N(R^{ae})(R^{af}) or —SO₂N(R^{ae})(R^{af}), wherein R^{ae} and R^{af} are independently H, OH (R^{ae} and R^{af} are not both OH), C₁₋₃ alkyl, C₁₋₆ hydroxyalkyl, or C₁₋₆ alkyl, or R^{ae} and R^{af} taken together with the nitrogen they are attached to form a 3, 4, 5 or 6-membered optionally substituted heterocycle; and
(G) —N(R^{ag})(R^{ah}) where R^{ag} and R^{ah} are independently H, hydroxyl, C₁₋₆ alkyl, optionally substituted C₁₋₆ hydroxyalkyl or aminoalkyl, or R^{ag} and R^{ah} can be taken together with the nitrogen they are attached to form a 3, 4, 5 or 6-membered optionally substituted heterocycle; and
(9) —CON(R^{ak})(R^{al}) wherein R^{ak} and R^{al} are independently H, OH (R^{ak} and R^{al} are not both OH) or a chemical moiety chosen from the group consisting of C₁₋₁₀ alkyl, C₂₋₁₀ alkenyl, C₂₋₁₀ alkynyl, C₁₋₁₀ hydroxyalkyl, C₁₋₁₀ alkoxy, C₁₋₁₀ alkylthiol, C₂₋₁₀ alkenyloxy, C₂₋₁₀ alkynyloxy, C₁₋₁₀ alkoxyalkyl, C₁₋₁₀ alkylthioalkyl, carboxyalkyl, aminoalkyl, carbocycle, heterocycle, aryl, arylakly, heteroaryl, heteroarylalkyl, or R^{ak} and R^{al} together with the nitrogen atom to which they are both linked form a 3, 4, 5 or 6-membered heterocycle, wherein the chemical moiety is optionally substituted with one or more substituents each being independently
(A) hydroxyl;
(B) halo;
(C) C₁₋₆ hydroxyalkyl;
(D) C₁₋₆ alkoxy;
(E) C₁₋₆ alkylthiol;
(F) C-carboxy, O-carboxy, or carboxyalkyl;
(G) —N(R^{am})(R^{an}) where R^{am} and R^{an} are independently H, hydroxyl, optionally substituted C₁₋₆ alkyl, optionally substituted C₁₋₆ hydroxyalkyl, or R^{am} and R^{an} together with the nitrogen atom they are attached to form a 3, 4, 5 or 6-membered optionally substituted heterocycle;
(H) heterocycle optionally substituted with one or more substituents; and
(I) aryl or heteroaryl, optionally substituted with 1, 2, or 3 substituents each being independent halo, hydroxyl, optionally substituted C₁₋₆ alkyl (e.g., methyl, C₁₋₆ haloalkyl), C-carboxy, O-carboxy carboxyalkyl, —N(R^{ao})(R^{ap}) or —SO₂N(R^{ao})(R^{ap}), wherein R^{ao} and R^{ap} are independently H, OH (R^{ao} and R^{ap} are not both OH), C₁₋₃ alkyl, C₁₋₆ hydroxyalkyl, or C₁₋₆ alkyl, or R^{ao} and R^{ap} taken together with the nitrogen they are attached to form a 3, 4, 5 or 6-membered heterocycle; or
(10) carbocycle, heterocycle, aryl, heteroaryl, arylalkyl or heteroarylalkyl optionally substituted by R¹³.

18. A pharmaceutical composition comprising:
a therapeutically effective amount of a compound of claim 1; and
a pharmaceutically acceptable carrier.

19. A method of treating HIV infection comprising administering to a patient in need of treatment a therapeutically effective amount of compound of claim 1.

20. A method of treating cancer comprising administering to a patient in need of treatment a therapeutically effective amount of compound of claim 1.
